(19) United States
(12) Patent Application Publication
(10) Pub. No.: US 2024/0398993 A1
(43) Pub. Date: Dec. 5, 2024

(54) SMALL MOLECULE-REGULATED GENE EXPRESSION SYSTEM

(71) Applicants: Glenna FOIGHT, Seattle, WA (US); TJ BRUNETTE, Seattle, WA (US); Outpace Bio, Inc., Seattle, WA (US)

(72) Inventors: Glenna FOIGHT, Seattle, WA (US); TJ BRUNETTE, Seattle, WA (US)

(21) Appl. No.: 18/261,554

(22) PCT Filed: Jan. 17, 2022
(86) PCT No.: PCT/US2022/012688

§ 371 Date: Jul. 14, 2023

Related U.S. Application Data
(60) Provisional application No. 63/164,866, filed on Mar. 23, 2021.
(60) Provisional application No. 63/143,735, filed on Jan. 29, 2021.
(60) Provisional application No. 63/143,026, filed on Jan. 28, 2021.
(60) Provisional application No. 63/137,803, filed on Jan. 15, 2021.

(51) Int. Cl. A61K 48/00; C07K 14/47; C07K 14/81; C12N 9/50; C12N 15/86

(52) U.S. Cl. CPC A61K 48/0066; C07K 14/4705; C07K 14/811; C12N 9/506; C12N 15/86; C07K 2319/50; C07K 2319/80; C12N 2740/15043; C12N 2830/002

(57) ABSTRACT

A fusion protein comprising a DNA binding domain operably-linked to a dimerization domain, wherein the DNA binding domain specifically binds to a response element.

### RELATED APPLICATIONS

This application is a 35 U.S.C. § 371 national stage entry of PCT/US2022/012688, filed Jan. 17, 2022, titled the same, which claims priority to U.S. Provisional Application Nos. 63/137,803, filed Jan. 15, 2021; 63/143,026, filed Jan. 28, 2021; 63/143,735, filed Jan. 29, 2021; and 63/164,866, filed Mar. 23, 2021. The entire contents of each of the prior applications are incorporated by reference herein.

### INCORPORATION OF THE SEQUENCE LISTING

This application contains a Sequence Listing that has been submitted ASCII format via U.S. Postal Service Express Mail and is hereby incorporated by reference in its entirety. The ACSII file, created on Mar. 9, 2024 is named “OTPC_022_04US_SeqList_ST25.txt” and is 360,410,705 bytes in size.

### FIELD OF THE DISCLOSURE

The disclosure relates to small molecule-regulated gene expression systems as well as the fields of small molecules, gene therapy, protein design, and cell signaling. The expression systems localize regulatory elements via dimerization of fusion proteins mediated by a small molecule, and thereby mediate expression of a gene of interest.

### BACKGROUND

Post-translational control systems have been designed to facilitate temporal modulation using small molecules as extrinsic inputs. Such systems are useful for a variety of in vitro, ex vivo and in vivo applications. Chemically induced dimerization (CID) is one mechanism by which a small molecule can be used to effect post translational control of expression of the gene of interest. These systems make use of a small molecule to induce dimerization of proteins and thereby localize components required for transcription. In designing such systems, it is desirable to reduce background expression of the gene of interest. The disclosure provides modified post-translational control systems with reduced background expression. The disclosure also provides a variety of other improvements including, inter alia, improvements in packaging, transduction, promoter design and vector design.

### SUMMARY

The disclosure provides a fusion protein comprising a DNA binding domain operably-linked to a dimerization domain, wherein the DNA binding domain specifically binds to a response element.

In some embodiments of the fusion proteins of the disclosure, including those in which the fusion protein comprising a DNA binding domain operably-linked to a dimerization domain, the DNA binding domain comprises one or more of a DNA sequence, an RNA sequence, and an amino acid sequence. In some embodiments, the DNA binding domain comprises an amino acid sequence. In some embodiments, the DNA binding domain comprises a sequence derived from one or more of a galactose-activated transcription factor 4 (Gal4) sequence, a zinc-finger 1 (ZF1) sequence, a zinc-finger 2 (ZF2) sequence, a zinc-finger 3 (ZF3) sequence, a zinc finger HIV2 (ZFHIV2) sequence, a zinc-finger homeodomain 1 (ZFHD1) sequence, a catalytically inactive Cas12a (dCas12a) sequence, a catalytically inactive Cas9 (dCas9) sequence, a catalytically inactive CasPhi (dCasPhi) sequence, and a TAL (transcription activator-like) effector (TALE) sequence. In some embodiments, the DNA binding domain comprises a sequence of one or more of Gal4 (SEQ ID NO: 56), ZF1 (SEQ ID NO: 57), ZF2 (SEQ ID NO: 58), ZF3 (SEQ ID NO: 59), ZFHIV2 (SEQ ID NO: 60), and ZFHD1 (SEQ ID NO: 165).

In some embodiments of the fusion proteins of the disclosure, including those in which the fusion protein comprising a DNA binding domain operably-linked to a dimerization domain, the DNA binding domain comprises one or more of a DNA sequence, an RNA sequence, and an amino acid sequence. In some embodiments, the DNA binding domain comprises an amino acid sequence. In some embodiments, the DNA binding domain comprises a sequence derived from a Cas12a sequence (SEQ ID NO: 166), wherein the DNA binding domain sequence comprises a substitution at one or more of the following positions compared to SEQ ID NO 166: 176, 192, 382, 548, 604, 607, 780, 783, 908, 951, 955, 958, 993, 1226, 1238 and 1263. In some embodiments, the DNA binding domain sequence comprises one or more of the following substitutions compared to SEQ ID NO 166: R176A, R192A, W382A, K548A, M604A, K607A, K780A, G783P, D908P, R951A, R955A, W958A, E993P, R1226A, D1238A and D1263A.

In some embodiments of the fusion proteins of the disclosure, including those in which the fusion protein comprising a DNA binding domain operably-linked to a dimerization domain, the DNA binding domain comprises one or more of a DNA sequence, an RNA sequence, and an amino acid sequence. In some embodiments, the DNA binding domain comprises an amino acid sequence. In some embodiments, the DNA binding domain comprises sequence derived from a Cas9 sequence (SEQ ID NO: 167), wherein the DNA binding domain sequence comprises a substitution at one or more of the following positions compared to SEQ ID NO 167: 10, 15, 66, 70, 74, 78, 165, 475-477, 762, 840, 854, 863, 982, 983, 986, 1125-1127, 1132, and 1333-1335. In some embodiments, the DNA binding domain sequence comprises one or more of the following substitutions compared to SEQ ID NO 167: D10A, S15A, R66A, R70A, R74A, R78A, R165A, 475-477 PWN-AAA, E762A, H840A, N854A, N863A, H982A, H983A, D986A, 1125-1127 DWD-AAA, G1132C, R1333A, R1335A, and 1333-1335 RKR-AKA. In some embodiments, the DNA binding domain sequence comprises the following substitutions compared to SEQ ID NO 167: D10A and H840A. In some embodiments, the DNA binding domain comprises sequence derived from a Cas9 sequence (SEQ ID NO: 167), wherein the DNA binding domain sequence comprises one or more of the following deletions compared to SEQ ID NO 167: 97-150, 175-307, 312-409, and 1099-1368. In some embodiments, the Cas9 sequence (SEQ ID NO: 167) is isolated or derived from Streptococcus pyogenes. In some embodiments, the Cas9 sequence (SEQ ID NO: 167) is isolated or derived from another species, with substitutions or deletions occurring in homologous locations in the Cas9 sequence.

In some embodiments of the fusion proteins of the disclosure, including those in which the fusion protein comprising a DNA binding domain operably-linked to a dimerization domain, the DNA binding domain comprises one or more of a DNA sequence, an RNA sequence, and an amino acid sequence. In some embodiments, the DNA binding domain comprises an amino acid sequence. In some embodiments, the DNA binding domain comprises sequence derived from a CasPhi sequence (SEQ ID NO: 168), and the DNA binding domain sequence comprises a substitution at one or more of the following positions compared to SEQ ID NO 168: 33, 126, 127, 130, 367, 371, 373, 394, and 606. In some embodiments, the DNA binding domain sequence comprises one or more of the following substitutions compared to SEQ ID NO 168: K33A, V126A, Q127A, N130A, V126A/Q127A/N130A, K367A, K371A, K373A, K367A/K371A/K373A, D394A, and E606Q. In some embodiments wherein the DNA binding domain comprises sequence derived from a CasPhi sequence (SEQ ID NO: 168), the DNA binding domain sequence comprises one or more of the following deletions compared to SEQ ID NO 168: 1-45.

In some embodiments of the fusion proteins of the disclosure, including those in which the fusion protein comprising a DNA binding domain operably-linked to a dimerization domain, the DNA binding domain comprises one or more of a DNA sequence, an RNA sequence, and an amino acid sequence. In some embodiments, the DNA binding domain comprises an amino acid sequence. In some embodiments, the DNA binding domain comprises a sequence derived from a TALE sequence (SEQ ID NO: 169).

In some embodiments of the fusion proteins of the disclosure, a cell comprises the response element. In some embodiments, the response element comprises an endogenous sequence. In some embodiments, the response element comprises an exogenous sequence. In some embodiments, the response element comprises at least one repeat of a sequence of the response element. In some embodiments, the response element comprises at least 2, 3, 4, 5, 6, 7, 8, 9, or 10 repeats of a sequence of the response element.

In some embodiments of the fusion proteins of the disclosure, a cell nucleus comprises the response element. In some embodiments, the response element comprises an endogenous sequence. In some embodiments, the response element comprises an exogenous sequence. In some embodiments, the response element comprises at least one repeat of a sequence of the response element. In some embodiments, the response element comprises at least 2, 3, 4, 5, 6, 7, 8, 9, or 10 repeats of a sequence of the response element.

In some embodiments of the fusion proteins of the disclosure, a chromosome comprises the response element. In some embodiments, the response element comprises an endogenous sequence. In some embodiments, the response element comprises an exogenous sequence. In some embodiments, the response element comprises at least one repeat of a sequence of the response element. In some embodiments, the response element comprises at least 2, 3, 4, 5, 6, 7, 8, 9, or 10 repeats of a sequence of the response element.

In some embodiments of the fusion proteins of the disclosure, the response element comprises one or more of 5×Gal4RE (SEQ ID NO: 84), 6×ZFIRE (SEQ ID NO: 85), 6×ZF2RE (SEQ ID NO: 86), 6×ZF3v1RE (SEQ ID NO: 87), 6×ZF3vRE (SEQ ID NO: 88), 12×ZF3veRE (SEQ ID NO: 89), and 12×ZFHIV2RE (SEQ ID NO: 90).

In some embodiments of the fusion proteins of the disclosure, (a) the DNA binding domain comprises Gal4DBD (SEQ ID NO: 56) and the response element comprises 5×Gal4RE (SEQ ID NO: 84); or (b) the DNA binding domain comprises ZF1 (SEQ ID NO: 57) and the response element comprises 6×ZF1RE (SEQ ID NO: 85); or (c) the DNA binding domain comprises ZF2 (SEQ ID NO: 58) and the response element comprises 6×ZF2RE (SEQ ID NO: 86); or (d) the DNA binding domain comprises ZF3 (SEQ ID NO: 59) and the response element comprises one or more of 6×ZF3v1RE (SEQ ID NO: 87), 6×ZF3vRE (SEQ ID NO: 88), and 12×ZF3veRE (SEQ ID NO: 89); or (e) the DNA binding domain comprises ZFHIV2 (SEQ ID NO: 60) and the response element comprises 12×ZFHIV2RE (SEQ ID NO: 90).

In some embodiments of the fusion proteins of the disclosure, the fusion protein comprises, from amino to carboxy termini, the DNA binding domain, a linker, and the dimerization domain. In some embodiments, the linker comprises one or more of a DNA sequence, an RNA sequence, an amino acid sequence, and a polymer. In some embodiments, the linker: (a) comprises a sequence of GGGGS (SEQ ID NO: 243); or (b) comprises a length of between 2 and 20 amino acids; or (c) comprises a sequence comprising glycine (G) and serine (S). In some embodiments, the linker comprises an oligomerization domain. In some embodiments, the oligomerization domain comprises the sequence of SEQ ID NO: 1, 2, 3, 4, or 5.

In some embodiments of the fusion proteins of the disclosure, the dimerization domain comprises an NS3a polypeptide. In some embodiments, the NS3a polypeptide comprises a sequence of SEQ ID NO: 6, 7, 8, 9, 66, 133, or 134. In some embodiments, the NS3a polypeptide comprises a sequence of SEQ ID NO: 65, 68-73 or 153.

In some embodiments of the fusion proteins of the disclosure, the dimerization domain comprises a DNCR polypeptide. In some embodiments, the DNCR polypeptide comprises a sequence of SEQ ID NO: 11-46. In some embodiments, the DNCR polypeptide comprises a sequence of SEQ ID NO: 55.

In some embodiments of the fusion proteins of the disclosure, the dimerization domain comprises a GNCR polypeptide. In some embodiments, the GNCR polypeptide comprises a sequence of SEQ ID NO: 47-50.

In some embodiments of the fusion proteins of the disclosure, the fusion protein further comprises a degradation domain. In some embodiments, the degradation domain comprises a sequence of SEQ ID NO: 156 or 160.

In some embodiments of the fusion proteins of the disclosure, the fusion protein further comprises a cleavable peptide. In some embodiments, the cleavable peptide comprises a P2A sequence or a T2A sequence. In some embodiments, the P2A sequence comprises the sequence of SEQ ID NO: 74. In some embodiments, the T2A sequence comprises the sequence of SEQ ID NO: 75. In some embodiments, the cleavable peptide comprises the sequence of SEQ ID NO: 135 or 136. As used throughout the disclosure, the terms “separation element” and “cleavable peptide” may be used interchangeably.

The disclosure provides a nucleic acid encoding a fusion protein of the disclosure, including those fusion proteins comprising a DNA binding domain operably-linked to a dimerization domain.

The disclosure provides a fusion protein comprising a regulation domain operably-linked to a dimerization domain, wherein the regulation domain is capable of modulating a transcriptional activity or an epigenetic activity of one or more target sequences.

In some embodiments of the fusion proteins of the disclosure, including a fusion protein comprising a regulation domain operably-linked to a dimerization domain, the regulation domain comprises one or more of a DNA sequence, an RNA sequence, and an amino acid sequence. In some embodiments, the regulation domain activates transcription. In some embodiments, the regulation domain deactivates transcription. In some embodiments, the regulation domain blocks transcription. In some embodiments, the regulation domain reconfigures chromatin comprising the one or more target sequences. In some embodiments, the regulation domain comprises a sequence derived from one or more of a Kruppel associated box (KRAB) sequence, a Methyl-CpG-binding protein 2 (MeCP2) sequence, a p65 sequence, a minimal p65 (p65mini) sequence, a p65mini-Heat shock factor protein 1 (HSF1) (p65mini-HSF1) sequence, a VP16 sequence, a VP64 sequence, a VP64-RTAmini sequence, a VP64-p65-RTA (VPR) sequence, and a minimal VPR (VPRmini) sequence. In some embodiments, the regulation domain comprises a sequence of one or more of a KRAB sequence (SEQ ID NO: 155), a MeCP2 sequence (SEQ ID NO: 170 or 171), a p65 sequence (SEQ ID NOs:172-175), a p65mini sequence (SEQ ID NO: 61), a p65mini-HSF1 sequence (SEQ ID NO: 62), a VP16 sequence (SEQ ID NO: 176), a VP64 sequence (SEQ ID NO: 177), a VP64-RTAmini sequence (SEQ ID NO: 63), and a VPRmini sequence (SEQ ID NO: 64).

In some embodiments of the fusion proteins of the disclosure, including a fusion protein comprising a regulation domain operably-linked to a dimerization domain, the fusion protein comprises, from amino to carboxy termini, the dimerization domain, a linker and the regulation domain. In some embodiments, the linker comprises one or more of a DNA sequence, an RNA sequence, an amino acid sequence, and a polymer. In some embodiments, the linker: (a) comprises a sequence of GGGGS (SEQ ID NO: 243); or (b) comprises a length of between 2 and 20 amino acids; or (c) comprises a sequence comprising glycine (G) and serine (S). In some embodiments, the linker comprises an oligomerization domain. In some embodiments, the oligomerization domain comprises the sequence of SEQ ID NO: 1, 2, 3, 4, or 5.

In some embodiments of the fusion proteins of the disclosure, including a fusion protein comprising a regulation domain operably-linked to a dimerization domain, the dimerization domain comprises an NS3a polypeptide. In some embodiments, the NS3a polypeptide comprises a sequence of SEQ ID NO: 6, 7, 8, 9, 66, 133, or 134. In some embodiments, the NS3a polypeptide comprises a sequence of SEQ ID NO: 67.

In some embodiments of the fusion proteins of the disclosure, including a fusion protein comprising a regulation domain operably-linked to a dimerization domain, the dimerization domain comprises a DNCR polypeptide. In some embodiments, the DNCR polypeptide comprises a sequence of SEQ ID NO: 11-46. In some embodiments, the DNCR polypeptide comprises a sequence of SEQ ID NO: 51-54 or 162.

In some embodiments of the fusion proteins of the disclosure, including a fusion protein comprising a regulation domain operably-linked to a dimerization domain, the dimerization domain comprises a GNCR polypeptide. In some embodiments, the GNCR polypeptide comprises a sequence of SEQ ID NO: 47-50.

In some embodiments of the fusion proteins of the disclosure, including a fusion protein comprising a regulation domain operably-linked to a dimerization domain, the fusion protein further comprises a degradation domain. In some embodiments, the degradation domain comprises a sequence of SEQ ID NO: 160.

In some embodiments of the fusion proteins of the disclosure, including a fusion protein comprising a regulation domain operably-linked to a dimerization domain, the one or more target sequences comprises a sequence isolated or derived from a sequence encoding a protein provided in Table A or any isoform thereof. In some embodiments, the one or more target sequences comprises a sequence isolated or derived from a sequence encoding a protein provided in Table A or a sequence at least 50%, 55%, 60%, 65%, 70%, 75%, 80%, 85%, 90%, 95%, 97%, 99% or a sequence having at least any percentage of identity in between.

The disclosure provides a nucleic acid encoding a fusion protein of the disclosure comprising a regulation domain operably-linked to a dimerization domain.

The disclosure provides a composition comprising: (a) a first fusion protein of the disclosure comprising a DNA binding domain operably-linked to a dimerization domain, wherein the DNA binding domain specifically binds to a response element; and (b) a second fusion protein of the disclosure comprising a regulation domain operably-linked to a dimerization domain, wherein the regulation domain is capable of modulating a transcriptional activity or an epigenetic activity of one or more target sequences.

In some embodiments of the compositions of the disclosure, including those comprising a first fusion protein and a second fusion protein, the composition further comprises a small molecule, wherein the dimerization domain of the first fusion protein and the dimerization domain of the second fusion protein are capable of forming a complex in the presence of the small molecule.

In some embodiments of the compositions of the disclosure, including those comprising a first fusion protein and a second fusion protein, the composition further comprises a target composition, wherein the target composition comprises a nucleic acid sequence comprising a promoter and one or more target sequences, wherein the promoter is capable of driving expression of the one or more target sequences. In some embodiments, the target composition comprises a nucleic acid sequence further comprising a response element capable of binding the DNA binding domain of the first fusion protein. In some embodiments, the response element comprises two or more response elements. In some embodiments, the response element comprises at least 2, 3, 4, 5, 6, 7, 8, 9, or 10 repeats of a sequence of the response element. In some embodiments, the response element comprises one or more of 5×Gal4RE (SEQ ID NO: 84), 6×ZFIRE (SEQ ID NO: 85), 6×ZF2RE (SEQ ID NO: 86), 6×ZF3v1RE (SEQ ID NO: 87), 6×ZF3vRE (SEQ ID NO: 88), 12×ZF3veRE (SEQ ID NO: 89), and 12×ZFHIV2RE (SEQ ID NO: 90).

In some embodiments of the compositions of the disclosure, including those comprising a first fusion protein and a second fusion protein, either the first fusion protein or the second fusion protein comprises a dimerization domain comprising a DNCR sequence of the disclosure.

In some embodiments of the compositions of the disclosure, including those comprising a first fusion protein and a second fusion protein, either the first fusion protein or the second fusion protein comprises a dimerization domain comprising a GNCR sequence of the disclosure.

In some embodiments of the compositions of the disclosure, including those comprising a first fusion protein and a second fusion protein, either the first fusion protein or the second fusion protein comprises a dimerization domain comprising a NS3a sequence of the disclosure.

In some embodiments of the compositions of the disclosure, including those comprising a first fusion protein and a second fusion protein, (a) the first fusion protein comprises a dimerization domain comprising an NS3a sequence and the second fusion protein comprises a dimerization domain comprising a DNCR sequence; or (b) the second fusion protein comprises a dimerization domain comprising an NS3a sequence and the first fusion protein comprises a dimerization domain comprising a DNCR sequence. In some embodiments, the small molecule comprises danoprevir.

In some embodiments of the compositions of the disclosure, including those comprising a first fusion protein and a second fusion protein, the small molecule comprises danoprevir.

In some embodiments of the compositions of the disclosure, including those comprising a first fusion protein and a second fusion protein, (a) the first fusion protein comprises a dimerization domain comprising an NS3a sequence and the second fusion protein comprises a dimerization domain comprising a GNCR sequence; or (b) the second fusion protein comprises a dimerization domain comprising an NS3a sequence and the first fusion protein comprises a dimerization domain comprising a GNCR sequence. In some embodiments, the small molecule comprises grazoprevir.

In some embodiments of the compositions of the disclosure, including those comprising a first fusion protein and a second fusion protein, the small molecule comprises grazoprevir.

In some embodiments of the compositions of the disclosure, including those comprising a first fusion protein and a second fusion protein, the one or more target sequences comprise(s) a sequence isolated or derived from a sequence encoding a gene of Table A. In some embodiments, the one or more target sequences comprises a sequence isolated or derived from a sequence encoding a protein provided in Table A or a sequence at least 50%, 55%, 60%, 65%, 70%, 75%, 80%, 85%, 90%, 95%, 97%, 99% or a sequence having at least any percentage of identity in between.

In some embodiments of the nucleic acids of the disclosure, the nucleic acid further comprises an Internal Ribosome Entry Sequence (IRES). In some embodiments, the IRES comprises the sequence of SEQ ID NO: 163.

In some embodiments of the nucleic acids of the disclosure, the nucleic acid further comprises one or more of a promoter, an enhancer, an intron, an exon, an untranslated region (UTR), and a posttranslational regulatory element (PRE). In some embodiments, the promoter comprises an inducible promoter. In some embodiments, the inducible promoter comprises a sequence isolated or derived from a YB_TATA promoter (SEQ ID NO: 77), human beta globin promoter (huBG) (SEQ ID NO: 78), minIL2 promoter (SEQ ID NO: 79), minimalCMV (minCMV) promoter (SEQ ID NO: 80), and TRE3G promoter (SEQ ID NO: 81). In some embodiments, the promoter comprises a constitutive promoter. In some embodiments, the constitutive promoter comprises a sequence isolated or derived from a MND promoter (SEQ ID NO: 82), a hPGK promoter (SEQ ID NO: 83), a CMV promoter(SEQ ID NO: 137), a CAG promoter(SEQ ID NO: 138), a SFFV promoter (SEQ ID NO: 139), an EF1alpha promoter (SEQ ID NO: 140), a UBC promoter(SEQ ID NO: 141), and a CD43 promoter (SEQ ID NO: 142).

The disclosure provides a vector comprising a nucleic acid of the disclosure. In some embodiments, the vector comprises a nucleic acid sequence of the disclosure, optionally, wherein the nucleic acid sequence encodes a fusion protein of the disclosure comprising a DNA binding domain operably-linked to a dimerization domain. In some embodiments, the vector comprises a nucleic acid sequence of the disclosure, optionally, wherein the nucleic acid sequence encodes a fusion protein of the disclosure comprising a regulation domain operably-linked to a dimerization domain, wherein the regulation domain is capable of modulating a transcriptional activity or an epigenetic activity of one or more target sequences. In some embodiments, the vector comprises (a) a nucleic acid sequence of the disclosure encoding a fusion protein of the disclosure comprising a DNA binding domain operably-linked to a dimerization domain and (b) a nucleic acid sequence of the disclosure encoding a regulation domain operably-linked to a dimerization domain, wherein the regulation domain is capable of modulating a transcriptional activity or an epigenetic activity of one or more target sequences.

In some embodiments of the vectors of the disclosure, the vector comprises an expression vector capable of driving expression of the nucleic acid in a mammalian cell. In some embodiments, the expression vector comprises a plasmid.

In some embodiments of the vectors of the disclosure, the vector comprises a delivery vector capable of introducing the nucleic acid to a mammalian cell. In some embodiments, the delivery vector comprises one or more of a plasmid, viral vector, a non-viral vector, a liposome, a micelle, a polymersome, and a nanoparticle. In some embodiments, the viral vector comprises one or more sequences isolated or derived from a viral genome. In some embodiments, the viral vector is replication-deficient.

The disclosure provides a cell comprising a fusion protein of the disclosure, a nucleic acid of the disclosure or a vector of the disclosure. In some embodiments, the cell is a mammalian cell. In some embodiments, the cell is a human cell. In some embodiments, the cell is a somatic cell. In some embodiments, the cell is a stem cell. In some embodiments, the cell is not a human embryonic stem cell. In some embodiments, the cell is an immune cell. In some embodiments, the immune cell is a Hematopoietic Stem Cell (HSC), a Myeloid Progenitor cell or a Lymphoid Progenitor cell. In some embodiments, the Myeloid Progenitor cell is a mast cell, a myeloblast, an erythrocyte or a platelet. In some embodiments, the Lymphoid Progenitor cell is a lymphocyte. In some embodiments, the lymphocyte is a Natural Killer (NK) cell, a B lymphocyte (B cell), or a T lymphocyte (T cell). In some embodiments, the B cell is a naive B cell or memory B cell. In some embodiments, the T cell is a gamma delta T cell (γδT-cell) a MAIT T-cell, a memory CD4 T-cell, a memory CD8 T-cell, a naive CD4 T-cell, a naive CD8 T-cell or a regulatory T cell (T-reg). In some embodiments, the cell is ex vivo or in vitro. In some embodiments, the cell is in vivo.

The disclosure provides a composition comprising a cell of the disclosure, a fusion protein of the disclosure, a nucleic acid of the disclosure or a vector of the disclosure.

The disclosure provides a pharmaceutical composition comprising a composition of the disclosure and a pharmaceutically-acceptable carrier.

The disclosure provides a use of a fusion protein of the disclosure, a nucleic acid of the disclosure, a vector of the disclosure, a cell of the disclosure, a composition of the disclosure, or the pharmaceutical composition of the disclosure in the manufacture of a medicament for the treatment of a disease or disorder.

A use of a fusion protein of the disclosure, a nucleic acid of the disclosure, a vector of the disclosure, a cell of the disclosure, a composition of the disclosure, or the pharmaceutical composition of the disclosure for the treatment of a disease or disorder.

In some embodiments of the uses of the disclosure, the disease or disorder comprises one or more of an autoimmune disease or disorder; an inflammatory disease or disorder; an immunodeficiency disease or disorder; an ischemic disease or disorder; a blood disease or disorder; a bone disease or disorder; a neurological disease or disorder; a cardiac disease or disorder; a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

In some embodiments of the uses of the disclosure, the disease or disorder comprises a cancer. In some embodiments of the uses of the disclosure, the cancer comprises one or more of Acute Lymphocytic Leukemia (ALL) in Adults, Acute Myeloid Leukemia (AML) in Adults, Adrenal Cancer, Anal Cancer, Basal and Squamous Cell Skin Cancer, Bile Duct Cancer, Bladder Cancer, Bone Cancer, Brain and Spinal Cord Tumors in Adults, Brain and Spinal Cord Tumors in Children, Breast Cancer, Breast Cancer in Men, Cancer in Adolescents. Cancer in Children, Cancer in Young Adults, Cancer of Unknown Primary, Cervical Cancer, Chronic Lymphocytic Leukemia (CLL), Chronic Myeloid Leukemia (CML), Chronic Myelomonocytic Leukemia (CMML), Colorectal Cancer, Endometrial Cancer, Esophagus Cancer, Ewing Family of Tumors, Eye Cancer (Ocular Melanoma), Gallbladder Cancer, Gastrointestinal Neuroendocrine (Carcinoid) Tumors, Gastrointestinal Stromal Tumor (GIST), Head and Neck Cancers, Hodgkin Lymphoma, Kaposi Sarcoma, Kidney Cancer, Laryngeal and Hypopharyngeal Cancer, Leukemia, Leukemia in Children, Liver Cancer, Lung Cancer, Lung Carcinoid Tumor, Lymphoma, Lymphoma of the Skin, Malignant Mesothelioma, Melanoma Skin Cancer, Merkel Cell Skin Cancer, Multiple Myeloma, Myelodysplastic Syndromes, Nasal Cavity and Paranasal Sinuses Cancer, Nasopharyngeal Cancer, Neuroblastoma, Non-Hodgkin Lymphoma, Non-Hodgkin Lymphoma in Children, Oral Cavity (Mouth) and Oropharyngeal (Throat) Cancer, Osteosarcoma, Ovarian Cancer, Pancreatic Cancer, Pancreatic Neuroendocrine Tumor (NET), Penile Cancer, Pituitary Tumors, Prostate Cancer, Retinoblastoma, Rhabdomyosarcoma, Salivary Gland Cancer, Skin Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia and Wilms Tumor. In some embodiments of the uses of the disclosure, the disease or disorder is cancer and the one or more target genes comprises one or more of a gene provided in Table 4.

In some embodiments of the uses of the disclosure, the disease or disorder comprises an infection or a disease or disorder caused by the infectious disease.

In some embodiments of the uses of the disclosure, the disease or disorder comprises a genetic disease or disorder.

In some embodiments, administering to a subject an effective amount of a fusion protein, nucleic acid, vector or cell, composition or pharmaceutical composition results in the severity of a sign or symptom of the disease or disorder being decreased, thereby treating the disease or disorder.

In some embodiments, administering to a subject an effective amount of a fusion protein, nucleic acid, vector or cell, composition or pharmaceutical composition results in onset or a relapse of a sign or symptom of the disease or disorder being delayed or inhibited, thereby preventing the disease or disorder.

The disease or disorder may, for example, include one or more of an autoimmune disease or disorder; an inflammatory disease or disorder; an immunodeficiency disease or disorder; an ischemic disease or disorder; a blood disease or disorder; a bone disease or disorder; a neurological disease or disorder; a cardiac disease or disorder; a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

The disease or disorder may, for example, include a cancer.

The disease or disorder may, for example, include an infection or a disease or disorder caused by the infectious disease.

The disease or disorder may, for example, include a genetic disease or disorder.

The disclosure provides a polynucleotide set comprising: (a) a first polynucleotide comprising: (i) a promoter sequence operatively linked to one or more genes of interest; or (ii) an inducible promoter sequence operatively linked to one or more genes of interest; and (b) a second polynucleotide comprising: (i) a polynucleotide encoding a first fusion protein comprising a first dimerization polypeptide linked to a DNA binding domain specific for the promoter sequence of one or more genes of interest; and (ii) a polynucleotide encoding a second fusion protein comprising a transcriptional or epigenetic regulation domain linked to a second dimerization polypeptide; and wherein the first and second dimerization polypeptides are selected so that interaction of the first and second dimerization polypeptides is mediated by the presence of a small molecule. In some embodiments, the first polynucleotide comprises a promoter sequence operatively linked to one or more genes of interest. In some embodiments, the first polynucleotide comprises an inducible promoter sequence operatively linked to one or more genes of interest. In some embodiments, the second polynucleotide is operatively linked to a polynucleotide component encoding at least one promoter sequence. In some embodiments, the second polynucleotide is operatively linked to a polynucleotide component encoding at least one constitutive promoter sequence. In some embodiments, the first or second dimerization polypeptide comprises NS3a. In some embodiments, the first or second dimerization polypeptide is selected from the group consisting of DNCR2 and GNCR1. In some embodiments, the first or second dimerization polypeptide comprises NS3a; and the other of the first or second dimerization polypeptide is selected from the group consisting of DNCR2 and GNCR1. In some embodiments, the first or second dimerization polypeptide is selected from the group consisting of: DNCR2_1 through DNCR2_34, DNCR2-3rep, GNCR1-3rep, G33, and G38.

In some embodiments of the polynucleotide sets of the disclosure, expression of the one or more genes of interest in a cell is titratable relative to administration of the small molecule to the cell. In some embodiments, the cell comprises a prokaryotic cell. In some embodiments, the cell comprises a yeast cell. In some embodiments, the cell comprises a mammalian cell. In some embodiments, the cell comprises a human cell. In some embodiments, the cell comprises a human cell in vivo. In some embodiments, the cell comprises a human cell ex vivo. In some embodiments, the small molecule mediates binding of the first and second dimerization polypeptides. In some embodiments, the small molecule disrupts binding of the first and second dimerization polypeptides. In some embodiments, the small molecule is selected from the group consisting of: danoprevir and grazoprevir and their analogs. In some embodiments, a second small molecule disrupts binding of the first and second dimerization polypeptides by out-competing the first small molecule.

In some embodiments of polynucleotide sets the disclosure, a vector comprises the first polynucleotide and the second polynucleotide.

In some embodiments of polynucleotide sets the disclosure, a first vector comprises the first polynucleotide and a second vector comprises the second polynucleotide. In some embodiments, the first vector lacks a constitutive promoter. In some embodiments, the first vector lacks a transduction marker. In some embodiments, the vector is selected from the group consisting of adenoviral vectors, lentiviral vectors, baculoviral vectors, Epstein Barr viral vectors, papovaviral vectors, vaccinia viral vectors, herpes simplex viral vectors, adeno associated virus (AAV) vectors, and transposon vectors. In some embodiments, the vector comprises a homology directed repair vector.

In some embodiments of the disclosure, a chromosome comprises the first polynucleotide or the second polynucleotide.

In some embodiments of the polynucleotide sets of the disclosure, the polynucleotide encoding a first fusion protein and the polynucleotide encoding the second fusion protein are separated by a separation element comprising a polynucleotide sequence that prevents fusion of the first fusion protein and the second fusion protein. In some embodiments, the separation element comprises a polynucleotide sequence comprising a ribosomal skipping sequence. In some embodiments, the separation element comprises a polynucleotide sequence comprising at least two ribosomal skipping sequences. In some embodiments, the separation element comprises a polynucleotide sequence comprising P2a and/or T2a. In some embodiments, the separation element comprises a polynucleotide sequence selected from the group consisting of: P2a, T2a, T2a-RFP-P2a, P2a-T2a, T2a-P2a, and IRES. In some embodiments, the separation element comprises a polynucleotide sequence comprising a second constitutive promoter.

In some embodiments of the polynucleotide sets of the disclosure, the constitutive promoter sequence is selected from the group consisting of: MND, hPGK, CMV, CAG, SFFV, EF1alpha, UBC, and CD43. In some embodiments, the constitutive promoter sequence comprises an hPGK promoter.

In some embodiments of the polynucleotide sets of the disclosure, the transcriptional activation domain is selected from the group consisting of: KRAB, MeCP2, p65, p65mini, p65mini-HSF1, VP16, VP64, VP64-RTAmini, VPR, and VPRmini.

In some embodiments of the polynucleotide sets of the disclosure, the DNA binding domain is selected from the group consisting of: dCas12a, dCas9, dCasPhi, Gal4, TALEs, ZF1, ZF2, ZF3, ZFHD1, and ZFHIV2.

In some embodiments of the polynucleotide sets of the disclosure, the inducible polynucleotide component comprises a transcription factor-specific recognition sequence comprising a transcription factor-specific response element. In some embodiments, the transcription factor response element comprises a polynucleotide selected from the group consisting of: 5×Gal4, 6×RE for ZF1, ZF2, ZF3v1, ZF3v2, ZFHIV2, 12×RE for ZF3v3, and ZFHIV2, and repeats or combinations of any of the foregoing. In some embodiments, the transcription factor response element is repeated. In some embodiments, the transcription factor response element is repeated 2, 3, 4, 5, 6, 7, 8, 9, 10, or more times.

The disclosure provides a cell comprising the polynucleotide set of the disclosure. In some embodiments, the cell is a prokaryotic cell. In some embodiments, the cell is a yeast cell. In some embodiments, the cell is a mammalian cell. In some embodiments, the cell is a human cell. In some embodiments, the cell is a human cell in vivo. In some embodiments, the cell is a human cell ex vivo. In some embodiments, the cell is a stem cell. In some embodiments, the cell is a pluripotent stem cell. In some embodiments, the cell is a multipotent stem cell. In some embodiments, the cell is a hematopoietic stem cell. In some embodiments, the cell is a mesenchymal stromal cell. In some embodiments, the cell is a mesenchymal cell. In some embodiments, the cell is an autologous cell selected for a cell therapy or is the progeny of an autologous cell selected for a cell therapy. In some embodiments, the cell is an allogeneic cell selected for a cell therapy or is the progeny of an allogeneic cell selected for a cell therapy.

The disclosure provides a method of effecting stem cell differentiation comprising modifying a stem cell using a polypeptide set of the polynucleotide set of the disclosure. In some embodiments, the cell is a cancer cell. In some embodiments, the cell is a non-cancer cell from a human subject diagnosed with cancer. In some embodiments, the cell is an immune cell. In some embodiments, the cell is selected from the group consisting of: leukocyte, lymphocyte, T cell, regulatory T cell, effector T cell, CD4+ effector T cell, CD8+ effector T cell, memory T cell, autoreactive T cell, exhausted T cell, natural killer T cell, B cell, dendritic cell, and macrophage. In some embodiments, the cell is selected from the group consisting of: cardiac cell, lung cell, muscle cell, epithelial cell, pancreatic cell, skin cell, CNS cell, neuron, myocyte, skeletal muscle cell, smooth muscle cell, liver cell, kidney cell and glial cell.

The disclosure provides a cell genetically modified to express a CAR, comprising the polynucleotide set of the disclosure. In some embodiments, the cell is a T cell, a natural killer (NK) cell, a natural killer T (NKT) cell, or an ILC cell.

The disclosure provides a producer cell line wherein cells of the cell line comprise the polynucleotide set of the disclosure.

The disclosure provides a method of producing a polypeptide product of interest from a gene of interest, the method comprising: modifying a cell line using the polynucleotide set of the disclosure to yield a producer cell line; and culturing the producer cell line to produce the product of interest. In some embodiments, the polypeptide product of interest comprises a therapeutic protein or peptide.

The disclosure provides a producer cell line wherein cells of the cell line produce the polynucleotide set of the disclosure packaged in a viral capsid. The disclosure provides a viral capsid comprising the polynucleotide set of the disclosure. The disclosure provides a cell producing the viral capsid of the disclosure. In some embodiments of the disclosure, the viral capsid is selected from capsids of an adenovirus, lentivirus, baculovirus, Epstein Barr virus, papovavirus, vaccinia virus, herpes virus, herpes simplex virus, and adeno-associated virus.

The disclosure provides a composition comprising the polynucleotide set the disclosure.

The disclosure provides a composition of the disclosure for use in treating a subject in need of a CAR therapy.

The disclosure provides a kit comprising the polynucleotide set of the disclosure.

The disclosure provides a method of making an engineered cell, the method comprising introducing the polynucleotide of any the polynucleotide set of the disclosure into a cell. In some embodiments, the polypeptide is expressed in the cell. In some embodiments, the method further comprises administering the cell in a subject in need thereof. In some embodiments, the method further comprises administering the small molecule to the subject.

The disclosure provides a method of controlling a T cell-mediated immune response in a subject in need thereof comprising administering to the subject an effective amount of the cell of the disclosure.

The disclosure provides a method of stimulating a T cell-mediated immune response to a target cell population or tissue in a subject, comprising administering to the subject an effective amount of the cell of the disclosure.

The disclosure provides a method of providing an anti-tumor immunity in a subject in need thereof, the method comprising administering to the subject an effective amount of the cell of the disclosure.

The disclosure provides a method of treating cancer in a subject in need thereof comprising administering to the subject an effective amount of the cell of the disclosure. In some embodiments, the cell is a T cell. In some embodiments, the cell is an autologous T cell. In some embodiments, the cell is allogeneic. In some embodiments, the method further comprises administering to the subject the small molecule.

The disclosure provides a gene therapy method, wherein: a first polynucleotide comprises an inducible promoter sequence operatively linked to one or more genes of interest; and the one or more genes of interest comprise a therapeutic polypeptide; the method comprising administering to a subject in need thereof a therapeutically effective amount of the polynucleotide set of the disclosure. In some embodiments, the method further comprises administering to the subject the small molecule. In some embodiments, the method further comprises adjusting dosage of the small molecule to adjust production of the therapeutic polypeptide in the subject. In some embodiments, the method further comprises monitoring production of the therapeutic polypeptide in the subject; and adjusting dosage of the small molecule to adjust production of the therapeutic polypeptide in the subject to a desired level. In some embodiments, the subject has a condition selected from the group consisting of: cancer, cystic fibrosis, heart disease, diabetes, hemophilia and AIDS.

The disclosure provides a use of the polynucleotide set of the disclosure for the manufacture of a medicament for treating cancer in a subject in need thereof.

### BRIEF DESCRIPTION OF DRAWINGS

FIG. 1 is a schematic diagram depicting an exemplary small molecule-regulated gene expression system of the disclosure in operation.

FIG. 2 is a series of schematic diagrams depicting examples of a unidirectional forward, unidirectional reverse, and bidirectional head-to-toe configurations for encoding an inducible polynucleotide component and a constitutive polynucleotide component on a single vector.

FIG. 3 is a schematic diagram depicting an exemplary small molecule-regulated gene expression system that includes a first vector that includes an inducible polynucleotide component for expression of a gene of interest and a second vector that includes a constitutive polynucleotide component for expression of a split transcription factor;

FIG. 4 is a series of schematic diagrams depicting exemplary all-in-one vectors in lentiviral backbones in unidirectional forward, unidirectional reverse, and bidirectional head-to-head orientations.

FIG. 5A is a plot showing transduction results for the three vector orientations of FIG. 4 using different volumes of 10× concentrated lentivirus in Jurkat cells.

FIG. 5B is a plot showing titration of danoprevir on Jurkat cells expressing the unidirectional forward or bidirectional vectors of FIG. 4.

FIG. 6 is a schematic diagram depicting an exemplary two-vector system with the constitutive transcription factor component and inducible promoter component on separate lentiviral vectors.

FIG. 7A is a plot showing GFP intensity in transduction positive Jurkat cells in response to increasing concentrations of danoprevir.

FIG. 7B is a plot showing median GFP intensity in primary CD4+ T cells.

FIG. 8A is a panel of histogram plots showing EGFP expressed from untransduced Jurkat cells or Jurkat cells co-transduced with the transcription factor vector TFV1 and one of the inducible promoter vectors (IPV2-IPV6) exposed to 500 nM danoprevir.

FIG. 8B is a pair of plots showing maximal EGFP mean fluorescence intensity data (gMFI) and fold induction, respectively, for induction GFP expression in response to 500 nM danoprevir in Jurkat cells co-transduced with the transcription factor vector TFV1 and one of the inducible promoter vectors (IPV2-IPV6).

FIG. 8C is a pair of plots showing EGFP expression levels in response to titration of danoprevir on the weakest minimal promoter, YB_TATA (i.e., IPV3).

FIG. 8D is a pair of plots showing EGFP expression levels in response of the strongest minimal promoters minCMV (IPV2), huBG (IPV5), TRE3G (IPV6) to danoprevir titration and EGFP levels for huBG, respectively.

FIG. 9A is a schematic diagram depicting an exemplary inducible promoter vector (IPV5) showing the constitutive promoter MND driving the expression of the transduction marker BFP and the minimal inducible promoter huBG driving expression of EGFP.

FIG. 9B is a pair of plots showing normalized GFP expression levels in Jurkat cells co-transformed with TFV1 and either IPV5 or IPV7, which utilize the MND and hPCK promoters, respectively.

FIG. 9C is a pair of plots showing EGFP expression levels in response to titration of danoprevir on the hPGK vector (i.e., IPV7) in Jurkat cells co-transduced with TFV1.

FIG. 10 is a series of histogram plots showing GFP levels in cells co-transduced with IPV1 and either TFV1, TFV2, or TFV3, respectively, and exposed to danoprevir or DMSO.

FIG. 11 is a plot showing GFP expression (gMFI) for the four zinc finger (ZF) DBD-NS3a fusion proteins and the four DNCR2-TAD fusion proteins in response to treatment with 500 nM danoprevir.

FIG. 12A is a plot showing GFP expression (gMFI) induced by DNCR2-VPRmini on inducible promoters includes 6×RE or 12×RE for ZFHIV2.

FIG. 12B is a plot showing GFP expression (gMFI) induced by DNCR2-VPRmini on inducible promoters includes 6×RE or 12×RE for ZF3.

FIG. 13A is a schematic diagram showing the crystal structure of DNCR2/danoprevir/NS3a and models of D-1, D-9, and D-20 designs.

FIG. 13B is a plot showing the median NS3a binding intensity (PE) for titration of NS3a/danoprevir binding to the four DNCR2 variants displayed on yeast.

FIG. 14A is a series of schematic diagrams showing exemplary models of GNCR1 (with G-3rep truncation indicated), G-33, and G-38.

FIG. 14B is a pair of plots depicting a titration of NS3a/grazoprevir binding the GNCR1 (left) and a titration of NS3a/grazoprevir on G-3rep, G-33, and G-38 displayed on yeast (right).

FIG. 15 is a schematic diagram depicting an exemplary modified two-vector system with transduction markers removed from the constitutive transcription factor and inducible promoter lentiviral vectors.

FIG. 16 is a panel of histogram plots showing GFP levels in Jurkat and HEK293 cells co-transduced with IPV16 and either TFV1 or TFV21.

FIG. 17 is a panel of histogram plots showing EGFP expression in HEK293 cells transduced with the normal IPV16 and TFV1 vectors or with vectors expressing elements designed to reduce EGFP output.

FIG. 18 is a panel of plots showing a comparison of EGFP background levels and titratable EGFP expression from the normal IPV16/TFV1 combination and IPV16 with the transcription factor vector TFV23 expressing ANR-SPOP.

### DETAILED DESCRIPTION

### Nucleic Acids

In some embodiments of the disclosure, the terms “Nucleic acid,” “nucleic acid molecule,” “nucleotide,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible.

In some embodiments of the disclosure, “Nucleic acid,” and in particular a DNA or RNA molecule, refers only to the primary and secondary structure of the molecule, and does not limit it to any particular tertiary forms. In some embodiments of the disclosure, “Nucleic acid,” includes double-stranded DNA found, inter alia, in linear or circular DNA molecules (e.g., restriction fragments), plasmids, supercoiled DNA and chromosomes. In discussing the structure of particular double-stranded DNA molecules, sequences are provided according to the normal convention of writing the sequence left to right in the 5′ to 3′ direction along the non-transcribed strand of DNA (i.e., the strand having a sequence homologous to the messenger RNA or mRNA). Unless otherwise indicated, all nucleic acid and nucleotide sequences are written left to right in 5′ to 3′ orientation.

Nucleotides are referred to by their commonly known one-letter symbols recommended by the IUPAC-IUB Biochemical Nomenclature Commission. Accordingly, ‘A’ represents adenine, ‘C’ represents cytosine, ‘G’ represents guanine, ‘T’ represents thymine, and ‘U’ represents uracil.

In some embodiments of the disclosure, the term “polynucleotide” refers to polymers of nucleotides of any length or type, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”) and ribonucleic acid (“RNA”). It also includes modified, for example by alkylation and/or by capping, and unmodified forms of the polynucleotide. More particularly, “polynucleotide” includes polydeoxyribonucleotides (containing 2-deoxy-D-ribose) and polyribonucleotides (containing D-ribose), including mRNA, whether spliced or unspliced, any other type of polynucleotide which is an N- or C-glycoside of a purine or pyrimidine base, and other polymers containing nucleotide backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some embodiments of the disclosure, a polynucleotide comprises a DNA sequence. In some embodiments of the disclosure, a polynucleotide comprises a DNA sequence inserted in a vector or a vector comprising a DNAsequence.

In some embodiments of the disclosure, a polynucleotide comprises an mRNA. In some embodiments, the mRNA is a synthetic mRNA or the mRNA comprises a synthetic nucleotide.

In some embodiments of the disclosure, a polynucleotide comprises at least one unnatural, non-naturally occurring or modified nucleic acid. In some embodiments, the polynucleotide comprises a plurality of unnatural, non-naturally occurring or modified nucleic acids. In some embodiments, all nucleic acids of a certain class are unnatural, non-naturally occurring or modified nucleic acids (e.g., all uridines in a polynucleotide can be replaced with an unnatural nucleobase, e.g., 5-methoxy uridine).

In some embodiments of the disclosure, “expression” refers to the transcription and/or translation of a particular nucleotide sequence driven by a promoter.

In some embodiments of the disclosure, “expression vector” refers to a plasmid, virus, or other nucleic acid designed for polypeptide expression in a cell. The vector or construct is used to introduce a gene into a host cell whereby the vector will interact with polymerases in the cell to express the protein encoded in the vector/construct. The expression vector may exist in the cell extrachromosomally or may be integrated into the chromosome. Expression vectors may include additional sequences which render the vector suitable for replication and integration in prokaryotes, eukaryotes, or preferably both (e.g., shuttle vectors). The polynucleotides of the disclosure may be provided as components of expression vectors.

In some embodiments of the disclosure, “cloning vector” refers to a plasmid, virus, or other nucleic acid designed for producing copies of a polynucleotide. Cloning vectors may contain transcription and translation initiation sequences, transcription and translation termination sequences and a polyadenylation signal. Such constructs will typically include a 5′ LTR, a tRNA binding site, a packaging signal, an origin of second-strand DNA synthesis, and a 3′ LTR or a portion thereof. The polynucleotides of the disclosure may be provided as components of cloning vectors, which may be used to produce the polynucleotides of the disclosure.

In some embodiments of the disclosure, “promoter” refers to a nucleotide sequence which indicates where transcription of a gene is initiated and in which direction transcription will continue.

In some embodiments of the disclosure, “encoding” or the like refers to the capacity of specific sequences of nucleotides in a polynucleotide (e.g. a gene, cDNA, or mRNA) to serve as templates for synthesis of other polymers and macromolecules in biological processes having either a defined sequence of nucleotides (e.g., rRNA, tRNA and mRNA) or a defined sequence of amino acids. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding an amino acid sequence,” e.g., a polynucleotide “encoding” a chimeric polypeptide, defined below of the present disclosure, includes all nucleotide sequences that are degenerate versions of each other and that encode the same amino acid sequence.

### Polypeptides

Amino acids are referred to by either their commonly known three letter symbols or by the one-letter symbols recommended by the IUPAC-IUB Biochemical Nomenclature Commission. The amino acid residues are abbreviated as follows, where the abbreviations are shown in parentheses: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid sequences are written left to right in amino to carboxy orientation.

In some embodiments of the disclosure, “Polypeptide” may refer to a sequence of amino acid subunits. In some embodiments, a “peptide” can be less than or equal to 50 amino acids long, e.g., about 5, 10, 15, 20, 25, 30, 35, 40, 45, or 50 amino acids long. “Polypeptide,” refers to proteins, polypeptides, and peptides of any length, size, structure, or function. “Polypeptide,” “peptide,” and “protein” are used interchangeably to refer to polymers of amino acids of any length.

Polypeptides of the disclosure may comprise naturally or synthetically created or modified amino acids, for example, disulfide bond formation, glycosylation, lipidation, acetylation, phosphorylation, or any other manipulation or modification, such as conjugation with a labeling component. Also included within the definition are, for example, polypeptides in which one or more amino acid residues are artificial chemical analogs of a corresponding naturally occurring amino acid (including, for example, synthetic amino acids such as homocysteine, ornithine, p-acetylphenylalanine, D-amino acids, and creatine), as well as other modifications known in the art. Polypeptides also include gene products, homologs, orthologs, paralogs, fragments and other equivalents, variants, and analogs of the foregoing.

A polypeptide may comprises a single polypeptide or can be a multi-molecular complex such as a dimer, trimer or tetramer. Polypeptides of the disclosure may comprise single-chain or multi-chain polypeptides. Most commonly disulfide linkages are found in multi-chain polypeptides.

The polypeptides of the disclosure may comprise L-amino acids+glycine, D-amino acids+glycine (which are resistant to L-amino acid-specific proteases in vivo), or a combination of D- and L-amino acids+glycine. Polypeptides described may be chemically synthesized or recombinantly expressed.

The polypeptides of the disclosure can include additional residues at the N-terminus, C-terminus, internal to the polypeptide, or a combination thereof; these additional residues are not included in determining the percent identity of the polypeptides of the disclosure relative to the reference polypeptide. Such residues may be any residues suitable for an intended use, including but not limited to tags.

In some embodiments of the disclosure, “tags” include general detectable moieties (e.g., fluorescent proteins, antibody epitope tags, etc.), therapeutic agents, purification tags (His tags, etc.), linkers, ligands suitable for purposes of purification, ligands to drive localization of the polypeptide, and peptide domains that add functionality to the polypeptides, etc.

In some embodiments of the disclosure, “chimeric polypeptide” may refer to any polypeptide comprised of a first amino acid sequence derived from a first source, bonded, covalently or non-covalently, to a second amino acid sequence derived from a second source, wherein the first and second source are not the same. In some embodiments, a first source and a second source that are not the same can include two different biological entities, or two different proteins from the same biological entity, or a biological entity and a non-biological entity. A chimeric protein can include for example, a protein derived from at least 2 different biological sources. In some embodiments, the chimeric polypeptide may include sequences from similar proteins derived from two distinct species. In some embodiments, the chimeric polypeptide may include sequences from dissimilar proteins derived from the same species. A biological source can include any non-synthetically produced nucleic acid or amino acid sequence (e.g. a genomic or cDNA sequence, a plasmid or viral vector, a native virion or a mutant or analog of any of the above). A synthetic source can include a protein or nucleic acid sequence produced chemically and not by a biological system (e.g. solid phase synthesis of amino acid sequences). A chimeric protein can also include a protein derived from at least 2 different synthetic sources or a protein derived from at least one biological source and at least one synthetic source. A chimeric protein may also comprise a first amino acid sequence derived from a first source, covalently or non-covalently linked to a nucleic acid, derived from any source or a small organic or inorganic molecule derived from any source. The chimeric protein can comprise a linker molecule between the first and second amino acid sequence or between the first amino acid sequence and the nucleic acid, or between the first amino acid sequence and the small organic or inorganic molecule.

In some embodiments of the disclosure, a “fragment” of a polypeptide, or a “truncated polypeptide” may refers to an amino acid sequence of a polypeptide that is shorter than the sequence of a reference polypeptide (which may be a naturally-occurring sequence). In comparison to the reference polypeptide, the fragment may comprise an N- and/or C-terminal deletion. In comparison to the reference polypeptide, the fragment may comprise a deletion of any part of the sequence, whether or not the deletion is contiguous. A polypeptide in which internal amino acids have been deleted with respect to the naturally occurring sequence is also considered a fragment. The various polypeptide components of the disclosure may be provided as fragments or truncated versions of a reference protein.

In some embodiments of the disclosure, a “functional fragment” may refer to a polypeptide fragment that retains a function of the polypeptide. In some embodiments, a functional fragment of a bioactive peptide (e.g., an enzyme), retains the ability to catalyze a biological action because the functional fragment comprises a catalytic domain of the enzyme. Polypeptides of the disclosure may be provided as functional fragments or truncated versions.

In some embodiments of the disclosure, “amino acid substitution” may refer to replacing an amino acid residue present in a parent or reference sequence with another amino acid residue. In some embodiments, the parent or reference sequence comprises a wildtype sequence. An amino acid can be substituted, for example, via chemical peptide synthesis or through recombinant methods known in the art. For example, substituting an amino acid residue with an alternative amino acid residue is conducted by substituting the codon encoding the first amino acid with a codon encoding the second amino acid. Polypeptides of the disclosure may be provided with one or more amino acid substitutions.

In some embodiments of the disclosure, a “conservative amino acid substitution” is one in which one amino acid residue is replaced with an amino acid residue having a chemically similar side chain. Families of amino acid residues having similar side chains have been defined in the art, including acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some embodiments, a string of amino acids can be conservatively replaced with a chemically similar string that differs in order and/or composition of side chain family members. The various polypeptide components of the disclosure may be provided with conservative amino acid substitutions.

In some embodiments of the disclosure, non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly). The various polypeptide components of the disclosure may be provided with non-conservative amino acid substitutions. The likelihood that one of the foregoing non-conservative substitutions can alter functional properties of the protein is also correlated to the position of the substitution with respect to functionally important regions of the protein: some non-conservative substitutions can accordingly have little or no effect on biological properties. The various polypeptide components of the disclosure may be provided with non-conservative amino acid substitutions that do not significantly alter the functionality of the altered components.

In some embodiments of the disclosure, “transmembrane element” or “transmembrane domain” may refer to the polypeptide element between the extracellular element and the intracellular element. A portion of the transmembrane element exists within the cell membrane. Chimeric antigen receptors (CARs) of the disclosure include transmembrane elements.

In some embodiments of the disclosure, “intracellular element” or “intracellular domain” may refer to the polypeptide element that resides on the cytoplasmic side of the eukaryotic cell's cytoplasmic membrane, and transmits a signal into the eukaryotic cell. CARs of the disclosure include intracellular elements.

In some embodiments of the disclosure, “intracellular signaling element” or “intracellular signaling domain” may refer to a portion of the intracellular element which transduces the effector function signal which directs the eukaryotic cell to perform a specialized function.

In some embodiments of the disclosure, “extracellular element” or “extracellular element” may refer to a polypeptide element that resides outside a eukaryotic cell's cytoplasmic membrane. In a CAR-expressing cell, the extracellular element comprises an antigen binding element of the CAR.

### Sequence Analyses

In some embodiments of the disclosure, “conserved” may refer to nucleotides of a polynucleotide sequence or amino acid residues of a polypeptide sequence that occur unaltered in the same position of two or more sequences being compared. Nucleotides or amino acids that are relatively conserved are those that are conserved amongst more related sequences than nucleotides or amino acids appearing elsewhere in the sequences. In some embodiments, two or more sequences are said to be “conserved” if they are at least about 30% identical, at least about 35% identical, at least about 40% identical, at least about 45% identical, at least about 50% identical, at least about 55%, at least about 60% identical, at least about 65% identical, at least about 70% identical, at least about 75% identical, at least about 80% identical, at least about 85% identical, at least about 90% identical, at least about 95% identical to one another, at least about 98% identical, or at least about 99% identical to one another. Conservation of sequence may apply to the entire length of a polynucleotide or polypeptide or may apply to a portion, region or feature thereof.

In some embodiments of the disclosure, two or more sequences may be “completely conserved” or “identical” if they are 100% identical to one another. In some embodiments, two or more sequences are said to be “highly conserved” if they are at least about 70% identical, at least about 75% identical, at least about 80% identical, at least about 85% identical, at least about 90% identical, at least about 95% identical to one another at least about 98% identical, or at least about 99% identical to one another.

In some embodiments of the disclosure, “identity” refers to the overall monomer conservation between polymeric molecules, e.g., between polypeptide molecules or polynucleotide molecules. “Identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

In certain embodiments, the percentage identity (% ID) of a first amino acid (or nucleic acid) sequence to a second amino acid (or nucleic acid) sequence is calculated as % ID=100 (Y/Z), where Y is the number of amino acid (or nucleobase) residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Calculation of the percent identity of two polypeptide sequences, for example, can be performed by aligning the two sequences for optimal comparison purposes. For example, gaps can be introduced in one or both of a first and a second polypeptide sequences for optimal alignment and non-identical sequences can be disregarded for comparison purposes. In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

Generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the European Bioinformatics Institute (EBI) at web site ebi.ac.uk/Tools/psa. It will also be appreciated that the final alignment used to calculate percent sequence identity can be curated either automatically or manually.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the EBI. Sequence alignments can be conducted using methods known in the art such as MAFFT, Clustal (ClustalW, Clustal X or Clustal Omega), MUSCLE, etc. Different regions within a single polynucleotide or polypeptide target sequence that aligns with a polynucleotide or polypeptide reference sequence can each have their own percent sequence identity. It is noted that the percent sequence identity value is rounded to the nearest tenth. For example, values from 80.11 to 80.14 are rounded down to 80.1, while values from 80.15 to 80.19 are rounded up to 80.2. It also is noted that the length value will always be an integer.

In some embodiments of the disclosure, “linked” may refer to not only a fusion of a first moiety to a second moiety at the C-terminus or the N-terminus, but also includes insertion of the whole first moiety (or the second moiety) into any two points, e.g., amino acids, in the second moiety (or the first moiety, respectively). In some embodiments, the first moiety is linked to a second moiety by a peptide bond or a linker. The first moiety can be linked to a second moiety by a phosphodiester bond or a linker. The linker can be a peptide, a polypeptide, a nucleotide, a nucleotide chain or any chemical moiety.

In some embodiments of the disclosure, “non-naturally occurring” means a polypeptide or a polynucleotide sequence that does not exist in nature. In some embodiments, the non-naturally occurring sequence does not exist in nature because the sequence is altered relative to a naturally occurring sequence. In some embodiments, the non-naturally occurring sequence does not exist in nature because it is a combination of two known, naturally-occurring, sequences (e.g., chimeric polypeptide) that do not occur together in nature. In some embodiments, a non-naturally occurring polypeptide is a chimeric polypeptide. In some embodiments, a polypeptide or a polynucleotide is not naturally occurring because the sequence contains a portion (e.g., a fragment) that cannot be found in nature, i.e., a novel sequence. Any of the polynucleotides described herein may be provided as non-naturally occurring sequences, e.g., having sequences which are altered relative to native sequences or provided as polynucleotides which are linked to other polynucleotides in a manner that does not exist in nature. Any of the polypeptides described herein may be provided as non-naturally occurring sequences, e.g., having sequences which are altered relative to native sequences or provided as polypeptides which are linked to other polypeptides in a manner that does not exist in nature.

### Antibodies

In some embodiments of the disclosure, “antibody” comprises various antibody structures, including but not limited to monoclonal antibodies, polyclonal antibodies, and antibody fragments so long as they exhibit the desired antigen-binding activity.

In some embodiments of the disclosure, “antibody fragment” may refers to a molecule other than an intact antibody that comprises a portion of an intact antibody that binds the antigen to which the intact antibody binds. Examples of antibody fragments include, but are not limited to, Fab, Fab′, F(ab′)2, and Fv fragments, scFv antibody fragments, linear antibodies, single domain antibodies such as sdAb (either V_{L} or V_{H}), camelid VHH domains, and multi-specific antibodies formed from antibody fragments. Genes of interest of the disclosure, may for example, include antibody fragments.

In some embodiments of the disclosure, “single chain antibody” (scFv) may refer to an antibody fragment that includes variable regions of heavy (VH) and light (VL) chains, which are linked by a flexible peptide linker.

In some embodiments of the disclosure, “antigen binding molecule” may refer to a molecule that specifically binds an antigenic determinant. Genes of interest of the disclosure, may for example, include antigen binding molecules.

In some embodiments of the disclosure, “antigen” may refer to a molecule that provokes an immune response.

In some embodiments of the disclosure, “Chimeric Antigen Receptor” or “CAR” refer to a fusion protein comprising antigen recognition moieties and cell-activation elements. Polynucleotides of the disclosure may include genes of interest that encode or produce CARs.

In some embodiments of the disclosure, a “CAR T cell” or a “CAR T lymphocyte” refers to a T cell capable of expressing or producing a CAR polypeptide. For example, a cell that is capable of expressing a CAR is a T cell containing nucleic acid sequences for the expression of the CAR in the cell. Cells of the disclosure may be CAR T-cells.

In some embodiments of the disclosure, a “costimulatory element” or “costimulatory signaling domain” or “costimulatory polypeptide” refers to the intracellular portion of a costimulatory polypeptide. Costimulatory signals may enhance CAR T cell expansion, function, persistence and antitumor activity. Costimulatory signals may be provided in CARs of the disclosure by incorporating intracellular signaling domains from one or more T cell costimulatory molecules, such as CD28 or 4-1BB.

In some embodiments of the disclosure, a costimulatory polypeptide comprises a sequence isolated or derived from a protein belonging to one or more of the following protein families: TNF receptor proteins, Immunoglobulin-like proteins, cytokine receptors, integrins, signaling lymphocytic activation molecules (SLAM proteins), and activating natural killer cell receptors. Examples of such costimulatory polypeptides of the disclosure include, but are not limited to, CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and MyD88.

### Therapeutic Uses

In some embodiments of the disclosure, the term “therapeutically effective” may refer to imparting a beneficial effect on the recipient, e.g., providing some alleviation, mitigation, or decrease in at least one clinical symptom in the subject. Therapeutic effects of the disclosure need not be complete or curative, as long as some benefit is provided to the subject. For example, a therapeutic regimen that incorporates the polynucleotides, gene therapy vectors or cells of the disclosure with the small molecules of the disclosure may be structured such that the regimen is therapeutically effective as a whole.

In some embodiments of the disclosure, the term “therapeutically effective amount” refers to a dose or an amount of a nucleic acid, vector, polypeptide, composition, pharmaceutical composition or cell of the disclosure sufficient to impart a therapeutically effective benefit on the recipient. For example, polynucleotides, gene therapy vectors or cells of the disclosure may be administered in a therapeutically effective amount. A subject who has been administered polynucleotides, gene therapy vectors or cells of the disclosure may subsequently be administered a therapeutically effective amount of a small molecule of the disclosure, i.e., an amount sufficient to impart a beneficial effect on the recipient given the previous administration of polynucleotides, gene therapy vectors or cells.

The specific dose level of polynucleotides, gene therapy vectors or cells of the disclosure for any particular subject may depend upon a variety of factors, for example, the disorder being treated; the stage or severity of the disorder being treated; the effectiveness of the polynucleotides, gene therapy vectors or cells; the effectiveness of the small molecule; the route of administration of the polynucleotides, gene therapy vectors, cells, or small molecule; the rate of clearance of the polynucleotides, gene therapy vectors, cells, or small molecule; the duration of treatment; the drugs used in combination or coincident with the cellular therapy or gene therapy; the age, body weight, sex, diet and general health of the subject; and like factors well known in the medical arts and sciences.

### Cellular Therapies

In some embodiments of the disclosure, the term “stem cell” may refer to an undifferentiated or partially differentiated cell that can differentiate into various types of cells and proliferate indefinitely to produce more of the same stem cell.

In some embodiments of the disclosure, the term “Pluripotent stem cell” (PSC) may refer to a cell that can maintain an undifferentiated state indefinitely and can differentiate into most, if not all cells of the body.

In some embodiments of the disclosure, the term “Induced pluripotent stem cell” (iPS or iPSC) may refer to a pluripotent stem cell that can be generated directly from a somatic cell. This includes, but is not limited to, specialized cells such as skin or blood cells derived from an adult.

In some embodiments of the disclosure, the term “multipotent” may refer to a cell that can develop into more than one cell type but is more limited than a pluripotent cell. For example, adult stem cells and cord blood stem cells may be considered as multipotent.

In some embodiments of the disclosure, the term “hematopoietic cell” may refer to a cell that arises from a hematopoietic stem cell (HSC). Hematopoietic cells of the disclosure include, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

In some embodiments of the disclosure, the term “T-lymphocyte” or “T-cell” may refer to a hematopoietic cell that normally develops in the thymus. T-lymphocytes or T-cells include, but are not limited to, natural killer T cells, regulatory T cells, helper T cells, cytotoxic T cells, memory T cells, gamma delta T cells, and mucosal invariant T cells.

In some embodiments of the disclosure, the term “mesenchyme” may refer to a type of animal tissue comprising loose cells embedded in a mesh of proteins and fluid, i.e., the extracellular matrix. Mesenchyme directly gives rise to most of the body's connective tissues including bones, cartilage, lymphatic system, and circulatory system.

In some embodiments of the disclosure, the term “mesenchymal cell” may refer to a cell that is derived from a mesenchymal tissue. In some embodiments, cells of the disclosure may be mesenchymal cells.

In some embodiments of the disclosure, the term “mesenchymal stromal cell” (MSC) may refer to a spindle shaped plastic-adherent cell isolated from bone marrow, adipose, and other tissue sources, with multipotent differentiation capacity in vitro. For example, a mesenchymal stromal cell can differentiate into osteoblasts (bone cells), chondrocytes (cartilage cells), myocytes (muscle cells), and adipocytes (fat cells which give rise to marrow adipose tissue). The term mesenchymal stromal cell is suggested in the scientific literature to replace the term “mesenchymal stem cell”. In some cases, cells of the disclosure may be mesenchymal stromal cells.

In some embodiments of the disclosure, an “autologous cell” is a cell obtained from the same individual to whom it may be administered as a therapy (the cell is autologous to the subject). Autologous cells of the disclosure include, but are not limited to, hematopoietic cells and stem cells, such as hematopoietic stem cells.

In some embodiments of the disclosure, an allogeneic cell is a cell obtained from an individual who is not the intended recipient of the cell as a therapy (the cell is allogeneic to the subject). Allogeneic cells of the disclosure may be selected from immunologically compatible donors with respect to the subject of the methods of the disclosure. Allogeneic cells of the disclosure may be modified to produce “universal” allogeneic cells, suitable for administration to any subject without unintended immunogenicity. Allogeneic cells of the disclosure include, but are not limited to, hematopoietic cells and stem cells, such as hematopoietic stem cells.

In some embodiments of the disclosure, the term “Transfect” or “transform” or “transduce” may refer to a process by which exogenous nucleic acid is transferred or introduced into a host cell. In some embodiments, a “transfected” or “transformed” or “transduced” cell is one which has been transfected, transformed or transduced with exogenous nucleic acid or progeny of the cell.

In some embodiments of the disclosure, the term “Cell therapy” may refer to the provision or delivery of cells into a recipient for therapeutic purposes.

### Small Molecule Terminology

In some embodiments of the disclosure, the term “analog” means a chemically modified form of a compound, or member of a class of compounds, which maintains the binding properties of the compound or class. In some embodiments, an analog of danoprevir includes chemically modified forms of danoprevir that retains the ability to bind DNCR2 and NS3a.

In some embodiments of the disclosure, the term “prodrug” refers to a covalently bonded carriers that release a small molecule of the disclosure in vivo when such prodrug is administered to a patient. Prodrugs of the disclosure may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs within the scope of the disclosure include compounds wherein a hydroxy, amino, or sulfhydryl group is bonded to any group that, when the prodrug of the disclosure is administered to a mammalian subject, it cleaves to form a free hydroxyl, free amino, or free sulfhydryl group, respectively. Functional groups that may be rapidly transformed, by metabolic cleavage, in vivo form a class of groups reactive with the carboxyl group of the compounds of this disclosure. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. The small molecules of the disclosure may be administered as prodrugs. The small molecules of the disclosure may be administered to a subject as a prodrugs. A therapeutically effective amount of such a prodrug of the disclosure may be administered. The prodrug may be administered contemporaneously with the administration of the polynucleotides, gene therapy vectors or cells of the disclosure or following the administration of the polynucleotides, gene therapy vectors or cells of the disclosure.

### Compositions

In some embodiments of the disclosure, “pharmaceutically acceptable” refers to compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio. For example, the small molecules, polynucleotides, polypeptides, gene therapy vectors or cells of the disclosure may be administered as part of a composition together with other pharmaceutically acceptable components, including pharmaceutically acceptable carriers.

In some embodiments of the disclosure, the term “pharmaceutically acceptable salts” refers to derivatives of the small molecules of the disclosure wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like. For example, the small molecules of the disclosure may be provided as pharmaceutically acceptable salts.

In some embodiments of the disclosure, the term “controlled release” refers to part or all of a dosage form that can release one or more active pharmaceutical agents over a prolonged period of time (i.e., over a period of more than 1 hour). The characteristic of controlled release (CR) may also be referred to as sustained release (SR), prolonged release (PR), or extended release (ER). When used in association with the dissolution profiles discussed herein, the term “controlled release” refers to that portion of a dosage form according to the disclosure that delivers active agent over a period of time greater than 1 hour. For example, the small molecules of the disclosure may be administered in a controlled release composition.

In some embodiments of the disclosure, the term “immediate release” refers to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the disclosure that delivers active agent over a period of time less than 1 hour. The small molecules of the disclosure may be administered in an immediate release composition.

In some embodiments of the disclosure, the term “excipients” refer to pharmacologically inert ingredients that are not active in the body. See, for example, Hancock, B. C., Moss, G. P., &amp; Goldfarb, D. J. (2020). Handbook of pharmaceutical excipients. London: Pharmaceutical Press, the entire disclosure of which is incorporated herein by reference. The small molecules of the disclosure may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols. For example, the small molecules, polynucleotides, gene therapy vectors or cells of the disclosure may be provided and administered in compositions that include pharmaceutically acceptable excipients.

### Definitions

In some embodiments of the disclosure, the term “subject” refers to any mammal, including without limitation, humans.

The terms “a”, “an” and “the” include their plural forms unless the context clearly dictates otherwise.

The term “and” is used interchangeably with “or” unless expressly stated otherwise.

The term “And/or” is to be taken as specific disclosure of each of the two specified features or components with or without the other. Thus, “and/or” as used in a phrase such as “A and/or B,” includes “A and B,” “A or B,” “A” (alone), and “B” (alone). Likewise, “and/or,” as used in a phrase such as “A, B, and/or C,” is intended to encompass each of the following embodiments: A, B, and C; A, B, or C; A or C; A or B; B or C; A and C; A and B; B and C; A (alone); B (alone); and C (alone).

In some embodiments of the disclosure, the term “about” is used interchangeably with the term “approximately” or “substantially”. When “about” is used with a numerical range, it modifies that range by extending the boundaries above and below the numerical values set forth. In some embodiments, the term “about” may modify a numerical value above and below the stated value by a variance of, e.g., 10 percent up or down (higher or lower).

Numeric ranges are inclusive of the numbers defining the range. Where a range of values is stated, each intervening integer value, and each fraction thereof, between the recited upper and lower limits of that range is also specifically disclosed, as is each subrange between such values. The upper and lower limits of any range can independently be included in or excluded from the range, and each range where either, neither or both limits are included is also encompassed within the disclosure. Thus, ranges are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly stated, it is to be understood that values which are about the same quantity or amount as the stated value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

Singular or plural words also include the plural and singular number, respectively. Thus, for example, where the specification describes a gene of interest, the disclosure includes polynucleotides with a single gene of interest or multiple genes of interest.

“Above,” and “below” and words of similar import refer to this application as a whole and not to any particular portions of the application.

“Set” includes sets of one or more elements or objects.

Units, prefixes, and symbols are denoted in their Systeme International de Unites (SI) accepted form.

Headings are included herein for reference and to aid in locating the various sections. These headings are not intended to limit the scope of the concepts described with respect to the headings. Such concepts may have applicability throughout the present specification.

Although the disclosure is described in some detail by way of illustration and example for purposes of clarity and understanding, it will be apparent to those skilled in the art that certain changes and modifications may be practiced. Reference to “the disclosure” or the like is intended as a reference to any of a wide variety of embodiments of, or aspects of, the disclosure, and not as limiting the disclosure to a single embodiment or aspect. As used throughout the disclosure, the terms “aspect” and “embodiment” are interchangeable. Features discussed in the context of “certain”, “some”, or “other” aspects or embodiments of the disclosure may be found in any embodiment of the disclosure, however, in these instances, the feature may be considered a preferred feature in these highlighted embodiments.

The description and examples should not be construed as limiting the scope of the disclosure to the embodiments and examples described herein, but rather as encompassing all modifications and alternatives falling within the true scope and spirit of the disclosure.

### Small Molecule-Regulated Gene Expression System

The disclosure provides a small molecule-regulated gene expression system. The system generally includes a polynucleotide set that includes a first polynucleotide and a second polynucleotide. The first and second polynucleotides may be provided as a single polynucleotide or as a set of two or more polynucleotides. The first polynucleotide generally includes a regulatory element operatively linked to a gene of interest. For example, the first polynucleotide may include a promoter sequence, or an inducible promoter sequence, operatively linked to a gene of interest. The second polynucleotide encodes components of a polypeptide dimerization system that forms a dimerization complex in the presence of a small molecule. The dimerization complex can be used to localize polypeptide components that interact with the regulatory elements to modulate expression of the gene of interest.

The second polynucleotide encodes each dimerization polypeptide as a fusion protein together with other polypeptide components. For example, each dimerization polypeptide may include a dimerization polypeptide linked to a regulatory element. In one embodiment, the second polynucleotide encodes:
(i) a first fusion protein that may include a first dimerization polypeptide linked to a DNA binding domain specific for the promoter sequence of a gene of interest; and
(ii) a second fusion protein that may include a transcriptional or epigenetic regulation domain linked to a second dimerization polypeptide.

The first and second dimerization polypeptides may be selected so that interaction of the first and second dimerization polypeptides is mediated by the presence of a small molecule. For example, the first and second dimerization polypeptides may assemble, together with the small molecule, to form a dimerization complex.

As noted, the first polynucleotide may include an inducible promoter sequence operatively linked to a gene of interest. For example, the first polynucleotide may include:
(i) a transcription factor-specific recognition sequence that includes a transcription factor-specific response element,
(ii) a minimal promoter sequence linked to the one or more response elements, and
(iii) one or more optional regulatory sequences.

The response elements, minimal promoter, and optional regulatory sequences may be configured in a vector backbone for expression of a gene of interest.

The second polynucleotide may, for example, include:
(i) a constitutive promoter sequence,
(ii) a polynucleotide encoding the first fusion protein;
(iii) a polynucleotide encoding the second fusion protein;
(iv) a separation element that prevents fusion of the first fusion protein and the second fusion protein; and
(v) one or more optional regulatory sequences.

The constitutive promoter sequence, the polynucleotides encoding the first and second fusion proteins, separation element, and optional regulatory sequence may be configured in a vector backbone for expression of the first and second fusion proteins.

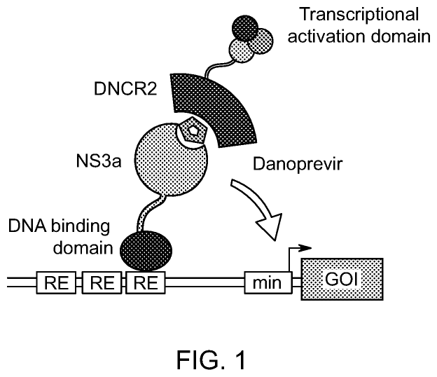

FIG. 1 illustrates a schematic diagram of an example of a small molecule-regulated gene expression system of the disclosure in operation. The figure illustrates expressed components of the system (first and second fusion proteins) binding to response elements RE and driving expression of a gene of interest (GOI) from an inducible promoter (min) from the first polynucleotide. Three response elements (RE) and a minimal promoter (min) are shown linked to the gene or interest (GOI). A first fusion protein includes an NS3a protein fused to a DNA binding domain that recognizes and binds the three REs. A second fusion protein includes a reader protein (DNCR2) fused to a transcriptional activation domain. In the presence of the small molecule drug danoprevir, the DNCR2 reader protein recognizes and binds the NS3a/danoprevir complex, thereby colocalizing the transcriptional activation domain to the minimal promoter (min) for transcription of the gene of interest. In this example, the reader protein, DNCR2, can be modularly replaced with an alternative reader that responds to a different NS3a inhibitor small molecule drug (e.g., a grazoprevir/NS3 complex reader (GNCR) protein).

### Chemically Induced Dimers

The disclosure makes use of small molecule regulated polypeptide dimers to colocalize regulatory elements and thereby modulate expression of a gene of interest. For example, the dimers may colocalize a DNA binding domain and a transcriptional regulation domain for an inducible promoter that is linked to a gene of interest. The dimers are formed when dimerization polypeptides assemble together with the small molecule to form a dimerization complex.

The dimers may be used to colocalize split transcription factors. For example, the split transcription factor may include:
(i) a first fusion protein that includes a first dimerization polypeptide linked to a DNA binding domain (DBD), and
(ii) a second fusion protein that includes a second dimerization polypeptide linked to a transcriptional or epigenetic regulation domain.

The first and second dimerization polypeptides may be selected so that interaction of the first and second dimerization polypeptides is mediated by the presence of the small molecule. In some cases, the small molecule may mediate assembly of the dimer. In other cases, the small molecule may mediate disassembly of the dimer. In still other cases, a first small molecule may mediate assembly of the dimer while a second small molecule may displace the first small molecule and thereby mediate disassembly of the dimer.

As an example, a small molecule regulated polypeptide dimer may include the hepatitis C virus protease NS3a/4a protein (hereafter referred to as NS3a) or a modification thereof as a first dimerization polypeptide and a “reader” protein as a second dimerization polypeptide. The reader protein may, for example, be selected to recognize a specific drug-bound state of the NS3a protein. NS3a proteins and NS3a reader proteins have been described in Baker et al., International Patent Publication WO2020117778, entitled “Reagents and Methods for Controlling Protein Function and Interaction,” published on Jun. 11, 2020, which is incorporated herein by reference in its entirety.

NS3a can integrate multiple drug inputs and translate the drug inputs into diverse outputs using different engineered reader proteins as dimerization partners. NS3a proteins and pleiotropic response outputs from danoprevir/NS3a complex readers, grazoprevir/NS3a complex readers, and ANR/NS3a complex readers have been been described in Foight, G. W., et al., Nature Biotechnology (2019) 37:1209-1216; Cunningham-Bryant, D. et al., Journal of the American Chemical Society (2019) 141: 3352-3355; and Kugler, J., et al., Journal of Biological Chemistry (2012) 287:39224-39232, which are incorporated herein by reference in their entireties.

In one example, the split transcription factor that forms the dimer includes:
(i) a first fusion protein that includes an NS3a polypeptide and a DNA binding domain (DBD); and
(ii) a second fusion protein that includes a reader polypeptide and a transcriptional activation domain (TAD).

Interaction between the NS3a and reader binding partners may be controlled by the presence of a small molecule drug. A reader may be selected to recognize and bind a specific NS3a/drug complex.

In some embodiments, the reader selected for the dimer is a danoprevir/NS3 complex reader (DNCR) polypeptide (or minimized/modified variants thereof) designed to recognize and bind NS3a in the presence of the small molecule drug danoprevir, thereby providing a drug-inducible transcription system. In one example the DNCR polypeptide is DNCR2. See Foight, G. W., et al., Nature Biotechnology (2019) 37:1209-1216.

In some embodiments, the reader selected for the dimer is a grazoprevir/NS3 complex reader (GNCR) polypeptide (or minimized/modified variants thereof) designed to recognize and bind NS3a in the presence of the small molecule drug grazoprevir, thereby providing a drug-inducible transcription system. In one example, the GNCR protein is GNCR1. See Foight, G. W., et al., Nature Biotechnology (2019) 37:1209-1216.

In some embodiments, the reader selected for the dimer is an apoNS3a complex reader (ANR) peptide (or minimized/modified variants thereof). ANR forms a basal complex with NS3a, which is disrupted by NS3a-targeting drugs, thereby providing a drug-disreputable transcription system. See Cunningham-Bryant, D., et al., Journal of the American Chemical Society (2019) 141:3352-3355, Kügler, J., et al., Journal of Biological Chemistry (2012) 287:39224-39232, and Foight, G. W., et al., Nature Biotechnology (2019) 37:1209-1216. Transcription Factor-Specific Recognition Sequences

In some embodiments, the first polynucleotide includes an inducible polynucleotide component that includes a transcription factor-specific recognition sequence.

In some embodiments, the transcription factor-specific recognition sequence may include a Gal4 response element.

In some embodiments, the transcription factor-specific recognition sequence may include a zinc finger (ZF) response element (e.g., a ZF1, ZF2, ZF3, and/or ZFHIV2 response element) or any modifications thereof.

In some embodiments, the transcription factor-specific recognition sequence may include a response element that is repeated 2, 3, 4, 5, 6, 7, 8, 9, 10, or more times.

In some embodiments, the transcription factor response element may include a polynucleotide selected from the group consisting of: 5×Gal4RE (SEQ ID NO: 84), 6×ZF1RE (SEQ ID NO: 85), 6×ZF2RE (SEQ ID NO: 86), 6×ZF3v1RE (SEQ ID NO: 87), 6×ZF3vRE (SEQ ID NO: 88), 12×ZF3veRE (SEQ ID NO: 89), and 12×ZFHIV2RE (SEQ ID NO: 90), and repeats or combinations thereof.

### Minimal Promoter Sequences

In some embodiments, the first polynucleotide encodes an inducible polynucleotide component that includes a minimal promoter sequence operatively linked to the gene of interest. The minimal promoter may, for example, be a minimal core promoter. In some embodiments, the minimal promoter sequence may be selected from the group consisting of: YB_TATA (SEQ ID NO: 77), human beta globin (huBG) (SEQ ID NO: 78), minIL2 (SEQ ID NO: 79), minimalCMV (minCMV) (SEQ ID NO: 80), and TRE3G (SEQ ID NO: 81).

### Regulatory Domains and Elements

In some embodiments, the first polynucleotide includes an inducible polynucleotide component that includes an optional regulatory element, such as a post-transcriptional regulatory element. For example, post-transcriptional regulatory elements may be included to increase expression of the gene of interest. Examples include bGHpA (SEQ ID NO: 91), SV40pA (SEQ ID NO: 92), and synpA (SEQ ID NO: 93).

### Constitutive Polynucleotide Component

In some embodiments, the second polynucleotide includes a constitutive polynucleotide component that may include:
(i) a first polynucleotide encoding a first fusion protein that includes a first dimerization polypeptide and a DNA binding domain (DBD),
(ii) a second polynucleotide encoding a second fusion protein that includes a second dimerization polypeptide and transcriptional activation domain (TAD),
(iii) a separation element that includes a polynucleotide sequence that prevents fusion of the first fusion protein and the second fusion protein,
(iv) a constitutive promoter sequence operatively linked to the first and second polynucleotides, and
(v) one or more optional regulatory sequences,
   wherein the first and second polynucleotides, separation element, constitutive promoter sequence, and optional regulatory elements are configured for expression of a split transcription factor.

### Dimerization Polypeptide

In various embodiments, the first or second polynucleotide may encode a dimerization polypeptide that includes NS3a (or a modification thereof) and the other of the first or second polynucleotide may encode a dimerization polypeptide selected from the group consisting of DNCR2 (or a modification thereof) and GNCR1 (or modification thereof).

In some embodiments, the first or second polynucleotide encodes a dimerization polypeptide which may include an NS3a polypeptide that includes: NS3aopt S139A (SEQ ID NO: 66), NS3a1b (SEQ ID NO: 133), NS3aH1 (SEQ ID NO: 134). The NS3a polypeptides may be designed to be either catalytically active or catalytically inactive as listed herein.

In some embodiments, the first or second polynucleotide encodes a dimerization polypeptide which may include a homo-oligomeric NS3a fusion polypeptide that includes: dimer-NS3aH1 (SEQ ID NO: 6), hexamer-NS3a (SEQ ID NO: 7), pentamer-NS3aH1 (Seq ID NO: 8), or trimer-NS3aH1 (SEQ ID NO: 9).

In some embodiments, the first or second polynucleotide encodes a dimerization polypeptide which may include a DNCR2 polypeptide that includes: DNCR2 (SEQ ID NO: 11), DNCR2_1 (SEQ ID NO: 12), DNCR2_2 (SEQ ID NO: 13), DNCR2_3 (SEQ ID NO: 14), DNCR2_4 (SEQ ID NO: 15), DNCR2_5 (SEQ ID NO: 16), DNCR2_6 (SEQ ID NO: 17), DNCR2_7 (SEQ ID NO: 18), DNCR2_8 (SEQ ID NO: 19), DNCR2_9 (SEQ ID NO: 20), DNCR2_10 (SEQ ID NO: 21), DNCR2_11 (SEQ ID NO: 22), DNCR2_12 (SEQ ID NO: 23), DNCR2_13 (SEQ ID NO: 24), DNCR2_14 (SEQ ID NO: 25), DNCR2_15 (SEQ ID NO: 26), DNCR2_16 (SEQ ID NO: 27), DNCR2_17 (SEQ ID NO: 28), DNCR2_18 (SEQ ID NO: 29), DNCR2_19 (SEQ ID NO: 30), DNCR2_20 (SEQ ID NO: 31), DNCR2_21 (SEQ ID NO: 32), DNCR2_22 (SEQ ID NO: 33), DNCR2_23 (SEQ ID NO: 34), DNCR2_24 (SEQ ID NO: 35), DNCR2_25 (SEQ ID NO: 36), DNCR2_26 (SEQ ID NO: 37), DNCR2_27 (SEQ ID NO: 38), DNCR2_28 (SEQ ID NO: 39), DNCR2_29 (SEQ ID NO: 40), DNCR2_30 (SEQ ID NO: 41), DNCR2_31 (SEQ ID NO: 42), DNCR2_32 (SEQ ID NO: 43), DNCR2_33 (SEQ ID NO: 44), DNCR2_34 (SEQ ID NO: 45), or DNCR2-3rep (SEQ ID NO: 46).

In some embodiments, the first or second polynucleotide encodes a dimerization polypeptide which may include a GNCR1 polypeptide that includes: GNCR1 (SEQ ID NO: 47), GNCR1-3rep (SEQ ID NO: 48), G33 (SEQ ID NO: 49), or G38 (SEQ ID NO: 50).

### Dimerization Peptide+DNA Binding Domain

In various embodiments, the first polynucleotide encodes a fusion protein which may include:
(i) a first dimerization polypeptide that includes: NS3aopt S139A (SEQ ID NO: 66), NS3a1b (SEQ ID NO: 133), NS3aH1 (SEQ ID NO: 134), dimer-NS3aH1 (SEQ ID NO: 6), hexamer-NS3a (SEQ ID NO: 7), pentamer-NS3aH1 (SEQ ID NO: 8), trimer-NS3aH1 (SEQ ID NO: 9), DNCR2 (SEQ ID NO: 11), DNCR2_1 (SEQ ID NO: 12), DNCR2_2 (SEQ ID NO: 13), DNCR2_3 (SEQ ID NO: 14), DNCR2_4 (SEQ ID NO: 15), DNCR2_5 (SEQ ID NO: 16), DNCR2_6 (SEQ ID NO: 17), DNCR2_7 (SEQ ID NO: 18), DNCR2_8 (SEQ ID NO: 19), DNCR2_9 (SEQ ID NO: 20), DNCR2_10 (SEQ ID NO: 21), DNCR2_11 (SEQ ID NO: 22), DNCR2_12 (SEQ ID NO: 23), DNCR2_13 (SEQ ID NO: 24), DNCR2_14 (SEQ ID NO: 25), DNCR2_15 (SEQ ID NO: 26), DNCR2_16 (SEQ ID NO: 27), DNCR2_17 (SEQ ID NO: 28), DNCR2_18 (SEQ ID NO: 29), DNCR2_19 (SEQ ID NO: 30), DNCR2_20 (SEQ ID NO: 31), DNCR2_21 (SEQ ID NO: 32), DNCR2_22 (SEQ ID NO: 33), DNCR2_23 (SEQ ID NO: 34), DNCR2_24 (SEQ ID NO: 35), DNCR2_25 (SEQ ID NO: 36), DNCR2_26 (SEQ ID NO: 37), DNCR2_27 (SEQ ID NO: 38), DNCR2_28 (SEQ ID NO: 39), DNCR2_29 (SEQ ID NO: 40), DNCR2_30 (SEQ ID NO: 41), DNCR2_31 (SEQ ID NO: 42), DNCR2_32 (SEQ ID NO: 43), DNCR2_33 (SEQ ID NO: 44), DNCR2_34 (SEQ ID NO: 45), DNCR2-3rep (SEQ ID NO: 46), GNCR1 (SEQ ID NO: 47), GNCR1-3rep (SEQ ID NO: 48), G33 (SEQ ID NO: 49), or G38 (SEQ ID NO: 50); and
(ii) a DNA binding domain (DBD) that includes: Gal4DBD (SEQ ID NO: 56), ZF1 (SEQ ID NO: 57), ZF2 (SEQ ID NO: 58), ZF3 (SEQ ID NO: 59), or ZFHIV2 (SEQ ID NO: 60).

In certain embodiments, the first polynucleotide encodes a Gal4-NS3a fusion protein that includes the Gal4 DNA binding domain and an NS3a dimerization polypeptide (SEQ ID NO: 65).

In certain embodiments, the first polynucleotide encodes an NS3a-ZF1 fusion protein that includes an NS3a dimerization polypeptide and the ZF1 DNA binding domain (SEQ ID NO: 68).

In certain embodiments, the first polynucleotide encodes an NS3a-ZF2 fusion protein that includes an NS3a dimerization polypeptide and the ZF2 DNA binding domain (SEQ ID NO: 69).

In certain embodiments, the first polynucleotide encodes an NS3a-ZF3 fusion protein that includes an NS3a dimerization polypeptide and the ZF3 DNA binding domain (SEQ ID NO: 70).

In certain embodiments, the first polynucleotide encodes an NS3a-ZFHIV2 fusion protein that includes an NS3a dimerization polypeptide and the ZFHIV2 DNA binding domain (SEQ ID NO: 71).

In certain embodiments, the first polynucleotide encodes a homodimerized NS3a-LZ-ZF3 fusion protein that includes an NS3a dimerization polypeptide and the ZF3 DNA binding domain (SEQ ID NO: 72).

In certain embodiments, the first polynucleotide encodes a homodimerized NS3a-LZ-ZFHIV2 fusion protein that includes an NS3a dimerization polypeptide and the ZFHIV2 DNA binding domain (SEQ ID NO: 73).

In certain embodiments, the first polynucleotide encodes a Gal4-DNCR2 fusion protein that includes the Gal4 DNA binding domain and a DNCR2 dimerization polypeptide (SEQ ID NO: 55).

### Dimerization Polypeptide+Transcriptional Activation Domain

In various embodiments, the second polynucleotide encodes a fusion protein which may include:
(i) a second dimerization polypeptide that includes: NS3aopt S139A (SEQ ID NO: 66), NS3a1b (SEQ ID NO: 133), NS3aH1 (SEQ ID NO: 134), dimer-NS3aH1 (SEQ ID NO: 6), hexamer-NS3a (SEQ ID NO: 7), pentamer-NS3aH1 (Seq ID NO: 8), trimer-NS3aH1 (SEQ ID NO: 9), DNCR2 (SEQ ID NO: 11), DNCR2_1 (SEQ ID NO: 12), DNCR2_2 (SEQ ID NO: 13), DNCR2_3 (SEQ ID NO: 14), DNCR2_4 (SEQ ID NO: 15), DNCR2_5 (SEQ ID NO: 16), DNCR2_6 (SEQ ID NO: 17), DNCR2_7 (SEQ ID NO: 18), DNCR2_8 (SEQ ID NO: 19), DNCR2_9 (SEQ ID NO: 20), DNCR2_10 (SEQ ID NO: 21), DNCR2_11 (SEQ ID NO: 22), DNCR2_12 (SEQ ID NO: 23), DNCR2_13 (SEQ ID NO: 24), DNCR2_14 (SEQ ID NO: 25), DNCR2_15 (SEQ ID NO: 26), DNCR2_16 (SEQ ID NO: 27), DNCR2_17 (SEQ ID NO: 28), DNCR2_18 (SEQ ID NO: 29), DNCR2_19 (SEQ ID NO: 30), DNCR2_20 (SEQ ID NO: 31), DNCR2_21 (SEQ ID NO: 32), DNCR2_22 (SEQ ID NO: 33), DNCR2_23 (SEQ ID NO: 34), DNCR2_24 (SEQ ID NO: 35), DNCR2_25 (SEQ ID NO: 36), DNCR2_26 (SEQ ID NO: 37), DNCR2_27 (SEQ ID NO: 38), DNCR2_28 (SEQ ID NO: 39), DNCR2_29 (SEQ ID NO: 40), DNCR2_30 (SEQ ID NO: 41), DNCR2_31 (SEQ ID NO: 42), DNCR2_32 (SEQ ID NO: 43), DNCR2_33 (SEQ ID NO: 44), DNCR2_34 (SEQ ID NO: 45), DNCR2-3rep (SEQ ID NO: 46), GNCR1 (SEQ ID NO: 47), GNCR1-3rep (SEQ ID NO: 48), G33 (SEQ ID NO: 49), or G38 (SEQ ID NO: 50); and
(ii) a transcriptional activation domain (TAD) that includes: p65mini (SEQ ID NO: 61), p65mini-HSF1 (SEQ ID NO: 62), VP64-RTAmini (SEQ ID NO: 63), or VPRmini (SEQ ID NO: 64).

In certain embodiments, the second polynucleotide encodes an NS3a-VPRmini fusion protein that includes an NS3a dimerization polypeptide and the VPRmini transcriptional activation domain (SEQ ID NO: 67).

In certain embodiments, the second polynucleotide encodes a DNCR2-p65mini fusion protein that includes a DNCR2 dimerization polypeptide and the p65mini transcriptional activation domain (SEQ ID NO: 51).

In certain embodiments, the second polynucleotide encodes a DNCR2-p65mini-HSF1 fusion protein that includes a DNCR2 dimerization polypeptide and the p65mini-HSF1 transcriptional activation domain (SEQ ID NO: 52).

In certain embodiments, the second polynucleotide encodes a DNCR2-VP64-RTAmini fusion protein that includes a DNCR2 dimerization polypeptide and the VP64-RTAmini transcriptional activation domain (SEQ ID NO: 53).

In certain embodiments, the second polynucleotide encodes a DNCR2-VPRmini fusion protein that includes a DNCR2 dimerization polypeptide and the VPRmini transcriptional activation domain (SEQ ID NO: 54).

### Separation Element

In various embodiments, the second polynucleotide encoding the fusion proteins may include a polynucleotide sequence encoding a separation element separating the fusion proteins.

In some embodiments, the separation element may include a ribosomal skipping sequence selected from the group consisting of: P2a (SEQ ID NO: 74) and T2a (SEQ ID NO: 75).

In some embodiments, the separation element may include a polynucleotide sequence that includes at least two ribosomal skipping sequences selected from the group consisting of T2a-RFP-P2a (SEQ ID NO: 76), P2a-T2a (SEQ ID NO: 135), and T2a-P2a (SEQ ID NO: 136).

In some embodiments, the separation element may include an internal ribosome entry site (IRES).

In some embodiments, the separation element may include a second constitutive promoter sequence.

### Constitutive Promoter Sequence

In various embodiments, the constitutive polynucleotide component may include a constitutive promoter sequence selected from the group consisting of: MND (SEQ ID NO: 82), hPGK (SEQ ID NO: 83), CMV (SEQ ID NO: 137), CAG (SEQ ID NO: 138), SFFV (SEQ ID NO: 139), EF1alpha (SEQ ID NO: 140), UBC (SEQ ID NO: 141), and CD43 (SEQ ID NO: 142).

### Regulatory Sequence

In some embodiments, the constitutive polynucleotide component may include one or more optional regulatory sequence selected from the group consisting of: bGHpA (SEQ ID NO: 91), SV40pA (SEQ ID NO: 92), and synpA (SEQ ID NO: 93).

### Target Sequences (Genes of Interest (GOI))

The polynucleotides of the disclosure encode genes of interest. The genes of interest may encode polypeptides conferring beneficial therapeutic effects. The genes of interest may, for example, encode antibodies, subcomponents of antibodies, enzymes, viral packaging polypeptides, and other polypeptides. The genes of interest may be therapeutic polypeptides. The genes of interest expressing therapeutic polypeptides may be expressed in vivo to provide a therapeutic effect to a subject, i.e., gene therapy. The genes of interest expressing therapeutic polypeptides may be expressed in vitro and purified for subsequent administration to a subject. Genes of interest may encode single polypeptides or multiple polypeptides.

### Chimeric Antigen Receptors

Genes of interest may include chimeric antigen receptors (CARs). CARs can be fused proteins including an extracellular antigen-binding/recognition element, a transmembrane element that anchors the receptor to the cell membrane and at least one intracellular element. These CAR elements are known in the art, for example as described in patent application US20140242701, entitled “Chimeric Antigen Receptors”, published on Aug. 28, 2014, which is incorporated by reference in its entirety. The CAR can be a recombinant polypeptide expressed from a polynucleotide comprising at least an extracellular antigen binding element, a transmembrane element and an intracellular signaling element comprising a functional signaling element derived from a stimulatory molecule.

The stimulatory molecule can, for example, be the zeta chain associated with the T cell receptor complex.

The cytoplasmic signaling element may, for example, include one or more functional signaling elements derived from at least one costimulatory molecule.

The costimulatory molecule can, for example, be chosen from 4-1BB (i.e., CD137), CD27 and/or CD28.

The CAR may be a chimeric fusion protein comprising an extracellular antigen recognition element, a transmembrane element and an intracellular signaling element comprising a functional signaling element derived from a stimulatory molecule.

The CAR may include a chimeric fusion protein comprising an extracellular antigen recognition element, a transmembrane element and an intracellular signaling element comprising a functional signaling element derived from a co-stimulatory molecule and a functional signaling element derived from a stimulatory molecule.

The CAR may be a chimeric fusion protein comprising an extracellular antigen recognition element, a transmembrane element and an intracellular signaling element comprising two functional signaling elements derived from one or more co-stimulatory molecule(s) and a functional signaling element derived from a stimulatory molecule.

The CAR may include a chimeric fusion protein comprising an extracellular antigen recognition element, a transmembrane element and an intracellular signaling element comprising at least two functional signaling elements derived from one or more co-stimulatory molecule(s) and a functional signaling element derived from a stimulatory molecule.

The CAR may include an optional leader sequence at the amino-terminus (N-term) of the CAR fusion protein. The CAR may further comprise a leader sequence at the N-terminus of the extracellular antigen recognition element, wherein the leader sequence is optionally cleaved from the antigen recognition element (e.g., a scFv) during cellular processing and localization of the CAR to the cellular membrane.

### Therapeutic Uses

Genes of interest may encode therapeutic polypeptides, such as polypeptides useful for treating one or more of the following conditions:
Autoimmune system disorders, such as
   Adenosine deaminase deficiency (ADA)
   AIDS (soluble CD4)
   Ankylosing spondylitis
   Autoimmune diseases (interleukin-1 receptor antagonist)
   Chronic inflammatory demyelinating polyneuropathy (CIDP)
   DADA2 vasculitis
   Diabetes mellitus Type 1 (insulin, PGC-al, GLP-1, myostatin propeptide, glucose transporter 4)
   Generalized myasthenia gravis (GMG)
   Hashimoto's thyroiditis (experimental autoimmune thyroiditis (EAT))
   Inflammatory bowel disease (IBD)
   Limb ischemia (VEGF, FGF, PGC-la, EC-SOD, HIF)
   Lupus erythematosus
   Mucosal-dominant pemphigus vulgaris
   Multiple sclerosis (β-interferon)
   Rheumatoid arthritis
   Severe combined immune deficiency (ADA-SCID)
   X-linked Severe combined immune deficiency (XSCID)
Blood cell disorders, such as
   Anemia (erythropoietin)
   Chronic granulomatous disease (CGD)
   Familial hypercholesterolemia
   Fanconi Anemia
   Glucose-6-phosphate dehydrogenase deficiency (G6PD)
   Hb S/Beta-Thalassemia (Hb S/Th)
   Hemophilia A (Factor VIII deficiency)
   Hemophilia B (Factor IX deficiency)
   Homozygous familial hypercholesterolemia (HoFH)
   Hyperlipoproteinemia type 1
   LDL receptor deficiency (LDL receptor)
   Ornithine transcarbamylase (OTC) deficiency
   Sickle cell anemia (Hb SS)>1 in 5,000;
   Sickle-cell disease (Hb S/C)
   Thalassemia (β-globin)
   Variant hemoglobinopathies (including Hb E)
   and other blood disorders
Bone disorders and fractures, such as osteodysplasia
   Alveolar bone atrophy
   Congenital and acquired maxillofacial defects
   Hip fracture
   Maxillofacial bone regeneration
   Tooth extraction, osteogenesis
Brain disorders, such as
   Osteodysplasia (also located in bone disorders)
   Schizophrenia
Cardiovascular disorders, such as
   Acute myocardial infarction
   Anemia of end stage renal disease (ESRD)
   Angina (class 2-4)
   Chronic heart failure
   Chronic kidney disease patients suffering from anemia
   Coronary artery bypass grafting
   Coronary artery disease
   Critical congenital heart defects (screened using pulse oximetry)
   Critical limb ischemia (leg)
   Critical limb ischemia with skin lesions
   Diffuse coronary artery disease
   Erectile dysfunction
   Heart disease
   Heart failure, advanced heart failure, with reduced left ventricular ejection fraction
   Heart transplants (improve survival of) (superoxide dismutase)
   Incomplete revascularisation
   Intermittent claudication
   Intimal hyperplasia (e.g., by delivering enos, inos)
   Ischemic heart disease
   Kuopio Angioplasty
   Myocardial angiogenesis
   Myocardial ischemia
   Painful diabetic peripheral neuropathy
   Peripheral artery disease
   Peripheral vascular disease
   Pulmonary hypertension
   Refractory angina pectoris
   Refractory coronary artery disease
   Restenosis
   Secondary Raynaud's Phenomenon
   Severe angina
   Severe peripheral artery occlusive disease (PAOD)
   Severe peripheral artery occlusive disease (PAOD) Fontaine stage 3
   Stable (severe) angina pectoris
   Stable exertional angina
   Stenosis prevention
   Systemic scleroderma
   Unstable angina
   Vascular access graft survival in hemodialysis patients
   Venous leg ulcer
Cancer, such as
   Cancer (endostatin, angiostatin, TRAIL, FAS-ligand, cytokines including interferons; inhibitory RNA including without limitation RNAi (such as siRNA or shRNA), antisense RNA and microRNA including inhibitory RNA against VEGF, the multiple drug resistance gene product or a cancer immunogen).
   EBV+Hodgkin's disease
   EBV+lymphoma after allo-BMT
   Follicular non-Hodgkin's lymphoma
   Graft-versus-host disease
   Leukemia
   Lymphoid malignancies
   Malignant melanoma
   Neuroblastoma
   Non-small cell lung cancer
   Oral Mucositis (associated with cancer therapy)
   Retinoblastoma
   Sarcoma
   Secondary lymphedema associated with the treatment of breast cancer
Dermatological disorders, such as
   Murine psoriasiform skin lesions
   Psoriasis
Digestive disorders, such as
   Crohn's disease
   Ulcerative colitis
Ear disorders, such as
   Inner ear disorders
   Severe hearing loss
Infectious diseases, such as
   Adenovirus infection
   COVID-19
   Cytomegalovirus (CMV) infection
   Epstein-bar virus
   Hepatitis B, C
   HIV-AIDS
   Influenza
   Malaria
   Parainfluenza virus type 3 (PIV3)
   Plasmodium falciparum infection
   Respiratory syncytial virus (RSV) infection
   Tetanus
   Tuberculosis
Inborn errors of amino acid metabolism, such as
   Argininemia
   Argininosuccinic aciduria (ASA)
   Benign hyperphenylalaninemia
   Citrullinemia (CIT)
   Citrullinemia type II
   Defects of biopterin cofactor biosynthesis
   Defects of biopterin cofactor regeneration
   Homocystinuria (HCY)
   Hypermethioninemia
   Maple syrup urine disease (MSUD)
   Phenylketonuria (PKU)
   Tyrosinemia I (TYR I)
   Tyrosinemia II
   Tyrosinemia III
Inborn errors of organic acid metabolism, such as
   2-Methyl 3-hydroxy butyric aciduria
   2-Methylbutyryl-CoA dehydrogenase deficiency
   3-Methylcrotonyl-CoA carboxylase deficiency (3MCC)
   3-Methylglutaconyl-CoA hydratase deficiency
   Adenosylcobalamin synthesis defects
   Beta-ketothiolase deficiency (BKT)
   Beta-methyl crotonyl carboxylase deficiency
   Glutaric acidemia type I (GA I)
   Glutaric acidemia type II
   HHH syndrome (Hyperammonemia, hyperornithinemia, homocitrullinuria syndrome)
   Hydroxymethylglutaryl lyase deficiency (HMG)
   Isobutyryl-CoA dehydrogenase deficiency
   Isovaleric acidemia (IVA)
   Malonic acidemia
   Methylmalonic acidemia (Cbl C,D)
   Methylmalonic aciduria, cblA and cblB forms (MMA, Cbl A,B)
   Methylmalonyl-CoA mutase deficiency (MUT)
   Multiple-CoA carboxylase deficiency (MCD)
   Propionic acidemia (PROP)
Inborn errors of fatty acid metabolism, such as
   Carnitine palmityl transferase deficiency type 1
   Carnitine palmityl transferase deficiency type 2
   Carnitine uptake defect (CUD)
   Carnitine/acylcarnitine Translocase Deficiency (Translocase)
   Dienoyl-CoA reductase deficiency
   Glutaric acidemia type II
   Long-chain acyl-CoA dehydrogenase deficiency (LCAD)
   Long-chain hydroxyacyl-CoA dehydrogenase deficiency (LCHAD)
   Medium-chain acyl-CoA dehydrogenase deficiency (MCAD)
   Medium-chain ketoacyl-CoA thiolase deficiency
   Medium/short-chain L-3-hydroxy acyl-CoA dehydrogenase deficiency
   Multiple acyl-CoA dehydrogenase deficiency (MADD)
   Short-chain acyl-CoA dehydrogenase deficiency (SCAD)
   Short-chain hydroxy Acyl-CoA dehydrogenase deficiency (SCHAD)
   Trifunctional protein deficiency (TFP)
   Very-long-chain acyl-CoA dehydrogenase deficiency (VLCAD)
   X-linked adrenoleukodystrophy
Inflammatory diseases, such as
   Degenerative joint disease of the knee
   Herpes simplex virus
   Inflammatory arthritis
   Osteoarthritis of the knee (Kellgren & Lawrence grade 2-3)
   Severe inflammatory disease of the rectum
Kidney disorders, such as kidney deficiency (erythropoietin)
   Chronic renal insufficiency
   Hemodialysis arteriovenous fistula maturation
   Kidney transplantation
Liver disorders, such as Hepatitis (a-interferon)
Lung disorders, such as
   Alpha-1 antitrypsin
   Chronic obstructive pulmonary disease (COPD)
   Lung transplant
Metabolic disorders, such as
   Hyperammonemia (ornithine transcarbamylase)
   Lysosomal storage diseases (Gaucher disease)
   Phenylketonuria (phenylalanine hydroxylase)
   Pompe disease
   Mucopolysaccharidosis type 1
Miscellaneous multisystem diseases, such as
   Biotinidase deficiency (BIOT)
   Classical galactosemia (GALT)
   Congenital adrenal hyperplasia (CAH)
   Congenital hypothyroidism (CH)
   Cystic fibrosis (CF) (cystic fibrosis transmembrane regulator protein)
   Galactokinase deficiency
   Galactose epimerase deficiency
   POEMS syndrome
Mitochondrial conditions, such as
   Ethylmalonic encephalopathy
   Leber's hereditary optic neuropathy (LHON)
Muscle disorders, such as
   Muscular dystrophies including Duchenne and Becker (e.g., dystrophin, mini-dystrophin, micro-dystrophin, insulin-like growth factor I, a sarcoglycan (e.g., α, β, γ) inhibitory RNA (e.g, RNAi, antisense RNA or microRNA) against myostatin or myostatin propeptide, laminin-alpha2, Fukutin-related protein, dominant negative myostatin, follistatin, activin type II soluble receptor, antiinflammatory polypeptides such as the Ikappa B dominant mutant, sarcospan, utrophin, mini-utrophin, inhibitory RNA [e.g, RNAi, antisense RNA or microRNA] against splice junctions in the dystrophin gene to induce exon skipping [see, e.g., WO/2003/095647], inhibitory RNA (e.g., RNAi, antisense RNA or micro RNA) against U7 snRNAs to induce exon skipping [see, e.g, WO/2006/021724], and antibodies or antibody fragments against myostatin or myostatin propeptide)
   Muscle wasting (insulin-like growth factor I, myostatin propeptide, an anti-apoptotic factor, follistatin)
   Detruser overactivity
   Overactive bladder syndrome
Nervous system disorders, such as spinal cerebral ataxias including SCA1, SCA2 and SCA3
Neurodegenerative disorders, such as
   Huntington's disease (inhibitory RNA including without limitation RNAi such as siRNA or shRNA, antisense RNA or microRNA to remove repeats)
   Parkinson's disease (glial-cell line derived neurotrophic factor [GDNF])
Neurological conditions and pathologies, such as
   Alzheimer's disease (GDF, neprilysin)
   Amyotrophic lateral sclerosis (ALS)
   Aromatic L-amino acid decarboxylase (AADC) deficiency
   Cerebral adrenoleukodystrophy (CALD)
   Charcot-marie-tooth Neuropathy type 1A
   Chronic traumatic brain injury (TBI)
   Cubital tunnel syndrome
   Developed metachromatic leukodystrophy and adrenoleukodystrophy
   Diabetic foot
   Diabetic insensate foot ulcer
   Epilepsy (galanin, neurotrophic factors)
   Intractable Pain
   Mucolopolysaccharidosis 3A (Sanfilippo Type A syndrome)
   Neuromyelitis optica spectrum disorders (NMOSD)
   Peripheral neuropathy
   Spinal muscular atrophy (SMA)
   Traumatic brain injury (TBI)
Ophthalmologic disorders and diseases, such as
   Achromatopsia
   Age-related macular degeneration (AMD)
   AMD (exudative)
   Blindness (retinitis pigmentosa) (rp)
   Choroideremia
   CNGA3-linked achromatopsia
   Congenital achromatopsia
   Diabetic macular edema
   Glaucoma
   Leber congenital amaurosis (LCA)
   Leber hereditary optic neuropathy (LHON)
   Macular degeneration
   Macular telangiectasia type 2
   Myopia
   Neovascular AMD
   Retinal disease
   Retinal dystrophy
   Retinoschisis
   Stargardt's disease
   Superficial corneal opacity/corneal scarring
   Usher syndrome (1B)
   X-linked rp (xlrp)
   All the retinal diseases listed at University of Texas RetNet website
Rheumatic conditions, such as
   Arthritis (anti-inflammatory factors such as IRAP and TNFa soluble receptor)
   Other joint disorders (insulin-like growth factors)
   Rheumatoid arthritis
   Degenerative arthritis
   Osteoarthritis
Other disorders and treatments, such as
   Allogenic stem cell transplantation
   Flexor tendon injury
   Peanut allergy
   Wound healing

### Vectors and Vector Configurations

The polynucleotides of the disclosure may be provided as part of a vector. Examples of suitable vectors include expression vectors, viral vectors, and plasmid vectors. Expression vectors can include plasmids, phagemids, viruses, and derivatives thereof. The type of vector used by some embodiments of the disclosure will depend on the cell type transformed. The ability to select suitable vectors according to the cell type transformed is well within the capabilities of the ordinary skilled artisan.

In some embodiments, the viral vectors may include polynucleotides encoding gene editing polypeptides, such as polypeptides useful for implementation of gene editing techniques. Examples of such gene editing techniques include RNA/DNA guided endonucleases (e.g., CRISPR (clustered regularly interspaced short palindromic repeats)), TALEN (transcription activator-like effector nucleases), ZFN (zinc finger nucleases), recombinase, meganucleases, or viral integration.

In some embodiments, the polynucleotides of the disclosure may be provided as part of a homology directed repair (HDR) vector. A homology directed repair mechanism may be used to integrate a polynucleotide set into a chromosome. Examples of mechanisms that may be used to integrate a polynucleotide set into a chromosome include sequence-specific nucleases such as transposase, CRISPR/Cas9, ZF nucleases, TALE nucleases, recombinases, and other homologous recombination targeting vectors known in the art.

Vector components generally include, but are not limited to, one or more of the following: a signal sequence, an origin of replication, one or more marker genes, an enhancer element, a promoter, and a transcription termination sequence. A vector for use in a eukaryotic host cell may also encode a signal sequence or other polypeptide having a specific cleavage site at the N-terminus of the mature protein or polypeptide of interest. The signal sequence selected preferably is one that is recognized and processed (i.e., cleaved by a signal peptidase) by the host cell. In mammalian cell expression, mammalian signal sequences as well as viral secretory leaders may be used. Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. One useful transcription termination component is the bovine growth hormone polyadenylation region.

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

The polynucleotides of the disclosure may in some cases be provided as part of a single vector. The polynucleotides of the disclosure may be provided as part of a set of at least two vectors; a first vector including the first polynucleotide and a second vector comprising the second polynucleotide. In some cases, inducible and constitutive parts of the system are provided on separate vectors, i.e., a first vector comprising the inducible polynucleotide component; and a second vector comprising the constitutive polynucleotide component.

Examples of vectors suitable for use with the polynucleotides of the disclosure include adenoviral vectors, lentiviral vectors, baculoviral vectors, Epstein Barr viral vectors, papovaviral vectors, vaccinia viral vectors, herpes simplex viral vectors, adeno associated virus (AAV) vectors, and transposon vectors. The polynucleotides of the disclosure may be provided as part of a homology directed repair vector.

The disclosure provides a polynucleotide set that includes the following as part of one or more vectors:
(i) a first polynucleotide that includes a promoter sequence operatively linked to a gene of interest; and
(ii) a second polynucleotide that includes a polynucleotide encoding a first fusion protein that includes a first dimerization polypeptide linked to a DNA binding domain specific for the promoter sequence of the gene of interest and a polynucleotide encoding a second fusion protein that includes a transcriptional or epigenetic regulation domain linked to a second dimerization polypeptide; wherein interaction of the first and second dimerization polypeptides is mediated by the presence of a small molecule.

The disclosure provides a polynucleotide set that includes the following as part of one or more vectors:
(i) an inducible polynucleotide component encoding an inducible promoter sequence operatively linked to a gene of interest, and
(ii) a constitutive polynucleotide component encoding at least one constitutive promoter sequence operatively linked to a polynucleotide encoding a split transcription factor, wherein the split transcription factor may include (a) a first fusion protein that includes an NS3a polypeptide and a DNA binding domain (DBD) and (b) a second fusion protein that includes a reader polypeptide and a transcriptional activation domain (TAD), wherein interaction between the NS3a polypeptide and reader polypeptide is controlled by the presence of a small molecule.

The disclosure provides a polynucleotide set that includes the following as part of one or more vectors:
(i) an inducible polynucleotide component encoding an inducible promoter sequence operatively linked to a gene of interest; and
(ii) a constitutive polynucleotide component encoding at least one constitutive promoter sequence operatively linked to a polynucleotide encoding a split transcription factor, wherein the split transcription factor may include (a) a first fusion protein that includes an NS3a polypeptide and transcriptional activation domain (TAD) and (b) a second fusion protein that includes a reader polypeptide and a DNA binding domain (DBD), wherein interaction between the NS3a polypeptide and reader polypeptide is controlled by the presence of a small molecule.

In various embodiments, the inducible polynucleotide component may include a polynucleotide that includes:
(i) a transcription factor-specific recognition sequence that includes a transcription factor-specific response element,
(ii) a minimal promoter sequence operatively linked to a gene of interest, and
(iii) one or more optional regulatory sequences, wherein the response element, minimal promoter, and optional regulatory sequence are configured for expression of the gene of interest.

### Single Vector Configuration

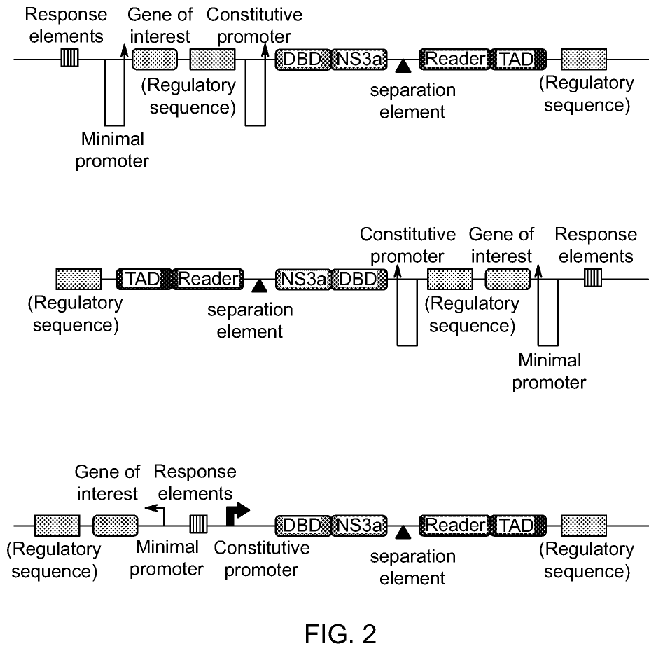

FIG. 2 illustrates a schematic diagram of examples of a unidirectional forward configuration 200, a unidirectional reverse configuration 210, and a bidirectional head-to-toe configuration 215 for encoding an inducible polynucleotide component and a constitutive polynucleotide component on a single vector. Each vector configuration 200, 210, and 215 is an example of a small molecule-regulated gene expression system consisting of a constitutive polynucleotide component configured for expressing a split transcription factor and an inducible polynucleotide component that is bound by that transcription factor to regulate the expression of a gene of interest. The encoded split transcription factor may include two polypeptide chains: (1) a DNA binding domain (DBD) fused to a first dimerization polypeptide, NS3a, and (2) a transcriptional activation domain (TAD) fused to second dimerization polypeptide, designated as “Reader.” In one example, the reader polypeptide is a DNCR2 polypeptide. The first and second dimerization polypeptides are selected so that interaction of the first and second dimerization polypeptides is mediated by the presence of a small molecule. A separation element includes a polynucleotide sequence that prevents fusion of the two polypeptide chains is positioned between the sequences encoding the split transcription factor. The constitutive promoter component may also include optional regulatory sequences such as a polyA sequence. The inducible promoter component consists of a minimal promoter with one or more 5′ response element repeats (RE) that are recognized and bound by the DBD. The inducible promoter component may also include optional regulatory sequences such as a polyA sequence.

### Two-Vector Configuration

In some embodiments, the polynucleotide set that includes the inducible and constitutive polynucleotide components is integrated on two vectors, wherein: (i) a first vector may include the inducible polynucleotide component, and (ii) a second vector may include the constitutive polynucleotide component. The vector that includes the inducible polynucleotide component may be referred to as an “inducible promoter vector” (IPV). The vector that includes the constitutive polynucleotide components may be referred to as a “transcription factor vector” (TFV).

In some embodiments, the first vector that includes the inducible polynucleotide component lacks a constitutive promoter and/or a transduction marker.

In some embodiments, the first vector that includes the inducible polynucleotide component further includes a constitutive promoter and/or a transduction marker.

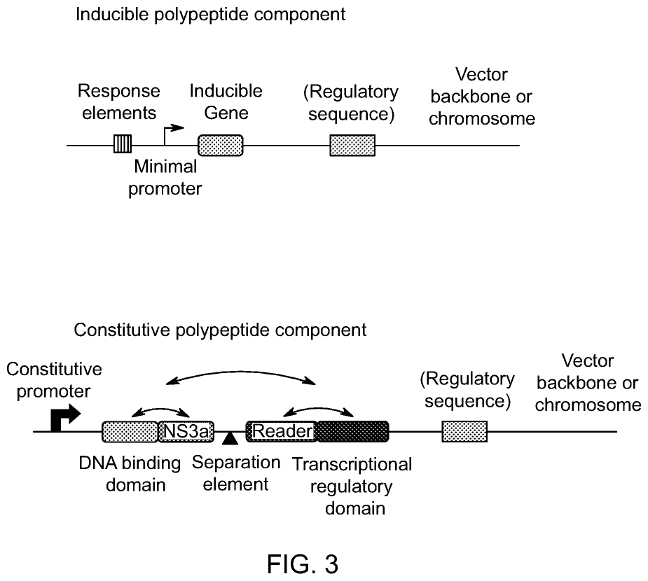

FIG. 3 illustrates a schematic diagram of an example of a small molecule-regulated gene expression system that includes a first vector that includes an inducible polynucleotide component for expression of a gene of interest and a second vector that includes a constitutive polynucleotide component for expression of a split transcription factor. On a first vector backbone, the inducible polypeptide component includes one or more response elements (e.g., 5 response elements) and a minimal promoter sequence linked to an inducible gene of interest. The inducible polynucleotide component may also include regulatory sequences such as a polyA sequence, insulators, or posttranscriptional regulatory elements such as WPRE placed 5′ or 3′ to the coding region to improve system performance.

Referring still to FIG. 3, on a second vector backbone, the constitutive polynucleotide component includes a separation element (P2a, etc.) or a second constitutive promoter can be used to produce separate polypeptide chains of the split transcription factor, which can be composed of different fusion variants of DNA binding domain, transcriptional regulatory domain, NS3a, and a reader protein (DNCR2, ANR, GNCR1, or minimized/modified variants thereof). Optional regulatory sequences such as polyAs, insulators, or WPRE can be placed 5′ or 3′ to the coding regions to improve system performance (see Table 1).

The disclosure provides compositions comprising a polynucleotide set that includes a constitutive polynucleotide component encoding a split transcription factor and an inducible polynucleotide component that is bound by that transcription factor to regulate the expression of a gene of interest.

A polynucleotide set of the disclosure may be provided as part of a vector. In some embodiments, the inducible and constitutive polynucleotide components of the polynucleotide set may be provided as part of a single vector.

The disclosure provides a composition that includes a single vector comprising an inducible polynucleotide component linked to a gene of interest and a constitutive polynucleotide component encoding a split transcription for regulating the expression of the gene of interest. In some embodiments, the composition may be used for producing a polypeptide product of interest.

In some embodiments, the composition may be used for treating a subject in need of a therapy. The disclosure provides a pharmaceutical composition that includes: (i) a single vector comprising an inducible polynucleotide component linked to a gene of interest and a constitutive polynucleotide component encoding a split transcription for regulating the expression of the gene of interest, and (ii) a pharmaceutically acceptable carrier, excipient, and/or stabilizer.

In some embodiments, the constitutive and inducible polynucleotide components may be provided as part of a set of at least two vectors, wherein, for example, a first vector includes the inducible polynucleotide component, and a second vector includes the constitutive polynucleotide component.

In some embodiments, the disclosure provides a composition that includes: (i) a first vector comprising an inducible polynucleotide component, and (ii) a second vector that includes a constitutive polynucleotide component encoding a split transcription for regulating the expression of the gene of interest. In some embodiments, the composition may be used for producing a polypeptide product of interest.

In some embodiments, the composition may be used for treating a subject in need of a therapy. The disclosure provides a composition that includes: (i) a first vector comprising an inducible polynucleotide component, (ii) a second vector that includes a constitutive polynucleotide component encoding a split transcription for regulating the expression of the gene of interest, and (iii) a pharmaceutically acceptable carrier, excipient, and/or stabilizer.

### Host Cells

Expression vectors of the disclosure may be expressed in host cells. Host cells may, for example, be prokaryotic cells, such as bacteria cells; or eukaryotic cells, such as yeast cells, plant cells, or mammalian cells. Examples of mammalian cells suitable for use with the disclosure include human, mouse, rat, pig, rabbit, sheep, and goat cells. In some cases, the cells are synthetic cells.

A host cell may, for example, be selected from the group consisting of: cardiac cell, lung cell, muscle cell, epithelial cell, pancreatic cell, skin cell, CNS cell, neuron, myocyte, skeletal muscle cell, smooth muscle cell, liver cell, kidney cell and glial cell.

In some embodiments, a host cell is a human cell ex vivo. In some embodiments, a host cell is a human cell in vivo.

In some embodiments, a host cell is a stem cell such as a pluripotent stem cell or a hematopoietic stem cell.

In some embodiments, a host cell is a multipotent cell or a mesenchymal cell or a mesenchymal stromal cell (MSC).

In some embodiments, a host cell is a stem cell and the polynucleotides of the disclosure are used to control differentiation for cell products being generated from pluripotent cells, such as pluripotent stem cells. The drug-inducible gene expression system may, for example, be used to control the timing/dosage of transcription factors driving the differentiation.

In some embodiments, a host cell is not pluripotent and the polynucleotides of the disclosure are used to control reprogramming of the cell to induce pluripotency. The drug-inducible gene expression system may, for example, be used to control the timing/dosage of transcription factors driving the reprogramming.

In some embodiments, a host cell is part of an organism. In addition to the therapeutic embodiments described elsewhere herein, the cells may be part of a model organism. The drug-inducible gene expression system may, for example, be used to control expression producing a characteristic for scientific study, such as a disease characteristic or a biological enhancement. Examples of suitable model organisms include yeast, fruit flies, nematodes, frogs, mice and fish (such as zebrafish). The gene of interest may, for example, be a dysfunctional polypeptide, or a polypeptide that interacts with or modulates a gene of the organism, or that interferes with a metabolic process. The small molecules of the disclosure may be administered to modulate or titrate expression and thus produce variation in the characteristic being studied.

In some embodiments, a host cell is a cancer cell and/or a non-cancer cell from a human subject diagnosed with cancer.

In some embodiments, a host cell is an immune cell selected from the group consisting of: leukocyte, lymphocyte, T cell, regulatory T cell, effector T cell, CD4+ effector T cell, CD8+ effector T cell, memory T cell, autoreactive T cell, exhausted T cell, natural killer T cell, B cell, dendritic cell, and macrophage.

In some embodiments, a host cell is a producer cell line wherein cells of the cell line comprise a polynucleotide set configured for producing a product of interest.

Host cells may be transformed with one or more polynucleotides or vectors of the disclosure and cultured in nutrient media. Nutrient media may be formulated for inducing promoters, selecting transformants, or amplifying the genes of interest.

In some embodiments, the cell is a mammalian cell or cell line. Non-limiting examples include African green monkey kidney cells (VERO-76, ATCC CRL-1587); baby hamster kidney cells (BHK, ATCC CCL 10); BALB/c mouse myeloma lines (NSO/I, ECACC No: 85110503); buffalo rat liver cells (BRL 3A, ATCC CRL 1442); canine kidney cells (MDCK, ATCC CCL 34); Chinese hamster ovary (CHO) cell or cell line, CHO-K1 cell line (see, e.g., ATCC catalog no. CCL-61™ and Lewis, N. E. et al. (2013) Nat. Biotechnol. 31:759-765); Chinese hamster ovary cells +/−DHFR (see. e.g., Urlaub, G. and Chasin, L. A. (1980) Proc. Natl. Acad. Sci. 77:4216-4220); FS4 cells; HEK 293 cells; HT-1080 cells (ATCC® CCL-121™); human cervical carcinoma cells (HeLa, ATCC CCL-2); human embryonic kidney cell lines (293 or 293 cells subcloned for growth in suspension culture, Graham et al, J. Gen Virol. 36:59 (1977)); human hepatoma line (Hep G2); human liver cells (Hep G2, HB 8065); human lung cells (W138, ATCC CCL 75); human retinoblasts (PER.C6, CruCell, Leiden, The Netherlands); monkey kidney cells (CV1 ATCC CCL 70); monkey kidney CV1 line transformed by SV40 (COS-7, ATCC CRL 1651); mouse Sertoli cells (TM4, Mather, Biol. Reprod. 23:243-251 (1980)); mouse mammary tumor (MMT 060562, ATCC CCL51); MRC 5 cells; TRI cells (Mather et al., Annals N. Y. Acad. Sci. 383:44-68 (1982)); and engineered T cells and engineered natural killer cells.

A polynucleotide set of the disclosure may be provided in a host cell. The cells can be transiently or stably engineered to incorporate the polynucleotide set of the disclosure. The disclosure provides a cell comprising a polynucleotide set that includes a constitutive polynucleotide component encoding a split transcription factor and an inducible polynucleotide component that is bound by that transcription factor to regulate the expression of a gene of interest.

The disclosure provides a composition comprising a cell modified to express a polynucleotide set. In some embodiments, the cell composition may be used for producing a polypeptide product of interest. The expressed polypeptide can be recovered from the cell free extract or recovered from the culture medium.

In some embodiments, the composition may be used for treating a subject in need of a therapy. The disclosure provides a pharmaceutical composition that includes: (i) a cell which has been modified to express a polynucleotide set, and (ii) a pharmaceutically acceptable carrier, excipient, or stabilizer.

The cells may include polynucleotides of the disclosure expressing a gene of interest that provides a therapeutic benefit. Expression of the gene of interest may confer the cells with ability to attack tumor cells. The gene of interest may be a chimeric antigen receptor (CAR), e.g., a chimeric antigen receptor that targets tumor cells. The gene of interest may express a single-chain antibody fragment linked to a hinge linked to a transmembrane region. The transmembrane region may be linked to an intracellular signaling domain. The transmembrane region may be linked to a costimulatory domain.

The cells of the composition may, for example, be T cells. The cells of the composition may, for example, be CAR-T cells.

In some embodiments, the disclosure provides a cell composition comprising a means for reducing, ameliorating, or inhibiting exhaustion and/or dysfunction in a population of immune cells, e.g., immune cells expressing a CAR. In some embodiments, the means comprise expressing the CAR as a gene of interest in a polynucleotide set.

### Methods of Making Small Molecules

The small molecules of the disclosure may be synthesized using known techniques. Danoprevir ((2R,6S,12Z,13aS,14aR,16aS)-14a-[(Cyclopropylsulfonyl)carbamoyl]-6-({[(2-methyl-2-propanyl)oxy]carbonyl}amino)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoro-1,3-dihydro-2H-isoindole-2-carboxylate) may be synthesized using known techniques. See for example, Carreira, Erick Moran, Hisashi Yamamoto, and N. K. Yee. “Industrial Applications of Asymmetric Synthesis.” In Comprehensive Chirality 9, Amsterdam: Elsevier, 2012. Section 9.19.6, Danoprevir, the disclosure of which is incorporated herein by reference.

### Methods of Making Polynucleotides

The disclosure provides methods of producing the polynucleotides of the disclosure, such as DNA vectors of the disclosure and their subcomponents, as well as packaging vectors and plasmids of the disclosure. Standard molecular biology techniques may be used to assemble the polynucleotides of the disclosure. Polynucleotides can be chemically synthesized.

### Methods of Making Packaged Viral Capsids

The disclosure includes methods of making viral capsids containing polynucleotides of the disclosure. In general, viral capsids of the disclosure may be produced by supplying cells with packaging polynucleotides of the disclosure. The packaging polynucleotides may be supplied to packaging cells as plasmids. The packaging cells may be cultured to produce the viral capsids containing polynucleotides of the disclosure. Preferably the packaged viral capsids are replication incompetent.

A variety of commercially available kits are suitable for producing packaged viral capsids of the disclosure. Examples include: MISSION® Lentiviral Packaging Mix (available from Millipore Sigma); LV-Max Lentiviral Packaging Mix (available from ThermoFisher Scientific).

Viral capsid produced by packaging cells may be purified for use in downstream methods, such as delivery to cells for use in production of polypeptides, delivery to cells for use in cell-based therapies, or delivery to subjects for gene therapy methods. Purification may include processing to eliminate contaminants from host cells or culture media. Purification steps may include steps based on physical and/or chemical characteristics of the plasmids. Chemical characteristics may include, for example, hydrophilicity-hydrophobicity. Physical characteristics may include, for example, size. Examples of purification strategies based on particle size include density-gradient ultracentrifugation, ultrafiltration, precipitation, two-phase extraction systems and size exclusion chromatography. In some cases, precipitation may be employed together with centrifugation, e.g., using polyethylene glycol, ammonium sulfate or calcium phosphate. In some cases, aqueous two-phase separation systems with PEG, dextran or polyvinyl alcohol may be used. In some cases, membrane-based tangential flow filtration techniques are used; examples include ultrafiltration, diafiltration and microfiltration. In other embodiments, chromatographic means may be used for purifying viral capsids. In still other embodiments, immunoaffinity methods may be used to capture capsids using monoclonal antibodies having specificity to the relevant capsids. See Morenweiser, R., “Downstream processing of viral vectors and vaccines,” Gene Therapy (2005) 12, S103-S110 (2005), the entire disclosure of which is incorporated herein by reference.

Examples of suitable viral capsids include, but are not limited to, adenovirus, retrovirus, Lentivirus, Sendai virus vector, a baculovirus, Epstein Barr virus, a papovavirus, a vaccinia virus, a herpes simplex virus, and an adeno-associated virus (AAV).

### Methods of Making Cells

The disclosure provides methods of making a modified cell to express a gene of interest.

In some embodiments, the disclosure provides a method of making a modified cell that expresses a polynucleotide set for isolation of a polypeptide product of interest. In one embodiment, the disclosure provides a method of generating or preparing cells for expression and isolation of a polypeptide product of interest from a polynucleotide set integrated into a single vector. In one embodiment, the disclosure provides a method of generating or preparing cells for expression and isolation of a polypeptide product of interest from a polynucleotide set integrated into two (or more) vectors.

In some embodiments, the disclosure provides a method of making a therapeutic cell that expresses a polynucleotide set for use in treating a subject in need of a cell therapy. In one embodiment, the disclosure provides a method of generating or preparing a therapeutic cell that expresses a gene of interest from a polynucleotide set integrated into a single vector. In one embodiment, the disclosure provides a method of generating or preparing a therapeutic cell that expresses a gene of interest from a polynucleotide set integrated into two (or more) vectors.

In some embodiments, the polynucleotides of the disclosure are maintained as extrachromosomal polynucleotides in the host cell. In some embodiments, the polynucleotides of the disclosure are present in a vector (e.g., expression vector) in the host cell. In some embodiments, the polynucleotides of the disclosure or subcomponents thereof, are integrated into a chromosome of the host cell.

Various methods can be used to introduce the expression vector of some embodiments of the disclosure into cells to produce cells of the disclosure. See for example, Green, et al., Molecular cloning: A laboratory manual. Cold Spring Harbor, NY: Cold Spring Harbor Laboratory Press (2014).

Methods of introducing nucleic acid alterations to a gene of interest are well known in the art. Examples include targeted homologous recombination (e.g. “Hit and run”, “double-replacement”), site specific recombinases (e.g. the Cre recombinase and the Flp recombinase), PB transposases (e.g. Sleeping Beauty, piggyBac, To12 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform. Agents for introducing nucleic acid alterations to a gene of interest can be designed using publicly available sources or obtained commercially from Transposagen, Addgene and Sangamo Biosciences. Vectors of the disclosure may make use of these methods for integrating polynucleotides of the disclosure into a host genome. Vectors of the disclosure may include polynucleotides encoding polypeptides required for implementation of these methods for integrating polynucleotides of the disclosure into a host genome.

Various approaches suitable for integrating a polynucleotide(s) into a host cell genome are known in the art, including random integration or site-specific integration (e.g., a“landing pad” approach); see, e.g., Zhao, M. el al. (2018) Appl. Microbiol. Biotechnol. 102:6105-6117; Lee, J. S. et al. (2015) Sci. Rep. 5:8572; and Gaidukov, L. et al. (2018) Nucleic Acids Res. 46:4072-4086. Vectors of the disclosure may make use of these methods for integrating polynucleotides of the disclosure into a host genome. Vectors of the disclosure may include polynucleotides encoding polypeptides required for implementation of these methods for integrating polynucleotides of the disclosure into a host genome.

Examples of commercially available media suitable for culturing host cells of the disclosure include Ham's F10 (Sigma), Minimal Essential Medium ((MEM), (Sigma), RP MI-1640 (Sigma), and Dulbecco's Modified Eagle's Medium ((DMEM), Sigma).

Culture media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. Culture conditions, such as temperature, pH, and the like, will be apparent to the ordinarily skilled artisan.

### Methods of Making Polypeptides and Cellular Metabolites

The disclosure provides methods of manufacturing polypeptides. The methods may make use of cells of the disclosure treated with the small molecules of the disclosure.

The disclosure provides methods of producing a vector comprising a polynucleotide set, delivering the vector into a cell (e.g., in vivo, in vitro, or ex vivo), and expressing the polynucleotide set to provide and/or control a cellular function. Expression may be modulated by a small molecule of the disclosure.

In one embodiment, the method comprises the steps of (a) modifying a cell using a polynucleotide set encoding a polypeptide product of interest to yield a producer cell line; (b) culturing the producer cell line under conditions conducive for expression of the polypeptide product, (c) modulating production of the polypeptide product by delivering to the cell line a small molecule of the disclosure; and (d) optionally, recovering the expressed polypeptide.

In one embodiment, the method comprises the steps of (a) modifying a cell using a polynucleotide set encoding a polypeptide product of interest to yield a producer cell line; (b) culturing the producer cell line under conditions conducive for expression of the polypeptide product, (c) measuring the polypeptide of interest; (d) modulating production of the polypeptide product by delivering to the cell line a small molecule of the disclosure; and (d) optionally, recovering the expressed polypeptide.

The expressed polypeptide may, for example, be recovered from a cell free extract or recovered from the culture medium.

In one example, the polypeptide product of interest is a therapeutic protein or peptide.

Polypeptide products of interest may be produced intracellularly, or directly secreted into the medium. If the polypeptide is produced intracellularly, cells may be lysed. Particulate debris may be removed, for example, by centrifugation or ultrafiltration. Where the polypeptide is secreted into the medium, supernatants from such expression systems may optionally be concentrated, e.g., using a commercially available protein concentration filter, for example, an Ami con or Millipore Pellicon ultrafiltration unit. A protease inhibitor such as PMSF may be included in any of the foregoing steps to inhibit proteolysis and antibiotics may be included to prevent the growth of adventitious contaminants.

Polypeptides may be purified using, for example, hydroxylapatite chromatography, gel electrophoresis, dialysis, and affinity chromatography, fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), low pH hydrophobic interaction chromatography, chromatofocusing, SDS-PAGE, ammonium sulfate precipitation, fractionation on immunoaffmity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration

Polypeptide products of interest may be purified to obtain preparations that are substantially homogeneous for further assays and uses. Polypeptide products of interest may be purified to obtain preparations that are sufficiently homogenous for pharmaceutical uses.

Embodiments of the disclosure may make use of cells transformed with the polynucleotides of the disclosure for making cellular metabolites. For example, cells transformed with the polynucleotides of the disclosure may be used to transform substrates into products, e.g., alcohol products, such as ethanol, acetone, and butanol. Metabolites include, for example, products of metabolic pathways, such as glycolysis, fatty acid synthesis, the TCA cycle, phosphorylation pathways and the pentose phosphate pathway.

### Cell Therapy Methods

The disclosure provides methods of treating a subject in need of a cell therapy. The method comprises the steps of (a) administering to the subject an effective amount of a pharmaceutical composition comprising a therapeutic cell encoding a polypeptide product of interest; and (b) administering a therapeutically effective amount of a small molecule to the subject.

In one embodiment, the disclosure provides a method for treating a cancer, e.g., a tumor, in a subject in need thereof. Examples of cancers that can be treated using a pharmaceutical composition disclosed herein include, but are not limited to, melanomas, lymphomas, sarcomas, and cancers of the colon, kidney, stomach, bladder, brain (e.g., gliomas, glioblastomas, astrocytomas, medulloblastomas), prostate, bladder, rectum, esophagus, pancreas, liver, lung, breast, uterus, cervix, ovary, blood (e.g., acute myeloid leukemia, acute lymphoid leukemia, chronic myeloid leukemia, chronic lymphocytic leukemia, Burkitt's lymphoma, EBV-induced B-cell lymphoma).

In one embodiment, the disclosure provides a method of controlling a T cell-mediated immune response in a subject in need thereof.

In one embodiment, the disclosure provides a method of stimulating a T cell-mediated immune response to a target cell population or tissue in a subject.

In one embodiment, the disclosure provides a method of providing an anti-tumor immunity in a subject.

### Gene Therapy Methods

The disclosure provides methods of delivering a polynucleotide set of the disclosure to a subject. A polynucleotide set of the disclosure may be delivered into a cell of a subject. The method may include administering a pharmaceutically effective amount of the polynucleotide set to the subject. Administration may be via administration of viral particles including one or more polynucleotides of the disclosure. Administration may be via administration of a pharmaceutical composition including one or more polynucleotides of the disclosure.

The method comprises the steps of (a) administering to the subject an effective amount of a pharmaceutical composition comprising a polynucleotide set encoding a polypeptide product of interest; (b) administering a therapeutically effective amount of a small molecule to the subject; (c) monitoring the production of the therapeutic polypeptide in the subject; and (d) optionally, adjusting the dosage of the small molecule to adjust production of the polypeptide product to the subject to a desired level.

The subject may be a mammalian subject. The subject may be a human subject.

Examples of conditions that may be selected for gene therapy include, but are not limited to, cancer, cystic fibrosis, heart disease, diabetes, hemophilia, and AIDS.

### Kits

The disclosure provides kits or articles of manufacture comprising polynucleotides of the disclosure and a preparation for delivery of the polynucleotides to cells. The polynucleotides may be provided as part of a vector of the disclosure. In some embodiments, the kit or article of manufacture further comprises instructions for using the set of the polynucleotides to transform cells to express a gene of interest to produce a polypeptide of interest.

In some cases, the kits may also include a small molecule of the disclosure.

### Tables

TABLE A
Target sequences.

| Target Name | Sequence |
|---|---|
| Nuclear receptor | MPCIQAQYGTPAPSPGPRDHLASDPLTPEFIKPTMDLASPEAA |
| subfamily 4 group A | PAAPTALPSFSTFMDGYTGEFDTFLYQLPGTVQPCSSASSSAS |
| member 1 (NR4A1), | STSSSSATSPASASFKFEDFQVYGCYPGPLSGPVDEALSSSGS |
| isoform 1, | DYYGSPCSAPSPSTPSFQPPQLSPWDGSFGHFSPSQTYEGLRA |
| UniProtKB | WTEQLPKASGPPQPPAFFSFSPPTGPSPSLAQSPLKLFPSQATH |
| Accession No. | QLGEGESYSMPTAFPGLAPTSPHLEGSGILDTPVTSTKARSGA |
| P22736-1 | PGGSEGRCAVCGDNASCQHYGVRTCEGCKGFFKRTVQKNA |
| SEQ ID NO: 182 | KYICLANKDCPVDKRRRNRCQFCRFQKCLAVGMVKEVVRT |
| | DSLKGRRGRLPSKPKQPPDASPANLLTSLVRAHLDSGPSTAK |
| | LDYSKFQELVLPHFGKEDAGDVQQFYDLLSGSLEVIRKWAE |
| | KIPGFAELSPADQDLLLESAFLELFILRLAYRSKPGEGKLIFCS |
| | GLVLHRLQCARGFGDWIDSILAFSRSLHSLLVDVPAFACLSA |
| | LVLITDRHGLQEPRRVEELQNRIASCLKEHVAAVAGEPQPAS |
| | CLSRLLGKLPELRTLCTQGLQRIFYLKLEDLVPPPPIIDKIFMD |
| | TLPF |
| Nuclear receptor | MWLAKACWSIQSEMPCIQAQYGTPAPSPGPRDHLASDPLTP |
| subfamily 4 group A | EFIKPTMDLASPEAAPAAPTALPSFSTFMDGYTGEFDTFLYQ |
| member 1 (NR4A1), | LPGTVQPCSSASSSASSTSSSSATSPASASFKFEDFQVYGCYP |
| isoform 2, | GPLSGPVDEALSSSGSDYYGSPCSAPSPSTPSFQPPQLSPWDG |
| UniProtKB | SFGHFSPSQTYEGLRAWTEQLPKASGPPQPPAFFSFSPPTGPS |
| Accession No. | PSLAQSPLKLFPSQATHQLGEGESYSMPTAFPGLAPTSPHLEG |
| P22736-2 | SGILDTPVTSTKARSGAPGGSEGRCAVCGDNASCQHYGVRT |
| SEQ ID NO: 183 | CEGCKGFFKRTVQKNAKYICLANKDCPVDKRRRNRCQFCRF |
| | QKCLAVGMVKEVVRTDSLKGRRGRLPSKPKQPPDASPANLL |
| | TSLVRAHLDSGPSTAKLDYSKFQELVLPHFGKEDAGDVQQF |
| | YDLLSGSLEVIRKWAEKIPGFAELSPADQDLLLESAFLELFIL |
| | RLAYRSKPGEGKLIFCSGLVLHRLQCARGFGDWIDSILAFSRS |
| | LHSLLVDVPAFACLSALVLITDRHGLQEPRRVEELQNRIASC |
| | LKEHVAAVAGEPQPASCLSRLLGKLPELRTLCTQGLQRIFYL |
| | KLEDLVPPPPIIDKIFMDTLPF |
| Nuclear receptor | MPCIQAQYGTPAPSPGPRDHLASDPLTPEFIKPTMDLASPEAA |
| subfamily 4 group A | PAAPTALPSFSTFMDGYTGEFDTFLYQLPGTVQPCSSASSSAS |
| member 1 (NR4A1), | STSSSSATSPASASFKFEDFQVYGCYPGPLSGPVDEALSSSGS |
| isoform 3, | DYYGSPCSAPSPSTPSFQPPQLSPWDGSFGHFSPSQTYEGLRA |
| UniProtKB | WTEQLPKASGPPQPPAFFSFSPPTGPSPSLAQSPLKLFPSQATH |
| Accession No. | QLGEGESYSMPTAFPGLAPTSPHLEGSGILDTPVTSTKARSGA |
| P22736-3 | PGGSEGRCAVCGDNASCQHYGVRTCEGCKGFFKVPRSPRW |
| SEQ ID NO: 184 | GLLLEMERGWPHPIGTCGLPLGSPPS |
| Nuclear receptor | MPCVQAQYGSSPQGASPASQSYSYHSSGEYSSDFLTPEFVKF |
| subfamily 4 group A | SMDLTNTEITATTSLPSFSTFMDNYSTGYDVKPPCLYQMPLS |
| member 2 (NR4A2), | GQQSSIKVEDIQMHNYQQHSHLPPQSEEMMPHSGSVYYKPS |
| isoform 1, | SPPTPTTPGFQVQHSPMWDDPGSLHNFHQNYVATTHMIEQR |
| UniProtKB | KTPVSRLSLFSFKQSPPGTPVSSCQMRFDGPLHVPMNPEPAG |
| Accession No. | SHHVVDGQTFAVPNPIRKPASMGFPGLQIGHASQLLDTQVPS |
| P43354-1 | PPSRGSPSNEGLCAVCGDNAACQHYGVRTCEGCKGFFKRTV |
| SEQ ID NO: 185 | QKNAKYVCLANKNCPVDKRRRNRCQYCRFQKCLAVGMVK |
| | EVVRTDSLKGRRGRLPSKPKSPQEPSPPSPPVSLISALVRAHV |
| | DSNPAMTSLDYSRFQANPDYQMSGDDTQHIQQFYDLLTGS |
| | MEIIRGWAEKIPGFADLPKADQDLLFESAFLELFVLRLAYRS |
| | NPVEGKLIFCNGVVLHRLQCVRGFGEWIDSIVEFSSNLQNMN |
| | IDISAFSCIAALAMVTERHGLKEPKRVEELQNKIVNCLKDHV |
| | TFNNGGLNRPNYLSKLLGKLPELRTLCTQGLQRIFYLKLEDL |
| | VPPPAIIDKLFLDTLPF |
| Nuclear receptor | MDNYSTGYDVKPPCLYQMPLSGQQSSIKVEDIQMHNYQQH |
| subfamily 4 group A | SHLPPQSEEMMPHSGSVYYKPSSPPTPTTPGFQVQHSPMWD |
| member 2 (NR4A2), | DPGSLHNFHQNYVATTHMIEQRKTPVSRLSLFSFKQSPPGTP |
| isoform 2, | VSSCQMRFDGPLHVPMNPEPAGSHHVVDGQTFAVPNPIRKP |
| UniProtKB | ASMGFPGLQIGHASQLLDTQVPSPPSRGSPSNEGLCAVCGDN |
| Accession No. | AACQHYGVRTCEGCKGFFKRTVQKNAKYVCLANKNCPVD |
| P43354-2 | KRRRNRCQYCRFQKCLAVGMVKEVVRTDSLKGRRGRLPSK |
| SEQ ID NO: 186 | PKSPQEPSPPSPPVSLISALVRAHVDSNPAMTSLDYSRFQANP |
| | DYQMSGDDTQHIQQFYDLLTGSMEIIRGWAEKIPGFADLPK |
| | ADQDLLFESAFLELFVLRLAYRSNPVEGKLIFCNGVVLHRLQ |
| | CVRGFGEWIDSIVEFSSNLQNMNIDISAFSCIAALAMVTERHG |
| | LKEPKRVEELQNKIVNCLKDHVTFNNGGLNRPNYLSKLLGK |
| | LPELR |
| Nuclear receptor | MPCVQAQYSPSPPGSSYAAQTYSSEYTTEIMNPDYTKLTMD |
| subfamily 4 group A | LGSTEITATATTSLPSISTFVEGYSSNYELKPSCVYQMQRPLIK |
| member 3 (NR4A3), | VEEGRAPSYHHHHHHHHHHHHHHQQQHQQPSIPPASSPEDE |
| isoform alpha, | VLPSTSMYFKQSPPSTPTTPAFPPQAGALWDEALPSAPGCIAP |
| UniProtKB | GPLLDPPMKAVPTVAGARFPLFHFKPSPPHPPAPSPAGGHHL |
| Accession No. | GYDPTAAAALSLPLGAAAAAGSQAAALESHPYGLPLAKRA |
| Q92570-1 | APLAFPPLGLTPSPTASSLLGESPSLPSPPSRSSSSGEGTCAVC |
| SEQ ID NO: 187 | GDNAACQHYGVRTCEGCKGFFKRTVQKNAKYVCLANKNC |
| | PVDKRRRNRCQYCRFQKCLSVGMVKEVVRTDSLKGRRGRL |
| | PSKPKSPLQQEPSQPSPPSPPICMMNALVRALTDSTPRDLDYS |
| | RYCPTDQAAAGTDAEHVQQFYNLLTASIDVSRSWAEKIPGF |
| | TDLPKEDQTLLIESAFLELFVLRLSIRSNTAEDKFVFCNGLVL |
| | HRLQCLRGFGEWLDSIKDFSLNLQSLNLDIQALACLSALSMI |
| | TERHGLKEPKRVEELCNKITSSLKDHQSKGQALEPTESKVLG |
| | ALVELRKICTLGLQRIFYLKLEDLVSPPSIIDKLFLDTLPF |
| Nuclear receptor | MPCVQAQYSPSPPGSSYAAQTYSSEYTTEIMNPDYTKLTMD |
| subfamily 4 group A | LGSTEITATATTSLPSISTFVEGYSSNYELKPSCVYQMQRPLIK |
| member 3 (NR4A3), | VEEGRAPSYHHHHHHHHHHHHHHQQQHQQPSIPPASSPEDE |
| isoform beta, | VLPSTSMYFKQSPPSTPTTPAFPPQAGALWDEALPSAPGCIAP |
| UniProtKB | GPLLDPPMKAVPTVAGARFPLFHFKPSPPHPPAPSPAGGHHL |
| Accession No. | GYDPTAAAALSLPLGAAAAAGSQAAALESHPYGLPLAKRA |
| Q92570-2 | APLAFPPLGLTPSPTASSLLGESPSLPSPPSRSSSSGEGTCAVC |
| SEQ ID NO: 188 | GDNAACQHYGVRTCEGCKGFFKRTVQKNAKYVCLANKNC |
| | PVDKRRRNRCQYCRFQKCLSVGMVKEVVRTDSLKGRRGRL |
| | PSKPKSPLQQEPSQPSPPSPPICMMNALVRALTDSTPRDLDYS |
| | RVSFMISCFQMNDQGLYLWLLVIRVD |
| Nuclear receptor | MHDSIRFGNVDMPCVQAQYSPSPPGSSYAAQTYSSEYTTEIM |
| subfamily 4 group A | NPDYTKLTMDLGSTEITATATTSLPSISTFVEGYSSNYELKPS |
| member 3 (NR4A3), | CVYQMQRPLIKVEEGRAPSYHHHHHHHHHHHHHHQQQHQ |
| isoform 3, | QPSIPPASSPEDEVLPSTSMYFKQSP |
| UniProtKB | PSTPTTPAFPPQAGALWDEALPSAPGCIAPGPLLDPPMKAVP |
| Accession No. | TVAGARFPLFHFKPSPPHPPAPSPAGGHHLGYDPTAAAALSL |
| Q92570-3 | PLGAAAAAGSQAAALESHPYGLPLAKRAAPLAFPPLGLTPSP |
| SEQ ID NO: 189 | TASSLLGESPSLPSPPSRSSSSGEGTCAVCGDNAACQHYGVR |
| | TCEGCKGFFKRTVQKNAKYVCLANKNCPVDKRRRNRCQYC |
| | RFQKCLSVGMVKEVVRTDSLKGRRGRLPSKPKSPLQQEPSQ |
| | PSPPSPPICMMNALVRALTDSTPRDLDYSRYCPTDQAAAGTD |
| | AEHVQQFYNLLTASIDVSRSWAEKIPGFTDLPKEDQTLLIESA |
| | FLELFVLRLSIRSNTAEDKFVFCNGLVLHRLQCLRGFGEWLD |
| | SIKDFSLNLQSLNLDIQALACLSALSMITERHGLKEPKRVEEL |
| | CNKITSSLKDHQSKGQALEPTESKVLGALVELRKICTLGLQRI |
| | FYLKLEDLVSPPSIIDKLFLDTLPF |
| Thymocyte | MDVRFYPPPAQPAAAPDAPCLGPSPCLDPYYCNKFDGENMY |
| selection-associated | MSMTEPSQDYVPASQSYPGPSLESEDFNIPPITPPSLPDHSLVH |
| high mobility group | LNEVESGYHSLCHPMNHNGLLPFHPQNMDLPEITVSNMLGQ |
| box protein TOX | DGTLLSNSISVMPDIRNPEGTQYSSHPQMAAMRPRGQPADIR |
| (TOX), isoform 1, | QQPGMMPHGQLTTINQSQLSAQLGLNMGGSNVPHNSPSPPG |
| UniProtKB | SKSATPSPSSSVHEDEGDDTSKINGGEKRPASDMGKKPKTP |
| Accession No. | KKKKKKDPNEPQKPVSAYALFFRDTQAAIKGQNPNATFGEV |
| 094900-1 | SKIVASMWDGLGEEQKQVYKKKTEAAKKEYLKQLAAYRAS |
| SEQ ID NO: 190 | LVSKSYSEPVDVKTSQPPQLINSKPSVFHGPSQAHSALYLSSH |
| | YHQQPGMNPHLTAMHPSLPRNIAPKPNNQMPVTVSIANMA |
| | VSPPPPLQISPPLHQHLNMQQHQPLTMQQPLGNQLPMQVQS |
| | ALHSPTMQQGFTLQPDYQTIINPTSTAAQVVTQAMEYVRSG |
| | CRNPPPQPVDWNNDYCSSGGMQRDKALYLT |
| TOX high mobility | MQQTRTEAVAGAFSRCLGFCGMRLGLLLLARHWCIAGVFP |
| group box family | QKFDGDSAYVGMSDGNPELLSTSQTYNGQSENNEDYEIPPIT |
| member 2 (TOX2), | PPNLPEPSLLHLGDHEASYHSLCHGLTPNGLLPAYSYQAMDL |
| isoform 1, | PAIMVSNMLAQDSHLLSGQLPTIQEMVHSEVAAYDSGRPGP |
| UniProtKB | LLGRPAMLASHMSALSQSQLISQMGIRSSIAHSSPSPPGSKSA |
| Accession No. | TPSPSSSTQEEESEVHFKISGEKRPSADPGKKAKNPKKKKKK |
| Q96NM4-1 | DPNEPQKPVSAYALFFRDTQAAIKGQNPSATFGDVSKIVASM |
| SEQ ID NO: 191 | WDSLGEEQKQSSPDQGETKSTQANPPAKMLPPKQPMYAMP |
| | GLASFLTPSDLQAFRSGASPASLARTLGSKSLLPGLSASPPPPP |
| | SFPLSPTLHQQLSLPPHAQGALLSPPVSMSPAPQPPVLPTPMA |
| | LQVQLAMSPSPPGPQDFPHISEFPSSSGSCSPGPSNPTSSGDW |
| | DSSYPSGECGISTCSLLPRDKSLYLT |
| TOX high mobility | MQQTRTEAVAGAFSRCLGFCGMRLGLLLLARHWCIAGVFP |
| group box family | QKFDGDSAYVGMSDGNPELLSTSQTYNGQSENNEDYEIPPIT |
| member 2 (TOX2), | PPNLPEPSLLHLGDHEASYHSLCHGLTPNGLLPAYSYQAMDL |
| isoform 2, | PAIMVSNMLAQDSHLLSGQLPTIQEMVHSEVAAYDSGRPGP |
| UniProtKB | LLGRPAMLASHMSALSQSQLISQMGIRSSIAHSSPSPPGSKSA |
| Accession No. | TPSPSSSTQEEESEVHFKISGEKRPSADPGKKAKNPKKKKKK |
| Q96NM4-2 | DPNEPQKPVSAYALFFRDTQAAIKGQNPSATFGDVSKIVASM |
| SEQ ID NO: 192 | WDSLGEEQKQAYKRKTEAAKKEYLKALAAYRASLVSKSSP |
| | DQGETKSTQANPPAKMLPPKQPMYAMPGLASFLTPSDLQAF |
| | RSGASPASLARTLGSKSLLPGLSASPPPPPSFPLSPTLHQQLSL |
| | PPHAQGALLSPPVSMSPAPQPPVLPTPMALQVQLAMSPSPPG |
| | PQDFPHISEFPSSSGSCSPGPSNPTSSGDWDSSYPSGECGISTC |
| | SLLPRDKSLYLT |
| TOX high mobility | MSDGNPELLSTSQTYNGQSENNEDYEIPPITPPNLPEPSLLHL |
| group box family | GDHEASYHSLCHGLTPNGLLPAYSYQAMDLPAIMVSNMLA |
| member 2 (TOX2), | QDSHLLSGQLPTIQEMVHSEVAAYDSGRPGPLLGRPAMLAS |
| isoform 3, | HMSALSQSQLISQMGIRSSIAHSSPSPPGSKSATPSPSSSTQEE |
| UniProtKB | ESEVHFKISGEKRPSADPGKKAKNPKKKKKKDPNEPQKPVS |
| Accession No. | AYALFFRDTQAAIKGQNPSATFGDVSKIVASMWDSLGEEQK |
| Q96NM4-3 | QAYKRKTEAAKKEYLKALAAYRASLVSKSSPDQGETKSTQ |
| SEQ ID NO: 193 | ANPPAKMLPPKQPMYAMPGLASFLTPSDLQAFRSGASPASL |
| | ARTLGSKSLLPGLSASPPPPPSFPLSPTLHQQLSLPPHAQGALL |
| | SPPVSMSPAPQPPVLPTPMALQVQLAMSPSPPGPQDFPHISEF |
| | PSSSGSCSPGPSNPTSSGDWDSSYPSGECGISTCSLLPRDKSLY |
| | LT |
| TOX high mobility | MDVRLYPSAPAVGARPGAEPAGLAHLDYYHGGKFDGDSAY |
| group box family | VGMSDGNPELLSTSQTYNGQSENNEDYEIPPITPPNLPEPSLL |
| member 2 (TOX2), | HLGDHEASYHSLCHGLTPNGLLPAYSYQAMDLPAIMVSNM |
| isoform 4, | LAQDSHLLSGQLPTIQEMVHSEVAAYDSGRPGPLLGRPAML |
| UniProtKB | ASHMSALSQSQLISQMGIRSSIAHSSPSPPGSKSATPSPSSSTQ |
| Accession No. | EEESEVHFKISGEKRPSADPGKKAKNPKKKKKKDPNEPQKP |
| Q96NM4-4 | VSAYALFFRDTQAAIKGQNPSATFGDVSKIVASMWDSLGEE |
| SEQ ID NO: 194 | QKQAYKRKTEAAKKEYLKALAAYRASLVSKSSPDQGETKS |
| | TQANPPAKMLPPKQPMYAMPGLASFLTPSDLQAFRSGASPA |
| | SLARTLGSKSLLPGLSASPPPPPSFPLSPTLHQQLSLPPHAQGA |
| | LLSPPVSMSPAPQPPVLPTPMALQVQLAMSPSPPGPQDFPHIS |
| | EFPSSSGSCSPGPSNPTSSGDWDSSYPSGECGISTCSLLPRDKS |
| | LYLT |
| Interferon regulatory | MNLEGGGRGGEFGMSAVSCGNGKLRQWLIDQIDSGKYPGL |
| factor 4 (IRF4), | VWENEEKSIFRIPWKHAGKQDYNREEDAALFKAWALFKGK |
| isoform 1, | FREGIDKPDPPTWKTRLRCALNKSNDFEELVERSQLDISDPY |
| UniProtKB | KVYRIVPEGAKKGAKQLTLEDPQMSMSHPYTMTTPYPSLPA |
| Accession No. | QQVHNYMMPPLDRSWRDYVPDQPHPEIPYQCPMTFGPRGH |
| Q15306-1 | HWQGPACENGCQVTGTFYACAPPESQAPGVPTEPSIRSAEAL |
| SEQ ID NO: 195 | AFSDCRLHICLYYREILVKELTTSSPEGCRISHGHTYDASNLD |
| | QVLFPYPEDNGQRKNIEKLLSHLERGVVLWMAPDGLYAKR |
| | LCQSRIYWDGPLALCNDRPNKLERDQTCKLFDTQQFLSELQ |
| | AFAHHGRSLPRFQVTLCFGEEFPDPQRQRKLITAHVEPLLAR |
| | QLYYFAQQNSGHFLRGYDLPEHISNPEDYHRSIRHSSIQE |
| Interferon regulatory | MNLEGGGRGGEFGMSAVSCGNGKLRQWLIDQIDSGKYPGL |
| factor 4 (IRF4), | VWENEEKSIFRIPWKHAGKQDYNREEDAALFKAWALFKGK |
| isoform 2, | FREGIDKPDPPTWKTRLRCALNKSNDFEELVERSQLDISDPY |
| UniProtKB | KVYRIVPEGAKKGAKQLTLEDPQMSMSHPYTMTTPYPSLPA |
| Accession No. | QVHNYMMPPLDRSWRDYVPDQPHPEIPYQCPMTFGPRGHH |
| Q15306-2 | WQGPACENGCQVTGTFYACAPPESQAPGVPTEPSIRSAEALA |
| SEQ ID NO: 196 | FSDCRLHICLYYREILVKELTTSSPEGCRISHGHTYDASNLDQ |
| | VLFPYPEDNGQRKNIEKLLSHLERGVVLWMAPDGLYAKRLC |
| | QSRIYWDGPLALCNDRPNKLERDQTCKLFDTQQFLSELQAF |
| | AHHGRSLPRFQVTLCFGEEFPDPQRQRKLITAHVEPLLARQL |
| | YYFAQQNSGHFLRGYDLPEHISNPEDYHRSIRHSSIQE |
| Basic leucine zipper | MPHSSDSSDSSFSRSPPPGKQDSSDDVRRVQRREKNRIAAQK |
| transcriptional factor | SRQRQTQKADTLHLESEDLEKQNAALRKEIKQLTEELKYFTS |
| ATF-like (BATF), | VLNSHEPLCSVLAASTPSPPEVVYSAHAFHQPHVSSPRFQP |
| isoform 1, | |
| UniProtKB | |
| Accession No. | |
| Q16520-1 | |
| SEQ ID NO: 197 | |
| Basic leucine zipper | MHLCGGNGLLTQTDPKEQQRQLKKQKNRAAAQRSRQKHT |
| transcriptional factor | DKADALHQQHESLEKDNLALRKEIQSLQAELAWWSRTLHV |
| ATF-like 2 | HERLCPMDCASCSAPGLLGCWDQAEGLLGPGPQGQHGCRE |
| (BATF2), isoform 1, | QLELFQTPGSCYPAQPLSPGPQPHDSPSLLQCPLPSLSLGPAV |
| UniProtKB | VAEPPVQLSPSPLLFASHTGSSLQGSSSKLSALQPSLTAQTAP |
| Accession No. | PQPLELEHPTRGKLGSSPDNPSSALGLARLQSREHKPALSAA |
| Q8NIL9-1 | TWQGLVVDPSPHPLLAFPLLSSAQVHF |
| SEQ ID NO: 198 | |
| Basic leucine zipper | MDCASCSAPGLLGCWDQAEGLLGPGPQGQHGCREQLELFQ |
| transcriptional factor | TPGSCYPAQPLSPGPQPHDSPSLLQCPLPSLSLGPAVVAEPPV |
| ATF-like 2 | QLSPSPLLFASHTGSSLQGSSSKLSALQPSLTAQTAPPQPLELE |
| (BATF2), isoform 2, | HPTRGKLGSSPDNPSSALGLARLQSREHKPALSAATWQGLV |
| UniProtKB | VDPSPHPLLAFPLLSSAQVHF |
| Accession No. | |
| Q8NIL9-2 | |
| SEQ ID NO: 199 | |
| Basic leucine zipper | MSQGLPAAGSVLQRSVAAPGNQPQPQPQQQSPEDDDRKVR |
| transcriptional factor | RREKNRVAAQRSRKKQTQKADKLHEEYESLEQENTMLRREI |
| ATF-like 3 | GKLTEELKHLTEALKEHEKMCPLLLCPMNFVPVPPRPDPVA |
| (BATF3), isoform 1, | GCLPR |
| UniProtKB | |
| Accession No. | |
| Q9NR55-1 | |
| SEQ ID NO: 200 | |
| X-box-binding | MVVVAAAPNPADGTPKVLLLSGQPASAAGAPAGQALPLMV |
| protein 1 (XBP1), | PAQRGASPEAASGGLPQARKRQRLTHLSPEEKALRRKLKNR |
| isoform 1, | VAAQTARDRKKARMSELEQQVVDLEEENQKLLLENQLLRE |
| UniProtKB | KTHGLVVENQELRQRLGMDALVAEEEAEAKGNEVRPVAGS |
| Accession No. | AESAALRLRAPLQQVQAQLSPLQNISPWILAVLTLQIQSLISC |
| P17861-1 | WAFWTTWTQSCSSNALPQSLPAWRSSQRSTQKDPVPYQPPF |
| SEQ ID NO: 201 | LCQWGRHQPSWKPLMN |
| X-box-binding | MVVVAAAPNPADGTPKVLLLSGQPASAAGAPAGQALPLMV |
| protein 1 (XBP1), | PAQRGASPEAASGGLPQARKRQRLTHLSPEEKALRRKLKNR |
| isoform 2, | VAAQTARDRKKARMSELEQQVVDLEEENQKLLLENQLLRE |
| UniProtKB | KTHGLVVENQELRQRLGMDALVAEEEAEAKGNEVRPVAGS |
| Accession No. | AESAAGAGPVVTPPEHLPMDSGGIDSSDSESDILLGILDNLDP |
| P17861-2 | VMFFKCPSPEPASLEELPEVYPEGPSSLPASLSLSVGTSSAKLE |
| SEQ ID NO: 202 | AINELIRFDHIYTKPLVLEIPSETESQANVVVKIEEAPLSPSEN |
| | DHPEFIVSVKEEPVEDDLVPELGISNLLSSSHCPKPSSCLLDA |
| | YSDCGYGGSLSPFSDMSSLLGVNHSWEDTFANELFPQLISV |
| Transcription factor | MTAKMETTFYDDALNASFLPSESGPYGYSNPKILKQSMTLN |
| AP-1 (c-Jun), | LADPVGSLKPHLRAKNSDLLTSPDVGLLKLASPELERLIIQSS |
| isoform 1, | NGHITTTPTPTQFLCPKNVTDEQEGFAEGFVRALAELHSQNT |
| UniProtKB | LPSVTSAAQPVNGAGMVAPAVASVAGGSGSGGFSASLHSEP |
| Accession No. | PVYANLSNFNPGALSSGGGAPSYGAAGLAFPAQPQQQQQPP |
| P05412-1 | HHLPQQMPVQHPRLQALKEEPQTVPEMPGETPPLSPIDMESQ |
| SEQ ID NO: 203 | ERIKAERKRMRNRIAASKCRKRKLERIARLEEKVKTLKAQNS |
| | ELASTANMLREQVAQLKQKVMNHVNSGCQLMLTQQLQTF |
| Proto-oncogene c- | MMFSGFNADYEASSSRCSSASPAGDSLSYYHSPADSFSSMGS |
| Fos (Fos), isoform 1, | PVNAQDFCTDLAVSSANFIPTVTAISTSPDLQWLVQPALVSS |
| UniProtKB | VAPSQTRAPHPFGVPAPSAGAYSRAGVVKTMTGGRAQSIGR |
| Accession No. | RGKVEQLSPEEEEKRRIRRERNKMAAAKCRNRRRELTDTLQ |
| P01100-1 | AETDQLEDEKSALQTEIANLLKEKEKLEFILAAHRPACKIPDD |
| SEQ ID NO: 204 | LGFPEEMSVASLDLTGGLPEVATPESEEAFTLPLLNDPEPKPS |
| | VEPVKSISSMELKTEPFDDFLFPASSRPSGSETARSVPDMDLS |
| | GSFYAADWEPLHSGSLGMGPMATELEPLCTPVVTCTPSCTA |
| | YTSSFVFTYPEADSFPSCAAAHRKGSSSNEPSSDSLSSPTLLA |
| | L |
| Proto-oncogene c- | MTGGRAQSIGRRGKVEQLSPEEEEKRRIRRERNKMAAAKCR |
| Fos (Fos), isoform 2, | NRRRELTDTLQAETDQLEDEKSALQTEIANLLKEKEKLEFIL |
| UniProtKB | AAHRPACKIPDDLGFPEEMSVASLDLTGGLPEVATPESEEAF |
| Accession No. | TLPLLNDPEPKPSVEPVKSISSMELKTEPFDDFLFPASSRPSGS |
| P01100-2 | ETARSVPDMDLSGSFYAADWEPLHSGSLGMGPMATELEPLC |
| SEQ ID NO: 205 | TPVVTCTPSCTAYTSSFVFTYPEADSFPSCAAAHRKGSSSNEP |
| | SSDSLSSPTLLAL |
| Proto-oncogene c- | MMFSGFNADYEASSSRCSSASPAGDSLSYYHSPADSFSSMGS |
| Fos (Fos), isoform 3, | PVNAQDFCTDLAVSSANFIPTVTAISTSPDLQWLVQPALVSS |
| UniProtKB | VAPSQTRAPHPFGVPAPSAGAYSRAGVVKTMTGGRAQSIGR |
| Accession No. | RGKVEQETDQLEDEKSALQTEIANLLKEKEKLEFILAAHRPA |
| P01100-3 | CKIPDDLGFPEEMSVASLDLTGGLPEVATPESEEAFTLPLLND |
| SEQ ID NO: 206 | PEPKPSVEPVKSISSMELKTEPFDDFLFPASSRPSGSETARSVP |
| | DMDLSGSFYAADWEPLHSGSLGMGPMATELEPLCTPVVTCT |
| | PSCTAYTSSFVFTYPEADSFPSCAAAHRKGSSSNEPSSDSLSSP |
| | TLLAL |
| AP-1 Complex | MQFMLLFSRQGKLRLQKWYVPLSDKEKKKITRELVQTVLA |
| Subunit sigma 2 | RKPKMCSFLEWRDLKIVYKRYASLYFCCAIEDQDNELITLEII |
| (AP1S2), isoform 1, | HRYVELLDKYFGSVCELDIIFNFEKAYFILDEFLLGGEVQETS |
| UniProtKB | KKNVLKAIEQADLLQEEAETPRSVLEEIGLT |
| Accession No. | |
| P56377-1 | |
| SEQ ID NO: 207 | |
| AP-1 Complex | MPAGCPPHSTTASLPQHGDRGFPFAAAAAAGQAPPRPRPAA |
| Subunit sigma 2 | AMQFMLLFSRQGKLRLQKWYVPLSDKEKKKITRELVQTVL |
| (AP1S2), isoform 2, | ARKPKMCSFLEWRDLKIVYKRYASLYFCCAIEDQDNELITLE |
| UniProtKB | IIHRYVELLDKYFGSVCELDIIFNFEKAYFILDEFLLGGEVQET |
| Accession No. | SKKNVLKAIEQADLLQEKTETMYHSKSFIGFKKAY |
| P56377-2 | |
| SEQ ID NO: 208 | |
| AP-1 complex | MMRFMLLFSRQGKLRLQKWYLATSDKERKKMVRELMQVV |
| subunit sigma-1A | LARKPKMCSFLEWRDLKVVYKRYASLYFCCAIEGQDNELIT |
| (AP1S1), isoform 1, | LELIHRYVELLDKYFGSVCELDIIFNFEKAYFILDEFLMGGDV |
| UniProtKB | QDTSKKSVLKAIEQADLLQEEDESPRSVLEEMGLA |
| Accession No. | |
| P61966-1 | |
| SEQ ID NO: 209 | |
| AP-1 complex | MMRFMLLFSRQGKLRLQKWYLATSDKERKKMVRELMQVV |
| subunit sigma-1A | LARKPKMCSFLEWRDLKVVYKRYASLYFCCAIEGQDNELIT |
| (AP1S1), isoform 2, | LELIHRYVELLDKYFGSVCELDIIFNFEKAYFILDEFLMGGDV |
| UniProtKB | QDTSTFPFSH |
| Accession No. | |
| P61966-2 | |
| SEQ ID NO: 210 | |
| AP-1 Complex | MIHFILLFSRQGKLRLQKWYITLPDKERKKITREIVQIILSRGH |
| Subunit sigma 3 | RTSSFVDWKELKLVYKRYASLYFCCAIENQDNELLTLEIVHR |
| (AP1S3), isoform 1, | YVELLDKYFGNVCELDIIFNFEKAYFILDEFIIGGEIQETSKKI |
| UniProtKB | AVKAIEDSDMLQEVSTVSQTMGER |
| Accession No. | |
| Q96PC3-1 | |
| SEQ ID NO: 211 | |
| AP-1 Complex | MIHFILLFSRQGKLRLQKWYITLPDKERKKITREIVQIILSRGH |
| Subunit sigma 3 | RTSSFVDWKELKLVYKRYASLYFCCAIENQDNELLTLEIVHR |
| (AP1S3), isoform 2, | YVELLDKYFGNVCELDIIFNFEKAYFILDEFIIGGEIQETSKKI |
| UniProtKB | AVKAIEDSDMLQENRLSPRGRDCSEPRSCHCTLA |
| Accession No. | |
| Q96PC3-2 | |
| SEQ ID NO: 212 | |
| AP-1 Complex | MIHFILLFSRQGKLRLQKWYITLPDKERKKITREIVQIILSRGH |
| Subunit sigma 3 | RTSSFVDWKELKLVYKRYASLYFCCAIENQDNELLTLEIVHR |
| (AP1S3), isoform 3, | YVELLDKYFGNTWPFARA |
| UniProtKB | |
| Accession No. | |
| Q96PC3-3 | |
| SEQ ID NO: 213 | |
| AP-1 Complex | MIHFILLFSRQGKLRLQKWYITLPDKERKKITREIVQIILSRGH |
| Subunit sigma 3 | RTSSFVDWKELKLVYKRYASLYFCCAIENQDNELLTLEIVHR |
| (AP1S3), isoform 4, | YVELLDKYFGNVCELDIIFNFEKAYFILDEFIIGGEIQETSKKI |
| UniProtKB | AVKAIEDSDMLQETMEEYMNKPTF |
| Accession No. | |
| Q96PC3-4 | |
| SEQ ID NO: 214 | |
| AP-1 Complex | MPAPIRLRELIRTIRTARTQAEEREMIQKECAAIRSSFREEDNT |
| Subunit gamma-1 | YRCRNVAKLLYMHMLGYPAHFGQLECLKLIASQKFTDKRIG |
| (AP1G1), isoform 1, | YLGAMLLLDERQDVHLLMTNCIKNDLNHSTQFVQGLALCT |
| UniProtKB | LGCMGSSEMCRDLAGEVEKLLKTSNSYLRKKAALCAVHVIR |
| Accession No. | KVPELMEMFLPATKNLLNEKNHGVLHTSVVLLTEMCERSPD |
| 043747-1 | MLAHFRKLVPQLVRILKNLIMSGYSPEHDVSGISDPFLQVRIL |
| SEQ ID NO: 215 | RLLRILGRNDDDSSEAMNDILAQVATNTETSKNVGNAILYET |
| | VLTIMDIKSESGLRVLAINILGRFLLNNDKNIRYVALTSLLKT |
| | VQTDHNAVQRHRSTIVDCLKDLDVSIKRRAMELSFALVNGN |
| | NIRGMMKELLYFLDSCEPEFKADCASGIFLAAEKYAPSKRW |
| | HIDTIMRVLTTAGSYVRDDAVPNLIQLITNSVEMHAYTVQRL |
| | YKAILGDYSQQPLVQVAAWCIGEYGDLLVSGQCEEEEPIQV |
| | TEDEVLDILESVLISNMSTSVTRGYALTAIMKLSTRFTCTVNR |
| | IKKVVSIYGSSIDVELQQRAVEYNALFKKYDHMRSALLERM |
| | PVMEKVTTNGPTEIVQTNGETEPAPLETKPPPSGPQPTSQAN |
| | DLLDLLGGNDITPVIPTAPTSKPSSAGGELLDLLGDINLTGAP |
| | AAAPAPASVPQISQPPFLLDGLSSQPLENDIAAGIPSITAYSKN |
| | GLKIEFTFERSNTNPSVTVITIQASNSTELDMTDFVFQAAVPK |
| | TFQLQLLSPSSSIVPAFNTGTITQVIKVLNPQKQQLRMRIKLT |
| | YNHKGSAMQDLAEVNNFPPQSWQ |
| AP-1 Complex | MPAPIRLRELIRTIRTARTQAEEREMIQKECAAIRSSFREEDNT |
| Subunit gamma-1 | YRCRNVAKLLYMHMLGYPAHFGQLECLKLIASQKFTDKRIG |
| (AP1G1), isoform 2, | YLGAMLLLDERQDVHLLMTNCIKNDLNHSTQFVQGLALCT |
| UniProtKB | LGCMGSSEMCRDLAGEVEKLLKTSNSYLRKKAALCAVHVIR |
| Accession No. | KVPELMEMFLPATKNLLNEKNHGVLHTSVVLLTEMCERSPD |
| 043747-2 | MLAHFRKNEKLVPQLVRILKNLIMSGYSPEHDVSGISDPFLQ |
| SEQ ID NO: 216 | VRILRLLRILGRNDDDSSEAMNDILAQVATNTETSKNVGNAI |
| | LYETVLTIMDIKSESGLRVLAINILGRFLLNNDKNIRYVALTS |
| | LLKTVQTDHNAVQRHRSTIVDCLKDLDVSIKRRAMELSFAL |
| | VNGNNIRGMMKELLYFLDSCEPEFKADCASGIFLAAEKYAP |
| | SKRWHIDTIMRVLTTAGSYVRDDAVPNLIQLITNSVEMHAY |
| | TVQRLYKAILGDYSQQPLVQVAAWCIGEYGDLLVSGQCEEE |
| | EPIQVTEDEVLDILESVLISNMSTSVTRGYALTAIMKLSTRFT |
| | CTVNRIKKVVSIYGSSIDVELQQRAVEYNALFKKYDHMRSA |
| | LLERMPVMEKVTTNGPTEIVQTNGETEPAPLETKPPPSGPQP |
| | TSQANDLLDLLGGNDITPVIPTAPTSKPSSAGGELLDLLGDIN |
| | LTGAPAAAPAPASVPQISQPPFLLDGLSSQPLENDIAAGIPSIT |
| | AYSKNGLKIEFTFERSNTNPSVTVITIQASNSTELDMTDFVFQ |
| | AAVPKTFQLQLLSPSSSIVPAFNTGTITQVIKVLNPQKQQLRM |
| | RIKLTYNHKGSAMQDLAEVNNFPPQSWQ |
| AP-1 Complex | MSASAVYVLDLKGKVLICRNYRGDVDMSEVEHFMPILMEK |
| Subunit mu-1 | EEEGMLSPILAHGGVRFMWIKHNNLYLVATSKKNACVSLVF |
| (AP1M1), isoform 1, | SFLYKVVQVFSEYFKELEEESIRDNFVIIYELLDELMDFGYPQ |
| UniProtKB | TTDSKILQEYITQEGHKLETGAPRPPATVTNAVSWRSEGIKY |
| Accession No. | RKNEVFLDVIESVNLLVSANGNVLRSEIVGSIKMRVFLSGMP |
| Q9BXS5-1 | ELRLGLNDKVLFDNTGRGKSKSVELEDVKFHQCVRLSRFEN |
| SEQ ID NO: 217 | DRTISFIPPDGEFELMSYRLNTHVKPLIWIESVIEKHSHSRIEY |
| | MIKAKSQFKRRSTANNVEIHIPVPNDADSPKFKTTVGSVKW |
| | VPENSEIVWSIKSFPGGKEYLMRAHFGLPSVEAEDKEGKPPIS |
| | VKFEIPYFTTSGIQVRYLKIIEK |
| | SGYQALPWVRYITQNGDYQLRTQ |
| AP-1 Complex | MSASAVYVLDLKGKVLICRNYRGDVDMSEVEHFMPILMEK |
| Subunit mu-1 | EEEGMLSPILAHGGVRFMWIKHNNLYLVATSKKNACVSLVF |
| (AP1M1), isoform 2, | SFLYKVVQVFSEYFKELEEESIRDNFVIIYELLDELMDFGYPQ |
| UniProtKB | TTDSKILQEYITQEGHKLETGAPRPPATVTNAVSWRSEGIKY |
| Accession No. | RKNEVFLDVIESVNLLGKYPGVGWLGHTVSANGNVLRSEIV |
| Q9BXS5-2 | GSIKMRVFLSGMPELRLGLNDKVLFDNTGRGKSKSVELEDV |
| SEQ ID NO: 218 | KF |
| | HQCVRLSRFENDRTISFIPPDGEFELMSYRLNTHVKPLIWIES |
| | VIEKHSHSRIEYMIKAKSQFKRRSTANNVEIHIPVPNDADSPK |
| | FKTTVGSVKWVPENSEIVWSIKSFPGGKEYLMRAHFGLPSVE |
| | AEDKEGKPPISVKFEIPYFTTSGIQVRYLKIIEKSGYQALPWV |
| | RYITQNGDYQLRTQ |
| AP-1 Complex | MTDSKYFTTTKKGEIFELKAELNSDKKEKKKEAVKKVIASM |
| Subunit beta-1 | TVGKDVSALFPDVVNCMQTDNLELKKLVYLYLMNYAKSQP |
| (AP1B1), isoform A, | DMAIMAVNTFVKDCEDPNPLIRALAVRTMGCIRVDKITEYL |
| UniProtKB | CEPLRKCLKDEDPYVRKTAAVCVAKLHDINAQLVEDQGFLD |
| Accession No. | TLKDLISDSNPMVVANAVAALSEIAESHPSSNLLDLNPQSINK |
| Q10567-1 | LLTALNECTEWGQIFILDCLANYMPKDDREAQSICERVTPRL |
| SEQ ID NO: 219 | SHANSAVVLSAVKVLMKFMEMLSKDLDYYGTLLKKLAPPL |
| | VTLLSAEPELQYVALRNINLIVQKRPEILKHEMKVFFVKYND |
| | PIYVKLEKLDIMIRLASQANIAQVLAELKEYATEVDVDFVRK |
| | AVRAIGRCAIKVEQSAERCVSTLLDLIQTKVNYVVQEAIVVI |
| | KDIFRKYPNKYESVIATLCENLDSLDEPEARAAMIWIVGEYA |
| | ERIDNADELLESFLEGFHDESTQVQLQLLTAIVKLFLKKPTET |
| | QELVQQVLSLATQDSDNPDLRDRGYIYWRLLSTDPVAAKEV |
| | VLAEKPLISEETDLIEPTLLDELICYIGTLASVYHKPPSAFVEG |
| | GRGVVHKSLPPRTASSESAESPETAPTGAPPGEQPDVIPAQG |
| | DLLGDLLNLDLGPPVSGPPLATSSVQMGAVDLLGGGLDSLM |
| | GDEPEGIGGTNFVAPPTAAVPANLGAPIGSGLSDLFDLTSGV |
| | GTLSGSYVAPKAVWLPAMKAKGLEISGTFTRQVGSISMDLQ |
| | LTNKALQVMTDFAIQFNRNSFGLAPATPLQVHAPLSPNQTVE |
| | ISLPLSTVGSVMKMEPLNNLQVAVKNNIDVFYFSTLYPLHILF |
| | VEDGKMDRQMFLATWKDIPNENEAQFQIRDCPLNAEAASSK |
| | LQSSNIFTVAKRNVEGQDMLYQSLKLTNGIWVLAELRIQPG |
| | NPSCTDLELSLKCRAPEVSQHVYQAYETILKN |
| AP-1 Complex | MTDSKYFTTTKKGEIFELKAELNSDKKEKKKEAVKKVIASM |
| Subunit beta-1 | TVGKDVSALFPDVVNCMQTDNLELKKLVYLYLMNYAKSQP |
| (AP1B1), isoform B, | DMAIMAVNTFVKDCEDPNPLIRALAVRTMGCIRVDKITEYL |
| UniProtKB | CEPLRKCLKDEDPYVRKTAAVCVAKLHDINAQLVEDQGFLD |
| Accession No. | TLKDLISDSNPMVVANAVAALSEIAESHPSSNLLDLNPQSINK |
| Q10567-2 | LLTALNECTEWGQIFILDCLANYMPKDDREAQSICERVTPRL |
| SEQ ID NO: 220 | SHANSAVVLSAVKVLMKFMEMLSKDLDYYGTLLKKLAPPL |
| | VTLLSAEPELQYVALRNINLIVQKRPEILKHEMKVFFVKYND |
| | PIYVKLEKLDIMIRLASQANIAQVLAELKEYATEVDVDFVRK |
| | AVRAIGRCAIKVEQSAERCVSTLLDLIQTKVNYVVQEAIVVI |
| | KDIFRKYPNKYESVIATLCENLDSLDEPEARAAMIWIVGEYA |
| | ERIDNADELLESFLEGFHDESTQVQLQLLTAIVKLFLKKPTET |
| | QELVQQVLSLATQDSDNPDLRDRGYIYWRLLSTDPVAAKEV |
| | VLAEKPLISEETDLIEPTLLDELICYIGTLASVYHKPPSAFVEG |
| | GRGVVHKSLPPRTASSESAESPETAPTGAPPGEQPDVIPAQG |
| | DLLGDLLNLDLGPPVSGPPLATSSVQMGAVDLLGGGLDSLIG |
| | GTNFVAPPTAAVPANLGAPIGSGLSDLFDLTSGVGTLSGSYV |
| | APKAVWLPAMKAKGLEISGTFTRQVGSISMDLQLTNKALQV |
| | MTDFAIQFNRNSFGLAPATPLQVHAPLSPNQTVEISLPLSTVG |
| | SVMKMEPLNNLQVAVKNNIDVFYFSTLYPLHILFVEDGKMD |
| | RQMFLATWKDIPNENEAQFQIRDCPLNAEAASSKLQSSNIFT |
| | VAKRNVEGQDMLYQSLKLINGIWVLAELRIQPGNPSCTDLE |
| | LSLKCRAPEVSQHVYQAYETILKN |
| AP-1 Complex | MTDSKYFTTTKKGEIFELKAELNSDKKEKKKEAVKKVIASM |
| Subunit beta-1 | TVGKDVSALFPDVVNCMQTDNLELKKLVYLYLMNYAKSQP |
| (AP1B1), isoform C, | DMAIMAVNTFVKDCEDPNPLIRALAVRTMGCIRVDKITEYL |
| UniProtKB | CEPLRKCLKDEDPYVRKTAAVCVAKLHDINAQLVEDQGFLD |
| Accession No. | TLKDLISDSNPMVVANAVAALSEIAESHPSSNLLDLNPQSINK |
| Q10567-3 | LLTALNECTEWGQIFILDCLANYMPKDDREAQSICERVTPRL |
| SEQ ID NO: 221 | SHANSAVVLSAVKVLMKFMEMLSKDLDYYGTLLKKLAPPL |
| | VTLLSAEPELQYVALRNINLIVQKRPEILKHEMKVFFVKYND |
| | PIYVKLEKLDIMIRLASQANIAQVLAELKEYATEVDVDFVRK |
| | AVRAIGRCAIKVEQSAERCVSTLLDLIQTKVNYVVQEAIVVI |
| | KDIFRKYPNKYESVIATLCENLDSLDEPEARAAMIWIVGEYA |
| | ERIDNADELLESFLEGFHDESTQVQLQLLTAIVKLFLKKPTET |
| | QELVQQVLSLATQDSDNPDLRDRGYIYWRLLSTDPVAAKEV |
| | VLAEKPLISEETDLIEPTLLDELICYIGTLASVYHKPPSAFVEG |
| | GRGVVHKSLPPRTASSESAESPETAPTGAPPGEQPDVIPAQG |
| | DLLGDLLNLDLGPPVSGPPLATSSVQMGAVDLLGGGLDSLIG |
| | GTNFVAPPTAAVPANLGAPIGSGLSDLFDLTSGVGTLSGSYV |
| | APKAVWLPAMKAKGLEISGTFTRQVGSISMDLQLTNKALQV |
| | MTDFAIQFNRNSFGLAPATPLQVHAPLSPNQTVEISLPLSTVG |
| | SVMKMEPLNNLQVAVKNNIDVFYFSTLYPLHILFVEDGKMD |
| | RQMFLATWKDIPNENEAQFQIRDCPLNAEAASSKLQSSNIFT |
| | VAKRNVEGQDMLYQSLKLINGIWVLAELRIQPGNPSCTLSL |
| | KCRAPEVSQHVYQAYETILKN |
| AP-1 Complex | MTDSKYFTTTKKGEIFELKAELNSDKKEKKKEAVKKVIASM |
| Subunit beta-1 | TVGKDVSALFPDVVNCMQTDNLELKKLVYLYLMNYAKSQP |
| (AP1B1), isoform 4, | DMAIMAVNTFVKDCEDPNPLIRALAVRTMGCIRVDKITEYL |
| UniProtKB | CEPLRKCLKDEDPYVRKTAAVCVAKLHDINAQLVEDQGELD |
| Accession No. | TLKDLISDSNPMVVANAVAALSEIAESHPSSNLLDLNPQSINK |
| Q10567-4 | LLTALNECTEWGQIFILDCLANYMPKDDREAQSICERVTPRL |
| SEQ ID NO: 222 | SHANSAVVLSAVKVLMKFMEMLSKDLDYYGTLLKKLAPPL |
| | VTLLSAEPELQYVALRNINLIVQKRPEILKHEMKVFFVKYND |
| | PIYVKLEKLDIMIRLASQANIAQVLAELKEYATEVDVDFVRK |
| | AVRAIGRCAIKVEQSAERCVSTLLDLIQTKVNYVVQEAIVVI |
| | KDIFRKYPNKYESVIATLCENLDSLDEPEARAAMIWIVGEYA |
| | ERIDNADELLESFLEGFHDESTQVQLQLLTAIVKLFLKKPTET |
| | QELVQQVLSLATQDSDNPDLRDRGYIYWRLLSTDPVAAKEV |
| | VLAEKPLISEETDLIEPTLLDELICYIGTLASVYHKPPSAFVEG |
| | GRGVVHKSLPPRTASSESAESPETAPTGAPPGEQPDVIPAQG |
| | DLLGDLLNLDLGPPVSGPPLATSSVQMGAVDLLGGGLDSLIG |
| | GTNFVAPPTAAVPANLGAPIGSGLSDLFDLTSGVGTLSGSYV |
| | APKAVGSISMDLQLTNKALQVMTDFAIQFNRNSFGLAPATPL |
| | QVHAPLSPNQTVEISLPLSTVGSVMKMEPLNNLQVAVKNNI |
| | DVFYFSTLYPLHILFVEDGKMDRQMFLATWKDIPNENEAQF |
| | QIRDCPLNAEAASSKLQSSNIFTVAKRNVEGQDMLYQSLKLT |
| | NGIWVLAELRIQPGNPSCTLSLKCRAPEVSQHVYQAYETILK |
| | N |
| AP-1 Complex | MSASAVFILDVKGKPLISRNYKGDVAMSKIEHFMPLLVQREE |
| Subunit mu-2 | EGALAPLLSHGQVHFLWIKHSNLYLVATTSKNANASLVYSF |
| (AP1M2), isoform 1, | LYKTIEVFCEYFKELEEESIRDNFVIVYELLDELMDFGFPQTT |
| UniProtKB | DSKILQEYITQQSNKLETGKSRVPPTVTNAVSWRSEGIKYKK |
| Accession No. | NEVFIDVIESVNLLVNANGSVLLSEIVGTIKLKVFLSGMPELR |
| Q9Y6Q5-1 | LGLNDRVLFELTGRSKNKSVELEDVKFHQCVRLSRFDNDRTI |
| SEQ ID NO: 223 | SFIPPDGDFELMSYRLSTQVKPLIWIESVIEKFSHSRVEIMVKA |
| | KGQFKKQSVANGVEISVPVPSDADSPRFKTSVGSAKYVPER |
| | NVVIWSIKSFPGGKEYLMRAHFGLPSVEKEEVEGRPPIGVKF |
| | EIPYFTVSGIQVRYMKIIEKSGYQALPWVRYITQSGDYQLRTS |
| AP-1 Complex | MSASAVFILDVKGKPLISRNYKGDVAMSKIEHFMPLLVQREE |
| Subunit mu-2 | EGALAPLLSHGQVHFLWIKHSNLYLVATTSKNANASLVYSF |
| (AP1M2), isoform 2, | LYKTIEVFCEYFKELEEESIRDNFVIVYELLDELMDFGFPQTT |
| UniProtKB | DSKILQEYITQQSNKLETGKSRVPPTVTNAVSWRSEGIKYKK |
| Accession No. | NEVFIDVIESVNLLVNANGSVLLSEIVGTIKLKVFLSGMPELR |
| Q9Y6Q5-2 | LGLNDRVLFELTGLSGSKNKSVELEDVKFHQCVRLSRFDND |
| SEQ ID NO: 224 | RTISFIPPDGDFELMSYRLSTQVKPLIWIESVIEKFSHSRVEIM |
| | VKAKGQFKKQSVANGVEISVPVPSDADSPRFKTSVGSAKYV |
| | PERNVVIWSIKSFPGGKEYLMRAHFGLPSVEKEEVEGRPPIG |
| | VKFEIPYFTVSGIQVRYMKIIEKSGYQALPWVRYITQSGDYQ |
| | LRTS |
| AP-1 Complex | MVVPSLKLQDLIEEIRGAKTQAQEREVIQKECAHIRASFRDG |
| Subunit gamma-2 | DPVHRHRQLAKLLYVHMLGYPAHFGQMECLKLIASSRFTDK |
| (AP1G2), isoform 1, | RVGYLGAMLLLDERHDAHLLITNSIKNDLSQGIQPVQGLALC |
| UniProtKB | TLSTMGSAEMCRDLAPEVEKLLLQPSPYVRKKAILTAVHMI |
| Accession No. | RKVPELSSVFLPPCAQLLHERHHGILLGTITLITELCERSPAAL |
| 075843-1 | RHFRKVVPQLVHILRTLVTMGYSTEHSISGVSDPFLQVQILRL |
| SEQ ID NO: 225 | LRILGRNHEESSETMNDLLAQVATNTDTSRNAGNAVLFETV |
| | LTIMDIRSAAGLRVLAVNILGRFLLNSDRNIRYVALTSLLRLV |
| | QSDHSAVQRHRPTVVECLRETDASLSRRALELSLALVNSSNV |
| | RAMMQELQAFLESCPPDLRADCASGILLAAERFAPTKRWHI |
| | DTILHVLTTAGTHVRDDAVANLTQLIGGAQELHAYSVRRLY |
| | NALAEDISQQPLVQVAAWCIGEYGDLLLAGNCEEIEPLQVDE |
| | EEVLALLEKVLQSHMSLPATRGYALTALMKLSTRLCGDNNR |
| | IRQVVSIYGSCLDVELQQRAVEYDTLFRKYDHMRAAILEKM |
| | PLVERDGPQADEEAKESKEAAQLSEAAPVPTEPQASQLLDLL |
| | DLLDGASGDVQHPPHLDPSPGGALVHLLDLPCVPPPPAPIPD |
| | LKVFEREGVQLNLSFIRPPENPALLLITITATNFSEGDVTHFIC |
| | QAAVPKSLQLQLQAPSGNTVPARGGLPITQLFRILNPNKAPL |
| | RLKLRLTYDHFHQSVQEIFEVNNLPVESWQ |
| BTB and CNC | MSVDEKPDSPMYVYESTVHCTNILLGLNDQRKKDILCDVTLI |
| homolog 2 | VERKEFRAHRAVLAACSEYFWQALVGQTKNDLVVSLPEEV |
| (BACH2), isoform | TARGFGPLLQFAYTAKLLLSRENIREVIRCAEFLRMHNLEDS |
| 1, UniProtKB | CFSFLQTQLLNSEDGLFVCRKDAACQRPHEDCENSAGEEED |
| Accession No. | EEEETMDSETAKMACPRDQMLPEPISFEAAAIPVAEKEEALL |
| Q9BYV9-1 | PEPDVPTDTKESSEKDALTQYPRYKKYQLACTKNVYNASSH |
| SEQ ID NO: 226 | STSGFASTFREDNSSNSLKPGLARGQIKSEPPSEENEEESITLC |
| | LSGDEPDAKDRAGDVEMDRKQPSPAPTPTAPAGAACLERSR |
| | SVASPSCLRSLFSITKSVELSGLPSTSQQHFARSPACPFDKGIT |
| | QGDLKTDYTPFTGNYGQPHVGQKEVSNFTMGSPLRGPGLEA |
| | LCKQEGELDRRSVIFSSSACDQVSTSVHSYSGVSSLDKDLSEP |
| | VPKGLWVGAGQSLPSSQAYSHGGLMADHLPGRMRPNTSCP |
| | VPIKVCPRSPPLETRTRTSSSCSSYSYAEDGSGGSPCSLPLCEF |
| | SSSPCSQGARFLATEHQEPGLMGDGMYNQVRPQIKCEQSYG |
| | TNSSDESGSFSEADSESCPVQDRGQEVKLPFPVDQITDLPRND |
| | FQMMIKMHKLTSEQLEFIHDVRRRSKNRIAAQRCRKRKLDCI |
| | QNLECEIRKLVCEKEKLLSERNQLKACMGELLDNFSCLSQEV |
| | CRDIQSPEQIQALHRYCPVLRPMDLPTASSINPAPLGAEQNIA |
| | ASQCAVGENVPCCLEPGAAPPGPPWAPSNTSENCTSGRRLE |
| | GTD |
| | PGTFSERGPPLEPRSQTVTVDFCQEMTDKCTTDEQPRKDYT |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 4L, UniProtKB | EHFNSPHPTPAPADISQKQVHRPLQTPDLSGFYSLTSGSMGQ |
| Accession No. | LPHTVSWFTHPSLMLGSGVPGHPAAIPHP AIVPPSGKQELQPF |
| P36402-1 | DR |
| SEQ ID NO: 227 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTETNWPRELKDGNGQ |
| | ESLSMSSSSSPA |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 4S, UniProtKB | MRAKVIAECTLKESAAINQILGRRWHALSREEQAKYYELAR |
| Accession No. | KERQLHMQLYPGWSARDNYGKKKRRSREKHQESTTETNWP |
| P36402-2 | RELKDGNGQESLSMSSSSSPA |
| SEQ ID NO: 228 | |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 1L, UniProtKB | EHFNSPHPTPAPADISQKQVHRPLQTPDLSGFYSLTSGSMGQ |
| Accession No. | LPHTVSWFTHPSLMLGSGVPGHPAAIPHPAIVPPSGKQELQPF |
| P36402-3 | DR |
| SEQ ID NO: 229 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTDPGSPKKCRARFGL |
| | NQQTDWCGPCRRKKKCIRYLPGEGRCPSPVPSDDSALGCPG |
| | SPAPQDSPSYHLLPRFPTELLTSPAERHLHPQVSPLLSASQPQ |
| | GPHRPPAAPCRAHRYSNRNLRDRWPSRHRTPGRLQEPTP |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 1S, UniProtKB | MRAKVIAECTLKESAAINQILGRRWHALSREEQAKYYELAR |
| Accession No. | KERQLHMQLYPGWSARDNYGKKKRRSREKHQESTTDPGSP |
| P36402-4 | KKCRARFGLNQQTDWCGPCRRKKKCIRYLPGEGRCPSPVPS |
| SEQ ID NO: 230 | DDSALGCPGSPAPQDSPSYHLLPRFPTELLTSPAERHLHPQVS |
| | PLLSASQPQGPHRPPAAPCRAHRYSNRNLRDRWPSRHRTPG |
| | RLQEPTP |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 5L, UniProtKB | EHFNSPHPTPAPADISQKQVHRPLQTPDLSGFYSLTSGSMGQ |
| Accession No. | LPHTVSWFTHPSLMLGSGVPGHPAAIPHPAIVPPSGKQELQPF |
| P36402-9 | DR |
| SEQ ID NO: 231 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTDNSLHYS |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 5S, UniProtKB | MRAKVIAECTLKESAAINQILGRRWHALSREEQAKYYELAR |
| Accession No. | KERQLHMQLYPGWSARDNYGKKKRRSREKHQESTTDNSLH |
| P36402-10 | YS |
| SEQ ID NO: 232 | |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 6L, UniProtKB | EHFNSPHPTPAPADISQKQVHRPLQTPDLSGFYSLTSGSMGQ |
| Accession No. | LPHTVSWFTHPSLMLGSGVPGHPAAIPHPAIVPPSGKQELQPF |
| P36402-11 | DR |
| SEQ ID NO: 233 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTDPGSPKKCRARFGL |
| | NQQTDWCGPCRKKKCIRYLPGEGRCPSPVPSDDSALGCPGSP |
| | APQDSPSYHLLPRFPTELLTSPAERHLHPQVSPLLSASQPQGP |
| | HRPPAAPCRAHRYSNRNLRDRWPSRHRTPGRLQEPTP |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 6S, UniProtKB | MRAKVIAECTLKESAAINQILGRRWHALSREEQAKYYELAR |
| Accession No. | KERQLHMQLYPGWSARDNYGKKKRRSREKHQESTTDPGSP |
| P36402-12 | KKCRARFGLNQQTDWCGPCRKKKCIRYLPGEGRCPSPVPSD |
| SEQ ID NO: 234 | DSALGCPGSPAPQDSPSYHLLPRFPTELLTSPAERHLHPQVSP |
| | LLSASQPQGPHRPPAAPCRAHRYSNRNLRDRWPSRHRTPGR |
| | LQEPTP |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 7L, UniProtKB | EHFNSPHPTPAPADISQKQVHRPLQTPDLSGFYSLTSGSMGQ |
| Accession No. | LPHTVSWFTHPSLMLGSGVPGHPAAIPHP AIVPPSGKQELQPF |
| P36402-13 | DR |
| SEQ ID NO: 235 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTDGIPACTILSP |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 7S, UniProtKB | MRAKVIAECTLKESAAINQILGRRWHALSREEQAKYYELAR |
| Accession No. | KERQLHMQLYPGWSARDNYGKKKRRSREKHQESTTDGIPA |
| P36402-14 | CTILSP |
| SEQ ID NO: 236 | |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 8L, UniProtKB | EHFNSPHPTPAPADISQKQVHRPLQTPDLSGFYSLTSGSMGQ |
| Accession No. | LPHTVSWFTHPSLMLGSGVPGHPAAIPHP AIVPPSGKQELQPF |
| P36402-15 | DR |
| SEQ ID NO: 237 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTQLEDWDGWARKP |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 2L, UniProtKB | EHFNSPHPTPAPADISQKQVHRPLQTPDLSGFYSLTSGSMGQ |
| Accession No. | LPHTVSWFTHPSLMLGSGVPGHPAAIPHPAIVPPSGKQELQPF |
| P36402-5 | DR |
| SEQ ID NO: 239 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTGGKRNAFGTYPEKA |
| | AAPAPFLPMTVL |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHPAIVPPSG |
| (TCF-1)), isoform | KQELQPFDRNLKTQAESKAEKEAKKPTIKKPLNAFMLYMKE |
| 2S, UniProtKB | MRAKVIAECTLKESAAINQILGRRWHALSREEQAKYYELAR |
| Accession No. | KERQLHMQLYPGWSARDNYGKKKRRSREKHQESTTGGKR |
| P36402-6 | NAFGTYPEKAAAPAPFLPMTVL |
| SEQ ID NO: 240 | |
| Transcription factor | MPQLDSGGGGAGGGDDLGAPDELLAFQDEGEEQDDKSRDS |
| 7 (TCF7; also known | AAGPERDLAELKSSLVNESEGAAGGAGIPGVPGAGAGARGE |
| as T-cell factor 1 | AEALGREHAAQRLFPDKLPEPLEDGLKAPECTSGMYKETVY |
| (TCF-1)), isoform | SAFNLLMHYPPPSGAGQHPQPQPPLHKANQPPHGVPQLSLY |
| 3L, UniProtKB | EHFNSPHPTPAPADISQKQVHRPLQTPDLSGFYSLTSGSMGQ |
| Accession No. | LPHTVSWFTHPSLMLGSGVPGHPAAIPHP AIVPPSGKQELQPF |
| P36402-7 | DR |
| SEQ ID NO: 241 | NLKTQAESKAEKEAKKPTIKKPLNAFMLYMKEMRAKVIAEC |
| | TLKESAAINQILGRRWHALSREEQAKYYELARKERQLHMQL |
| | YPGWSARDNYGKKKRRSREKHQESTTDPGSPKKCRARFGL |
| | NQQTDWCGPCR |
| Transcription factor | MYKETVYSAFNLLMHYPPPSGAGQHPQPQPPLHKANQPPHG |
| 7 (TCF7; also known | VPQLSLYEHFNSPHPTPAPADISQKQVHRPLQTPDLSGFYSLT |
| as T-cell factor 1 | SGSMGQLPHTVSWFTHPSLMLGSGVPGHPAAIPHP AIVPPSG |
| (TCF-1)), isoform | KQELQPFDRNLKTQAESKAEKEAKKPTIKKPLNAFMLYMKE |
| 3S, UniProtKB | MRAKVIAECTLKESAAINQILGRRWHALSREEQAKYYELAR |
| Accession No. | KERQLHMQLYPGWSARDNYGKKKRRSREKHQESTTDPGSP |
| P36402-8 | KKCRARFGLNQQTDWCGPCR |
| SEQ ID NO: 242 | |
| Forkhead box | MMQESGTETKSNGSAIQNGSGGSNHLLECGGLREGRSNGET |
| protein P1 (FoxP1), | PAVDIGAADLAHAQQQQQQALQVARQLLLQQQQQQQVSGL |
| isoform 1, | KSPKRNDKQPALQVPVSVAMMTPQVITPQQMQQILQQQVLS |
| UniProKB | PQQLQVLLQQQQALMLQQQQLQEFYKKQQEQLQLQLLQQQ |
| Accession No. | HAGKQPKEQQQVATQQLAFQQQLLQMQQLQQQHLLSLQR |
| Q9H334-1 | QGLLTIQPGQPALPLQPLAQGMIPTELQQLWKEVTSAHTAEE |
| SEQ ID NO: 244 | TTGNNHSSLDLTTTCVSSSAPSKTSLIMNPHASTNGQLSVHTP |
| | KRESLSHEEHPHSHPLYGHGVCKWPGCEAVCEDFQSFLKHL |
| | NSEHALDDRSTAQCRVQMQVVQQLELQLAKDKERLQAMM |
| | THLHVKSTEPKAAPQPLNLVSSVTLSKSASEASPQSLPHTPTT |
| | PTAPLTPVTQGPSVITTTSMHTVGPIRRRYSDKYNVPISSADI |
| | AQNQEFYKNAEVRPPFTYASLIRQAILESPEKQLTLNEIYNWF |
| | TRMFAYFRRNAATWKNAVRHNLSLHKCFVRVENVKGAVW |
| | TVDEVEFQKRRPQKISGNPSLIKNMQSSHAYCTPLNAALQAS |
| | MAENSIPLYTTASMGNPTLGNLASAIREELNGAMEHTNSNES |
| | DSSPGRSPMQAVHPVHVKEEPLDPEEAEGPLSLVTTANHSPD |
| | FDHDRDYEDEPVNEDME |
| Forkhead box | MFQCVFSSSVLQPHSTSCLFKHLFYHSATPASQKQPEPIYSKK |
| protein P1 (FoxP1), | TEIQRQTVRAPFAKLFIFSALQVARQLLLQQQQQQQVSGLKS |
| isoform 3, | PKRNDKQPALQQQQVATQQLAFQQQLLQMQQLQQQHLLSL |
| UniProKB | QRQGLLTIQPGQPALPLQPLAQGMIPTELQQLWKEVTSAHTA |
| Accession No. | EETTGNNHSSLDLTTTCVSSSAPSKTSLIMNPHASTNGQLSV |
| Q9H334-3 | HTPKRESLSHEEHPHSHPLYGHGVCKWPGCEAVCEDFQSFL |
| SEQ ID NO: 245 | KHLNSEHALDDRSTAQCRVQMQVVQQLELQLAKDKERLQA |
| | MMTHLHVKSTEPKAAPQPLNLVSSVTLSKSASEASPQSLPHT |
| | PTTPTAPLTPVTQGPSVITTTSMHTVGPIRRRYSDKYNVPISS |
| | ADIAQNQEFYKNAEVRPPFTYASLIRQAILESPEKQLTLNEIY |
| | NWFTRMFAYFRRNAATWKNAVRHNLSLHKCFVRVENVKG |
| | AVWTVDEVEFQKRRPQKISGNPSLIKNMQSSHAYCTPLNAA |
| | LQ |
| | ASMAENSIPLYTTASMGNPTLGNLASAIREELNGAMEHTNS |
| | NESDSSPGRSPMQAVHPVHVKEEPLDPEEAEGPLSLVTTANH |
| | SPDFDHDRDYEDEPVNEDME |
| Forkhead box | MFQCVFSSSVLQPHSTSCLFKHLFYHSATPASQKQPEPIYSKK |
| protein P1 (FoxP1), | TEIQRQTVRAPFAKLFIFSALQVARQLLLQQQQQQQVSGLKS |
| isoform 4, | PKRNDKQPALQVPVSVAMMTPQVITPQQMQQILQQQVLSPQ |
| UniProKB | QLQVLLQQQQALMLQQQQLQEFYKKQQEQLQLQLLQQQH |
| Accession No. | AGKQPKEQQQVATQQLAFQQQLLQMQQLQQQHLLSLQRQ |
| Q9H334-4 | GLLTIQPGQPALPLQPLAQGMIPTELQQLWKEVTSAHTAEET |
| SEQ ID NO: 246 | TGNNHSSLDLTTTCVSSSAPSKTSLIMNPHASTNGQLSVHTP |
| | KRESLSHEEHPHSHPLYGHGVCKWPGCEAVCEDFQSFLKHL |
| | NSEHALDDRSTAQCRVQMQVVQQLELQLAKDKERLQAMM |
| | THLHVKSTEPKAAPQPLNLVSSVTLSKSASEASPQSLPHTPTT |
| | PTAPLTPVTQGPSVITTTSMHTVGPIRRRYSDKYNVPISSADI |
| | AQNQEFYKNAEVRPPFTYASLIRQAILESPEKQLTLNEIYNWF |
| | TRM |
| | FAYFRRNAATWKNAVRHNLSLHKCFVRVENVKGAVWTVD |
| | EVEFQKRRPQKISGNPSLIKNMQSSHAYCTPLNAALQASMAE |
| | NSIPLYTTASMGNPTLGNLASAIREELNGAMEHTNSNESDSS |
| | PGRSPMQAVHPVHVKEEPLDPEEAEGPLSLVTTANHSPDFD |
| | HDRDYEDEPVNEDME |
| Forkhead box | MMQESGTETKSNGSAIQNGSGGSNHLLECGGLREGRSNGET |
| protein P1 (FoxP1), | PAVDIGAADLAHAQQQQQQWHLINHQPSRSPSSWLKRLISSP |
| isoform 5, | WELEVLQVPLWGAVAETKMSGPVCQPNPSPF |
| UniProKB | |
| Accession No. | |
| Q9H334-5 | |
| SEQ ID NO: 247 | |
| Forkhead box | MMQESGTETKSNGSAIQNGSGGSNHLLECGGLREGRSNGET |
| protein P1 (FoxP1), | PAVDIGAADLAHAQQQQQQALQVARQLLLQQQQQQQVSGL |
| isoform 6, | KSPKRNDKQPALQQQQVATQQLAFQQQLLQMQQLQQQHLL |
| UniProKB | SLQRQGLLTIQPGQPALPLQPLAQGMIPTELQQLWKEVTSAH |
| Accession No. | TAEETTGNNHSSLDLTTTCVSSSAPSKTSLIMNPHASTNGQLS |
| Q9H334-6 | VHTPKRESLSHEEHPHSHPLYGHGVCKWPGCEAVCEDFQSF |
| SEQ ID NO: 248 | LKHLNSEHALDDRSTAQCRVQMQVVQQLELQLAKDKERLQ |
| | AMMTHLHVKSTEPKAAPQPLNLVSSVTLSKSASEASPQSLPH |
| | TPTTPTAPLTPVTQGPSVITTTSMHTVGPIRRRYSDKYNVPISS |
| | ADIAQNQEFYKNAEVRPPFTYASLIRQAILESPEKQLTLNEIY |
| | NWFTRMFAYFRRNAATWKNAVRHNLSLHKCFVRVENVKG |
| | AVWTVDEVEFQKRRPQKISGNPSLIKNMQSSHAYCTPLNAA |
| | LQASMAENSIPLYTTASMGNPTLGNLASAIREELNGAMEHT |
| | NSNESDSSPGRSPMQAVHPVHVKEEPLDPEEAEGPLSLVTTA |
| | NHSPDFDHDRDYEDEPVNEDME |
| Forkhead box | MMQESGTETKSNGSAIQNGSGGSNHLLECGGLREGRSNGET |
| protein P1 (FoxP1), | PAVDIGAADLAHAQQQQQQALQVARQLLLQQQQQQQVSGL |
| isoform 7, | KSPKRNDKQPALQVPVSVAMMTPQVITPQQMQQILQQQVLS |
| UniProKB | PQQLQVLLQQQQALMLQQQQLQEFYKKQQEQLQLQLLQQQ |
| Accession No. | HAGKQPKEQQQVATQQLAFQQQLLQMQQLQQQHLLSLQ |
| | RQGLLTIQPGQPALPLQPLAQGMIPTELQQLWKEVTSAHTAE |
| | ETTGNNHSSLDLTTTCVSSSAPSKTSLIMNPHASTNGQLSVH |
| | TPKRESLSHEEHPHSHPLYGHGVCKWPGCEAVCEDFQSFLK |
| | HLNSEHALDDRSTAQCRVQMQVVQQLELQLAKDKERLQA |
| | MMTHLHVKSTEPKAAPQPLNLVSSVTLSKSASEASPQSLPHT |
| | PTTPTAPLTPVTQGPSVITTTSMHTVGPIRRRYSDKYNVPISS |
| | D |
| Q9H334-7 | IAQNQEFYKNAEVRPPFTYASLIRQAILESPEKQLTLNEIYNW |
| SEQ ID NO: 249 | FTRMFAYFRRNAATWKNAVRHNLSLHKCFVRVENVKGAV |
| | WTVDEVEFQKRRPQKISGNPSLIKNMQSSHAYCTPLNAALQ |
| | ASMAENSIPLYTTASMGNPTLGNLASAIREELNGAMEHTNS |
| | NESDSSPGRSPMQAVHPVHVKEEPLDPEEAEGPLSLVTTANH |
| | SPDFDHDRDYEDEPVNEDME |
| Forkhead box | MMQESGTETKSNGSAIQNGSGGSNHLLECGGLREGRSNGET |
| protein P1 (FoxP1), | PAVDIGAADLAHAQQQQQQALQVARQLLLQQQQQQQVSGL |
| isoform 8, | KSPKRNDKQPALQVPVSVAMMTPQVITPQQMQQILQQQVLS |
| UniProKB | PQQLQVLLQQQQALMLQQQQLQEFYKKQQEQLQLQLLQQQ |
| Accession No. | HAGKQPKEQQQVATQQLAFQQQLLQMQQLQQQHLLSLQR |
| Q9H334-8 | QGLLTIQPGQPALPLQPLAQGMIPTELQQLWKEVTSAHTAEE |
| SEQ ID NO: 250 | TTGNNHSSLDLTTTCVSSSAPSKTSLIMNPHASTNGQLSVHTP |
| | KRESLSHEEHPHSHPLYGHGVCKWPGCEAVCEDFQSFLKHL |
| | NSEHALDDRSTAQCRVQMQVVQQLELQLAKDKERLQAMM |
| | THLHVKSTEPKAAPQPLNLVSSVTLSKSASEASPQSLPHTPTT |
| | PTAPLTPVTQGPSVITTTSMHTVGPIRRRYSDKYNVPISSADI |
| | AQNQEFYKNAEVRPPFTYASLIRQAILESPEKQLTLNEIYNWF |
| | TRMFAYFRRNAATWKGAIRTNLSLHK CFIRVEDEFGSFWTV |
| | DDEEFKRGRHIQRGRPRKYCPDENFDELVAHNPSLIKNMQSS |
| | HAYCTPLNAALQASMAENSIPLYTTASMGNPTLGNLASAIRE |
| | ELNGAMEHTNSNESDSSPGRSPMQAVHPVHVKEEPLDPEEA |
| | EGPLSLVTTANHSPDFDHDRDYEDEPVNEDME |
| Forkhead box | MPNPRPGKPSAPSLALGPSPGASPSWRAAPKASDLLGARGPG |
| protein P1 (FoxP3), | GTFQGRDLRGGAHASSSSLNPMPPSQLQLPTLPLVMVAPSG |
| isoform 1, | ARLGPLPHLQALLQDRPHFMHQLSTVDAHARTPVLQVHPLE |
| UniProKB | SPAMISLTPPTTATGVFSLKARPGLPPGINVASLEWVSREPAL |
| Accession No. | LCTFPNPSAPRKDSTLSAVPQSSYPLLANGVCKWPGCEKVFE |
| Q9BZS1-1 | EPEDFLKHCQADHLLDEKGRAQCLLQREMVQSLEQQLVLE |
| SEQ ID NO: 251 | K |
| | EKLSAMQAHLAGKMALTKASSVASSDKGSCCIVAAGSQGP |
| | VVPAWSGPREAPDSLFAVRRHLWGSHGNSTFPEFLHNMDYF |
| | KFHNMRPPFTYATLIRWAILEAPEKQRTLNEIYHWFTRMFAF |
| | FRNHPATWKNAIRHNLSLHKCFVRVES |
| | EKGAVWTVDELEFRKKRSQRPSRCSNPTPGP |
| Forkhead box | MPNPRPGKPSAPSLALGPSPGASPSWRAAPKASDLLGARGPG |
| protein P1 (FoxP3), | GTFQGRDLRGGAHASSSSLNPMPPSQLQLSTVDAHARTPVL |
| isoform 2, | QVHPLESPAMISLTPPTTATGVFSLKARPGLPPGINVASLEWV |
| UniProKB | SREPALLCTFPNPSAPRKDSTLSAVPQSSYPLLANGVCKWPG |
| Accession No. | CEKVFEEPEDFLKHCQADHLLDEKGRAQCLLQREMVQSLEQ |
| Q9BZS1-2 | QLVLEKEKLSAMQAHLAGKMALTKASSVASSDKGSCCIVA |
| SEQ ID NO: 252 | AGSQGPVVPAWSGPREAPDSLFAVRRHLWGSHGNSTFPEFL |
| | HNMDYFKFHNMRPPFTYATLIRWAILEAPEKQRTLNEIYHW |
| | FTRMFAFFRNHPATWKNAIRHNLSLHKCFVRVESEKGAVWT |
| | VDELEFRKKRSQRPSRCSNPTPGP |
| Forkhead box | MPNPRPGKPSAPSLALGPSPGASPSWRAAPKASDLLGARGPG |
| protein P1 (FoxP3), | GTFQGRDLRGGAHASSSSLNPMPPSQLQLSTVDAHARTPVL |
| isoform 3, | QVHPLESPAMISLTPPTTATGVFSLKARPGLPPGINVASLEWV |
| UniProKB | SREPALLCTFPNPSAPRKDSTLSAVPQSSYPLLANGVCKWPG |
| Accession No. | CEKVFEEPEDFLKHCQADHLLDEKGRAQCLLQREMVQSLEQ |
| Q9BZS1-3 | QLVLEKEKLSAMQAHLAGKMALTKASSVASSDKGSCCIVA |
| SEQ ID NO: 253 | AGSQGPVVPAWSGPREAPDSLFAVRRHLWGSHGNSTFPEFL |
| | HNMDYFKFHNMRPPFTYATLIRWAILEAPEKQRTLNEIYHW |
| | FTRMFAFFRNHPATWKVSSSEVAVTGMASSAIAAQSGQAW |
| | VWAHRHIGEERDVGCWWWLLASEVDAHLLPVPGLPQNAIR |
| | HNLSLHKCFVRVESEKGAVWTVDELEFRKKRSQRPSRCSNP |
| | TPGP |
| Forkhead box | MPNPRPGKPSAPSLALGPSPGASPSWRAAPKASDLLGARGPG |
| protein P1 (FoxP3), | GTFQGRDLRGGAHASSSSLNPMPPSQLQLPTLPLVMVAPSG |
| isoform 4, | ARLGPLPHLQALLQDRPHFMHQLSTVDAHARTPVLQVHPLE |
| UniProKB | SPAMISLTPPTTATGVFSLKARPGLPPGINVASLEWVSREPAL |
| Accession No. | LCTFPNPSAPRKDSTLSAVPQSSYPLLANGVCKWPGCEKVFE |
| Q9BZS1-4 | EPEDFLKHCQADHLLDEKGRAQCLLQREMVQSLEQQASSD |
| SEQ ID NO: 254 | K |
| | GSCCIVAAGSQGPVVPAWSGPREAPDSLFAVRRHLWGSHGN |
| | STFPEFLHNMDYFKFHNMRPPFTYATLIRWAILEAPEKQRTL |
| | NEIYHWFTRMFAFFRNHPATWKNAIRHNLSLHK CFVRVESE |
| | KGAVWTVDELEFRKKRSQRPSRCSNPTPGP |
| C/EBP-homologous | MAAESLPFSFGTLSSWELEAWYEDLQEVLSSDENGGTYVSP |
| protein (CHOP), | PGNEEEESKIFTTLDPASLAWLTEEEPEPAEVTSTSQSPHSPDS |
| isoform 1, | SQSSLAQEEEEEDQGRTRKRKQSGHSPARAGKQRMKEKEQE |
| UniProKB | NERKVAQLAEENERLKQEIERLTREVEATRRALIDRMVNLH |
| Accession No. | QA |
| P35638-1 | |
| SEQ ID NO: 255 | |
| C/EBP-homologous | MELVPATPHYPADVLFQTDPTAEMAAESLPFSFGTLSSWELE |
| protein (CHOP), | AWYEDLQEVLSSDENGGTYVSPPGNEEEESKIFTTLDPASLA |
| isoform 2, | WLTEEEPEPAEVTSTSQSPHSPDSSQSSLAQEEEEEDQGRTRK |
| UniProKB | RKQSGHSPARAGKQRMKEKEQENERKVAQLAEENERLKQE |
| Accession No. | IERLTREVEATRRALIDRMVNLHQA |
| P35638-2 | |
| SEQ ID NO: 256 | |
| Nuclear factor of | MNAPERQPQPDGGDAPGHEPGGSPQDELDFSILFDYEYINPNEEEPNAHK |
| activated T-cells, | VASPPSGPAYPDDVLDYGLKPYSPLASLSGEPPGRFGEPDRVGPQKFLSA |
| cytoplasmic 2 | AKPAGASGLSPRIEITPSHELIQAVGPLRMRDAGLLVEQPPLAGVAASPR |
| (NFATC2), isoform | FTLPVPGFEGYREPLCLSPASSGSSASFISDTFSPYTSPCVSPNNGGPDD |
| 1, UniProKB | LCPQFQNIPAHYSPRISPIMSPRTSLAEDSCLGRHSPVPRPASRSSSPGA |
| Accession No. | KRRHSCAEALVALPPGASPQRSRSPSPQPSSHVAPQDHGSPAGYPPVAGS |
| Q13469-1 | AVIMDALNSLATDSPCGIPPKMWKTSPDPSPVSAAPSKAGLPRHIYPAVE |
| SEQ ID NO: 257 | FLGPCEQGERRNSAPESILLVPPTWPKPLVPAIPICSIPVTASLPPLEWP |
| | LSSQSGSYELRIEVQPKPHHRAHYETEGSRGAVKAPTGGHPVVQLHGYME |
| | NKPLGLQIFIGTADERILKPHAFYQVHRITGKTVTTTSYEKIVGNTKVLE |
| | IPLEPKNNMRATIDCAGILKLRNADIELRKGETDIGRKNTRVRLVERVHI |
| | PESSGRIVSLQTASNPIECSQRSAHELPMVERQDTDSCLVYGGQQMILTG |
| | QNFTSESKVVFTEKTTDGQQIWEMEATVDKDKSQPNMLFVEIPEYRNKHI |
| | RTPVKVNFYVINGKRKRSQPQHFTYHPVPAIKTEPTDEYDPTLICSPTHG |
| | GLGSQPYYPQHPMVAESPSCLVATMAPCQQFRTGLSSPDARYQQQNPAAV |
| | LYQRSKSLSPSLLGYQQPALMAAPLSLADAHRSVIVHAGSQGQSSALLHP |
| | SPINQQASPVIHYSPINQQLRCGSHQEFQHIMYCENFAPGTTRPGPPPVS |
| | QGQRLSPGSYPTVIQQQNATSQRAAKNGPPVSDQKEVIPAGVTIKQEQNL |
| | DQTYLDDVNEIIRKEFSGPPARNQT |
| Nuclear factor of | MPSDFISLLSADLDLESPKSLYSRESVYDLLPKELQLPPSRETSVASMSQ |
| activated T-cells 5 | TSGGEAGSPPPAVVAADASSAPSSSSMGGACSSFTTSSSPTIYSTSVTDS |
| (NFAT5), isoform | KAMQVESCSSAVGVSNRGVSEKQLTSNTVQQHPSTPKRHTVLYISPPPED |
| 1, UniProKB | LLDNSRMSCQDEGCGLESEQSCSMWMEDSPSNFSNMSTSSYNDNTEVPRK |
| Accession No. | SRKRNPKQRPGVKRRDCEESNMDIFDADSAKAPHYVLSQLTTDNKGNSKA |
| 094916-1 | GNGTLENQKGTGVKKSPMLCGQYPVKSEGKELKIVVQPETQHRARYLTEG |
| SEQ ID NO: 258 | SRGSVKDRTQQGFPTVKLEGHNEPVVLQVFVGNDSGRVKPHGFYQACRVT |
| | GRNTTPCKEVDIEGTTVIEVGLDPSNNMTLAVDCVGILKLRNADVEARIG |
| | IAGSKKKSTRARLVFRVNIMRKDGSTLTLQTPSSPILCTQPAGVPEILKK |
| | SLHSCSVKGEEEVFLIGKNFLKGTKVIFQENVSDENSWKSEAEIDMELFH |
| | QNHLIVKVPPYHDQHITLPVSVGIYVVTNAGRSHDVQPFTYTPDPAAAGA |
| | LNVNVKKEISSPARPCSFEEAMKAMKTTGCNLDKVNIIPNALMTPLIPSS |
| | MIKSEDVTPMEVTAEKRSSTIFKTTKSVGSTQQTLENISNIAGNGSFSSP |
| | SSSHLPSENEKQQQIQPKAYNPETLTTIQTQDISQPGTFPAVSASSQLPN |
| | SDALLQQATQFQTRETQSREILQSDGTVVNLSQLTEASQQQQQSPLQEQA |
| | QTLQQQISSNIFPSPNSVSQLQNTIQQLQAGSFTGSTASGSSGSVDLVQQ |
| | VLEAQQQLSSVLFSAPDGNENVQEQLSADIFQQVSQIQSGVSPGMFSSTE |
| | PTVHTRPDNLLPGRAESVHPQSENTLSNQQQQQQQQQQVMESSAAMVMEM |
| | QQSICQAAAQIQSELFPSTASANGNLQQSPVYQQTSHMMSALSTNEDMQM |
| | QCELFSSPPAVSGNETSTTTTQQVATPGTTMFQTSSSGDGEETGTQAKQI |
| | QNSVFQTMVQMQHSGDNQPQVNLFSSTKSMMSVQNSGTQQQGNGIFQQGN |
| | EMMSLQSGNFLQQSSHSQAQLFHPQNPIADAQNLSQETQGSLFHSPNPIV |
| | HSQTSTTSSEQMQPPMFHSQSTIAVLQGSSVPQDQQSTNIELSQSPMNNL |
| | QTNTVAQEAFFAAPNSISPLQSTSNSEQQAAFQQQAPISHIQTPMLSQEQ |
| | AQPPQQGLFQPQVALGSLPPNPMPQSQQGTMFQSQHSIVAMQSNSPSQEQ |
| | QQQQQQQQQQQQQQQQSILFSNQNTNATMASPKQPPPNMIFNPNQNPMAN |
| | QEQQNQSIFHQQSNMAPMNQEQQPMQFQSQSTVSSLQNPGPTQSESSQTP |
| | LFHSSPQIQLVQGSPSSQEQQVTLFLSPASMSALQTSINQQDMQQSPLYS |
| | PQNNMPGIQGATSSPQPQATLFHNTAGGTMNQLQNSPGSSQQTSGMFLFG |
| | IQNNCSQLLTSGPATLPDQLMAISQPGQPQNEGQPPVTILLSQQMPENSP |
| | LASSINTNQNIEKIDLLVSLQNQGNNLTGSF |
| Nuclear factor of | MPSTSFPVPSKFPLGPAAAVFGRGETLGPAPRAGGTMKSAEEEHYGYASS |
| activated T-cells, | NVSPALPLPTAHSTLPAPCHNLQTSTPGIIPPADHPSGYGAALDGGPAGY |
| cytoplasmic 1 | FLSSGHIRPDGAPALESPRIEITSCLGLYHNNNQFFHDVEVEDVIPSSKR |
| (NFATC1), isoform | SPSTATLSLPSLEAYRDPSCLSPASSLSSRSCNSEASSYESNYSYPYASP |
| 1, UniProKB | QTSPWQSPCVSPKTTDPEEGFPRGLGACTLLGSPRHSPSTSPRASVTEES |
| Accession No. | WLGARSSRPASPCNKRKYSLNGRQPPYSPHHSPTPSPHGSPRVSVIDDSW |
| 095644-1 | IGNTTQYTSSAIVAAINALTTDSSLDLGDGVPVKSRKTTLEQPPSVALKV |
| SEQ ID NO: 259 | EPVGEDLGSPPPPADFAPEDYSSFQHIRKGGFCDQYLAVPQHPYQWAKPK |
| | PLSPTSYMSPTLPALDWQLPSHSGPYELRIEVQPKSHHRAHYETEGSRGA |
| | VKASAGGHPIVQLHGYLENEPLMLQLFIGTADDRLLRPHAFYQVHRITGK |
| | TVSTTSHEAILSNIKVLEIPLLPENSMRAVIDCAGILKLRNSDIELRKGE |
| | TDIGRKNTRVRLVFRVHVPQPSGRTLSLQVASNPIECSQRSAQELPLVEK |
| | QSTDSYPVVGGKKMVLSGHNFLQDSKVIFVEKAPDGHHVWEMEAKTDRDL |
| | CKPNSLVVEIPPERNQRITSPVHVSFYVQNGKRKRSQYQRFTYLPANVPI |
| | IKTEPTDDYEPAPTCGPVSQGLSPLPRPYYSQQLAMPPDPSSCLVAGFPP |
| | CPQRSTLMPAAPGVSPKLHDLSPAAYTKGVASPGHCHLGLPQPAGEAPAV |
| | QDVPRPVATHPGSPGQPPPALLPQQVSAPPSSSCPPGLEHSLCPSSPSPP |
| | LPPATQEPTCLQPCSPACPPATGRPQHLPSTVRRDESPTAGPRLLPEVHE |
| | DGSPNLAPIPVTVKREPEELDQLYLDDVNEIIRNDLSSTSTHS |
| Nuclear factor of | MGAASCEDEELEFKLVFGEEKEAPPLGAGGLGEELDSEDAPPCCRLALGE |
| activated T-cells, | PPPYGAAPIGIPRPPPPRPGMHSPPPRPAPSPGTWESQPARSVRLGGPGG |
| cytoplasmic 4 | GAGGAGGGRVLECPSIRITSISPTPEPPAALEDNPDAWGDGSPRDYPPPE |
| (NFATC4), isoform 1, | GFGGYREAGGQGGGAFFSPSPGSSSLSSWSFFSDASDEAALYAACDEVES |
| UniProKB Accession | ELNEAASRFGLGSPLPSPRASPRPWTPEDPWSLYGPSPGGRGPEDSWILL |
| No. Q14934-1 | SAPGPTPASPRPASPCGKRRYSSSGTPSSASPALSRRGSLGEEGSEPPPP |
| SEQ ID NO: 260 | PPLPLARDPGSPGPFDYVGAPPAESIPQKTRRTSSEQAVALPRSEEPASC |
| | NGKLPLGAEESVAPPGGSRKEVAGMDYLAVPSPLAWSKARIGGHSPIFRT |
| | SALPPLDWPLPSQYEQLELRIEVQPRAHHRAHYETEGSRGAVKAAPGGHP |
| | VVKLLGYSEKPLTLQMFIGTADERNLRPHAFYQVHRITGKMVATASYEAV |
| | VSGTKVLEMTLLPENNMAANIDCAGILKLRNSDIELRKGETDIGRKNTRV |
| | RLVFRVHVPQGGGKVVSVQAASVPIECSQRSAQELPQVEAYSPSACSVRG |
| | GEELVITGSNFLPDSKVVFIERGPDGKLQWEEEATVNRLQSNEVTLTLTV |
| | PEYSNKRVSRPVQVYFYVSNGRRKRSPTQSFRFLPVICKEEPLPDSSLRG |
| | FPSASATPFGTDMDFSPPRPPYPSYPHEDPACETPYLSEGEGYGMPPLYP |
| | QTGPPPSYRPGLRMFPETRGTTGCAQPPAVSFLPRPFPSDPYGGRGSSES |
| | LGLPFSPPAPFRPPPLPASPPLEGPFPSQSDVHPLPAEGYNKVGPGYGPG |
| | EGAPEQEKSRGGYSSGERDSVPIQGITLEEVSEIIGRDLSGFPAPPGEEP |
| | PA |
| Nuclear factor of | MITANCGAHDELDFKLVFGEDGAPAPPPPGSRPADLEPDDCASIYIFNVD |
| activated T-cells, | PPPSTLTTPLCLPHHGLPSHSSVLSPSFQLQSHKNYEGTCEIPESKYSPL |
| cytoplasmic 3 | GGPKPFECPSIQITSISPNCHQELDAHEDDLQINDPEREFLERPSRDHLY |
| (NFATC3), isoform 1, | LPLEPSYRESSLSPSPASSISSRSWFSDASSCESLSHIYDDVDSELNEAA |
| UniProKB Accession | ARFTLGSPLTSPGGSPGGCPGEETWHQQYGIGHSLSPRQSPCHSPRSSVT |
| No. Q12968-1 | DENWLSPRPASGPSSRPTSPCGKRRHSSAEVCYAGSLSPHHSPVPSPGHS |
| SEQ ID NO: 261 | PRGSVTEDTWLNASVHGGSGLGPAVFPFQYCVETDIPLKTRKTSEDQAAI |
| | LPGKLELCSDDQGSLSPARETSIDDGLGSQYPLKKDSCGDQFLSVPSPFT |
| | WSKPKPGHTPIFRISSLPPLDWPLPAHFGQCELKIEVQPKTHHRAHYETE |
| | GSRGAVKASTGGHPVVKLLGYNEKPINLQMFIGTADDRYLRPHAFYQVHR |
| | ITGKTVATASQEIIIASTKVLEIPLLPENNMSASIDCAGILKLRNSDIEL |
| | RKGETDIGRKNTRVRLVERVHIPQPSGKVLSLQIASIPVECSQRSAQELP |
| | HIEKYSINSCSVNGGHEMVVTGSNFLPESKIIFLEKGQDGRPQWEVEGKI |
| | IREKCQGAHIVLEVPPYHNPAVTAAVQVHFYLCNGKRKKSQSQRFTYTPV |
| | LMKQEHREEIDLSSVPSLPVPHPAQTQRPSSDSGCSHDSVLSGQRSLICS |
| | IPQTYASMVTSSHLPQLQCRDESVSKEQHMIPSPIVHQPFQVTPTPPVGS |
| | SYQPMQTNVVYNGPTCLPINAASSQEFDSVLFQQDATLSGLVNLGCQPLS |
| | SIPFHSSNSGSTGHLLAHTPHSVHTLPHLQSMGYHCSNTGQRSLSSPVAD |
| | QITGQPSSQLQPITYGPSHSGSATTASPAASHPLASSPLSGPPSPQLQPM |
| | PYQSPSSGTASSPSPATRMHSGQHSTQAQSTGQGGLSAPSSLICHSLCDP |
| | ASFPPDGATVSIKPEPEDREPNFATIGLQDITLDDVNEIIGRDMSQISVS |
| | QGAGVSRQAPLPSPESLDLGRSDGL |
| Von Hippel-Lindau | MPRRAENWDEAEVGAEEAGVEEYGPEEDGGEESGAEESGPEESGPEELGA |
| Tumor Suppressor | EEEMEAGRPRPVLRSVNSREPSQVIFCNRSPRVVLPVWLNEDGEPQPYPT |
| (VHL), isoform 1, | LPPGTGRRIHSYRGHLWLFRDAGTHDGLLVNQTELFVPSLNVDGQPIFAN |
| UniProKB Accession | ITLPVYTLKERCLQVVRSLVKPENYRRLDIVRSLYEDLEDHPNVQKDLER |
| No. P40337-1 | LTQERIAHQRMGD |
| SEQ ID NO: 262 | |
| Cytokine-inducible | MVLCVQGPRPLLAVERTGQRPLWAPSLELPKPVMQPLPAGAFLEEVAEGT |
| SH2-containing | PAQTESEPKVLDPEEDLLCIAKTFSYLRESGWYWGSITASEARQHLQKMP |
| protein (CISH), | EGTFLVRDSTHPSYLFTLSVKTTRGPINVRIEYADSSFRLDSNCLSRPRI |
| isoform 1, UniProKB | LAFPDVVSLVQHYVASCTADTRSDSPDPAPTPALPMPKEDAPSDPALPAP |
| Accession No. | PPATAVHLKLVQPFVRRSSARSLQHLCRLVINRLVADVDCLPLPRRMADY |
| Q9NSE2-1 | LRQYPFQL |
| SEQ ID NO: 263 | |
| Suppressor of | MVAHNQVAADNAVSTAAEPRRRPEPSSSSSSSPAAPARPRPCPAVPAPAPGD |
| cytokine signaling 1 | THERTFRSHADYRRITRASALLDACGFYWGPLSVHGAHERLRAEPVGTFLVR |
| (SOCS1), isoform 1, | DSRQRNCFFALSVKMASGPTSIRVHFQAGRFHLDGSRESFDCLFELLEHYVA |
| UniProKB Accession | APRRMLGAPLRQRRVRPLQELCRQRIVATVGRENLARIPLNPVLRDYLSSEP |
| No. 015524-1 | FQI |
| SEQ ID NO: 264 | |
| Suppressor of | MTLRCLEPSGNGGEGTRSQWGTAGSAEEPSPQAARLAKALRELGQTGWYWGS |
| cytokine signaling 2 | MTVNEAKEKLKEAPEGTFLIRDSSHSDYLLTISVKTSAGPTNLRIEYQDGKF |
| (SOCS2), isoform 1, | RLDSIICVKSKLKQFDSVVHLIDYYVQMCKDKRTGPEAPRNGTVHLYLTKPL |
| UniProKB Accession | YTSAPSLQHLCRLTINKCTGAIWGLPLPTRLKDYLEEYKFQV |
| No. 014508-1 | |
| SEQ ID NO: 265 | |
| Suppressor of | MVTHSKFPAAGMSRPLDTSLRLKTFSSKSEYQLVVNAVRKLQESGFYWSAVT |
| cytokine signaling 3 | GGEANLLLSAEPAGTFLIRDSSDQRHFFTLSVKTQSGTKNLRIQCEGGSFSL |
| (SOCS3), isoform 1, | QSDPRSTQPVPRFDCVLKLVHHYMPPPGAPSFPSPPTEPSSEVPEQPSAQPL |
| UniProKB Accession | PGSPPRRAYYIYSGGEKIPLVLSRPLSSNVATLQHLCRKTVNGHLDSYEKVT |
| No. 014543-1 | QLPGPIREFLDQYDAPL |
| SEQ ID NO: 266 | |
| Suppressor of | MAENNENISKNVDVRPKTSRSRSADRKDGYVWSGKKLSWSKKSESYSDAETV |
| cytokine signaling 4 | NGIEKTEVSIRNQERKHSCSSIELDLDHSCGHRFLGRSLKQKLQDAVGQCFP |
| (SOCS4), isoform 1, | IKNCSSRHSSGLPSKRKIHISELMLDKCPFPPRSDLAFRWHFIKRHTAPINS |
| UniProKB Accession | KSDEWVSTDLSQTELRDGQLKRRNMEENINCFSHINVQPCVITTDNALCREG |
| No. Q8WXH5-1 | PMTGSVMNLVSNNSIEDSDMDSDDEILTLCTSSRKRNKPKWDLDDEILQLET |
| SEQ ID NO: 267 | PPKYHTQIDYVHCLVPDLLQINNNPCYWGVMDKYAAEALLEGKPEGTFLLRD |
| | SAQEDYLFSVSFRRYSRSLHARIEQWNHNFSFDAHDPCVFHSPDITGLLEHY |
| | KDPSACMFFEPLLSTPLIRTFPFSLQHICRTVICNCTTYDGIDALPIPSSMK |
| | LYLKEYHYKSKVRVLRIDAPEQQC |
| Suppressor of | MDKVGKMWNNFKYRCQNLEGHEGGSRSENVDMNSNRCLSVKEKNISIGDSTP |
| cytokine signaling 5 | QQQSSPLRENIALQLGLSPSKNSSRRNQNCATEIPQIVEISIEKDNDSCVTP |
| (SOCS5), isoform 1, | GTRLARRDSYSRHAPWGGKKKHSCSTKTQSSLDADKKFGRTRSGLQRRERRY |
| UniProKB Accession | GVSSVHDMDSVSSRTVGSRSLRQRLQDTVGLCFPMRTYSKQSKPLFSNKRKI |
| No. 075159-1 | HISELMLEKCPFPAGSDLAQKWHLIKQHTAPVSPHSTFEDTFDPSLVSTEDE |
| SEQ ID NO: 268 | EDRIRERRRISIEEGVDPPPNAQIHTFEATAQVNPLYKLGPKLAPGMTEISG |
| | DSSAIPQANCDSEEDTTTLCLQSRRQKQRQISGDSHTHVSRQGAWKVHTQID |
| | YIHCLVPDLLQITGNPCYWGVMDRYEAEALLEGKPEGTFLLRDSAQEDYLES |
| | VSFRRYNRSLHARIEQWNHNFSFDAHDPCVFHSSTVTGLLEHYKDPSSCMFF |
| | EPLLTISLNRTFPFSLQYICRAVICRCTTYDGIDGLPLPSMLQDFLKEYHYK |
| | QKVRVRWLEREPVKAK |
| Suppressor of | MKKISLKTLRKSFNLNKSKEETDEMVVQQPSLASDFGKDDSLFGSCYGKDMA |
| cytokine signaling 6 | SCDINGED |
| (SOCS6), isoform 1, | EKGGKNRSKSESIMGTLKRRLSAKQKSKGKAGTPSGSSADEDTFSSSSAPIV |
| UniProKB Accession | FKDVRAQR |
| No. 014544-1 | PIRSTSLRSHHYSPAPWPLRPINSEETCIKMEVRVKALVHSSSPSPALNGVR |
| SEQ ID NO: 269 | KDFHDLQS |
| | ETTCQEQANSLKSSASHNGDLHLHLDEHVPVVIGIMPQDYIQYTVPIDEGMY |
| | PLEGSRSY |
| | CLDSSSPMEVSAVPPQVGGRAFPEDESQVDQDLVVAPEIFVDQSVNGLLIGT |
| | TGVMLQSP |
| | RAGHDDVPPLSPLLPPMQNNQIQRNFSGLIGTEAHVAESMRCHINFDPNSAP |
| | GVARVYDS |
| | VQSSGPMVVTSLTEELKKLAKQGWYWGPITRWEAEGKLANVPDGSFLVRDSS |
| | DDRYLLSL |
| | SFRSHGKTLHTRIEHSNGRFSFYEQPDVEGHTSIVDLIEHSIRDSENGAFCY |
| | SRSRLPGS |
| | ATYPVRLTNPVSRFMQVRSLQYLCRFVIRQYTRIDLIQKLPLPNKMKDYLQE |
| | KHY |
| Suppressor of | MVFRNVGRPPEEEDVEAAPEPGPSELLCPRHRCALDPKALPPGLALERTWGP |
| cytokine signaling 7 | AAGLEAQLAALGLGQPAGPGVKTVGGGCCPCPCPPQPPPPQPQPPAAAPQAG |
| (SOCS7), isoform 1, | EDPTETSDALLVLEGLESEAESLEINSCSEEELSSPGRGGGGGGRLLLQPPG |
| UniProKB Accession | PELPPVPFPLQDLVPLGRLSRGEQQQQQQQQPPPPPPPPGPLRPLAGPSRKG |
| No. 014512-1 | SFKIRLSRLFRTKSCNGGSGGGDGTGKRPSGELAASAASLTDMGGSAGRELD |
| SEQ ID NO: 270 | AGRKPKLTRTQSAFSPVSFSPLFTGETVSLVDVDISQRGLTSPHPPTPPPPP |
| | RRSISLIDDISGTLPTSVLVAPMGSSLQSFPLPPPPPPHAPDAFPRIAPIRA |
| | AESLHSQPPQHLQCPLYRPDSSSFAASLRELEKCGWYWGPMNWEDAEMKLKG |
| | KPDGSFLVRDSSDPRYILSLSFRSQGITHHTRMEHYRGTFSLWCHPKFEDRC |
| | QSVVEFIKRAIMHSKNGKFLYFLRSRVPGLPPTPVQLLYPVSRFSNVKSLQH |
| | LCRFRIRQLVRIDHIPDLPLPKPLISYIRKFYYYDPQEEVYLSLKEAQLISK |
| | QKQEVEPST |
| Tyrosine-protein | MPTTIEREFEELDTQRRWQPLYLEIRNESHDYPHRVAKFPENRNRNRYRDVS |
| phosphatase non- | PYDHSRVKLQNAENDYINASLVDIEEAQRSYILTQGPLPNTCCHFWLMVWQQ |
| receptor type 2 | KTKAVVMLNRIVEKESVKCAQYWPTDDQEMLFKETGFSVKLLSEDVKSYYTV |
| (PTPN2), isoform | HILQLENINSGETRTISHFHYTTWPDFGVPESPASFLNFLFKVRESGSINPD |
| 1, UniProKB | HGPAVIHCSAGIGRSGTFSLVDTCLVLMEKGDDINIKQVLLNMRKYRMGLIQ |
| Accession | TPDQLRFSYMAIIEGAKCIKGDSSIQKRWKELSKEDLSPAFDHSPNKIMTEK |
| No.P17706-1 | YNGNRIGLEEEKLIGDRCTGLSSKMQDTMEENSESALRKRIREDRKATTAQK |
| SEQ ID NO: 271 | VQQMKQRINENERKRKRWLYWQPILTKMGFMSVILVGAFVGWILFFQQNAL |
| Protein-tyrosine | MVRWFHRDISGIDAETLLKGRGVHGSFLARPSRKNQGDFSLSVRVGDQVTHI |
| phosphatase SHP-1 | RIQNSGDFYDLYGGEKFATLTELVEYYTQQQGVLQDRDGTIIHLKYPLNCSD |
| (SHP1), isoform 1, | PTSERWYHGHMSGGQAETLLQAKGEPWTFLVRESLSQPGDFVLSVLSDQPKA |
| UniProKB Accession | GPGSPLRVTHIKVMCEGGRYTVGGLETFDSLIDLVEHFKKTGIEEASGAFVY |
| No. P29350-1 | LRQPYYATRVNAADIENRVLELNKKQESEDTAKAGFWEEFESLQKQEVKNLH |
| SEQ ID NO: 272 | QRLEGQRPENKGKNRYKNILPFDHSRVILQGRDSNIPGSDYINANYIKNQLL |
| | GPDENAKTYIASQGCLEATVNDFWQMAWQENSRVIVMTTREVEKGRNKCVPY |
| | WPEVGMQRAYGPYSVINCGEHDTTEYKLRTLQVSPLDNGDLIREIWHYQYLS |
| | WPDHGVPSEPGGVLSFLDQINQRQESLPHAGPIIVHCSAGIGRIGTIIVIDM |
| | LMENISTKGLDCDIDIQKTIQMVRAQRSGMVQTEAQYKFIYVAIAQFIETTK |
| | KKLEVLQSQKGQESEYGNITYPPAMKNAHAKASRTSSKHKEDVYENLHTKNK |
| | REEKVKKQRSADKEKSKGSLKRK |
| Protein-tyrosine | MTSRRWFHPNITGVEAENLLLTRGVDGSFLARPSKSNPGDFTLSVRRNGA |
| phosphatase (SHP2), | VTHIKIQNTGDYYDLYGGEKFATLAELVQYYMEHHGQLKEKNGDVIELKY |
| isoform 1, UniProKB | PLNCADPTSERWFHGHLSGKEAEKLLTEKGKHGSFLVRESQSHPGDEVIS |
| Accession No. | VRIGDDKGESNDGKSKVTHVMIRCQELKYDVGGGERFDSLTDLVEHYKKN |
| Q06124-2 | PMVETLGTVLQLKQPLNTTRINAARIESRVRELSKLAETTDKVKQGEWEE |
| SEQ ID NO: 273 | FETLQQQECKLLYSRKEGQRQENKNKNRYKNILPFDHTRVVLHDGDPNEP |
| | VSDYINANIIMPEFETKCNNSKPKKSYIATQGCLQNTVNDFWRMVFQENS |
| | RVIVMTTKEVERGKSKCVKYWPDEYALKEYGVMRVRNVKESAAHDYTIRE |
| | LKLSKVGQGNTERTVWQYHFRTWPDHGVPSDPGGVLDFLEEVHHKQESIM |
| | DAGPVVVHCSAGIGRIGTFIVIDILIDIIREKGVDCDIDVPKTIQMVRSQ |
| | RSGMVQTEAQYRFIYMAVQHYIETLQRRIEEEQKSKRKGHEYTNIKYSLA |
| | DQTSGDQSPLPPCTPTPPCAEMREDSARVYENVGLMQQQKSER |
| GRB2-related adapter | MEAVAKFDFTASGEDELSFHTGDVLKILSNQEEWFKAELGSQEGYVPKNF |
| protein 2 (GRAP2; | IDIQFPKWFHEGLSRHQAENLIMGKEVGFFIIRASQSSPGDFSISVRHED |
| also known as | DVQHFKVMRDNKGNYFLWTEKFPSINKLVDYYRINSISRQKQIFLRDRTR |
| GADS), isoform 1, | EDQGHRGNSLDRRSQGGPHLSGAVGEEIRPSMNRKLSDHPPTLPLQQHQH |
| UniProKB Accession | QPQPPQYAPAPQQLQQPPQQRYLQHHHFHQERRGGSLDINDGHCGTGLGS |
| No. 075791-1 | EMNAALMHRRHTDPVQLQAAGRVRWARALYDFEALEDDELGFHSGEVVEV |
| SEQ ID NO: 274 | LDSSNPSWWTGRLHNKLGLFPANYVAPMTR |
| Growth factor | MEAIAKYDFKATADDELSFKRGDILKVINEECDQNWYKAELNGKDGFIPK |
| receptor-bound | NYIEMKPHPWFFGKIPRAKAEEMLSKQRHDGAFLIRESESAPGDFSLSVK |
| protein 2 (Grb2), | FGNDVQHFKVLRDGAGKYFLWVVKENSLNELVDYHRSTSVSRNQQIFLRD |
| isoform 1, UniProKB | IHQVPQQPTYVQALFDEDPQEDGELGFRRGDFIHVMDNSDPNWWKGACHG |
| Accession No. | QTGMFPRNYVTPVNRNV |
| P62993-1 | |
| SEQ ID NO: 275 | |
| 1-phosphatidylinositol | MAGAASPCANGCGPGAPSDAEVLHLCRSLEVGTVMTLFYSKKSQRPERKT |
| 4,5-bisphosphate | FQVKLETRQITWSRGADKIEGAIDIREIKEIRPGKTSRDFDRYQEDPAFR |
| phosphodiesterase | PDQSHCFVILYGMEFRLKTLSLQATSEDEVNMWIKGLTWLMEDTLQAPTP |
| gamma-1 (PLCy1), | LQIERWLRKQFYSVDRNREDRISAKDLKNMLSQVNYRVPNMRFLRERLTD |
| isoform 1, UniProKB | LEQRSGDITYGQFAQLYRSLMYSAQKTMDLPFLEASTLRAGERPELCRVS |
| Accession No. | LPEFQQFLLDYQGELWAVDRLQVQEFMLSFLRDPIREIEEPYFFLDEFVT |
| P19174-1 | FLFSKENSVWNSQLDAVCPDTMNNPLSHYWISSSHNTYLTGDQFSSESSL |
| SEQ ID NO: 276 | EAYARCLRMGCRCIELDCWDGPDGMPVIYHGHTLTTKIKFSDVLHTIKEH |
| | AFVASEYPVILSIEDHCSIAQQRNMAQYFKKVLGDTLLTKPVEISADGLP |
| | SPNQLKRKILIKHKKLAEGSAYEEVPTSMMYSENDISNSIKNGILYLEDP |
| | VNHEWYPHYFVLTSSKIYYSEETSSDQGNEDEEEPKEVSSSTELHSNEKW |
| | FHGKLGAGRDGRHIAERLLTEYCIETGAPDGSFLVRESETFVGDYTLSFW |
| | MRLSEPVPQTNAHESKEWYHASLTRAQAEHMLMRVPRDGAFLVRKRNEPN |
| | SYAISFRAEGKIKHCRVQQEGQTVMLGNSEFDSLVDLISYYEKHPLYRKM |
| | KLRYPINEEALEKIGTAEPDYGALYEGRNPGFYVEANPMPTFKCAVKALF |
| | DYKAQREDELTFIKSAIIQNVEKQEGGWWRGDYGGKKQLWFPSNYVEEMV |
| | NPVALEPEREHLDENSPLGDLLRGVIDVPACQIAIRPEGKNNRLFVFSIS |
| | MASVAHWSLDVAADSQEELQDWVKKIREVAQTADARLTEGKIMERRKKIA |
| | LELSELVVYCRPVPFDEEKIGTERACYRDMSSFPETKAEKYVNKAKGKKE |
| | LQYNRLQLSRIYPKGQRLDSSNYDPLPMWICGSQLVALNFQTPDKPMQMN |
| | QALFMTGRHCGYVLQPSTMRDEAFDPFDKSSLRGLEPCAISIEVLGARHL |
| | PKNGRGIVCPFVEIEVAGAEYDSTKQKTEFVVDNGLNPVWPAKPFHFQIS |
| | NPEFAFLRFVVYEEDMESDQNFLAQATFPVKGLKTGYRAVPLKNNYSEDL |
| | ELASLLIKIDIFPAKENGDLSPFSGTSLRERGSDASGQLFHGRAREGSFE |
| | SRYQQPFEDFRISQEHLADHEDSRERRAPRRTRVNGDNRL |
| Linker for activation | MEEAILVPCVIGLLLLPILAMLMALCVHCHRLPGSYDSTSSDSLYPRGIQ |
| of T-cells family | FKRPHTVAPWPPAYPPVTSYPPLSQPDLLPIPRSPQPLGGSHRTPSSRRD |
| member 1 (LAT), | SDGANSVASYENEGASGIRGAQAGWGVWGPSWTRLTPVSLPPEPACEDAD |
| isoform 1, UniProKB | EDEDDYHNPGYLVVLPDSTPATSTAAPSAPALSTPGIRDSAFSMESIDDY |
| Accession No. | VNVPESGESAEASLDGSREYVNVSQELHPGAAKTEPAALSSQEAEEVEEE |
| 043561-1 | GAPDYENLQELN |
| SEQ ID NO: 277 | |
| SH2 domain- | MALRNVPFRSEVLGWDPDSLADYFKKLNYKDCEKAVKKYHIDGARFLNLT |
| containing leukocyte | ENDIQKFPKLRVPILSKLSQEINKNEERRSIFTRKPQVPRFPEETESHEE |
| protein of 76 kDa | DNGGWSSFEEDDYESPNDDQDGEDDGDYESPNEEEEAPVEDDADYEPPPS |
| (SLP76), isoform 1, | NDEEALQNSILPAKPFPNSNSMYIDRPPSGKTPQQPPVPPQRPMAALPPP |
| UniProKB Accession | PAGRNHSPLPPPQTNHEEPSRSRNHKTAKLPAPSIDRSTKPPLDRSLAPF |
| No. Q13094-1 | DREPFTLGKKPPFSDKPSIPAGRSLGEHLPKIQKPPLPPTTERHERSSPL |
| SEQ ID NO: 278 | PGKKPPVPKHGWGPDRRENDEDDVHQRPLPQPALLPMSSNTFPSRSTKPS |
| | PMNPIPSSHMPGAFSESNSSFPQSASLPPYFSQGPSNRPPIRAEGRNFPL |
| | PLPNKPRPPSPAEEENSLNEEWYVSYITRPEAEAALRKINQDGTFLVRDS |
| | SKKTTTNPYVLMVLYKDKVYNIQIRYQKESQVYLIGTGLRGKEDFLSVSD |
| | IIDYFRKMPLLLIDGKNRGSRYQCTLTHAAGYP |
| Tyrosine-protein | MGCGCSSHPEDDWMENIDVCENCHYPIVPLDGKGTLLIRNGSEVRDPLVT |
| kinase Lck (Lck), | YEGSNPPASPLQDNLVIALHSYEPSHDGDLGFEKGEQLRILEQSGEWWKA |
| isoform 1, UniProKB | QSLTTGQEGFIPFNFVAKANSLEPEPWFFKNLSRKDAERQLLAPGNTHGS |
| Accession No. | FLIRESESTAGSFSLSVRDFDQNQGEVVKHYKIRNLDNGGFYISPRITFP |
| P06239-1 | GLHELVRHYTNASDGLCTRLSRPCQTQKPQKPWWEDEWEVPRETLKIVER |
| SEQ ID NO: 279 | LGAGQFGEVWMGYYNGHTKVAVKSLKQGSMSPDAFLAEANLMKQLQHQRL |
| | VRLYAVVTQEPIYIITEYMENGSLVDFLKTPSGIKLTINKLLDMAAQIAE |
| | GMAFIEERNYIHRDLRAANILVSDTLSCKIADFGLARLIEDNEYTAREGA |
| | KFPIKWTAPEAINYGTFTIKSDVWSFGILLTEIVTHGRIPYPGMINPEVI |
| | QNLERGYRMVRPDNCPEELYQLMRLCWKERPEDRPTFDYLRSVLEDFFTA |
| | TEGQYQPQP |
| Interleukin-2- | MNNFILLEEQLIKKSQQKRRTSPSNFKVRFFVLTKASLAYFEDRHGKKRT |
| inducible T-cell kinase | LKGSIELSRIKCVEIVKSDISIPCHYKYPFQVVHDNYLLYVFAPDRESRQ |
| (Itk), isoform 1, | RWVLALKEETRNNNSLVPKYHPNFWMDGKWRCCSQLEKLATGCAQYDPTK |
| UniProKB Accession | NASKKPLPPTPEDNRRPLWEPEETVVIALYDYQTNDPQELALRRNEEYCL |
| No. Q08881-1 | IDSSEIHWWRVQDRNGHEGYVPSSYLVEKSPNNLETYEWYNKSISRDKAE |
| SEQ ID NO: 280 | KLLIDTGKEGAFMVRDSRTAGTYTVSVETKAVVSENNPCIKHYHIKETND |
| | NPKRYYVAEKYVFDSIPLLINYHQHNGGGLVTRLRYPVCFGRQKAPVTAG |
| | LRYGKWVIDPSELTFVQEIGSGQFGLVHLGYWLNKDKVAIKTIREGAMSE |
| | EDFIEEAEVMMKLSHPKLVQLYGVCLEQAPICLVFEFMEHGCLSDYLRTQ |
| | RGLFAAETLLGMCLDVCEGMAYLEEACVIHRDLAARNCLVGENQVIKVSD |
| | FGMTRFVLDDQYTSSTGTKFPVKWASPEVESFSRYSSKSDVWSFGVLMWE |
| | VFSEGKIPYENRSNSEVVEDISTGFRLYKPRLASTHVYQIMNHCWKERPE |
| | DRPAFSRLLRQLAEIAESGL |
| B-cell progenitor | MAAVILESIFLKRSQQKKKTSPLNFKKRLFLLTVHKLSYYEYDFERGRRG |
| kinase (Btk), isoform | SKKGSIDVEKITCVETVVPEKNPPPERQIPRRGEESSEMEQISIIEREPY |
| 1, UniProKB | PFQVVYDEGPLYVFSPTEELRKRWIHQLKNVIRYNSDLVQKYHPCFWIDG |
| Accession No. | QYLCCSQTAKNAMGCQILENRNGSLKPGSSHRKTKKPLPPTPEEDQILKK |
| Q06187-1 | PLPPEPAAAPVSTSELKKVVALYDYMPMNANDLQLRKGDEYFILEESNLP |
| SEQ ID NO: 281 | WWRARDKNGQEGYIPSNYVTEAEDSIEMYEWYSKHMTRSQAEQLLKQEGK |
| | EGGFIVRDSSKAGKYTVSVFAKSTGDPQGVIRHYVVCSTPQSQYYLAEKH |
| | LFSTIPELINYHQHNSAGLISRLKYPVSQQNKNAPSTAGLGYGSWEIDPK |
| | DLTFLKELGTGQFGVVKYGKWRGQYDVAIKMIKEGSMSEDEFIEEAKVMM |
| | NLSHEKLVQLYGVCTKQRPIFIITEYMANGCLLNYLREMRHRFQTQQLLE |
| | MCKDVCEAMEYLESKQFLHRDLAARNCLVNDQGVVKVSDFGLSRYVLDDE |
| | YTSSVGSKFPVRWSPPEVIMYSKFSSKSDIWAFGVLMWEIYSLGKMPYER |
| | FINSETAEHIAQGLRLYRPHLASEKVYTIMYSCWHEKADERPTFKILLSN |
| | ILDVMDEES |
| BET family | |
| Ten-eleven | MSRSRHARPSRLVRKEDVNKKKKNSQLRKTTKGANKNVASVKTLSPGKLKQLIQ |
| translocation 1 | ERDVKKKTEPKPPVPVRSLLTRAGAARMNLDRTEVLFQNPESLTCNGFTMALRS |
| (TET1), isoform 1, | TSLSRRLSQPPLVVAKSKKVPLSKGLEKQHDCDYKILPALGVKHSENDSVPMQD |
| UniProKB Accession | TQVLPDIETLIGVQNPSLLKGKSQETTQFWSQRVEDSKINIPTHSGPAAEILPG |
| No. Q8NFU7-1 | PLEGTRCGEGLFSEETINDTSGSPKMFAQDTVCAPFPQRATPKVTSQGNPSIQL |
| SEQ ID NO: 282 | EELGSRVESIKLSDSYLDPIKSEHDCYPTSSLNKVIPDLNLRNCLALGGSTSPT |
| | SVIKFLLAGSKQATLGAKPDHQEAFEATANQQEVSDTTSELGQAFGAIPHQWEL |
| | PGADPVHGEALGETPDLPEIPGAIPVQGEVFGTILDQQETLGMSGSVVPDLPVE |
| | LPVPPNPIATFNAPSKWPEPQSTVSYGLAVQGAIQILPLGSGHTPQSSSNSEKN |
| | SLPPVMAISNVENEKQVHISFLPANTQGFPLAPERGLFHASLGIAQLSQAGPSK |
| | SDRGSSQVSVTSTVHVVNTTVVTMPVPMVSTSSSSYTTLLPTLEKKKRKRCGVC |
| | EPCQQKTNCGECTYCKNRKNSHQICKKRKCEELKKKPSVVVPLEVIKENKRPQR |
| | EKKPKVLKADFDNKPVNGPKSESMDYSRCGHGEEQKLELNPHTVENVTKNEDSM |
| | TGIEVEKWTQNKKSQLTDHVKGDFSANVPEAEKSKNSEVDKKRTKSPKLFVQTV |
| | RNGIKHVHCLPAETNVSFKKFNIEEFGKTLENNSYKFLKDTANHKNAMSSVATD |
| | MSCDHLKGRSNVLVFQQPGENCSSIPHSSHSIINHHASIHNEGDQPKTPENIPS |
| | KEPKDGSPVQPSLLSLMKDRRLTLEQVVAIEALTQLSEAPSENSSPSKSEKDEE |
| | SEQRTASLLNSCKAILYTVRKDLQDPNLQGEPPKLNHCPSLEKQSSCNTVVENG |
| | QTTTLSNSHINSATNQASTKSHEYSKVINSLSLFIPKSNSSKIDTNKSIAQGII |
| | TLDNCSNDLHQLPPRNNEVEYCNQLLDSSKKLDSDDLSCQDATHTQIEEDVATQ |
| | LTQLASIIKINYIKPEDKKVESTPTSLVTCNVQQKYNQEKGTIQQKPPSSVHNN |
| | HGSSLTKQKNPTQKKTKSTPSRDRRKKKPTVVSYQENDRQKWEKLSYMYGTICD |
| | IWIASKFQNFGQFCPHDFPTVEGKISSSTKIWKPLAQTRSIMQPKTVFPPLTQI |
| | KLQRYPESAEEKVKVEPLDSLSLFHLKTESNGKAFTDKAYNSQVQLTVNANQKA |
| | HPLTQPSSPPNQCANVMAGDDQIRFQQVVKEQIMHQRLPTLPGISHETPLPESA |
| | LTLRNVNVVCSGGITVVSTKSEEEVCSSSFGTSEFSTVDSAQKNENDYAMNFFT |
| | NPTKNIVSITKDSELPTCSCLDRVIQKDKGPYYTHLGAGPSVAAVREIMENRYG |
| | QKGNAIRIEIVVYTGKEGKSSHGCPIAKWVLRRSSDEEKVLCLVRQRTGHHCPT |
| | AVMVVLIMVWDGIPLPMADRLYTELTENLKSYNGHPTDRRCTLNENRTCTCQGI |
| | DPETCGASFSFGCSWSMYFNGCKFGRSPSPRRFRIDPSSPLHEKNLEDNLQSLA |
| | TRLAPIYKQYAPVAYQNQVEYENVARECRLGSKEGRPESGVTACLDECAHPHRD |
| | IHNMNNGSTVVCTLTREDNRSIGVIPQDEQLHVLPLYKLSDTDEFGSKEGMEAK |
| | IKSGAIEVLAPRRKKRTCFTQPVPRSGKKRAAMMTEVLAHKIRAVEKKPIPRIK |
| | RKNNSTTTNNSKPSSLPTLGSNTETVQPEVKSETEPHFILKSSDNTKTYSLMPS |
| | APHPVKEASPGFSWSPKTASATPAPLKNDATASCGFSERSSTPHCTMPSGRLSG |
| | ANAAAADGPGISQLGEVAPLPTLSAPVMEPLINSEPSTGVTEPLTPHQPNHQPS |
| | FLTSPQDLASSPMEEDEQHSEADEPPSDEPLSDDPLSPAEEKLPHIDEYWSDSE |
| | HIFLDANIGGVAIAPAHGSVLIECARRELHATTPVEHPNRNHPTRLSLVFYQHK |
| | NINKPQHGFELNKIKFEAKEAKNKKMKASEQKDQAANEGPEQSSEVNELNQIPS |
| | HKALTLTHDNVVTVSPYALTHVAGPYNHWV |
| Ten-eleven | MEQDRTNHVEGNRLSPFLIPSPPICQTEPLATKLQNGSPLPERAHPEVNGDTKW |
| translocation 2 | HSFKSYYGIPCMKGSQNSRVSPDFTQESRGYSKCLQNGGIKRTVSEPSLSGLLQ |
| (TET2), isoform 1, | IKKLKQDQKANGERRNFGVSQERNPGESSQPNVSDLSDKKESVSSVAQENAVKD |
| UniProKB Accession | FTSFSTHNCSGPENPELQILNEQEGKSANYHDKNIVLLKNKAVLMPNGATVSAS |
| No. Q6N021-1 | SVEHTHGELLEKTLSQYYPDCVSIAVQKTTSHINAINSQATNELSCEITHPSHT |
| SEQ ID NO: 283 | SGQINSAQTSNSELPPKPAAVVSEACDADDADNASKLAAMLNTCSFQKPEQLQQ |
| | QKSVFEICPSPAENNIQGTTKLASGEEFCSGSSSNLQAPGGSSERYLKQNEMNG |
| | AYFKQSSVFTKDSFSATTTPPPPSQLLLSPPPPLPQVPQLPSEGKSTINGGVLE |
| | EHHHYPNQSNTTLLREVKIEGKPEAPPSQSPNPSTHVCSPSPMLSERPQNNCVN |
| | RNDIQTAGTMTVPLCSEKTRPMSEHLKHNPPIFGSSGELQDNCQQLMRNKEQEL |
| | KGRDKEQTRDLVPPTQHYLKPGWIELKAPRFHQAESHLKRNEASLPSILQYQPN |
| | LSNQMTSKQYTGNSNMPGGLPRQAYTQKTTQLEHKSQMYQVEMNQGQSQGTVDQ |
| | HLQFQKPSHQVHFSKTDHLPKAHVQSLCGTRFHFQQRADSQTEKLMSPVLKQHL |
| | NQQASETEPFSNSHLLQHKPHKQAAQTQPSQSSHLPQNQQQQQKLQIKNKEEIL |
| | QTFPHPQSNNDQQREGSFFGQTKVEECFHGENQYSKSSEFETHNVQMGLEEVQN |
| | INRRNSPYSQTMKSSACKIQVSCSNNTHLVSENKEQTTHPELFAGNKTQNLHHM |
| | QYFPNNVIPKQDLLHRCEQEQEQKSQQASVLQGYKNRNQDMSGQQAAQLAQQRY |
| | LIHNHANVEPVPDQGGSHTQTPPQKDTQKHAALRWHLLQKQEQQQTQQPQTESC |
| | HSQMHRPIKVEPGCKPHACMHTAPPENKTWKKVTKQENPPASCDNVQQKSIIET |
| | MEQHLKQFHAKSLEDHKALTLKSQKQVKVEMSGPVTVLTRQTTAAELDSHTPAL |
| | EQQTTSSEKTPTKRTAASVINNFIESPSKLLDTPIKNLLDTPVKTQYDFPSCRC |
| | VEQIIEKDEGPFYTHLGAGPNVAAIREIMEERFGQKGKAIRIERVIYTGKEGKS |
| | SQGCPIAKWVVRRSSSEEKLLCLVRERAGHTCEAAVIVILILVWEGIPLSLADK |
| | LYSELTETLRKYGTLINRRCALNEERTCACQGLDPETCGASFSFGCSWSMYYNG |
| | CKFARSKIPRKFKLLGDDPKEEEKLESHLQNLSTLMAPTYKKLAPDAYNNQIEY |
| | EHRAPECRLGLKEGRPFSGVTACLDFCAHAHRDLHNMQNGSTLVCTLTREDNRE |
| | EGGKPEDEQLHVLPLYKVSDVDEFGSVEAQEEKKRSGAIQVLSSFRRKVRMLAE |
| | PVKTCRQRKLEAKKAAAEKLSSLENSSNKNEKEKSAPSRTKQTENASQAKQLAE |
| | LLRLSGPVMQQSQQPQPLQKQPPQPQQQQRPQQQQPHHPQTESVNSYSASGSTN |
| | PYMRRPNPVSPYPNSSHTSDIYGSTSPMNFYSTSSQAAGSYLNSSNPMNPYPGL |
| | LNQNTQYPSYQCNGNLSVDNCSPYLGSYSPQSQPMDLYRYPSQDPLSKLSLPPI |
| | HTLYQPREGNSQSFTSKYLGYGNQNMQGDGFSSCTIRPNVHHVGKLPPYPTHEM |
| | DGHFMGATSRLPPNLSNPNMDYKNGEHHSPSHIIHNYSAAPGMENSSLHALHLQ |
| | NKENDMLSHTANGLSKMLPALNHDRTACVQGGLHKLSDANGQEKQPLALVQGVA |
| | SGAEDNDEVWSDSEQSFLDPDIGGVAVAPTHGSILIECAKRELHATTPLKNPNR |
| | NHPTRISLVFYQHKSMNEPKHGLALWEAKMAEKAREKEEECEKYGPDYVPQKSH |
| | GKKVKREPAEPHETSEPTYLRFIKSLAERTMSVITDSTVTTSPYAFTRVTGPYN |
| | RYI |
| (DNA (cytosine-5)- | MPAMPSSGPGDTSSSAAEREEDRKDGEEQEEPRGKEERQEPSTTARKVGRPGRK |
| methyltransferase 3A) | RKHPPVESGDTPKDPAVISKSPSMAQDSGASELLPNGDLEKRSEPQPEEGSPAG |
| (DNMT3a), isoform 1, | GQKGGAPAEGEGAAETLPEASRAVENGCCTPKEGRGAPAEAGKEQKETNIESMK |
| UniProKB Accession | MEGSRGRLRGGLGWESSL |
| No. Q9Y6K1-1 | RQRPMPRLTFQAGDPYYISKRKRDEWLARWKREAEKKAKVIAGMNAVEENQGPG |
| SEQ ID NO: 284 | ESQKVEEASPPAVQQPTDPASPTVATTPEPVGSDAGDKNATKAGDDEPEYEDGR |
| | GFGIGELVWGKLRGFSWWPGRIVSWWMTGRSRAAEGTRWVMWFGDGKFSVVCVE |
| | KLMPLSSFCSAFHQATYNKQPMYRKAIYEVLQVASSRAGKLFPVCHDSDESDTA |
| | KAVEVQNKPMIEWALGGFQPSGPKGLEPPEEEKNPYKEVYTDMWVEPEAAAYAP |
| | PPPAKKPRKSTAEKPKVKEIIDERTRERLVYEVRQKCRNIEDICISCGSINVTL |
| | EHPLFVGGMCQNCKNCFLECAYQYDDDGYQSYCTICCGGREVLMCGNNNCCRCF |
| | CVECVDLLVGPGAAQAAIKEDPWNCYMCGHKGTYGLLRRREDWPSRLQMFFANN |
| | HDQEFDPPKVYPPVPAEKRKPIRVLSLFDGIATGLLVLKDLGIQVDRYIASEVC |
| | EDSITVGMVRHQGKIMYVGDVRSVTQKHIQEWGPFDLVIGGSPCNDLSIVNPAR |
| | KGLYEGTGRIFFEFYRLLHDARPKEGDDRPFFWLFENVVAMGVSDKRDISRFLE |
| | SNPVMIDAKEVSAAHRARYFWGNLPGMNRPLASTVNDKLELQECLEHGRIAKFS |
| | KVRTITTRSNSIKQGKDQHFPVEMNEKEDILWCTEMERVFGFPVHYTDVSNMSR |
| | LARQRLLGRSWSVPVIRHLFAPLKEYFACV |
| (DNA (cytosine-5)- | MKGDTRHLNGEEDAGGREDSILVNGACSDQSSDSPPILEAIRTPEIRGRRSSSR |
| methyltransferase 3B) | LSKREVSSLLSYTQDLTGDGDGEDGDGSDTPVMPKLFRETRTRSESPAVRTRNN |
| (DNMT3b), isoform | NSVSSRERHRPSPRSTRGRQGRNHVDESPVEFPATRSLRRRATASAGTPWPSPP |
| 1, UniProKB | SSYLTIDLTDDTEDTHGTPQSSSTPYARLAQDSQQGGMESPQVEADSGDGDSSE |
| Accession No. | YQDGKEFGIGDLVWGKIKGFSWWPAMVVSWKATSKRQAMSGMRWVQWEGDGKFS |
| Q9UBC3-1 | EVSADKLVALGLFSQHENLATENKLVSYRKAMYHALEKARVRAGKTFPSSPGDS |
| SEQ ID NO: 285 | LEDQLKPMLEWAHGGFKPTGIEGLKPNNTQPVVNKSKVRRAGSRKLESRKYENK |
| | TRRRTADDSATSDYCPAPKRLKTNCYNNGKDRGDEDQSREQMASDVANNKSSLE |
| | DGCLSCGRKNPVSFHPLFEGGLCQTCRDRFLELFYMYDDDGYQSYCTVCCEGRE |
| | LLLCSNTSCCRCFCVECLEVLVGTGTAAEAKLQEPWSCYMCLPQRCHGVLRRRK |
| | DWNVRLQAFFTSDTGLEYEAPKLYPAIPAARRRPIRVLSLEDGIATGYLVLKEL |
| | GIKVGKYVASEVCEESIAVGTVKHEGNIKYVNDVRNITKKNIEEWGPEDLVIGG |
| | SPCNDLSNVNPARKGLYEGTGRIFFEFYHLINYSRPKEGDDRPFFWMFENVVAM |
| | KVGDKRDISRFLECNPVMIDAIKVSAAHRARYFWGNLPGMNRPVIASKNDKLEL |
| | QDCLEYNRIAKLKKVQTITTKSNSIKQGKNQLFPVVMNGKEDVLWCTELERIFG |
| | FPVHYTDVSNMGRGARQKLLGRSWSVPVIRHLFAPLKDYFACE |
| Neurogenic locus | MPPLLAPLLCLALLPALAARGPRCSQPGETCLNGGKCEAANGTEACVCGGAFVG |
| notch homolog protein | PRCQDPNPCLSTPCKNAGTCHVVDRRGVADYACSCALGFSGPLCLTPLDNACLT |
| 1 (NOTCH1), isoform | NPCRNGGTCDLLTITEYKCRCPPGWSGKSCQQADPCASNPCANGGQCLPFEASY |
| 1, UniProKB | ICHCPPSFHGPTCRQDVNECGQKPGLCRHGGTCHNEVGSYRCVCRATHTGPNCE |
| Accession No. | RPYVPCSPSPCQNGGTCRPTGDVTHECACLPGFTGQNCEENIDDCPGNNCKNGG |
| Q9UBC3-1 | ACVDGVNTYNCRCPPEWTGQYCTEDVDECQLMPNACQNGGTCHNTHGGYNCVCV |
| SEQ ID NO: 286 | NGWTGEDCSENIDDCASAACFHGATCHDRVASFYCECPHGRIGLLCHLNDACIS |
| | NPCNEGSNCDINPVNGKAICTCPSGYTGPACSQDVDECSLGANPCEHAGKCINT |
| | LGSFECQCLQGYTGPRCEIDVNECVSNPCQNDATCLDQIGEFQCICMPGYEGVH |
| | CEVNTDECASSPCLHNGRCLDKINEFQCECPTGETGHLCQYDVDECASTPCKNG |
| | AKCLDGPNTYTCVCTEGYTGTHCEVDIDECDPDPCHYGSCKDGVATFTCLCRPG |
| | YTGHHCETNINECSSQPCRHGGTCQDRDNAYLCFCLKGTTGPNCEINLDDCASS |
| | PCDSGTCLDKIDGYECACEPGYTGSMCNINIDECAGNPCHNGGTCEDGINGFTC |
| | RCPEGYHDPTCLSEVNECNSNPCVHGACRDSLNGYKCDCDPGWSGINCDINNNE |
| | CESNPCVNGGTCKDMTSGYVCTCREGESGPNCQTNINECASNPCLNQGTCIDDV |
| | AGYKCNCLLPYTGATCEVVLAPCAPSPCRNGGECRQSEDYESFSCVCPTGWQGQ |
| | TCEVDINECVLSPCRHGASCQNTHGGYRCHCQAGYSGRNCETDIDDCRPNPCHN |
| | GGSCTDGINTAFCDCLPGFRGTFCEEDINECASDPCRNGANCTDCVDSYTCTCP |
| | AGFSGIHCENNTPDCTESSCENGGTCVDGINSFTCLCPPGFTGSYCQHDVNECD |
| | SQPCLHGGTCQDGCGSYRCTCPQGYTGPNCQNLVHWCDSSPCKNGGKCWQTHTQ |
| | YRCECPSGWTGLYCDVPSVSCEVAAQRQGVDVARLCQHGGLCVDAGNTHHCRCQ |
| | AGYTGSYCEDLVDECSPSPCQNGATCTDYLGGYSCKCVAGYHGVNCSEEIDECL |
| | SHPCQNGGTCLDLPNTYKCSCPRGTQGVHCEINVDDCNPPVDPVSRSPKCENNG |
| | TCVDQVGGYSCTCPPGFVGERCEGDVNECLSNPCDARGTQNCVQRVNDEHCECR |
| | AGHTGRRCESVINGCKGKPCKNGGTCAVASNTARGFICKCPAGFEGATCENDAR |
| | TCGSLRCLNGGTCISGPRSPTCLCLGPFTGPECQFPASSPCLGGNPCYNQGTCE |
| | PTSESPFYRCLCPAKENGLLCHILDYSFGGGAGRDI |
| | PPPLIEEACELPECQEDAGNKVCSLQCNNHACGWDGGDCSLNENDPWKNCTQSL |
| | QCWKYFSDGHCDSQCNSAGCLFDGFDCQRAEGQCNPLYDQYCKDHFSDGHCDQG |
| | CNSAECEWDGLDCAEHVPERLAAGTLVVVVLMPPEQIRNSSFHFLRELSRVLHT |
| | NVVFKRDAHGQQMIFPYYGREEELRKHPIKRAAEGWAAPDALLGQVKASLLPGG |
| | SEGGRRRRELDPMDVRGSIVYLEIDNRQCVQASSQCFQSATDVAAFLGALASLG |
| | SLNIPYKIEAVQSETVEPPPPAQLHEMYVAAAAFVLLFFVGCGVLLSRKRRRQH |
| | GQLWFPEGFKVSEASKKKRREPLGEDSVGLKPLKNASDGALMDDNQNEWGDEDL |
| | ETKKERFEEPVVLPDLDDQTDHRQWTQQHLDAADLRMSAMAPTPPQGEVDADCM |
| | DVNVRGPDGFTPLMIASCSGGGLETGNSEEEEDAPAVISDFIYQGASLHNQTDR |
| | TGETALHLAARYSRSDAAKRLLEASADANIQDNMGRTPLHAAVSADAQGVEQIL |
| | IRNRATDLDARMHDGTTPLILAARLAVEGMLEDLINSHADVNAVDDLGKSALHW |
| | AAAVNNVDAAVVLLKNGANKDMQNNREETPLFLAAREGSYETAKVLLDHFANRD |
| | ITDHMDRLPRDIAQERMHHDIVRLLDEYNLVRSPQLHGAPLGGTPTLSPPLCSP |
| | NGYLGSLKPGVQGKKVRKPSSKGLACGSKEAKDLKARRKKSQDGKGCLLDSSGM |
| | LSPVDSLESPHGYLSDVASPPLLPSPFQQSPSVPLNHLPGMPDTHLGIGHLNVA |
| | AKPEMAALGGGGRLAFETGPPRLSHLPVASGTSTVLGSSSGGALNFTVGGSTSL |
| | NGQCEWLSRLQSGMVPNQYNPLRGSVAPGPLSTQAPSLQHGMVGPLASSLAASA |
| | LSQMMSYQGLPSTRLATQPHLVQTQQVQPQNLQMQQQNLQPANIQQQQSLQPPP |
| | PPQPHLGVSSAASGHLGRSFLSGEPSQADVQPLGPSSLAVHTILPQESPALPTS |
| | LPSSLVPPVTAAQFLTPPSQHSYSSPVDNTPSHQLQVPEHPELTPSPESPDQWS |
| | SSSPHSNVSDWSEGVSSPPTSMQSQIARIPEAFK |

TABLE 1
Full vector insert examples

| ID | Description |
|---|---|
| AIO1 (SEQ ID NO: 95) | All-in-one Unidirectional-reverse |
| AIO2 (SEQ ID NO: 96) | All-in-one Unidirectional-forward |
| AIO3 (SEQ ID NO: 97) | All-in-one Bidirectional |
| IPV1 (SEQ ID NO: 98) | synPA-tagBFP-MND-bGHpA-sfGFP-minCMV-5xGal4RE |
| IPV2 (SEQ ID NO: 99) | synPA-tagBFP-MND-bGHpA-EGFP-minCMV-5xGal4RE |
| IPV3 (SEQ ID NO: 100) | synPA-tagBFP-MND-bGHpA-EGFP-YB_TATA-5xGal4RE |
| IPV4 (SEQ ID NO: 101) | synPA-tagBFP-MND-bGHpA-EGFP-minIL2-5xGal4RE |
| IPV5 (SEQ ID NO: 102) | synPA-tagBFP-MND-bGHpA-EGFP-huBG-5xGal4RE |
| IPV6 (SEQ ID NO: 103) | synPA-tagBFP-MND-bGHpA-EGFP-TRE3G-5xGal4RE |
| IPV7 (SEQ ID NO: 104) | synPA-tagBFP-hPGK-bGHpA-EGFP-huBG-5xGal4RE |
| IPV8 (SEQ ID NO: 105) | 5xGal4RE-YB_TATA-EGFP-SV40pA-MND-tagBFP |
| IPV9 (SEQ ID NO: 106) | 6xHIVRE-YB_TATA-EGFP-SV40pA-MND-tagBFP |
| IPV10 (SEQ ID NO: 107) | 6xZF1RE-YB_TATA-EGFP-SV40pA-MND-tagBFP |
| IPV11 (SEQ ID NO: 108) | 6xZF2RE-YB_TATA-EGFP-SV40pA-MND-tagBFP |
| IPV12 (SEQ ID NO: 109) | 6xZF3v1RE-YB_TATA-EGFP-SV40pA-MND-tagBFP |
| IPV13 (SEQ ID NO: 110) | 6xZF3v3RE-YB_TATA-EGFP-SV40pA-MND-tagBFP |
| IPV14 (SEQ ID NO: 111) | 12xHIVRE-YB_TATA-EGFP-SV40pA-MND-tagBFP |
| IPV15 (SEQ ID NO: 112) | 12xZF3v3RE-YB_TATA-EGFP-SV40pA-MND-tagBFP |
| TFV1 (SEQ ID NO: 113) | MND-Gal4DBD-NS3a-T2a-mCherry-P2a-DNCR2-VPR |
| TFV2 (SEQ ID NO: 114) | MND-mCherry-T2a-Gal4DBD-NS3a-P2a-DNCR2-VPR |
| TFV3 (SEQ ID NO: 115) | MND-NS3a-VPR-T2a-mCherry-P2a-Gal4DBD-DNCR2 |
| TFV4 (SEQ ID NO: 116) | MND-NS3a-ZFHIV2-T2a-mCherry-P2a-DNCR2-VPRmini |
| TFV5 (SEQ ID NO: 117) | MND-NS3a-ZFHIV2-T2a-mCherry-P2a-DNCR2-VP64- |
| | RTAmini |
| TFV6 (SEQ ID NO: 118) | MND-NS3a-ZFHIV2-T2a-mCherry-P2a-DNCR2-p65mini- |
| | HSF1 |
| TFV7 (SEQ ID NO: 119) | MND-NS3a-ZFHIV2-T2a-mCherry-P2a-DNCR2-p65mini |
| TFV8 (SEQ ID NO: 120) | MND-NS3a-ZF1-T2a-mCherry-P2a-DNCR2-VPRmini |
| TFV9 (SEQ ID NO: 121) | MND-NS3a-ZF1-T2a-mCherry-P2a-DNCR2-VP64-RTAmini |
| TFV10 (SEQ ID NO: 122) | MND-NS3a-ZF1-T2a-mCherry-P2a-DNCR2-p65mini-HSF1 |
| TFV11 (SEQ ID NO: 123) | MND-NS3a-ZF1-T2a-mCherry-P2a-DNCR2-p65mini |
| TFV12 (SEQ ID NO: 124) | MND-NS3a-ZF2-T2a-mCherry-P2a-DNCR2-VPRmini |
| TFV13 (SEQ ID NO: 125) | MND-NS3a-ZF2-T2a-mCherry-P2a-DNCR2-VP64-RTAmini |
| TFV14 (SEQ ID NO: 126) | MND-NS3a-ZF2-T2a-mCherry-P2a-DNCR2-p65mini-HSF1 |
| TFV15 (SEQ ID NO: 127) | MND-NS3a-ZF3-T2a-mCherry-P2a-DNCR2-VPRmini |
| TFV16 (SEQ ID NO: 128) | MND-NS3a-ZF3-T2a-mCherry-P2a-DNCR2-VP64-RTAmini |
| TFV17 (SEQ ID NO: 129) | MND-NS3a-ZF3-T2a-mCherry-P2a-DNCR2-p65mini-HSF1 |
| TFV18 (SEQ ID NO: 130) | MND-NS3a-ZF3-T2a-mCherry-P2a-DNCR2-p65mini |
| TFV19 (SEQ ID NO: 131) | MND-NS3a-LZ-ZFHIV2-T2a-mCherry-P2a-DNCR2-VPRmini |
| TFV20 (SEQ ID NO: 132) | MND-NS3a-LZ-ZF3-T2a-mCherry-P2a-DNCR2-VPRmini |

TABLE 2
IPV and TFV vectors for testing DNA binding domains
and transcriptional activation domains.

| ID | Description |
|---|---|
| IPV8 (SEQ ID NO: 105) | 5xGal4RE-YB_TATA-EGFP-SV40pA-MND-tagBFP |
| IPV9 (SEQ ID NO: 106) | 6xHIVRE-YB_TATA-EGFP-SV40pA-MND-tagBFP |
| IPV10 (SEQ ID NO: 107) | 6xZF1RE-YB_TATA-EGFP-SV40pA-MND-tagBFP |
| IPV11 (SEQ ID NO: 108) | 6xZF2RE-YB_TATA-EGFP-SV40pA-MND-tagBFP |
| IPV12 (SEQ ID NO: 109) | 6xZF3v1RE-YB_TATA-EGFP-SV40pA-MND-tagBFP |
| IPV13 (SEQ ID NO: 110) | 6xZF3v3RE-YB_TATA-EGFP-SV40pA-MND-tagBFP |
| IPV14 (SEQ ID NO: 111) | 12xHIVRE-YB_TATA-EGFP-SV40pA-MND-tagBFP |
| IPV15 (SEQ ID NO: 112) | 12xZF3v3RE-YB_TATA-EGFP-SV40pA-MND-tagBFP |
| TFV4 (SEQ ID NO: 116) | MND-NS3a-ZFHIV2-T2a-mCherry-P2a-DNCR2-VPRmini |
| TFV5 (SEQ ID NO: 117) | MND-NS3a-ZFHIV2-T2a-mCherry-P2a-DNCR2-VP64-RTAmini |
| TFV6 (SEQ ID NO: 118) | MND-NS3a-ZFHIV2-T2a-mCherry-P2a-DNCR2-p65mini-HSF1 |
| TFV7 (SEQ ID NO: 119) | MND-NS3a-ZFHIV2-T2a-mCherry-P2a-DNCR2-p65mini |
| TFV8 (SEQ ID NO: 120) | MND-NS3a-ZF1-T2a-mCherry-P2a-DNCR2-VPRmini |
| TFV19 (SEQ ID NO: 131) | MND-NS3a-LZ-ZFHIV2-T2a-mCherry-P2a-DNCR2-VPRmini |
| TFV20 (SEQ ID NO: 132) | MND-NS3a-LZ-ZF3-T2a-mCherry-P2a-DNCR2-VPRmini |

TABLE 3
IPV and TFV vectors for optimizing the two-vector system.

| ID | Description |
|---|---|
| IPV16 (SEQ ID | 5xGal4RE-huBG-EGFP |
| NO: 144) | |
| IPV17 (SEQ ID | 5xGal4RE-huBG-EGFP-P2a-Gal4DBD-KRAB |
| NO: 145) | |
| IPV18 (SEQ ID | 5xGal4RE-huBG-EGFP-T2a-ANR-SPOP |
| NO: 146) | |
| IPV19 (SEQ ID | 5xGal4RE-huBG-EGFP-P2a-DHD37-2A-SPOP |
| NO: 147) | |
| TFV21 (SEQ ID | MND-Gal4DBD-NS3a-T2a-P2a-DNCR2-VPR |
| NO: 148) | |
| TFV22 (SEQ ID | MND-Gal4DBD-NS3a-T2a-Gal4DBD-KRAB-P2a- |
| NO: 149) | DNCR2-VPR |
| TFV23 (SEQ ID | MND-Gal4DBD-NS3a-T2a-ANR-SPOP-P2a-DNCR2- |
| NO: 150) | VPR |
| TFV24 (SEQ ID | MND-Gal4DBD-NS3a-DHD37-2B-T2a-RFP-P2a- |
| NO: 151) | DNCR2-VPR |

TABLE 4

| Disease or disorder | One or more Genes Targeted |
|---|---|
| Autoimmune: | ABCC8, ADIPOQ, ADRB3, AGPAT2, AKT2, ALMS1, ANGPTL8, APPL1, |
| | AQP2, AVP, AVPR2, BANK1, BCAR1, BLK, BSCL2, C4A, C4B, CAPN10, |
| | CAV1, CAVIN1, CCR5, CD38, CDKAL1, CEL, CELA2A, CISD2, CLEC16A, |
| | CLPS, CR2, CTLA4, DCAF17, DMXL2, DNAJC3, DNASE1, DNASE1L3, |
| | DYRK1B, EIF2AK3, ENPP1, FCGR2B, FOXP3, GCGR, GCK, GLIS3, GSK3A, |
| | GSK3B, GYS1, HNF1B, HNF4A, IER3IP1, IFIH1, IL2RA, INPPL1, INS, INSR, |
| | IRF5, IRS1, ITGAM, KCNJ11, KCNQ1, KLF11, LEP, LIPE, LTK, MAFA, |
| | MAPK8IP1, MBP, MCF2L2, MT-ND1, NEUROD1, PAX4, PDCD1, PDX1, |
| | PLAGL1, PPARG, PPP1R15B, PPP1R3A, PTF1A, PTPN22, PTPRN, PTPRN2, |
| | RASGRP1, RETN, RFX6, SH2B3, SLC16A11, SLC16A13, SLC19A2, SLC2A4, |
| | SLC30A8, SSB, STAT3, STAT4, SUMO4, TBC1D4, TCF7L2, TLR5, TNFSF4, |
| | TREX1, TRMT10A, UCP1, UCP3, WFS1, XRCC5, XRCC6, ZFP57 |
| Blood | ACSL4, ADA, AK1, ALDOA, AMMECR1, ANK1, ATP11C, BPGM, BRCA1, |
| | BRCA2, BRIP1, CD59, CDAN1, CDIN1, CPOX, CYBA, CYBB, CYBC1, EPB41, |
| | EPB42, ERCC4, F8, F9, FANCA, FANCB, FANCC, FANCD2, FANCE, FANCF, |
| | FANCG, FANCI, FANCL, G6PD, GCLC, GPI, GSR, GSS, HBA1, HBA2, HBB, |
| | KCNE5, KCNN4, KLF1, LPIN2, MAD2L2, NCF1, NCF2, NCF4, PALB2, PGK1, |
| | PIEZO1, PKLR, RAD51, RAD51C, RFWD3, RHAG, RPL11, RPL15, RPL18, |
| | RPL26, RPL27, RPL35, RPL35A, RPL5, RPS10, RPS15A, RPS17, RPS19, |
| | RPS24, RPS26, RPS27, RPS28, RPS29, RPS7, SEC23B, SLC2A1, SLC4A1, |
| | SLX4, SPTA1, SPTB, TPI1, TSR2, UBE2T, XRCC2 |
| Bone | ANO5, BMP1, CA2, CLCN7, COL1A1, COL1A2, CREB3L1, CRTAP, FKBP10, |
| | IFITM5, IKBKG, LRP5, MBTPS2, MESD, MITF, OSTM1, P3H1, P4HB, |
| | PLEKHM1, PLOD2, PPIB, SEC24D, SERPINF1, SERPINH1, SNX10, SP7, |
| | SPARC, SPG7, TCIRG1, TENT5A, TMEM38B, TNFRSF11A, TNFSF11, WNT1 |
| Neurological | AARS1, AARS2, ABCA2, ABCA7, ABHD12, ACO2, ACOX1, ACTL6B, |
| | ADAM10, ADAM22, ADPRS, ADRA2B, ADSL, AFG3L2, AGTPBP1, AIFM1, |
| | ALDH18A1, ALDH7A1, ALG13, ALS2, AMPD2, ANG, ANO10, ANXA11, |
| | AP1S1, AP2M1, AP3B2, AP4B1, AP4E1, AP4M1, AP4S1, AP5Z1, APLP1, |
| | APOA1, APOE, APP, APTX, AR, ARHGEF9, ARL6IP1, ARSA, ARSB, ARV1, |
| | ARX, ASAH1, ASCC1, ATG5, ATIC, ATL1, ATL3, ATM, ATN1, ATP13A2, |
| | ATP1A1, ATP2A2, ATP2B3, ATP6AP2, ATP6V1A, ATP7A, ATXN1, ATXN10, |
| | ATXN2, ATXN3, ATXN7, B4GALNT1, BCKDK, BEAN1, BICD2, BRAT1, |
| | BSCL2, C12orf65, C19orf12, C1orf194, C9orf72, CACNA1A, CACNA1B, |
| | CACNA1D, CACNA1E, CACNA1G, CACNA1H, CACNA2D2, CACNB4, CAD, |
| | CAPN1, CASR, CCDC88A, CCDC88C, CCT5, CDK19, CDK5, CDKL5, CERS1, |
| | CHCHD10, CHCHD2, CHD2, CHMP1A, CHMP2B, CHP1, CHRNA2, |
| | CHRNA4, CHRNB2, CILK1, CLCN2, CLN3, CLN5, CLN6, CLN8, CLP1, CLPB, |
| | CNNM2, CNPY3, CNTN2, CNTNAP1, CNTNAP2, COA7, COASY, COQ2, |
| | COQ8A, COX6A1, CPA6, CPLX1, CPT1C, CRAT, CSF1R, CSNK2B, CSTB, |
| | CTDP1, CTSD, CTSF, CUX2, CWF19L1, CYFIP2, CYP2U1, CYP7B1, DAB1, |
| | DAGLA, DALRD3, DBN1, DCAF8, DCTN1, DCX, DDHD1, DDHD2, DEAF1, |
| | DENND5A, DEPDC5, DGUOK, DHDDS, DHTKD1, DHX16, DIAPH1, DIAPH3, |
| | DLL1, DMXL2, DNAJB2, DNAJC13, DNAJC3, DNAJC6, DNM1, DNM2, |
| | DNMT1, DOCK7, DPP6, DST, DSTYK, DYNC1H1, ECHS1, EEF1A2, EEF2, |
| | EFHC1, EGR2, EIF2S3, EIF4G1, ELOVL4, ELOVL5, ELP1, ELP3, EMC1, |
| | EML1, ENTPD1, EPM2A, EPRS1, ERBB4, ERLIN1, ERLIN2, EXOSC3, |
| | EXOSC8, EXOSC9, EXT2, FA2H, FAR1, FARS2, FARSB, FAT2, FBLN5, |
| | FBXO38, FBXO7, FDXR, FGD4, FGF12, FGF14, FGGY, FIG4, FLVCR1, |
| | FOLR1, FRRS1L, FTL, FUS, GABBR2, GABRA1, GABRA2, GABRA5, GABRB1, |
| | GABRB2, GABRB3, GABRD, GABRG2, GAL, GAN, GARS1, GBA, GBA2, |
| | GBE1, GDAP1, GDAP2, GIGYF2, GJB1, GJC2, GLS, GNAO1, GNB4, GOSR2, |
| | GOT2, GRIA2, GRID2, GRIN2A, GRIN2B, GRIN2D, GRM1, GRM7, GRN, |
| | GUF1, HACE1, HARS1, HCN1, HEXA, HEXB, HINT1, HIP1, HK1, HNRNPA1, |
| | HNRNPU, HSD17B10, HSPB1, HSPB3, HSPB8, HSPD1, HTRA2, HTT, IARS2, |
| | IBA57, IER3IP1, IGHMBP2, INF2, IREB2, IRF2BPL, ITM2B, ITPA, ITPR1, |
| | KARS1, KCNA2, KCNB1, KCNC1, KCNC3, KCND2, KCND3, KCNH1, KCNJ10, |
| | KCNK4, KCNMA1, KCNQ2, KCNQ3, KCNT1, KCNT2, KCTD7, KIDINS220, |
| | KIF1A, KIF1B, KIF1C, KIF5A, KLC2, L1CAM, LAGE3, LGI1, LITAF, LMNA, |
| | LMNB2, LNPK, LRRK2, LRSAM1, MAG, MAPK10, MAPT, MARCHF6, |
| | MARS1, MARS2, MATR3, MCM3AP, MDH1, MDH2, MED25, MEF2C, |
| | MFN2, MFSD8, MME, MORC2, MPV17, MPV17, MPZ, MT-ATP6, MT- |
| | CO1, MT-CO3, MT-CYB, MTHFS, MTMR2, MT-ND1, MT-ND2, MT-ND4, |
| | MT-ND4L, MT-ND5, MT-ND6, MTOR, MTPAP, MYH14, NACC1, NAGA, |
| | NAGLU, NALCN, NAXD, NAXE, NDRG1, NECAP1, NEFH, NEFL, NEK1, |
| | NEUROD2, NGF, NHLRC1, NHLRC2, NIPA1, NKX6-2, NOP56, |
| | NOTCH2NLC, NPRL2, NPRL3, NRROS, NT5C2, NTRK2, NUP107, NUP133, |
| | OPA1, OPTN, OSGEP, OTOF, OTUD6B, OXR1, P4HTM, PACS2, PAK1, |
| | PANK2, PARK7, PARS2, PCDH12, PCDH19, PCLO, PCNA, PCYT2, PDK3, |
| | PDXK, PDYN, PFN1, PHACTR1, PHF21A, PHF6, PIGA, PIGB, PIGH, PIGK, |
| | PIGN, PIGP, PIGQ, PIGS, PIGT, PIGU, PIK3R5, PINK1, PLA2G6, PLCB1, |
| | PLD3, PLEKHG5, PLP1, PLPBP, PMP2, PMP22, PMPCA, PMPCB, PNKP, |
| | PNPLA6, PNPO, POLG, PPP2R2B, PPP3CA, PPP5C, PPT1, PRDM12, |
| | PRDM12, PRDM8, PRICKLE1, PRICKLE2, PRICKLE3, PRKCG, PRKN, PRPH, |
| | PRPS1, PRRT2, PRUNE1, PRX, PSAP, PSEN1, PSEN2, PTPN23, PUM1, |
| | QARS1, RAB10, RAB7A, RAPGEF2, RARS2, REEP1, REEP2, RELN, REPS1, |
| | RETREG1, RFC1, RHOBTB2, RNF13, ROGDI, RORA, RORB, RPIA, RRM2B, |
| | RTN2, RUBCN, SACS, SAMD12, SARS1, SBF1, SBF2, SBF2, SCARB2, |
| | SCN11A, SCN1A, SCN1B, SCN2A, SCN3A, SCN8A, SCN9A, SCYL1, |
| | SELENOI, SEMA6B, SEPSECS, SERPINI1, SETD1A, SETX, SH3TC2, |
| | SIGMAR1, SIK1, SIRT2, SLC12A1, SLC12A5, SLC12A6, SLC13A5, SLC1A2, |
| | SLC1A4, SLC25A19, SLC25A22, SLC25A46, SLC2A1, SLC30A10, SLC33A1, |
| | SLC35A2, SLC35A3, SLC39A14, SLC44A1, SLC45A1, SLC5A6, SLC5A7, |
| | SLC6A1, SLC6A3, SLC9A1, SLC9A6, SMC1A, SMN1, SMN2, SMPD1, |
| | SNAP29, SNCA, SNCAIP, SNIP1, SNX14, SOD1, SORD, SORL1, SPART, |
| | SPAST, SPATA5, SPG11, SPG21, SPG7, SPTAN1, SPTBN2, SPTBN4, |
| | SPTLC1, SPTLC2, SQSTM1, SRPX2, ST3GAL3, ST3GAL5, STUB1, STX1B, |
| | STXBP1, SUMF1, SUOX, SURF1, SYN1, SYNE1, SYNJ1, SYT14, SZT2, |
| | TANGO2, TARDBP, TBC1D24, TBCD, TBCE, TBK1, TBP, TCF4, TDP1, TDP2, |
| | TECPR2, TFG, TGM6, THG1L, TIMM50, TMEM106B, TMEM175, |
| | TMEM230, TMEM240, TMX2, TNRC6A, TOE1, TP53RK, TPP1, TPRKB, |
| | TRAK1, TRAPPC2L, TRAPPC6B, TREM2, TREX1, TRIM2, TRIP4, TRPC3, |
| | TRPM7, TRPV4, TSC1, TSC2, TSEN2, TSEN34, TSEN54, TTBK2, TTR, |
| | TUBA4A, TUBGCP2, TWNK, TXN2, TYMP, UBA1, UBA5, UBAP1, UBQLN2, |
| | UBQLN4, UBTF, UCHL1, UGDH, UGP2, UNC5C, UNC80, VAC14, VAMP1, |
| | VAPB, VARS1, VCP, VPS13A, VPS13C, VPS13D, VPS35, VPS37A, VPS53, |
| | VRK1, VWA3B, WARS1, WASF1, WASHC5, WDR4, WDR45, WDR45B, |
| | WDR73, WNK1, WWOX, XRCC1, YARS1, YEATS2, YWHAG, ZEB2, |
| | ZFYVE26, ZFYVE27, ZNHIT3 |
| Cardiovascular | AARS2, ABCC9, ACADVL, ACTC1, ACTN2, AGK, ALPK3, BAG3, BRAF, |
| | CALR3, CAV3, CDH2, CRYAB, CSRP3, CTNNA3, DES, DMD, DNAJC19, |
| | DSC2, DSG2, DSP, DTNA, EMD, EYA4, FKRP, FKTN, FLNC, GATA4, |
| | GATAD1, GJA5, GRM7, GTPBP3, JPH2, JUP, KRAS, LAMA4, LDB3, LMNA, |
| | MAP2K1, MAP2K2, MIB1, MT-ATP6, MT-ATP8, MT-CYB, MTO1, MYBPC3, |
| | MYH6, MYH7, MYL2, MYL3, MYLK2, MYO6, MYOZ2, MYPN, NDUFB11, |
| | NEXN, NPPA, OPA1, PKP2, PLN, PPCS, PRDM16, PRKAG2, PSEN1, PSEN2, |
| | RAF1, RBM20, RYR2, SCN5A, SCO2, SDHA, SGCD, SLC25A4, TAZ, TBX20, |
| | TBX5, TCAP, TGFB3, TMEM43, TMPO, TNNC1, TNNI3, TNNI3K, TNNT2, |
| | TPM1, TSFM, TTN, VCL, ZFPM2 |
| Metabolic | AGL, ALDOA, ARSB, BCKDHA, BCKDHB, DBT, ENO3, EPM2A, ETFA, ETFB, |
| | ETFDH, G6PC, GAA, GALNS, GBA, GBE1, GCDH, GCH1, GLB1, GNS, GUSB, |
| | GYG1, GYS2, HGSNAT, HYAL1, IDS, IDUA, LAMP2, LDHA, NAGLU, PAH, |
| | PFKM, PGAM2, PGM1, PHKA1, PHKA2, PHKB, PHKG2, PRKAG2, PSAP, |
| | PTS, PYGL, PYGM, QDPR, SGSH, SUGCT, SUMF1, VPS33A |
| | AAGAB, ANAPC1, AQP5, BMS1, BRAF, CAST, CD151, CDH1, CDH3, |
| | COL11A1, COL17A1, COL7A1, CST6, CTNND1, CTSC, CYP26C1, DDB2, |
| | DPH1, DSG1, DSP, DST, EDA, EDAR, EDARADD, ERCC6, ERCC2, ERCC3, |
| | ERCC4, ERCC5, EVC, EVC2, EXPH5, FGF10, FGFR2, FGFR3, GJA1, GJB2, |
| | GJB3, GJB4, GJB6, GRHL2, HOXC13, IFT122, IFT43, IKBKG, ITGA3, ITGA6, |
| | ITGB4, JUP, KANK2, KDF1, KDSR, KLHL24, KRAS, KREMEN1, KRT1, KRT14, |
| | KRT16, KRT17, KRT5, KRT6A, KRT6B, KRT6C, KRT74, KRT83, KRT85, KRT9, |
| | LAMA3, LAMB3, LAMC2, LORICRIN, MAP2K1, MAP2K2, MBTPS2, MSX1, |
| | NECTIN1, NECTIN4, NFKBIA, NLRP1, PKP1, PLEC, POLH, POMP, PRKD1, |
| | RHBDF2, RHOA, RIPK4, RSPO1, SASH1, SERPINB7, SLURP1, SMARCAD1, |
| | SNAP29, TAT, TP63, TRPM4, TRPV3, TSPEAR, TWIST2, WDR19, WDR35, |
| | WNT10A, XPA, XPC |
| Mitochondrial | AARS2, ACAD9, AGK, AIFM1, ATP5F1A, ATP5F1D, ATP5F1E, ATP5MD, |
| | ATPAF2, BCS1L, BOLA3, C12orf65, C1QBP, CARS2, COA3, COA5, COA6, |
| | COA8, COQ2, COQ4, COQ6, COQ7, COQ8A, COQ9, COX10, COX14, |
| | COX15, COX20, COX411, COX5A, COX6A2, COX6B1, COX8A, CYC1, |
| | DGUOK, DNA2, EARS2, ELAC2, FARS2, FASTKD2, FBXL4, FDX2, FLAD1, |
| | FOXRED1, GATB, GATC, GFER, GFM1, GFM2, GTPBP3, IBA57, ISCA2, |
| | LRPPRC, LYRM4, LYRM7, MARS2, MGME1, MICOS13, MIPEP, MPV17, |
| | MRM2, MRPL12, MRPL3, MRPL44, MRPS14, MRPS16, MRPS2, MRPS22, |
| | MRPS23, MRPS28, MRPS34, MRPS7, MT-ATP6, MT-ATP8, MT-CO1, MT- |
| | CO2, MT-CO3, MT-CYB, MTFMT, MT-ND1, MT-ND2, MT-ND3, MT-ND4, |
| | MT-ND4L, MT-ND5, MT-ND6, MTO1, NARS2, NDUFA1, NDUFA10, |
| | NDUFA11, NDUFA12, NDUFA13, NDUFA2, NDUFA4, NDUFA6, NDUFA9, |
| | NDUFAF1, NDUFAF2, NDUFAF3, NDUFAF4, NDUFAF5, NDUFAF6, |
| | NDUFAF8, NDUFB11, NDUFB3, NDUFB8, NDUFB9, NDUFS1, NDUFS2, |
| | NDUFS3, NDUFS4, NDUFS6, NDUFS7, NDUFS8, NDUFV1, NDUFV2, NFU1, |
| | NSUN3, NUBPL, OPA1, PDHA1, PDSS1, PDSS2, PET100, PET117, PINK1, |
| | PNPLA4, PNPT1, POLG, POLG2, PRICKLE3, PUS1, QRSL1, RMND1, |
| | RNASEH1, RRM2B, SCO1, SCO2, SDHA, SDHAF1, SDHD, SFXN4, |
| | SLC25A10, SLC25A21, SLC25A26, SLC25A4, SUCLA2, SUCLG1, SURF1, |
| | TACO1, TARS2, TFAM, TIMM22, TIMMDC1, TK2, TMEM126B, TMEM70, |
| | TOP3A, TRIT1, TRMT10C, TRMT5, TSFM, TTC19, TUFM, TWNK, TXN2, |
| | TYMP, UQCC2, UQCC3, UQCRB, UQCRC2, UQCRFS1, UQCRQ, VARS2, |
| | WARS2, YARS2 |
| Muscle | ANO5, B3GALNT2, B4GAT1, BVES, CAPN3, CAV3, CHKB, COL12A1, |
| | COL6A1, COL6A2, COL6A3, CRPPA, DAG1, DES, DNAJB6, DPM1, DPM2, |
| | DPM3, DYSF, EMD, FHL1, FKRP, FKTN, GIPC1, GMPPB, HNRNPDL, |
| | INPP5K, ITGA7, LAMA2, LARGE1, LIMS2, LMNA, MYOT, PLEC, POGLUT1, |
| | POMGNT1, POMGNT2, POMK, POMT1, POMT2, POPDC3, PYROXD1, |
| | RXYLT1, SGCA, SGCB, SGCD, SGCG, SYNE1, SYNE2, TCAP, TMEM43, |
| | TNPO3, TOR1AIP1, TRAPPC11, TRIM32, TRIP4, TTN |
| Liver | ABCB11, ABCB4, AKR1D1, AMACR, ATP8B1, CYP7B1, HSD3B7, NR1H4, |
| | SLC25A13, TJP2 |
| Hearing | ABCC1, ABHD12, ABHD5, ACOX1, ACSL4, ACTB, ACTG1, ADAMTS17, |
| | ADCY1, ADGRV1, AIFM1, ALMS1, AMMECR1, ANKH, AP000812.4, |
| | AP1B1, AP1S1, ARSG, ATP1A3, ATP6V1B1, ATP6V1B2, BCAP31, BCS1L, |
| | BDP1, BRAF, BSND, CABP2, CACNA1D, CCDC50, CD151, CD164, CDC14A, |
| | CDH11, CDH23, CEACAM16, CEP250, CEP78, CIB2, CISD2, CLCNKA, |
| | CLCNKB, CLDN14, CLIC5, CLPP, CLRN1, COCH, COL11A1, COL11A2, |
| | COL2A1, COL4A3, COL4A4, COL4A5, COL4A6, COL9A1, COL9A2, COQ6, |
| | CRYM, DCAF17, DCDC2, DCHS1, DIABLO, DIAPH1, DIAPH3, DLX5, |
| | DMXL2, DNAJC3, DNMT1, DSPP, EDN3, EDNRB, ELMOD3, EPS8, EPS8L2, |
| | ERAL1, ERCC2, ERCC3, ERCC5, ERCC6, ERCC8, ESPN, ESRP1, ESRRB, |
| | EXOSC2, EYA1, EYA4, FAT4, FDXR, FGF9, FGFR3, FITM2, FKBP14, FLNA, |
| | FOXC1, GAB1, GATA3, GFER, GIPC3, GJB2, GJB3, GJB6, GPC4, GPRASP2, |
| | GPSM2, GRAP, GRHL2, GRXCR1, GRXCR2, GSDME, HARS1, HARS2, HGF, |
| | HOMER2, HSD17B4, IARS2, IGF1, ILDR1, ITM2B, JAG1, KARS1, KCNE1, |
| | KCNE5, KCNH2, KCNJ10, KCNQ1, KCNQ4, KITLG, KRAS, LARS2, LHFPL5, |
| | LMX1A, LONP1, LOXHD1, LOXL3, MAF, MARS2, MARVELD2, MCM2, |
| | MET, MGP, MITF, MPZ, MPZL2, MSRB3, MT-CO1, MT-CYB, MYH14, |
| | MYH9, MYO15A, MYO1F, MYO3A, MYO6, MYO7A, NARS2, NDP, NF2, |
| | NLRP3, NOG, OPA1, OSBPL2, OTOA, OTOF, OTOG, OTOGL, P2RX2, PAX1, |
| | PAX3, PCDH15, PCNA, PDE1C, PDZD7, PEX1, PEX6, PEX7, PHYH, PIGL, |
| | PISD, PJVK, PLS1, PMP22, PNPT1, POU4F3, PPIP5K2, PPP2R3C, PRPS1, |
| | PTPN11, RAB40AL, RAF1, RDX, REST, RIPOR2, ROR1, RPGR, RPS23, |
| | S1PR2, SALL1, SERAC1, SERPINB6, SIX1, SIX5, SLC17A8, SLC19A2, |
| | SLC26A4, SLC26A5, SLC33A1, SLC44A4, SLC4A11, SLC52A2, SLC52A3, |
| | SLC9A1, SLITRK6, SMPX, SNAI2, SOX10, SPATA5, SPNS2, SPTBN4, STRC, |
| | SYNE4, TBC1D24, TBL1X, TBL1Y, TBX22, TECTA, THRB, TIMM8A, TMC1, |
| | TMEM132E, TMEM67, TMIE, TMPRSS3, TNC, TNFRSF11A, TPRN, |
| | TRAPPC12, TRIOBP, TRMU, TRRAP, TSPEAR, TUBB4B, TWNK, TXNL4A, |
| | TYR, USH1C, USH1G, USH2A, WBP2, WFS1, WHRN |
| Opthalmic | ARMS2, ABCA4, ABCA4, ABHD12, ABHD5, ADGRV1, AGBL1, AGBL5, AGK, |
| | AGPS, AHR, AIPL1, ARHGEF18, ARL6, ARSG, ASB10, BBS2, BEST1, BFSP1, |
| | BFSP2, C2, C3, C8orf37, C9, CA4, CACNA1F, CDH23, CEP290, CERKL, CFB, |
| | CFH, CFI, CHMP4B, CHST6, CIB2, CLCC1, CLPB, CLRN1, CNGA1, CNGA3, |
| | CNGB1, CNGB3, COL11A1, COL18A1, COL2A1, COL8A2, CRB1, CRB2, |
| | CRX, CRYAA, CRYAA2, CRYAB, CRYBA1, CRYBA2, CRYBA4, CRYBB1, |
| | CRYBB2, CRYBB3, CRYGB, CRYGC, CRYGD, CRYGS, CST3, CTDP1, CWC27, |
| | CX3CR1, CYP1B1, CYP27A1, CYP4V2, DCN, DHDDS, DHX38, DMPK, |
| | DNMBP, EBP, ELOVL4, EPG5, EPHA2, ERCC1, ERCC2, ERCC6, ESPN, |
| | EXOSC2, EYS, FAM126A, FAM161A, FAR1, FBLN5, FLVCR1, FOXE3, |
| | FSCN2, FTL, FYCO1, GALK1, GALT, GCNT2, GDF6, GFER, GJA1, GJA3, |
| | GJA8, GLS, GNAT1, GNB3, GNPAT, GPATCH3, GRHL2, GRK1, GRM6, GSN, |
| | GUCA1B, GUCY2D, HARS1, HGSNAT, HK1, HMCN1, HMX1, HSF4, HTRA1, |
| | IARS2, IDH3B, IFT140, IFT172, IFT43, IMPDH1, IMPG2, INPP5K, IQCB1, |
| | KCNA4, KCNJ13, KIAA1549, KIF3B, KIZ, KLHL7, KRT12, KRT3, LCA5, LCAT, |
| | LEMD2, LIM2, LONP1, LOXL1, LRAT, LRIT3, LSS, LTBP2, MAF, MAK, |
| | MBNL1, MERTK, MFRP, MIP, MSMO1, MT-ATP6, MT-CO1, MT-CO3, MT- |
| | CYB, MT-ND1, MT-ND2, MT-ND4, MT-ND4L, MT-ND5, MT-ND6, MVK, |
| | MYH9, MYO7A, MYOC, NEK2, NHS, NMNAT1, NPHP1, NPHP4, NR2E3, |
| | NRL, NTF4, NUP188, NYX, OCRL, OFD1, OPA3, OPTN, OVOL2, PCARE, |
| | PCDH15, PDE6A, PDE6B, PDE6G, PDZD7, PEX7, PHYH, PIK3C2A, PIKFYVE, |
| | PISD, PITX3, PNPLA6, POMGNT1, PRCD, PRICKLE3, PROM1, PRPF3, |
| | PRPF31, PRPF4, PRPF6, PRPF8, PRPH2, PRPS1, RAB3GAP2, RAX2, RBP3, |
| | RD3, RDH11, RDH12, RECQL4, REEP6, RGR, RHO, ROM1, RP1, RP1L1, |
| | RP2, RP9, RPE65, RPGR, RPGRIP1, SAG, SCAPER, SDCCAG8, SEMA4A, |
| | SIPA1L3, SIX6, SLC16A12, SLC24A1, SLC2A1, SLC33A1, SLC4A11, SLC4A4, |
| | SLC7A14, SNRNP200, SPATA7, SRD5A3, TACSTD2, TBK1, TCF4, TDRD7, |
| | TEK, TGFBI, TKFC, TLR4, TMCO1, TMEM67, TOPORS, TRAF3IP1, TRNT1, |
| | TRPM1, TTC8, TUBB4B, TULP1, UBIAD1, UNC45B, USH1C, USH1G, |
| | USH2A, USP45, VCAN, VIM, VSX1, VSX2, WDR19, WDR36, WFS1, WHRN, |
| | ZEB1, ZNF408, ZNF513 |
| Cancer | A2M, AARS2, ABCB1, ABCC1, ABCC2, ABCC3, ABCC5, ABCC6, ABCG2, |
| | ABI1, ABL1, ABL2, ACAP1, ACKR3, ACLY, ACO1, ACP3, ACSL3, ACVR1, |
| | ACVR1B, ACVR2A, ACVR2B, ADAM10, ADAM9, ADAMTS1, ADAMTS14, |
| | ADAMTS18, ADAMTS20, ADAMTS3, ADAMTS4, ADAMTS5, ADAMTS6, |
| | ADAMTS8, ADAMTS9, ADCY1, ADGRB1, ADM, ADNP, ADORA2A, |
| | ADRA1B, AFDN, AFF1, AFF3, AFF4, AFP, AGER, AHNAK2, AHR, AHSG, |
| | AJUBA, AK9, AKAP12, AKAP9, AKR1B1, AKT1, AKT2, AKT3, ALB, ALCAM, |
| | ALDOA, ALDOB, ALDOC, ALK, ALKBH6, ALPK2, ALPL, ALPP, AMER1, |
| | AMPH, ANAPC1, ANG, ANGPT1, ANGPT2, ANK3, ANKRD12, ANXA1, |
| | ANXA11, ANXA2, ANXA4, ANXA7, AOC3, AP2B1, APAF1, APC, APEX1, |
| | APOA1, APOA2, APOBEC3B, APOC1, APOC3, APOD, APOE, APOL2, |
| | APPBP2, AR, AREG, ARG2, ARHGAP26, ARHGAP32, ARHGAP35, |
| | ARHGEF12, ARHGEF6, ARID1A, ARID1B, ARID2, ARID5B, ARNT, ASPH, |
| | ASPM, ASPSCR1, ASXL1, ASXL2, ATF1, ATG13, ATIC, ATM, ATOH1, |
| | ATP1A1, ATP2B3, ATP7B, ATR, ATRX, AURKA, AURKB, AXIN1, AXIN2, |
| | AZGP1, B2M, BAD, BAG1, BAP1, BARD1, BAX, BCL10, BCL11A, BCL11B, |
| | BCL2, BCL2A1, BCL2L1, BCL2L2, BCL2L2-PABPN1, BCL3, BCL6, BCL7A, |
| | BCL9, BCL9L, BCLAF1, BCOR, BCORL1, BCR, BDNF, BHMT2, BIRC2, BIRC3, |
| | BIRC5, BIRC6, BIVM-ERCC5, BLK, BLM, BLMH, BMI1, BMP2, BMP4, |
| | BMPR1A, BNIP3, BNIP3L, BRAF, BRCA1, BRCA2, BRD3, BRD4, BRIP1, |
| | BRMS1, BTG1, BTG2, BTK, BUB1B, C1QBP, C3orf70, C6, C7, CA8, |
| | CACNA1D, CAD, CALCA, CALR, CAMTA1, CANT1, CANX, CAP2, CAPN6, |
| | CARD11, CARM1, CARS1, CASC3, CASP1, CASP10, CASP2, CASP3, CASP4, |
| | CASP5, CASP6, CASP7, CASP8, CASP9, CAST, CAT, CAV1, CBFA2T3, CBFB, |
| | CBL, CBLB, CBLC, CCAR1, CCDC120, CCDC6, CCKBR, CCL11, CCL13, |
| | CCL14, CCL16, CCL18, CCL19, CCL2, CCL21, CCL23, CCL3, CCL4, CCL5, |
| | CCL7, CCL8, CCN2, CCN4, CCNA1, CCNA2, CCNB1, CCNB1IP1, CCNB2, |
| | CCND1, CCND2, CCND3, CCNE1, CCNE2, CCNG1, CCNG2, CCNH, CCR10, |
| | CCR7, CD14, CD1D, CD24, CD27, CD274, CD36, CD38, CD40, CD40LG, |
| | CD44, CD46, CD52, CD59, CD70, CD74, CD79A, CD79B, CD82, CD9, |
| | CDC16, CDC20, CDC25A, CDC25B, CDC25C, CDC27, CDC34, CDC37, |
| | CDC6, CDC73, CDH1, CDH11, CDH17, CDH5, CDK1, CDK12, CDK2, CDK4, |
| | CDK6, CDK7, CDKN1A, CDKN1B, CDKN1C, CDKN2A, CDKN2C, CDX2, |
| | CEACAM5, CEACAM6, CEBPA, CENPF, CEP43, CEP76, CFH, CFHR1, |
| | CFLAR, CFTR, CGA, CHCHD7, CHD4, CHD7, CHD8, CHEK1, CHEK2, CHFR, |
| | CHGA, CHI3L1, CHP2, CIB2, CIC, CIITA, CKB, CKS1B, CKS2, CLDN3, CLDN4, |
| | CLDN7, CLEC3B, CLIC1, CLIP1, CLSTN1, CLTC, CLTCL1, CLU, CNBD1, |
| | CNBP, CNKSR1, CNN1, CNOT3, CNTF, CNTRL, COL11A1, COL17A1, |
| | COL18A1, COL1A1, COL1A2, COL2A1, COL4A2, COL4A3, COL4A4, |
| | COL4A5, COL5A1, COL5A3, COL6A1, COX17, CP, CRABP1, CRADD, |
| | CREB1, CREB3L1, CREB3L2, CREBBP, CRLF2, CRP, CRTC1, CRTC3, CRYAB, |
| | CSDE1, CSE1L, CSF1, CSF1R, CSF2, CSF2RA, CSF3, CSF3R, CSN1S1, |
| | CSNK1E, CSNK2A2, CSNK2B, CST3, CST6, CSTA, CSTB, CTAG1A, CTAG1B, |
| | CTAG2, CTCF, CTNNB1, CTNNBL1, CTNND1, CTSB, CTSD, CTSH, CTSL, |
| | CTTN, CUL1, CUL2, CUL4B, CUL5, CUX1, CXCL1, CXCL10, CXCL13, CXCL2, |
| | CXCL5, CXCL8, CXCL9, CXCR1, CXCR2, CXCR4, CYB5R3, CYLD, CYP19A1, |
| | CYP1A2, CYP2C19, CYP2E1, CYP3A4, CYP3A5, DAD1, DAPK1, DAXX, DBI, |
| | DCC, DCN, DCTN1, DDB2, DDIT3, DDR2, DDX10, DDX3X, DDX5, DDX6, |
| | DEFA1, DEFA1B, DEFA3, DEK, DES, DHFR, DHX9, DIAPH1, DIAPH3, |
| | DICER1, DIS3, DLC1, DMD, DNAH12, DNAJB1, DNAJC2, DNER, DNM2, |
| | DNMT3A, DOCK2, DROSHA, DST, DUSP1, DUSP14, DUSP4, DVL3, |
| | DYNLL1, DYRK2, E2F1, E2F3, E2F5, EBAG9, EBF1, EDN1, EEF1A1, EEF2, |
| | EFNA1, EFNA2, EFNA5, EFNB1, EFNB2, EFNB3, EGF, EGFR, EGR1, EI24, |
| | EIF2S2, EIF3E, EIF3H, EIF4A2, EIF4E, EIF4EBP1, EIF4G1, EIF4H, EIF5A, |
| | ELANE, ELF3, ELF4, ELK3, ELK4, ELL, EML4, ENC1, ENG, ENO1, ENO2, |
| | ENPP2, EP300, EPAS1, EPCAM, EPHA1, EPHA2, EPHA3, EPHA4, EPHA7, |
| | EPHA8, EPHB2, EPHB3, EPHB4, EPHX1, EPO, EPOR, EPS15, ERBB2, |
| | ERBB3, ERBB4, ERC1, ERCC1, ERCC2, ERCC3, ERCC4, ERCC5, ERCC6, ERG, |
| | ESR1, ESR2, ETNK1, ETV1, ETV4, ETV5, ETV6, EWSR1, EXT1, EXT2, EZH1, |
| | EZH2, EZR, F13A1, F13B, F2, F3, FABP1, FABP2, FABP4, FABP5, FADD, |
| | FAF1, FAM166A, FANCA, FANCC, FANCD2, FANCE, FANCF, FANCG, FAP, |
| | FAS, FASLG, FASN, FAT1, FAT4, FBN2, FBXO11, FBXO6, FBXW7, FCER2, |
| | FCGR2B, FCRL4, FEN1, FES, FEV, FGA, FGB, FGF1, FGF17, FGF18, FGF19, |
| | FGF2, FGF23, FGF3, FGF4, FGF6, FGF7, FGF8, FGF9, FGFBP1, FGFR1, |
| | FGFR2, FGFR3, FGFR4, FGG, FH, FHIT, FIP1L1, FKBP5, FKBP8, FLCN, FLG, |
| | FLI1, FLT1, FLT3, FLT4, FMO5, FN1, FOLH1, FOS, FOSL1, FOXA1, FOXA2, |
| | FOXJ1, FOXL2, FOXM1, FOXO1, FOXO3, FOXO4, FOXP1, FOXQ1, FRMD7, |
| | FSCN1, FSHB, FST, FSTL3, FTH1, FTL, FUBP1, FUS, FZD1, FZD2, G6PD, |
| | GADD45A, GADD45G, GAS1, GAS7, GAST, GATA1, GATA2, GATA3, |
| | GCLM, GDF15, GDNF, GH1, GH2, GJA1, GJB5, GLO1, GMNN, GNA11, |
| | GNA13, GNAI1, GNAQ, GNAS, GNB1, GNPTAB, GOLGA5, GOPC, GOT1, |
| | GOT2, GPA33, GPC3, GPHN, GPI, GPS2, GPX1, GPX2, GRB10, GRB2, |
| | GRB7, GRIN2A, GSK3A, GSN, GSR, GSTM1, GSTM3, GSTP1, GTF2H1, |
| | GUSB, H2AC6, H3-3A, H3-3B, H3C2, H4C9, HDAC10, HDAC2, HDAC5, |
| | HERPUD1, HEY1, HGF, HGFAC, HIF1A, HIP1, HIP1R, HK1, HK2, HLA-A, |
| | HLA-B, HLA-G, HLF, HMGA1, HMGA2, HMGXB4, HMOX1, HNF1A, |
| | HNRNPA2B1, HOOK3, HOXA11, HOXA13, HOXA5, HOXA9, HOXC11, |
| | HOXC13, HOXD11, HOXD13, HP, HPGD, HPN, HRAS, HSF1, HSP90AA1, |
| | HSP90AB1, HSP90B1, HSPA1L, HSPA2, HSPA4, HSPA8, HSPB1, HSPD1, |
| | HSPE1, HSPH1, HUWE1, IBSP, ICAM1, ID1, ID2, ID3, IDH1, IDH2, IDO1, |
| | IFNA1, IFNA13, IFNAR1, IFNAR2, IFNB1, IFNG, IGF1R, IGF2, IGF2R, |
| | IGFBP2, IGFBP3, IKBKB, IKZF1, IL10, IL11, IL12A, IL13, IL13RA2, IL15, |
| | IL16, IL17A, IL17B, IL18, IL1A, IL1B, IL1R1, IL1R2, IL1RN, IL2, IL21R, IL24, |
| | IL2RA, IL2RB, IL2RG, IL4, IL4R, IL5, IL6, IL6R, IL6ST, IL7, IL7R, IL9, ILF3, |
| | ILK, ING1, INHBA, INHBB, INPPL1, INS, INTS12, IPO7, IRF1, IRF4, IRF6, |
| | IRS2, IRS4, ITGA1, ITGA2, ITGA2B, ITGA3, ITGA4, ITGA5, ITGA6, ITGAM, |
| | ITGAV, ITGB1, ITGB3, ITGB4, ITGB5, ITGB6, ITGB7, ITGB8, ITIH4, ITK, |
| | ITPKB, JAK1, JAK2, JAK3, JKAMP, JTB, JUN, JUND, JUP, KALRN, KAT2B, |
| | KAT6A, KAT6B, KCNJ5, KDM5A, KDM5C, KDM6A, KDR, KDSR, KEAP1, |
| | KEL, KIAA1109, KIF2A, KIF2C, KIF5B, KIFC3, KISS1, KIT, KITLG, KLF4, KLF5, |
| | KLF6, KLHL8, KLK10, KLK11, KLK13, KLK14, KLK15, KLK2, KLK3, KLK4, |
| | KLK5, KLK6, KLK7, KLK8, KLK9, KLRK1, KMT2A, KMT2B, KMT2C, KMT2D, |
| | KNL1, KRAS, KRT13, KRT14, KRT15, KRT17, KRT18, KRT19, KRT4, KRT8, |
| | KTN1, LALBA, LAMB1, LAMC1, LASP1, LATS1, LATS2, LCK, LCN1, LCP1, |
| | LCTL, LDHA, LEF1, LEP, LEPR, LGALS3, LGALS3BP, LGALS4, LGI1, LGMN, |
| | LHB, LHX1, LIF, LIFR, LIG4, LIMK1, LMNA, LMO1, LMO2, LPP, LRIG3, |
| | LRP1B, LRP6, LRRK2, LTA, LTA4H, LTB, LTBR, LTF, LUM, LYL1, LYN, LZTR1, |
| | MAD2L1, MAD2L2, MAF, MAFB, MAGEA3, MAGEA4, MAGEA6, |
| | MAGEB5, MAGEB6, MAGEC1, MAGEC2, MAGEC3, MAGED1, MAGED2, |
| | MAGI1, MALT1, MAML2, MAP2K1, MAP2K2, MAP2K4, MAP3K1, |
| | MAP3K13, MAP3K3, MAP3K4, MAP4K3, MAPK1, MAPK14, MAPK3, |
| | MAPK7, MAPK8, MAPK8IP1, MAPKAPK2, MAST2, MATK, MAX, MBD1, |
| | MBD2, MBD4, MCL1, MCM2, MCM3, MCM5, MCM7, MDC1, MDH1, |
| | MDK, MDM2, MDM4, MECOM, MECP2, MED1, MED12, MED13, |
| | MED17, MED23, MEF2A, MEN1, MET, METTL14, MFGE8, MGA, MGMT, |
| | MIA, MIF, MITF, MKI67, MLF1, MLH1, MLH3, MLLT1, MLLT10, MLLT11, |
| | MLLT3, MLLT6, MME, MMP1, MMP10, MMP11, MMP12, MMP13, |
| | MMP14, MMP15, MMP16, MMP2, MMP3, MMP7, MMP8, MMP9, |
| | MN1, MORC4, MPL, MPO, MRE11, MRTFA, MSH2, MSH6, MSI2, MSLN, |
| | MSMB, MSN, MSR1, MST1, MT1A, MT1G, MTA1, MTCP1, MTOR, MUC1, |
| | MUC17, MUTYH, MVP, MXI1, MXRA5, MYB, MYBL2, MYC, MYCL, MYCN, |
| | MYD88, MYH11, MYH9, MYO5A, MYOCD, MYOD1, MYOG, NAB2, NAGA, |
| | NAIP, NAMPT, NAT2, NAV3, NBN, NBPF1, NBPF10, NCAM1, NCOA1, |
| | NCOA2, NCOA3, NCOA4, NCOR1, NCOR2, NDRG1, NEB, NEDD4L, |
| | NEDD8, NEO1, NF1, NF2, NFATC2, NFE2L2, NFIB, NFKB1, NFKB2, |
| | NFKBIA, NFKBIE, NGF, NGFR, NIBAN1, NIN, NKX2-1, NKX3-1, NLRP3, |
| | NME1, NME2, NONO, NOS1, NOS2, NOS3, NOTCH1, NOTCH2, NOTCH3, |
| | NPM1, NQO1, NROB1, NR1H2, NR4A2, NR4A3, NRAS, NRG1, NRG2, |
| | NRG3, NRP1, NRP2, NSD1, NSD2, NSD3, NT5C2, NTF3, NTF4, NTHL1, |
| | NTN1, NTN4, NTRK1, NTRK2, NTRK3, NUCB2, NUDT1, NUMA1, NUMB, |
| | NUP210L, NUP214, NUP93, NUP98, NUTM1, NUTM2B, NUTM2D, |
| | ODAM, OGG1, OLIG2, OMA1, OR4A16, OR51E2, OR52N1, ORM1, OSM, |
| | OTUD7A, P2RY8, PABPC1, PAFAH1B2, PAGE4, PALB2, PAPPA, PARP1, |
| | PARVB, PATZ1, PAX3, PAX5, PAX7, PAX8, PBRM1, PBX1, PCBP1, PCM1, |
| | PCNA, PDAP1, PDCD1LG2, PDCD2L, PDE4DIP, PDGFA, PDGFB, PDGFRA, |
| | PDGFRB, PDSS2, PDZD4, PECAM1, PER1, PF4, PGC, PGF, PGR, PHF20, |
| | PHF6, PHLDA1, PHOX2B, PICALM, PIGR, PIK3CA, PIK3CB, PIK3CG, |
| | PIK3R1, PIK3R2, PIK3R3, PIM1, PIM2, PIM3, PIN1, PIP4K2B, PIP5K1A, |
| | PKM, PLAG1, PLAT, PLAU, PLAUR, PLCG1, PLCG2, PLEC, PLG, PLK1, PLP1, |
| | PMEPA1, PML, PMP22, PMS1, PMS2, PNMT, POLD1, POLE, POLQ, |
| | POMC, PON1, POSTN, POT1, POTEF, POU2AF1, POU2F2, POU5F1, PPA2, |
| | PPARG, PPARGC1A, PPFIBP1, PPM1D, PPP1R15A, PPP2R1A, PPP6C, PPY, |
| | PRCC, PRDM1, PRDM13, PRDM16, PRDX2, PRDX4, PREX2, PRF1, |
| | PRKACA, PRKAR1A, PRKCA, PRKCB, PRKCD, PRKCE, PRKCH, PRKCI, |
| | PRKCQ, PRKDC, PRL, PROC, PRPF8, PRRX1, PRSS1, PSCA, PSEN1, PSIP1, |
| | PSMD4, PTCH1, PTCH2, PTEN, PTGS1, PTGS2, PTH, PTHLH, PTK2, PTK6, |
| | PTN, PTPN11, PTPN13, PTPRB, PTPRC, PTPRK, PTPRO, PTPRT, PTTG1, |
| | PURA, PZP, QKI, RAB11FIP3, RAB18, RAB25, RAB40A, RABEP1, RAC1, |
| | RAD21, RAD23A, RAD23B, RAD51, RAD51B, RAD51D, RAD52, RAD54B, |
| | RAF1, RANBP2, RANBP3, RAP1GDS1, RARA, RARB, RARG, RASA1, RB1, |
| | RBBP4, RBL1, RBL2, RBM10, RBM15, RBM6, RBMX, RBP4, RECQL4, REL, |
| | RELA, RELB, RET, RHEB, RHOA, RHOB, RHOC, RHOH, RIT1, RMC1, RMI2, |
| | RNF213, RNF43, ROS1, RPA2, RPGR, RPL10, RPL22, RPL27, RPL5, RPN1, |
| | RPS15, RPS2, RPS3, RPS6KA1, RPS6KA3, RSBN1L, RSPO2, RSPO3, RUNX1, |
| | RUNX1T1, RXRA, RXRB, RXRG, S100A1, S100A2, S100A4, S100A6, |
| | S100A7, S100A8, S100A9, S100B, S1PR1, SACS, SALL4, SART1, SBDS, |
| | SCGB1A1, SCGB1D2, SCGB2A1, SCGB2A2, SDC1, SDC4, SDHA, SDHAF2, |
| | SDHB, SDHC, SDHD, SELE, SELL, SELP, SEMA3B, SEPTIN2, SERPINA1, |
| | SERPINA3, SERPINA5, SERPINB13, SERPINB2, SERPINB3, SERPINB4, |
| | SERPINE1, SERPINF1, SET, SETBP1, SETD2, SETDB1, SF3B1, SFN, SFPQ, |
| | SFRP4, SGK1, SH2B3, SH3GL1, SHBG, SIN3A, SIRT2, SIRT4, SIX1, SKP2, |
| | SLC19A1, SLC1A3, SLC26A3, SLC2A1, SLC34A2, SLC3A2, SLC44A3, |
| | SLC45A3, SLC4A5, SLPI, SMAD1, SMAD2, SMAD3, SMAD4, SMARCA4, |
| | SMARCB1, SMARCD1, SMARCE1, SMC1A, SMC3, SMO, SMYD3, SND1, |
| | SNX25, SOCS1, SOD1, SOD2, SOS1, SOX1, SOX17, SOX2, SOX9, SP1, |
| | SPARC, SPARCL1, SPATA6, SPEN, SPINK1, SPINT1, SPINT2, SPOP, SPP1, |
| | SPRR1B, SPRR3, SPRY1, SRC, SRD5A1, SRD5A2, SRSF2, SRSF3, SS18, |
| | SS18L1, SST, SSX1, SSX2, SSX2B, SSX4, ST14, STAG2, STARD3, STAT3, |
| | STAT4, STAT5A, STAT5B, STAT6, STEAP1, STIL, STK11, STK19, STMN1, |
| | STRAP, STRN, STT3A, STX2, SUFU, SULT1E1, SUZ12, SYK, SYNE1, TAF1, |
| | TAF15, TAGLN, TAL1, TAL2, TAP1, TBC1D12, TBL1XR1, TBX3, TCEA1, |
| | TCF12, TCF3, TCF7, TCF7L2, TCL1A, TCP11L2, TDRD10, TDRD6, TEK, |
| | TENT5C, TERT, TET1, TET2, TF, TFAP2B, TFDP1, TFDP2, TFE3, TFEB, TFF1, |
| | TFF2, TFF3, TFG, TFRC, TG, TGFA, TGFB2, TGFB3, TGFBR2, TGFBR3, |
| | TGIF1, TGM4, TGM7, THBS1, THBS2, THBS4, THPO, THRA, THRB, TIE1, |
| | TIMM17A, TIMP1, TIMP2, TIMP3, TJP2, TK1, TLX1, TLX3, TMEM127, |
| | TMF1, TMPRSS2, TMPRSS3, TNC, TNF, TNFAIP2, TNFAIP3, TNFRSF10A, |
| | TNFRSF10B, TNFRSF10C, TNFRSF10D, TNFRSF11B, TNFRSF12A, |
| | TNFRSF14, TNFRSF17, TNFRSF1A, TNFRSF1B, TNFRSF4, TNFRSF8, |
| | TNFRSF9, TNFSF10, TNFSF11, TNFSF13, TNFSF13B, TNFSF4, TNFSF8, |
| | TNK2, TOM1, TOP1, TOP2A, TOP3A, TP53, TP53BP1, TP53BP2, TP63, |
| | TPD52, TPI1, TPM1, TPM2, TPM3, TPM4, TPR, TPX2, TRAF1, TRAF2, |
| | TRAF3, TRAF4, TRAF7, TRIM23, TRIM24, TRIM25, TRIM27, TRIM33, |
| | TRIM7, TRIO, TRIP11, TRIP4, TRO, TRRAP, TSC1, TSC2, TSG101, TSHR, |
| | TSPAN8, TSPO, TTLL9, TTR, TUBA1A, TUSC2, TWIST1, TXLNA, TXNDC8, |
| | TXNIP, TXNRD1, TYMP, TYMS, TYRO3, U2AF1, UBA1, UBE2C, UBE2I, |
| | UBE2N, UBR5, UGT1A1, UGT1A10, UGT1A3, UGT1A4, UGT1A9, |
| | UHRF1BP1L, USH1C, USP6, USP8, VAMP3, VCAM1, VEGFA, VEGFB, |
| | VEGFC, VEGFD, VHL, VIL1, VIP, VTN, VWF, WAS, WASF3, WDCP, WEE1, |
| | WFDC2, WIF1, WNT1, WNT2, WRN, WT1, WWTR1, XBP1, XIAP, XIRP2, |
| | XPA, XPC, XPO1, XRCC1, XRCC2, XRCC3, XRCC4, XRCC5, XRCC6, YBX1, |
| | YWHAB, YWHAE, YWHAH, ZBTB16, ZFHX3, ZFP36L1, ZFP36L2, ZMYM2, |
| | ZNF132, ZNF180, ZNF300, ZNF331, ZNF384, ZNF471, ZNF483, ZNF521, |
| | ZNF620, ZNF750, ZNF814, ZNF844, ZNF91, ZRANB3, ZRSR2 |
| Genetic | AGPAT2, AGRN, AIPL1, ALG1, ALG11, ALG12, ALG13, ALG14, ALG2, |
| | ALG3, ALG6, ALG8, ALG9, ATP6AP1, ATP6AP2, B3GALNT2, B3GLCT, |
| | B4GALT1, B4GAT1, BPGM, BSCL2, CACNA1F, CAD, CAV1, CAVIN1, |
| | CCDC115, CDAN1, CDIN1, CEP290, CHAT, CHKB, CHRNA1, CHRNB1, |
| | CHRND, CHRNE, CNGA3, COG1, COG2, COG5, COG6, COG7, COG8, |
| | COL12A1, COL13A1, COL6A1, COL6A2, COL6A3, COLQ, CRB1, CRPPA, |
| | CRX, CYP11B1, CYP17A1, CYP21A2, DAG1, DDOST, DOK7, DOLK, |
| | DPAGT1, DPM1, DPM2, DPM3, DUOX2, DUOXA2, EGLN1, EPAS1, EPO, |
| | EPOR, FCSK, FKRP, FKTN, FOXE1, FUT8, GALNT2, GDF6, GFPT1, GIPC1, |
| | GLIS3, GMPPB, GNAT1, GNB3, GRK1, GRM6, GUCY2D, HBB, HSD3B2, |
| | IGSF1, IMPDH1, INPP5K, IQCB1, IRS4, ITGA7, IYD, KCNJ13, KCNJ6, KLF1, |
| | LAMA2, LARGE1, LCA5, LMNA, LPIN2, LRAT, LRIT3, LRP4, MAGT1, |
| | MGAT2, MPDU1, MPI, MUSK, MYO7A, MYO9A, NKX2-1, NMNAT1, |
| | NPHP1, NPHP4, NUS1, NKX2-5, NYX, PAX8, PDE6B, PGM1, PMM2, |
| | POMGNT1, POMGNT2, POMK, POMT1, POMT2, POR, PREPL, RAPSN, |
| | RD3, RDH12, RFT1, RHO, RPE65, RPGRIP1, RXYLT1, SAG, SCN4A, |
| | SDCCAG8, SEC23B, SLC18A3, SLC24A1, SLC25A1, SLC35A1, SLC35A2, |
| | SLC35C1, SLC39A8, SLC5A5, SLC5A7, SNAP25, SPATA7, SRD5A3, SSR4, |
| | STAR, STT3A, STT3B, SYT2, TBL1X, TG, THRA, TMEM165, TMEM199, |
| | TPO, TRAF3IP1, TRHR, TRIP4, TRPM1, TSHR, TUBB4B, TULP1, USP45, |
| | VAMP1, VHL, WDR19 |

### EXAMPLES

### Methods

Expression vectors for targeted regulation of gene expression were evaluated using lentiviral transduction of T cells and flow cytometry analysis. The Jurkat cell line was obtained from American Type Culture Collection (Manassas VA) and maintained in RPMI 1640 media with Glutamax (Gibco) containing 10% fetal calf serum (Gibco). For lentiviral transduction, Jurkat cells were incubated with lentivirus in complete media plus LentiBOOST at the manufacturers recommended concentration (Sirion Biotech). Eighteen hours after transduction, lentivirus and LentiBOOST were diluted by the addition of 3 volumes of fresh media.

Pre-selected, cryopreserved primary human CD4 T cells from normal donors were obtained from Bloodworks (Seattle WA). Human T cells were cultured in OpTimizer medium (Thermo Fisher) supplemented with Immune Cell Serum Replacement (Thermo Fisher), 2 mM L-glutamine (Gibco), 2 mM Glutamax (Gibco), 2001 U/ml IL-2 (R&D systems), 120 IU/ml IL-7 (R&D systems), and 20 IU/ml IL-15 (R&D systems).

Lentivirus was produced using standard protocols in a HEK293T suspension line. Viral supernatant was concentrated 10× using Lenti-X (Takara Bio) following the manufacturer's protocol.

For lentiviral transduction, T cells were stimulated with a 1:100 dilution of T cell TransAct (Miltenyi) for 30 hours. Virus was then added to T cells for 18-24 hours. Stimulation and viral infection were then terminated by addition of 7 volumes of fresh media without TransAct, and cells were cultured for 3-7 additional days before analysis.

Flow cytometry was performed on a Ze5 cytometer (Biorad). Cells were induced with danoprevir or equal volume DMSO for 24 hours prior to analysis. To determine expression of fluorescent proteins, between 1×10⁵-2×10⁵ total cells were transferred to a U-bottom 96 well culture dish (Corning). Cells were washed twice with flow cytometry staining buffer (eBioscience), then stained with eFluor-780 Fixable viability dye at 1:1000 dilution (eBioscience). After staining, cells were washed twice with flow cytometry staining buffer and analyzed immediately. Flow cytometry data was analyzed using FlowJo 10 (Tree Star). Where applicable during analysis, cells were gated on transduction positive cells based on BFP or RFP transduction markers and the GFP gMFI was determined for the live/transduction+/GFP+ cells.

### Inducible and Constitutive Polynucleotides

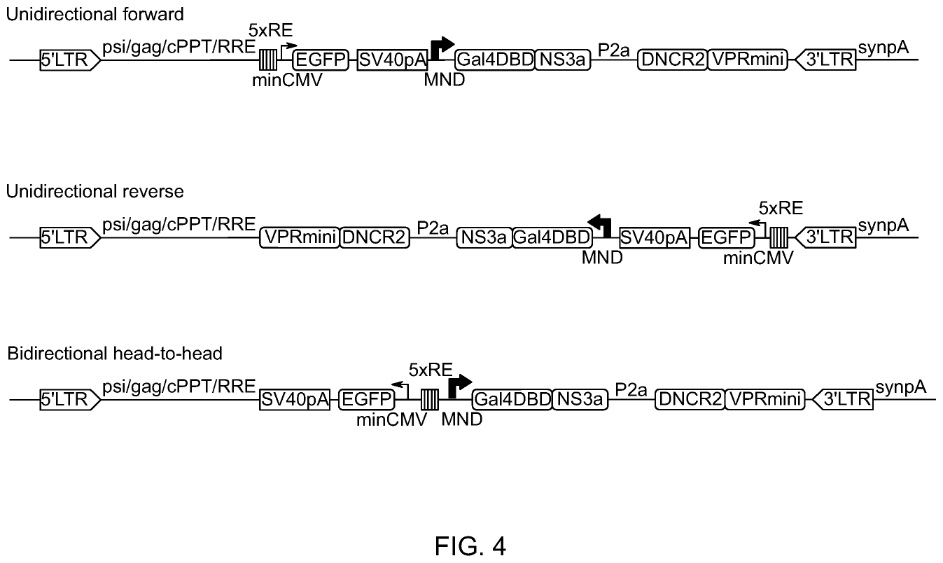

An initial split transcription factor comprised the Gal4 DBD fused to NS3a and DNCR2 fused to the VPRmini TAD, expressed under the control of a constitutive promoter (MND). Both of these transcriptional units (constitutive promoter and inducible promoter) were assembled into all-in-one vectors in lentiviral backbones in three different orientations: unidirectional forward, unidirectional reverse, and bidirectional head-to-head. FIG. 4 illustrates a schematic diagram 400 of an example of all-in-one vectors in lentiviral backbones in unidirectional forward (SEQ ID NO: 96), unidirectional reverse (SEQ ID NO: 95), and bidirectional head-to-head (SEQ ID NO: 97) orientations. In this example, the inducible gene expressed is EGFP, which encodes an enhanced GFP protein (EGFP or GFP). The expressed split transcription factor binds to a 5×Gal4-RE repeat to induce expression GFP from a minimal CMV promoter (minCMV).

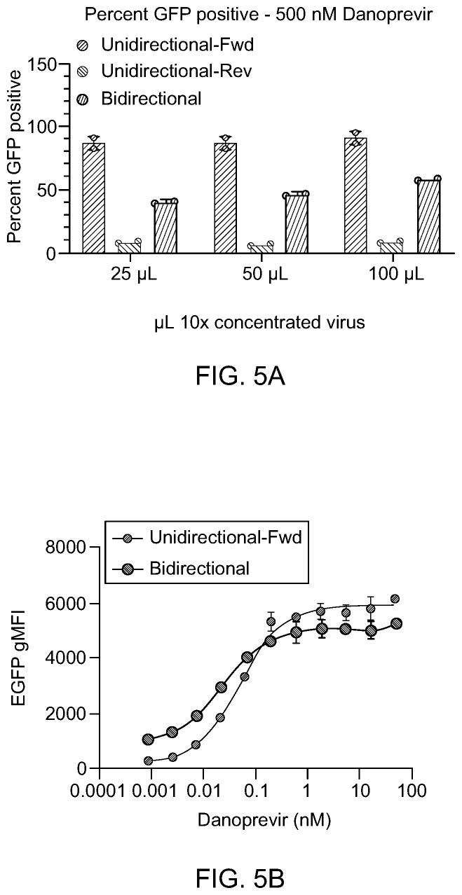

FIG. 5A is a plot 500 showing transduction results for the three vector orientations of FIG. 4 using different volumes of 10× concentrated lentivirus in Jurkat cells. The data show that the unidirectional forward vector had a distinct advantage in providing higher titer lentivirus, as seen by the higher percentage of Jurkat cells that were successfully transduced with the virus and expressed GFP upon danoprevir treatment. The bidirectional vector arrangement gave lentivirus of moderate titer, while the unidirectional reverse vector gave low titer virus.

FIG. 5B is a plot 510 showing titration of danoprevir on Jurkat cells expressing the unidirectional forward or bidirectional vectors of FIG. 4. The data show that the titration of danoprevir on the unidirectional forward and bidirectional vectors gave a similar dose-response of induced GFP expression, with the bidirectional vector exhibiting higher background levels of GFP in the absence of danoprevir, possibly due to the close proximity of the constitutive and inducible promoters.

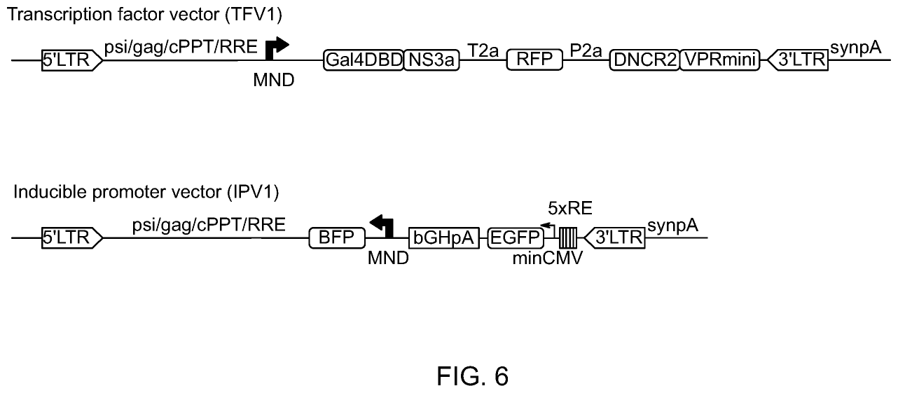

The inducible and constitutive transcriptional units (i.e., inducible polynucleotide and constitutive polynucleotide components) can be split across two lentivirus vectors to reduce crosstalk between the promoters and improve viral yields due to the smaller size of the vectors. FIG. 6 illustrates a schematic diagram 600 of an example of a two-vector system with the constitutive transcription factor component and inducible promoter component on separate lentiviral vectors. In this example, the transcription factor vector (TFV1, SEQ ID NO: 113) also encodes a constitutively expressed red fluorescent protein (RFP) as a transduction marker and the inducible promoter vector (IPV1, SEQ ID NO: 98) also encodes a constitutively expressed blue fluorescent protein (BFP) as a transduction marker. The inducible gene expressed in the inducible promoter vector is enhance green fluorescent protein (EGFP or GFP).

To evaluate the two-vector system, the two lentiviruses were produced separately and co-transduced into Jurkats or primary human CD4+ T cells. The split transcription factor expressed from TFV1 binds to a 5×Gal4-RE repeat on IPV1 to induce GFP expression from a minimal CMV promoter (minCMV).

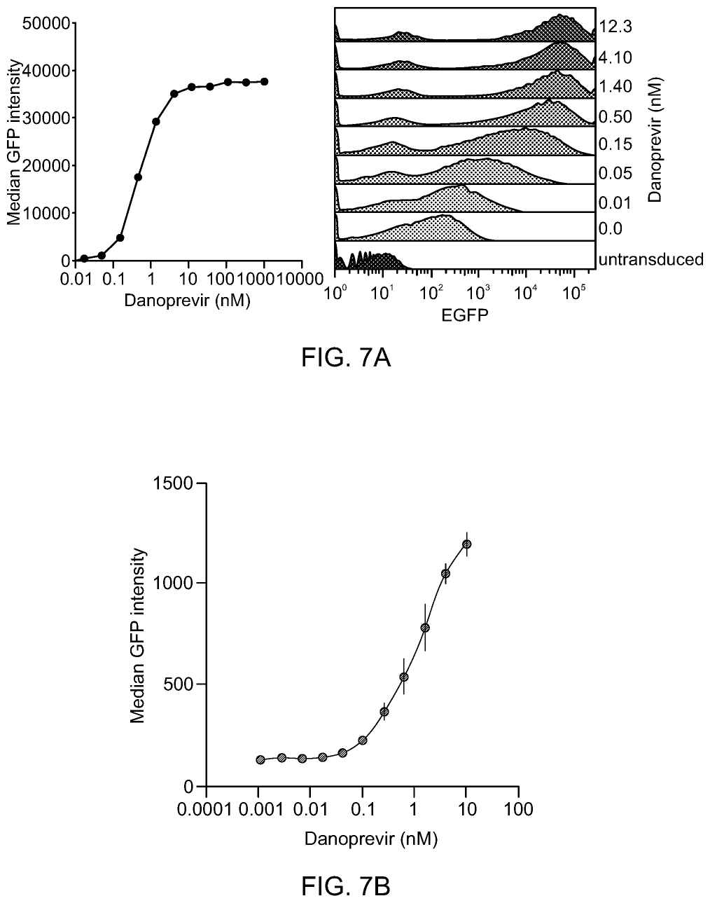

FIG. 7A is a plot 700 and a histogram 710 showing GFP intensity in transduction positive Jurkat cells in response to increasing concentrations of danoprevir. Cells were gated on transduction positive cells based on the transduction marker RFP and the EGFP gMFI was determined for the live/transduction+/GFP+ cells. The data show that in Jurkat cells, when gated on transduction positive cells, the median of the GFP peak shifts incrementally as danoprevir concentration increases. This indicates “titratability”, meaning that this system allows the intracellular concentration of a gene product (here GFP) to be modulated by the concentration of the inducer drug on a cell-by-cell basis. This observation contrasts with other small molecule systems (e.g., tet-inducible) that exhibit a binary response on a cell-by-cell basis (Loew, R., et al., Bmc Biotechnology (2010) 10, 81, which is incorporated herein by reference in its entirety).

FIG. 7B is a plot 715 showing median GFP intensity in primary CD4+ T cells. Cells were gated on transduction positive cells based on the transduction marker BFP and the EGFP gMFI was determined for the live/transduction+/GFP+ cells. The data shows high induction of GFP in primary human CD4+ T cells.

Background GFP expression was observed in the absence of danoprevir. Hypothesizing that the inducible promoter used had leaky constitutive expression, we sought to reduce this background by testing a panel of minimal promoters in the inducible promoter vector (IPV). The panel of minimal promoter tested included: minCMV (i.e., IPV2, SEQ ID NO: 99), YB_TATA (i.e., IPV3, SEQ ID NO: 100), the minimal IL2 promoter (minIL2) (i.e., IPV4, SEQ ID NO: 101), the minimal human beta globin promoter (huBG) (i.e., IPV5, SEQ ID NO: 102), and the promoter region from the tetracycline inducible system TRE3G (i.e., IPV6, SEQ ID NO: 103) (Ede, C., et al., ACS Synthetic Biology (2016) 5: 395-404, which is incorporated herein by reference in its entirety). Jurkat cells were co-transduced with the transcription factor vector TFV1 (SEQ ID NO: 113) and one of the inducible promoter vectors IPV2-IPV6.

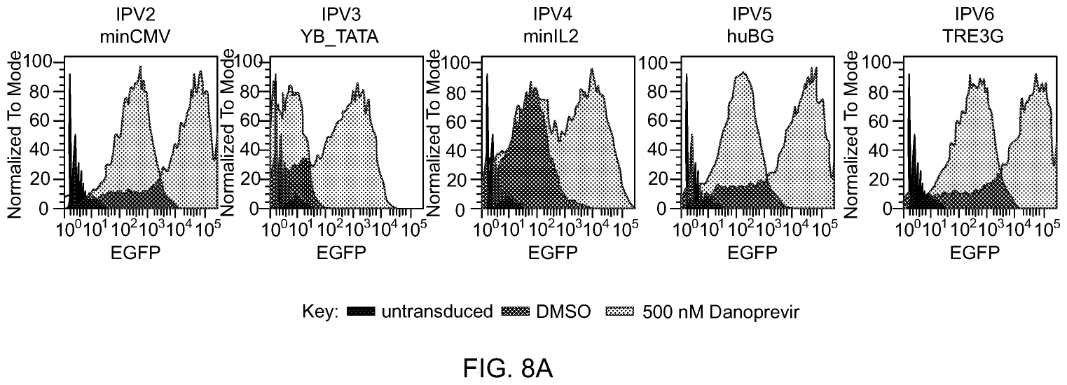

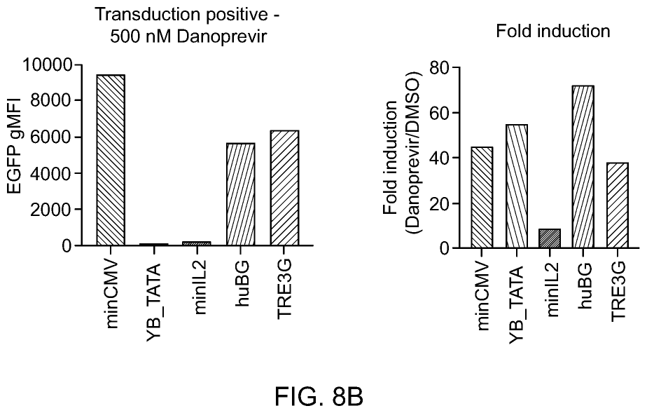

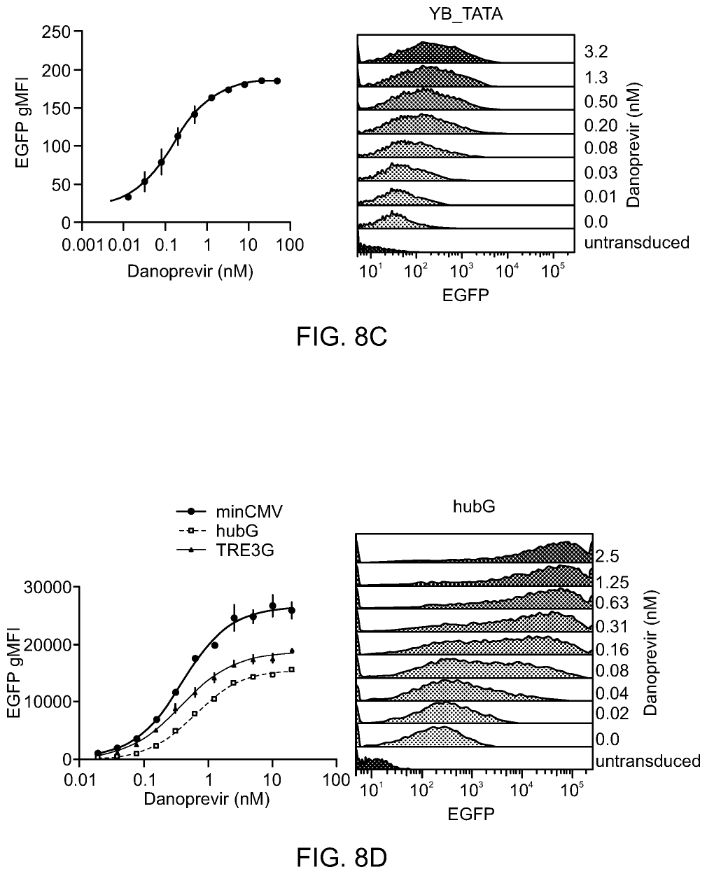

FIG. 8A is a panel of histogram plots 800 showing EGFP expressed from untransduced Jurkat cells or Jurkat cells co-transduced with the transcription factor vector TFV1 and one of the inducible promoter vectors (IPV2-IPV6) exposed to 500 nM danoprevir. Exposure of untransduced and co-transduced Jurkat cells to DMSO was used as a vehicle control. The data show that all minimal promoters tested induced expression of EGFP in response to danoprevir. The level of EGFP in the DMSO exposed cells indicates the increase in background GFP by the inducible promoter vector over untransduced cells.

FIG. 8B is a plot 810 and a plot 815 showing maximal EGFP mean fluorescence intensity data (gMFI) and fold induction, respectively, for induction GFP expression in response to 500 nM danoprevir in Jurkat cells co-transduced with the transcription factor vector TFV1 and one of the inducible promoter vectors (IPV2-IPV6). For plot 815, fold induction was computed as EGFP gMFI for danoprevir exposed cells relative to DMSO exposed cells (i.e., danoprevir/DMSO conditions).

FIG. 8C is a plot 820 and a histogram plot 825 showing EGFP expression levels in response to titration of danoprevir on the weakest minimal promoter, YB_TATA (i.e., IPV3, SEQ ID NO: 100).

FIG. 8D is a plot 830 and a histogram plot 835 showing EGFP expression levels in response of the strongest minimal promoters minCMV (IPV2, SEQ ID NO: 99), huBG (IPV5, SEQ ID NO: 102), TRE3G (IPV6, SEQ ID NO: 103) to danoprevir titration and EGFP levels for huBG, respectively.

Referring now to FIG. 8A through FIG. 8D, the data show that the minimal promoters YB_TATA and minIL2 were the weakest in their maximum induction level, with minIL2 showing incomplete activation (i.e., one population with drug-induced expression and the other with leaky constitutive expression). YB_TATA, a synthetic promoter, had an advantage in having the lowest background GFP level and good titratability but had the lowest maximum induction level. Of the three strongest promoters (minCMV, TRE3G, huBG), huBG had the lowest background level, resulting in the highest fold-induction of GFP.

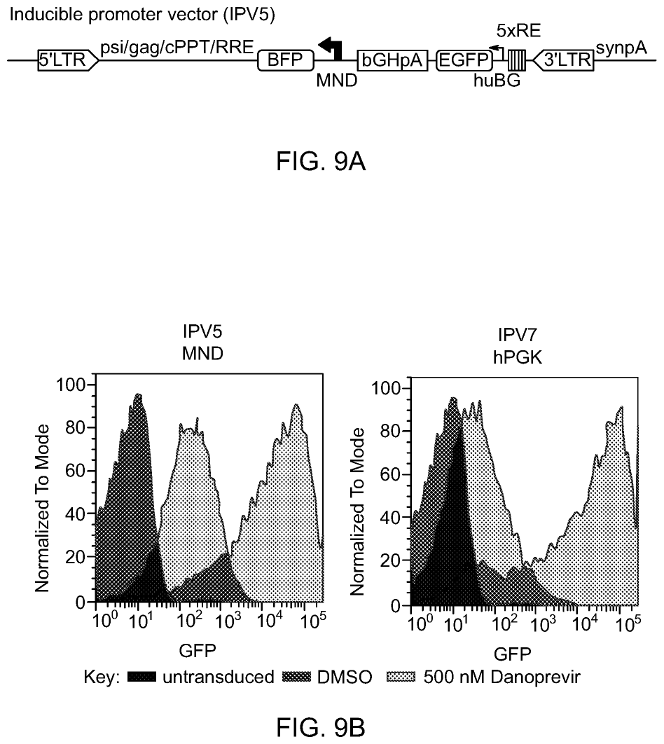

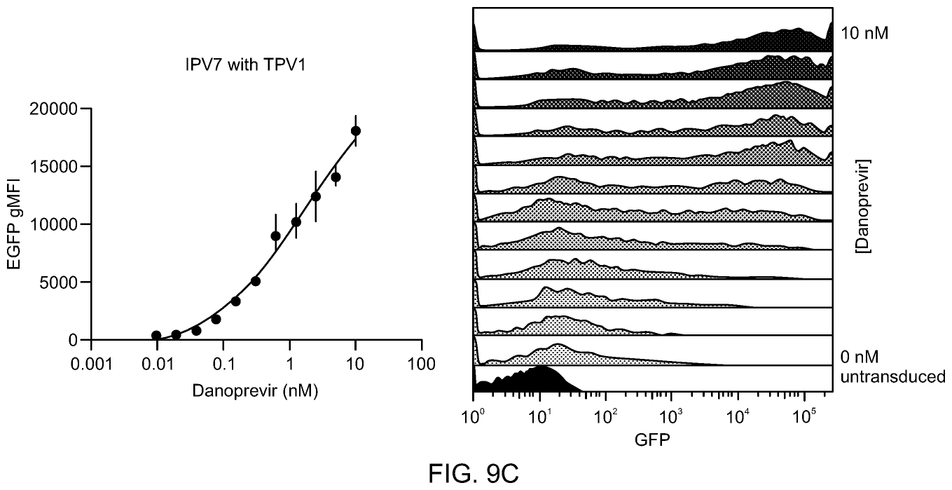

We further hypothesized that the remaining background GFP levels observed with the YB_TATA and huBG minimal promoters may have been caused by crosstalk with the enhancers of the constitutive MND promoter that was used to drive the expression of BFP as a transduction marker on the inducible promoter vectors. FIG. 9A illustrates a schematic diagram 900 of an example of an inducible promoter vector (IPV5, SEQ ID NO: 102) showing the constitutive promoter MND driving the expression of the transduction marker BFP and the minimal inducible promoter huBG driving expression of EGFP.

To investigate the possibility that the background GFP levels observed may be caused by crosstalk with the MND promoter, the constitutive MND promoter was replaced with a constitutive hPGK promoter. Jurkat cells were co-transduced with TFV1 (SEQ ID NO: 113) and either IPV5 (SEQ ID NO: 102) or IPV7 (SEQ ID NO: 104), which utilize the MND and hPCK promoters, respectively. Untransduced Jurkat cells and co-transformed Jurkat cells exposed to DMSO were used as controls.

FIG. 9B is a histogram plot 910 and a histogram plot 915 showing normalized GFP expression levels in Jurkat cells co-transformed with TFV1 and either IPV5 or IPV7, which utilize the MND and hPCK promoters, respectively. A comparison of the DMSO condition to the untransduced Jurkat cells shows that the constitutive hPCK promoter results in less crosstalk with the inducible promoter and lower background GFP levels.

FIG. 9C is a plot 920 and a histogram plot 925 showing EGFP expression levels in response to titration of danoprevir on the hPGK vector (i.e., IPV7) in Jurkat cells co-transduced with TFV1.

Referring now to FIG. 9B and FIG. 9C, the data show that replacing the MND promoter with the hPGK promoter decreased background GFP expression. The resulting IPV7 vector exhibited a large dynamic range of GFP expression when co-transduced with the transcription factor vector TFV1. We expect that removing the constitutive promoter and transduction marker from the reporter vector will further reduce leakiness.

### Transcription Factor Component

In addition to variations in minimal and constitutive promoters, the transcription factor component of the small molecule-inducible gene expression system was optimized. Because the transcription factor is a split transcription factor consisting of two polypeptide chains, the polynucleotide encoding the first fusion protein and the polynucleotide encoding the second fusion protein must be separated by a separation element such as ribosomal skipping sequence (e.g., P2a or T2a), an IRES, or expressed from two separate constitutive promoters. Briefly, primary CD4+ T cells were co-transduced with the inducible promoter vector IPV1 (synPA-tagBFP-MND-bGHpA-sfGFP-minCMV-5×Gal4RE; SEQ ID NO: 98) and either a transcription factor vector that includes:
(i) two 2a sequences separating a Gal4DBD-NS3a polynucleotide and a DNCR2-TAD polynucleotide (TFV1: ND-Gal4DBD-NS3a-T2a-mCherry-P2a-DNCR2-VPR; SEQ ID NO: 113),
(ii) a single 2a sequence separating aGal4DBD-NS3a polynucleotide and a DNCR2-TAD polynucleotide (TFV2: MND-mCherry-T2a-Gal4DBD-NS3a-P2a-DNCR2-VPR; SEQ ID NO: 114), or
(iii) two 2a sequences separating a NS3a-TAD polynucleotide and a Gal4DBD-DNCR2 polynucleotide (TFV3: MND-NS3a-VPR-T2a-mCherry-P2a-Gal4DBD-DNCR2; SEQ ID NO: 115).

In this example the transcriptional activation domain (TAD) is VPRmini (“VPR”). Co-transduced cells were exposed to 500 nM danoprevir or DMSO (control) and analyzed by flow cytometry for GFP expression.

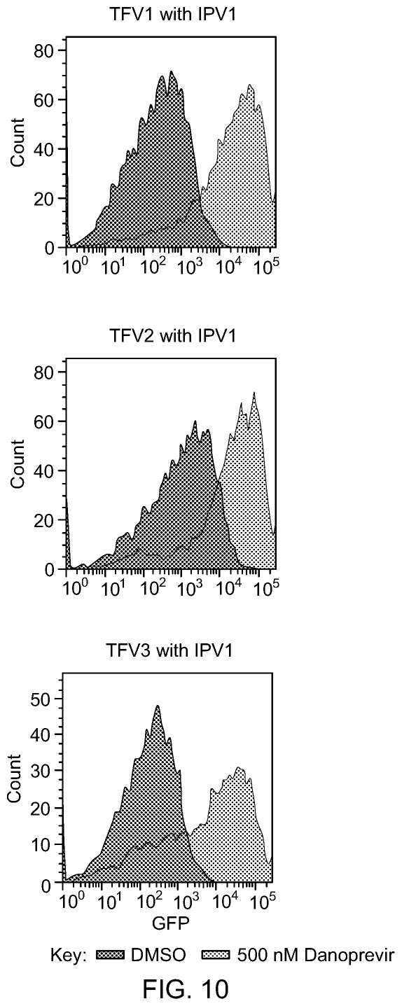

FIG. 10 is histogram plots 1000, 1010, and 1015 showing GFP levels in cells co-transduced with IPV1 and either TFV1, TFV2, or TFV3, respectively, and exposed to danoprevir or DMSO. The data show that a single 2a element (TFV2) between the transcription factor components resulted in higher background GFP expression than two 2a elements (TFV1), likely from incomplete translational skipping resulting in some production of fused NS3a-DBD-DNCR2-TAD protein. Additionally, the fusion partners in the transcription factor could be swapped, with DNCR2 fused to Gal4 and NS3a fused to VPRmini (TFV3). TFV3 had two 2a sequences separating the transcription factor components and yielded a similar background GFP level as TFV1 and successful induction of GFP upon danoprevir treatment.

To generalize the gene expression system to other DNA binding domains (DBDs) and transcriptional activation domains (TADs), a panel of four zinc fingers (ZFs) and four TADs were tested. The four ZFs tested (ZFHIV2, ZF1, ZF2, and ZF3) have been previously described (Lohmueller, J. J., et al., Nucleic Acids Research (2012) 40: 5180-5187; Donahue, P. S. et al., Nature Communications (2020) 11; and Khalil, A. S., et al., Cell (2012) 150: 647-658, which are incorporated herein by reference in their entireties). Each of the four ZFs were fused to NS3a. The NS3a fusion proteins tested were NS3a-ZFHIV2 (SEQ ID NO: 71), NS3a-ZF1 (SEQ ID NO: 68), NS3a-ZF2 (SEQ ID NO: 69), and NS3a-ZF3 (SEQ ID NO: 70). For the inducible promoter vector, six repeats of the ZF response elements HIV2RE (SEQ ID NO: 143), ZF1RE (6×ZFIRE; SEQ ID NO: 85), ZF2RE (6×ZF2RE; SEQ ID NO: 86), ZF3v1RE (6×ZF3v1RE; SEQ ID NO: 87), and ZF3v3RE (SEQ ID NO: 88) were encoded in front of the YB_TATA minimal promoter (SEQ ID NO: 77). Note that two different 6×RE encodings were used for ZF3 in which the nucleotides flanking the RE sequences varied: ZF3v1RE (SEQ ID NO: 87) and ZF3v3RE (SEQ ID NO: 88). The different zinc finger protein fusions were compared to an NS3a-Gal4 DBD fusion protein (Gal4-NS3a SEQ ID NO: 65), with the 5×Gal4RE and YB_TATA minimal promoter vector IPV8 (SEQ ID NO: 105). Briefly, Jurkat cells were co-transduced with an inducible promoter vector (IPV) and its cognate transcription factor vector (TFV). Cells were induced with 500 nM danoprevir or an equal volume of DMSO for 24 hours prior to analysis by flow cytometry. The vectors used are shown in Table 2.

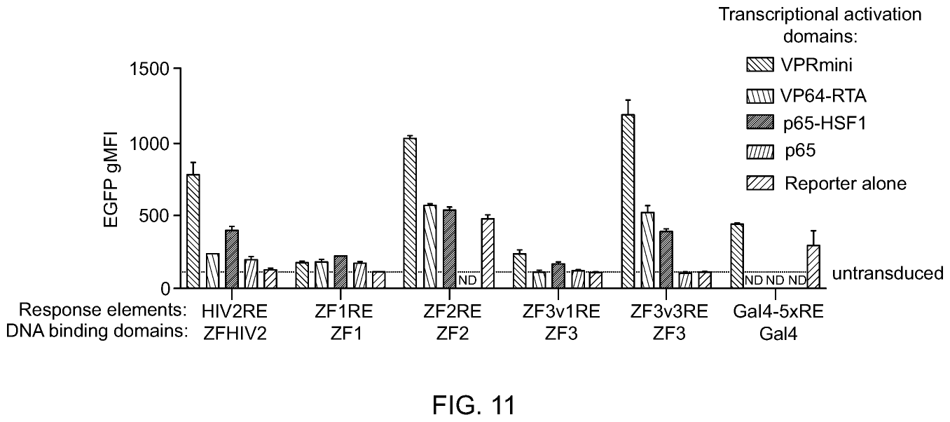

FIG. 11 is a plot 1100 showing GFP expression (gMFI) for the four zinc finger (ZF) DBD-NS3a fusion proteins and the four DNCR2-TAD fusion proteins in response to treatment with 500 nM danoprevir. All IPVs (IPV8-IPV13) utilize YB_TATA as the minimal promoter and are used with their cognate TFV (TFV4-TFV18). Reporter alone indicates the GFP level from Jurkats transduced with only the inducible promoter vectors. Gal4 with VPRmini is shown for comparison. The data show that ZFHIV2 and ZF3 (with ZF3v3RE) gave the highest induced GFP levels. ZF2 also produced high GFP levels, but its reporter sequence gave high background GFP levels (“reporter alone” condition). VPRmini was the strongest transcriptional activation domain, while VP64-RTA and p65-HSF1 (a TAD composed of all-human components) both showed moderate induction levels. p65 alone was very weak. In comparison, the Gal4 system with VPRmini gave weaker max induction than ZF3 and ZFHIV2, indicating that these human-derived zinc finger sequences offer comparable-or-better gene induction to the yeast-derived Gal4 DBD.

Additionally, to increase induction of GFP expression, two different strategies were used. In the first strategy, the number of RE repeats was increased from 6× (IPV9, IPV13) to 12× (IPV14, IPV15) for ZFHIV2 or ZF3. A second strategy to increase induction was to dimerize the NS3a-ZF construct with a leucine zipper homodimer sequence (LZ) (TFV19, TFV20).

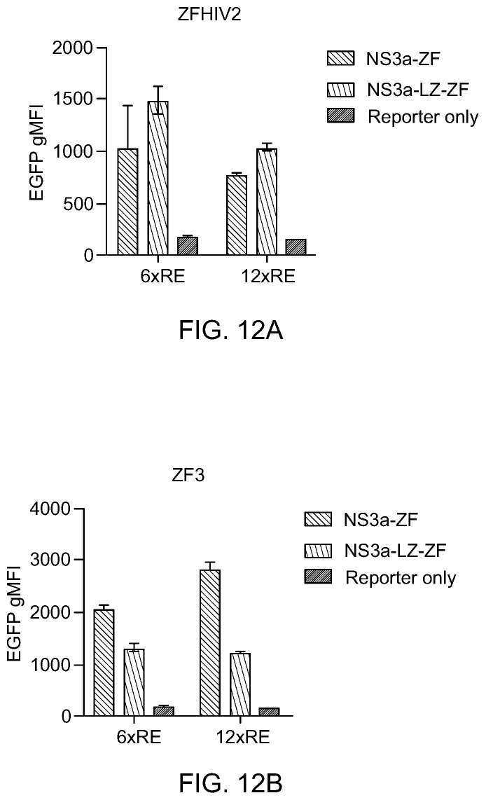

FIGS. 12A and 12B are a plot 1200 and a plot 1210 showing GFP expression (gMFI) induced by DNCR2-VPRmini on inducible promoters includes 6×RE or 12×RE for ZFHIV2 or ZF3, respectively. In this example, the zinc fingers were fused directly to NS3a or with a homodimeric leucine zipper (LZ) between the NS3a and ZF domain (TFV19, TFV20). The data show increased induction from ZF3, but lower induction from ZFHIV2. The data show a higher maximal induction for ZFHIV2, but lower induction for ZF3, indicating some dependence of this strategy on the DBD being used.

To improve encoding of the inducible gene expression system in viral vectors, which are limited in genetic cargo capacity, we used the Rosetta software package to generate new designs that reduce the size of our small molecule induced dimers (Leaver-Fay, A. et al., Methods Enzymology (2011) 487:545-74, which is incorporated herein by reference in its entirety). Specifically, we generated designs that reduce the number and length of the helices in DNCR2 and GNCR1 (sequences DNCR2_1 through DNCR2_34 (SEQ ID NOs: 12 through 45) and G-3rep (SEQ ID NO: 48), G33 (SEQ ID NO: 49), and G38 (SEQ ID NO: 50)), followed by redesigning the amino acid sequence of the regions surrounding these truncations using previously described design methods (Brunette, T. J., et al., Nature (2015) 528(7583):580-4, and Brunette, T. J., et al., PNAS (2020) 117(16) 8870-8875, which are incorporated herein by reference in their entirety).

A panel of these designs with a range of truncation degrees and sequence diversity were tested for their ability to bind NS3a/danoprevir. The minimized DNCR2 and GNCR1 designs were expressed on the surface of EBY 100 Saccharomyces cerevisiae and analysed by flow cytometry. Briefly, Avi-His6-tagged NS3a was co-expressed with biotin ligase BirA in BL21 E. coli, and biotinylated NS3a was purified from the lysed cells following standard His-tag purification procedures. DNCR2 and GNCR1 designs were expressed on the surface of EBY100 S. cerevisiae via fusion to Aga2p in the standard yeast display vector pETCON with a c-myc tag for expression detection. NS3a complexes were formed in PBS+0.5% w/v BSA with excess danoprevir or grazoprevir (ApexBio). NS3a/drug complexes were incubated with yeast expressing the designs for 1 hr at room temperature, then washed. Yeast cells were incubated with streptavidin-PE (Invitrogen, S866) and anti-myc-AlexaFluor647 (Cell Signaling Technologies, #2233S) for 10 min and washed before analysis on a BioRad ZE5 flow cytometer.

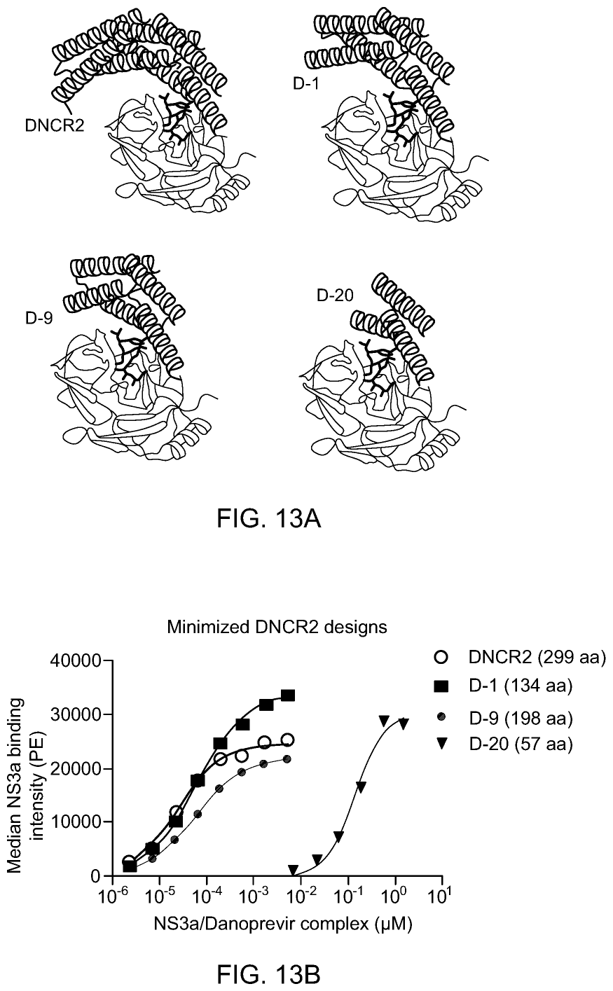

FIG. 13A is a schematic diagram showing the crystal structure of DNCR2/danoprevir/NS3a and models of D-1 (DNCR2_1; SEQ ID NO: 12), D-9 (DNCR2_9; SEQ ID NO: 20), and D-20 (DNCR2_20; SEQ ID NO: 31) designs. FIG. 13B is a plot 1310 showing the median NS3a binding intensity (PE) for titration of NS3a/danoprevir binding to the four DNCR2 variants displayed on yeast. Designs were displayed on the surface of yeast, and NS3a/danoprevir was titrated on yeast and observed by flow cytometry. A number of DNCR2 minimization designs were considerably smaller than the original DNCR2 (SEQ ID NO: 11) and maintained binding to NS3a/danoprevir. D-1 and D-9 showed equivalent binding as DNCR2, while D-20 (smallest successful design at 57 amino acids) exhibited weaker binding.

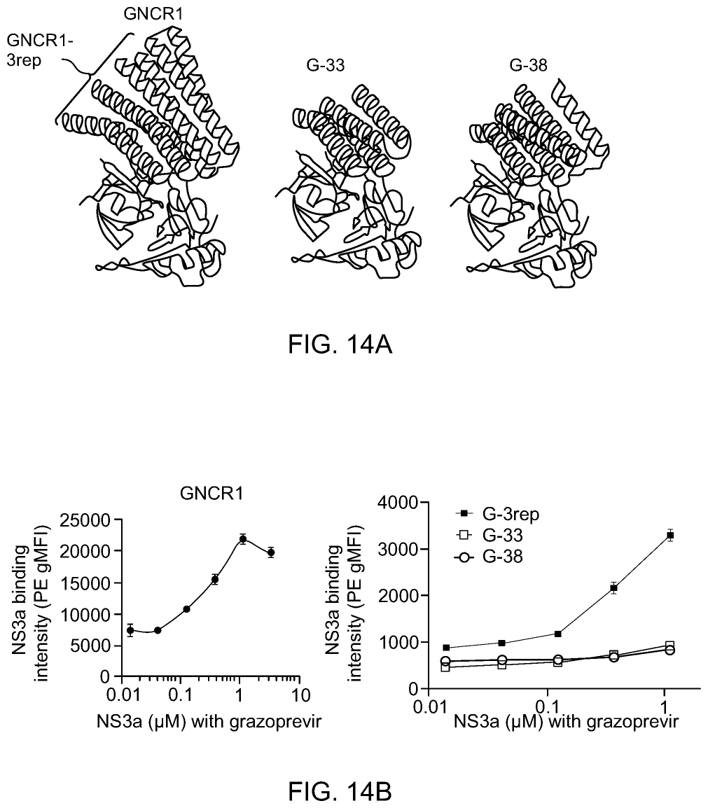

FIG. 14A is a schematic diagram 1400 showing models of GNCR1 (with G-3rep truncation indicated), G-33, and G-38. FIG. 14B is a plot 1410 and a plot 1415 titration of NS3a/grazoprevir binding the GNCR1 and titration of NS3a/grazoprevir on G-3rep, G-33, and G-38 displayed on yeast, respectively. For GNCR1, three designs were identified that retained moderate binding to NS3a (i.e., G-3rep, G33, and G38), albeit with reduced binding compared to the original GNCR1 (SEQ ID NO: 47).

### Two-Vector System Optimization

To further optimize the two-vector system we sought to: (i) reduce the size of the transcription factor and inducible promoter vector constructs, and (ii) reduce background expression (i.e., “leakiness”) from the inducible promoter construct.

To reduce the size of the transcription factor and inducible promoter vectors, transduction markers (i.e., RFP and BFP) were removed from both vectors. As described herein above with reference to FIG. 6, the original transcription factor vector TFV1 included a T2a-RFP-P2a sequence separating the transcription factor components and the inducible promoter vector included a constitutive promoter-BFP sequence. IPV and TFV vectors for optimizing the two-vector system are shown in Table 3.

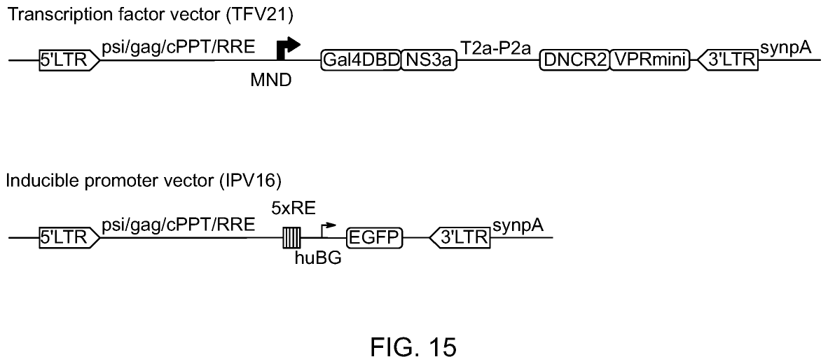

FIG. 15 illustrates a schematic diagram of an example of a modified two-vector system with transduction markers removed from the constitutive transcription factor and inducible promoter lentiviral vectors. In this example, the transcription factor vector TFV21 includes two sequential 2a ribosome skipping elements without the RFP sequence between them (T2a-P2a) separating the DNA binding domain (Gal4DBD-NS3a) and the transcriptional activation domain (DNCR2-VPRmini) components. The inducible promoter vector IPV16 the inducible promoter (huBG) and EGFP in the forward direction; sequences encoding the constitutive promoter and the BFP transduction marker were removed. Removal of the constitutive promoter-BFP sequences in IPV16 reduced the size of the vector and removed the potential for crosstalk between the constitutive promoter and the inducible promoter, which we have shown could influence background EGFP leakiness (see FIG. 9).

The modified expression vectors TFV21 and IPV16 were evaluated using lentiviral transduction of T cells (i.e., Jurkat and HEK293T cell lines) and flow cytometry analysis. The Jurkat cell line was obtained from American Type Culture Collection (Manassas VA) and maintained in RPMI 1640 media with Glutamax (Gibco) containing 10% fetal calf serum (Gibco). For lentiviral transduction, Jurkat cells were incubated with lentivirus in complete media plus LentiBOOST at the manufacturer's recommended concentration (Sirion Biotech). Eighteen hours after transduction, lentivirus and LentiBOOST were diluted by the addition of 3 volumes of fresh media. The HEK293T cell line was obtained from American Type Culture Collection (Manassas VA) (catalog number CRL-3216) and maintained in DMEM, high glucose media with Glutamax (Gibco) containing 10% fetal calf serum (Gibco). For lentiviral transduction, HEK293T cells were plated at about 30% confluency 24 hours prior to transduction, then incubated with lentivirus in complete media. 24-48 hours after transduction, cells were passaged up to larger volume wells. Flow cytometry was performed essentially as described herein above.

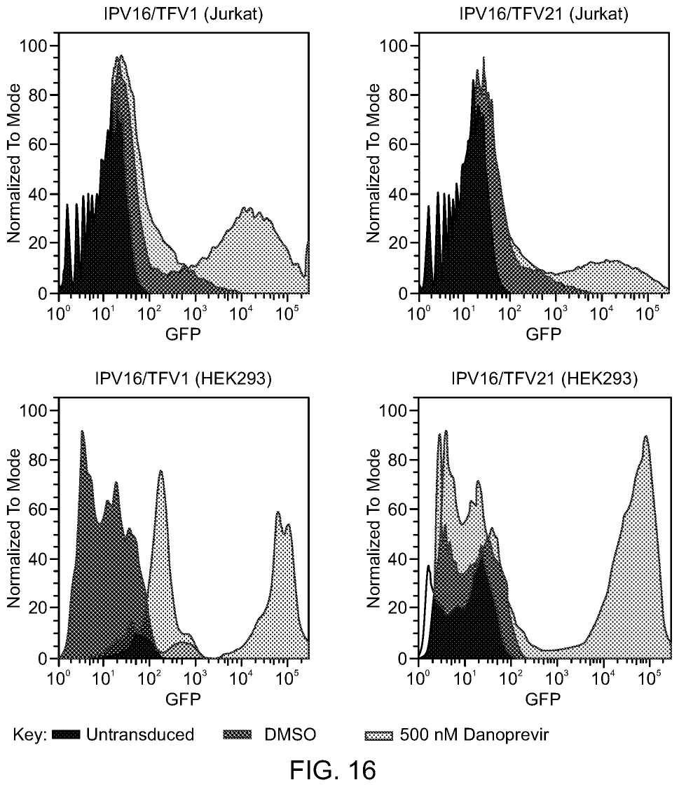

FIG. 16 is a panel of histogram plots showing GFP levels in Jurkat and HEK293 cells co-transduced with IPV16 and either TFV1 or TFV21. Transduced cells were treated with 500 nM danoprevir or 20 nM danoprevir and are compared to transduced cells treated with an equal volume of DMS and untransduced, wild type HEK293 cells. The histograms show live, single cells. The data show that in Jurkat cells and HEK293 cells IPV16 displayed very low levels of GFP leakiness when transduced with TRV1 or TFV21 compared to wild type cells. This result demonstrated that the removal of the constitutive promoter on the inducible promoter vector removes a significant source of leakiness. TFV1 and TFV21 exhibit very similar background GFP and induced GFP levels, indicating that the sequential T2a-P2a element is a viable alternative to the separation element containing a transduction marker.

To reduce any remaining leakiness in the system, we tested a panel of strategies designed to reduce EGFP expression in the absence of danoprevir by either degrading the Gal4-NS3a DNA binding domain or epigenetically blocking basal transcription with Gal4-KRAB (SEQ ID NO: 159). To degrade the Gal4-NS3a binding domain, we tried two approaches to localize the E3 ligase SPOP (SEQ ID NO: 156) to the inducible promoter via interaction with Gal4-NS3a. In one approach, we fused the two halves of a constitutive protein heterodimer binding pair (DHD37-2B and DHD37-2B) to Gal4-NS3a (Gal4-NS3a-DHD37-2B; SEQ ID NO: 161) and SPOP (DHD37-2A-SPOP; SEQ ID NO: 160) to create a system in which there would always be E3 ligase activity at the promoter regardless of danoprevir treatment. In another approach, we created a system in which SPOP would only be localized to the inducible promoter in the absence of danoprevir by fusing SPOP to the apo-NS3a reader ANR (ANR-SPOP; SEQ ID NO: 157). ANR binding to NS3a can be dissociated by any of the NS3a small molecule inhibitors. We compared background (DMSO control) and danoprevir-induced (100 nM danoprevir) EGFP expression levels from these approaches to the normal IPV16/TFV1 combination in HEK293 cells, and the level of autofluorescence of untransduced, wild type HEK293 cells for each vector pair.

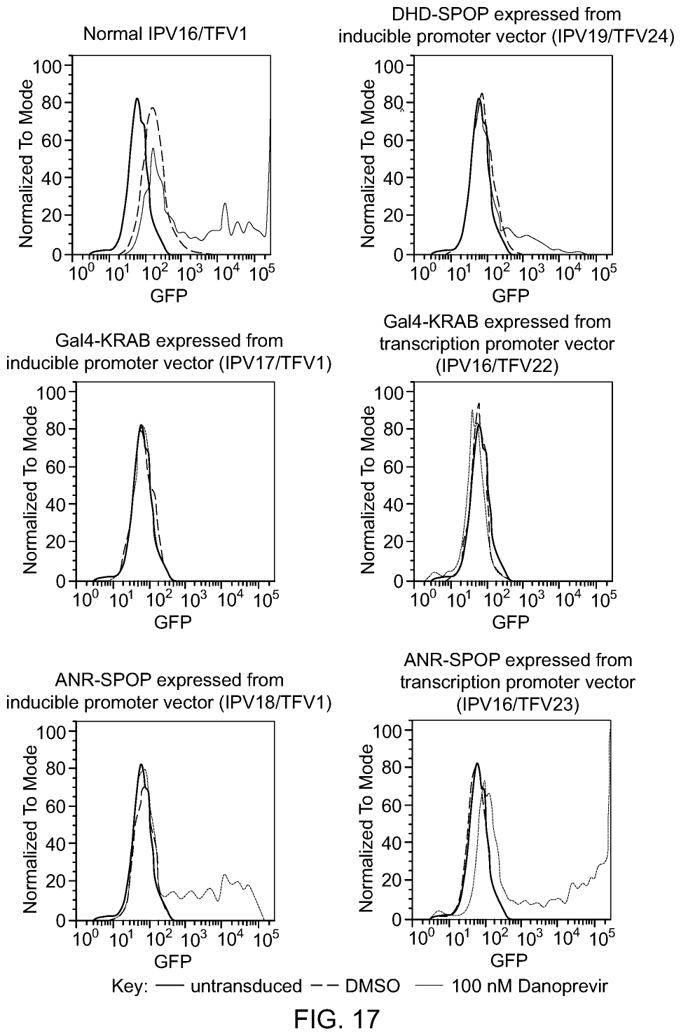

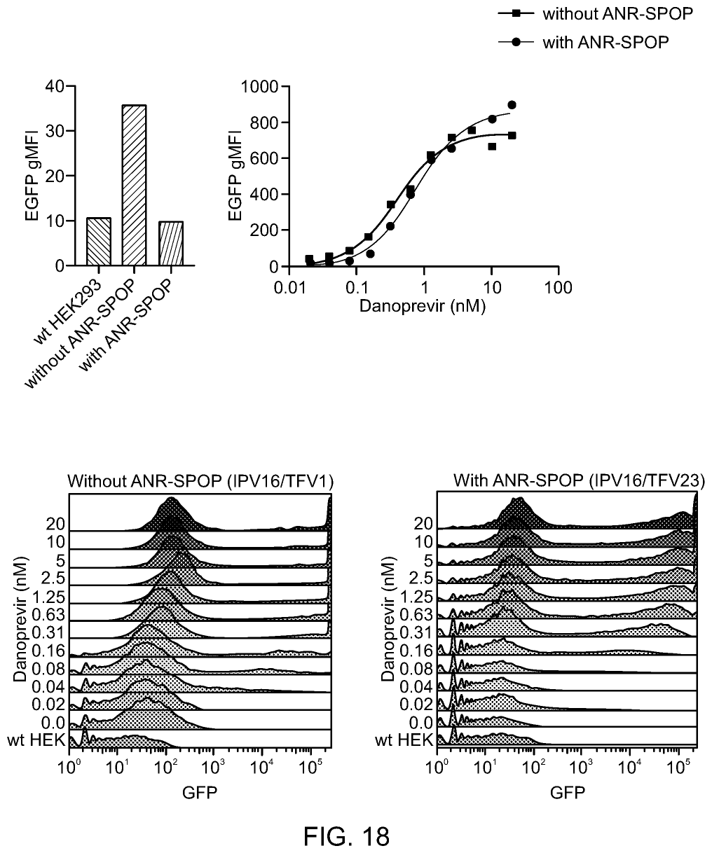

FIG. 17 is a panel of histogram plots showing EGFP expression in HEK293 cells transduced with the normal IPV16 and TFV1 vectors or with vectors expressing elements designed to reduce EGFP output. Plot 1700 shows GFP expression in cells co-transduced with the normal inducible promoter vector IPV16 and transcription factor vector TFV1. Plot 1710 shows GFP expression in cells co-transduced with DHD-SPOP expressed from the inducible promoter vector IPV19 and Gal4-NS3a-DHD expressed from the transcription factor vector TFV24. Plot 1715 shows GFP expression in cells co-transduced with Gal4-KRAB expressed from the inducible promoter vector IPV17 and the transcription factor vector TFV1. Plot 1720 shows GFP expression in cells co-transduced with the inducible transcription vector IPV16 and Gal4-KRAB expressed from the transcription factor vector TFV22. Plot 1725 shows GFP expression in cells co-transduced with ANR-SPOP expressed from the inducible promoter vector IPV18 and the transcription factor vector TFV1. Plot 1730 shows GFP expression in cell co-transduced with the inducible transcription vector IPV16 and ANR-SPOP expressed from the transcription factor vector TFV23. Plots 1700, 1710, 1715, and 1725 were gated on single, live, TFV transduction-positive events. Plots 1720 and 1730 were gated on live, single cells.

Plot 1700 of FIG. 17 shows background (DMSO control) and danoprevir-induced EGFP expression levels in the normal IPV16/TFV1 combination in HEK293 cells, which can display a small amount of leaky EGFP expression at higher transduction levels of IPV16.

Referring now to plots 1715 and 1720 of FIG. 17, Gal4-KRAB expressed either inducibly from the inducible expression vector (plot 1715) or constitutively from the transcription factor vector (1720) blocked both leaky and danoprevir-inducible EGFP expression, indicating that this epigenetic strategy is too strong.

Referring now to plot 1710 of FIG. 17, we fused the two halves of a constitutive protein heterodimer binding pair (DH1D37-2B and DH1D37-2B) to Gal4-NS3a (Gal4-NS3a-DH1D37-2B) and SPOP (DHD37-2A-SPOP) to create a system in which there would always be E3 ligase activity at the promoter regardless of danoprevir treatment. Plot 1710 shows that while effective in reducing leaky EGFP expression, this DHD-SPOP strategy also strongly reduced danoprevir-inducible EGFP expression.

Referring now to plots 1725 and 1730 of FIG. 17, we created a system in which SPOP would only be localized to the inducible promoter in the absence of danoprevir by fusing SPOP to the apo-NS3a reader ANR (ANR-SPOP). When ANR-SPOP was expressed inducibly (plot 1725) or constitutively (plot 1730) it effectively removed any leaky background expression of EGFP. Inducible ANR-SPOP expression also reduced the maximal danoprevir-induced EGFP expression, possibly due to higher ANR-SPOP expression levels. In contrast, ANR-SPOP expressed from the transcription factor vector (plot 1730) effectively reduced background EGFP expression in the absence of danoprevir while maintaining high danoprevir-induced expression. The slight shift in the fluorescence levels of the negative population in plot 1730 with danoprevir treatment may reflect that the suppressive effect of ANR-SPOP acts on transcriptional machinery that basally associates with the inducible promoter. Other E3 ligases fused to the DHD system or ANR would be expected to have a similar effect on reducing background expression.

To compare the performance of the system more closely with (IPV16/TFV23) and without (IPV16/TFV1) constitutive ANR-SPOP expression, we examined the dose response of EGFP expression to danoprevir titration. FIG. 18 is a panel of plots showing a comparison of EGFP background levels and titratable EGFP expression from the normal IPV16/TFV1 combination and IPV16 with the transcription factor vector TFV23 expressing ANR-SPOP. Plot 1800 shows background EGFP levels for wild type (wt) HEK293 cells compared to HEK293 cells transduced with the IPV16/TFV1 combination (without ANR-SPOP) or with the IPV16/TFV23 combination (with ANR-SPOP) treated with DMSO. Plot 1810 shows EGFP geometric mean fluorescence intensity (gMRI) plotted for a titration of danoprevir on the two construct combinations. Plots 1815 and 1820 show histograms of EGFP expression for the data plotted in plot 1810.

Referring now to plot 1800 of FIG. 18, we confirmed that the system with ANR-SPOP had background EGFP expression levels indistinguishable from wild type HEK293 cells, and about 3-times lower than that of the system without ANR-SPOP.

Referring now to plots 1810, 1815, and 1820, titration of danoprevir on the two systems demonstrated equivalent danoprevir EC50 levels and maximal expression levels.

1. A fusion protein comprising a DNA binding domain operably-linked to a dimerization domain, wherein the DNA binding domain specifically binds to a response element.

2.-41. (canceled)

42. A nucleic acid encoding the fusion protein of claim 1.

43.-49. (canceled)

50. A vector comprising the nucleic acid of claim 42.

51.-55. (canceled)

56. A cell comprising a fusion protein of claim 1.

57.-63. (canceled)

64. A composition comprising a cell of claim 56.

65. A pharmaceutical composition comprising a composition of claim 64 and a pharmaceutically acceptable carrier.

66. A fusion protein comprising a regulation domain operably-linked to a dimerization domain, wherein the regulation domain is capable of modulating a transcriptional activity or an epigenetic activity of one or more target sequences.

67.-95. (canceled)

96. A nucleic acid encoding a fusion protein of claim 66.

97.-103. (canceled)

104. A vector comprising a nucleic acid of claim 96.

105.-110. (canceled)

111. A cell comprising a fusion protein of claim 66.

112.-118. (canceled)

119. A composition comprising a cell of claim 111.

120. A pharmaceutical composition comprising a composition of claim 119 and a pharmaceutically-acceptable carrier.

121. A composition comprising:
(a) a first fusion protein comprising a DNA binding domain operably-linked to a dimerization domain, wherein the DNA binding domain specifically binds to a response element; and
(b) a second fusion protein comprising a regulation domain operably-linked to a dimerization domain, wherein the regulation domain is capable of modulating a transcriptional activity or an epigenetic activity of one or more target sequences.

122.-135. (canceled)

136. A cell comprising a composition of claim 121.

137.-143. (canceled)

144. A composition comprising the cell of claim 136.

145. A pharmaceutical composition comprising a composition of claim 121 and a pharmaceutically-acceptable carrier.

146.-151. (canceled)

152. A method of treating a disease or a disorder, comprising administering to a subject an effective amount of a fusion protein of claim 1, wherein a severity of a sign or symptom of the disease or disorder is decreased, thereby treating the disease or disorder.

153. (canceled)

154. The method of claim 152, wherein the disease or disorder comprises one or more of an autoimmune disease or disorder; an inflammatory disease or disorder; an immunodeficiency disease or disorder; an ischemic disease or disorder; a blood disease or disorder; a bone disease or disorder; a neurological disease or disorder; a cardiac disease or disorder; a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

155.-157. (canceled)

158. A cell comprising the nucleic acid of claim 42.

159. A cell comprising the vector of claim 50.

160. A composition comprising the fusion protein of claim 1.

161. A composition comprising the nucleic acid of claim 42.

162. A composition comprising the vector of claim 50.

163. A cell comprising the nucleic acid of claim 96.

164. A cell comprising the vector of claim 104.

165. A composition comprising the fusion protein of claim 66.

166. A composition comprising the nucleic acid of claim 96.

167. A composition comprising the vector of claim 104.

168. A method of treating a disease or a disorder, comprising administering to a subject an effective amount of the nucleic acid of claim 42, wherein a severity of a sign or symptom of the disease or disorder is decreased, thereby treating the disease or disorder.

169. A method of treating a disease or a disorder, comprising administering to a subject an effective amount of the vector of claim 50, wherein a severity of a sign or symptom of the disease or disorder is decreased, thereby treating the disease or disorder.

170. A method of treating a disease or a disorder, comprising administering to a subject an effective amount of the cell of claim 56, wherein a severity of a sign or symptom of the disease or disorder is decreased, thereby treating the disease or disorder.

171. A method of treating a disease or a disorder, comprising administering to a subject an effective amount of the composition of claim 64, wherein a severity of a sign or symptom of the disease or disorder is decreased, thereby treating the disease or disorder.

172. A method of treating a disease or a disorder, comprising administering to a subject an effective amount of the pharmaceutical composition of claim 145, wherein a severity of a sign or symptom of the disease or disorder is decreased, thereby treating the disease or disorder.

173. A pharmaceutical composition comprising a composition of claim 144 and a pharmaceutically-acceptable carrier.
